(19) United States
(12) Patent Application Publication
(10) Pub. No.: US 2020/0181210 A1
(43) Pub. Date: Jun. 11, 2020

(54) MODIFIED CYCLIC PEPTIDES AND THERAPEUTIC USE THEREOF

(71) Applicant: THE BROAD INSTITUTE, INC., Cambridge, MA (US)

(72) Inventors: Robert Nicol, Cambridge, MA (US); Ritika Dwivedi, Cambridge, MA (US)

(73) Assignee: THE BROAD INSTITUTE, INC., Cambridge, MA (US)

(21) Appl. No.: 16/608,536

(22) PCT Filed: Apr. 26, 2018
(86) PCT No.: PCT/US18/29573

§ 371 Date: Oct. 25, 2019

Related U.S. Application Data
(60) Provisional application No. 62/490,354, filed on Apr. 26, 2017.

(51) Int. Cl. C07K 14/415; C12Q 1/37

(52) U.S. Cl. CPC C07K 14/415; A61K 38/00; C12Q 1/37

(57) ABSTRACT

The present invention relates to cyclic peptide structures possessing the ability to incorporate and deliver peptides of sizable length (e.g., peptides of twenty amino acid residues or more in length), while retaining properties of cyclic peptides and/or cyclotides, including, e.g., protease resistance, heat resistance, resistance to low pH, etc. Methods for preparing cyclic peptide compositions, assessing the extent of cyclization within a peptide population, as well as methods for using cyclic peptide compositions are also provided.

### CROSS-REFERENCE TO RELATED APPLICATION

The present application is related to and claims priority under 35 U.S.C. § 119(e) to U.S. provisional patent application No. 62/490,354, entitled “Modified Cyclic Peptides and Therapeutic Use Thereof,” filed Apr. 26, 2017. The entire content of the aforementioned patent application is incorporated herein by this reference.

### BACKGROUND OF THE INVENTION

Cyclotides have been contemplated as delivery vehicles for small peptides. However, the utility of known cyclotides has been limited by their small insert capacity. In particular, extension of “loop 6” lengths of natural cyclotides beyond 20-22 amino acids (“loop 6” being the most common site of exogenous peptide introduction into natural cyclotides) has been observed to destabilize the cyclotide structure, rendering it less resistant to proteases, acids and/or heat stress (Colgrave and Craik, Biochemistry 43: 5965-75). There is therefore an unmet need for designing cyclotides that possess increased insert capacity yet retain the various advantages of the cyclotide structure.

### SUMMARY OF THE INVENTION

The present invention is based, at least in part, upon the identification of cyclic peptide compositions—specifically including cyclotide compositions—that can harbor exogenous peptide sequences of extended length while retaining certain advantageous properties of cyclic peptides (as compared to corresponding linearized peptides), as well as methods for design and use of such cyclic peptide compositions. Certain aspects of the disclosure relate to discovery of methods for improved drug delivery using cyclotides that possess modified loop sequences. Additional aspects of the invention relate to identification of a process for high-throughput assessment and quantitation of the extent of cyclization/cyclotide content in a sample that potentially also contains linear peptides (i.e., non-cyclized forms of polypeptide sequences corresponding to cyclotide forms of same).

In one aspect, the instant disclosure provides a method for stabilizing a cyclic peptide possessing two or more loop domain sequences, where a first loop domain sequence of the cyclic peptide is at least 25 amino acids in length, the method involving extending the length of a second loop domain sequence of the cyclic peptide by at least three amino acids, where the extending of the second loop domain sequence of the cyclic peptide improves the trypsin resistance of the cyclic peptide, thereby stabilizing the cyclic peptide possessing two or more loop domain sequences.

In certain embodiments, the length of a second loop domain sequence of the stabilized cyclic peptide is extended by at least four amino acids, at least five amino acids, at least six amino acids, at least 7 amino acids, at least 8 amino acids, at least 9 amino acids, or at least 10 amino acids.

In some embodiments, the extending of the second loop domain sequence of the cyclic peptide improves the trypsin resistance of the cyclic peptide, as compared to a control cyclic peptide possessing the first loop domain sequence of at least 25 amino acids in length yet not possessing the extended second loop domain sequence.

In one embodiment, the cyclic peptide is a cyclotide, optionally the cyclotide is a MCoTI-I cyclotide having an extended loop 6 as the first loop domain sequence of the cyclotide that is at least 25 amino acids in length.

In another embodiment, the at least three amino acid extension of the second loop domain sequence is a duplication of an at least three amino acid sequence found within the same second loop domain sequence.

Optionally, the at least three amino acid extension of the second loop domain sequence is an inverted duplication of an at least three amino acid sequence found within the same second loop domain sequence.

In certain embodiments, the cyclotide includes at least three loop domain sequences. Optionally, three or more of the at least three loop domain sequences are extended. In some embodiments, loop domain sequence extension for each loop domain sequence other than the first loop domain sequence is performed by duplication and/or inverted duplication of the original sequence of the same loop domain sequence.

In one embodiment, the second loop domain sequence of the cyclic peptide is extended by an amount of about the original length of the original second loop domain sequence of the cyclic peptide, about twice the original length of the original second loop domain sequence of the cyclic peptide, about three times the original length of the original second loop domain sequence of the cyclic peptide, about four times the original length of the original second loop domain sequence of the cyclic peptide or about five times the original length of the original second loop domain sequence of the cyclic peptide. Optionally, each loop domain sequence of the cyclic peptide other than the first loop domain sequence of the cyclic peptide is extended by an amount that is about the original length of the original loop domain sequence now extended of the cyclic peptide, about twice the original length of the original loop domain sequence now extended of the cyclic peptide, about three times the original length of the original loop domain sequence now extended of the cyclic peptide, about four times the original length of the original loop domain sequence now extended of the cyclic peptide or about five times the original length of the original loop domain sequence now extended of the cyclic peptide.

In certain embodiments, at least two loop domain sequences of the cyclic peptide other than the first loop domain sequence of the cyclic peptide are extended by duplication, inverted duplication, or both, of the original sequence of the same loop domain sequence now extended.

In some embodiments, two or more loop domain sequences of the cyclic peptide other than the first loop domain sequence of the cyclic peptide are extended, where each of the two or more loop domain sequences of the cyclic peptide other than the first loop domain sequence of the cyclic peptide is extended by an amount that is about the original length of the original loop domain sequence now extended of the cyclic peptide, about twice the original length of the original loop domain sequence now extended of the cyclic peptide, about three times the original length of the original loop domain sequence now extended of the cyclic peptide, about four times the original length of the original loop domain sequence now extended of the cyclic peptide or about five times the original length of the original loop domain sequence now extended of the cyclic peptide.

In one embodiment, two or more loop domain sequences of the cyclic peptide other than the first loop domain sequence of the cyclic peptide are extended, optionally where each of the two or more loop domain sequences of the cyclic peptide other than the first loop domain sequence of the cyclic peptide is extended by about the original length of the original loop domain sequence now extended of the cyclic peptide.

In some embodiments, two or more loop domain sequences of the cyclic peptide other than the first loop domain sequence of the cyclic peptide are extended, optionally where each of the two or more loop domain sequences of the cyclic peptide other than the first loop domain sequence of the cyclic peptide is extended by about twice the original length of the original loop domain sequence now extended of the cyclic peptide.

In one embodiment, two or more loop domain sequences of the cyclic peptide other than the first loop domain sequence of the cyclic peptide are extended, optionally where each of the two or more loop domain sequences of the cyclic peptide other than the first loop domain sequence of the cyclic peptide is extended by about three times the original length of the original loop domain sequence now extended of the cyclic peptide.

In certain embodiments, two or more loop domain sequences of the cyclic peptide other than the first loop domain sequence of the cyclic peptide are extended, where each of the two or more loop domain sequences of the cyclic peptide other than the first loop domain sequence of the cyclic peptide is extended by about four times the original length of the original loop domain sequence now extended of the cyclic peptide.

In some embodiments, two or more loop domain sequences of the cyclic peptide other than the first loop domain sequence of the cyclic peptide are extended, optionally where each of the two or more loop domain sequences of the cyclic peptide other than the first loop domain sequence of the cyclic peptide is extended by about five times the original length of the original loop domain sequence now extended of the cyclic peptide.

In one embodiment, the first loop domain sequence of the cyclic peptide includes a peptide sequence derived from a source exogenous to the base cyclic peptide sequence, e.g., a therapeutic peptide, optionally a polypeptide drug of 22-50 or more amino acids in length, an antibody molecule or fragment, optionally a monoclonal antibody, single domain antibodies such as camelid or cartilaginous fish antibody, scFv, antibody fragment such as Fv, Fab, Fab′ and F(ab′)₂ fragments, and other fragments, and/or a small molecule, optionally a small molecule attached to the cyclic peptide via a non-canonical amino acid and/or linker. Optionally, the therapeutic peptide is selected from Table 4.

In certain embodiments, the first loop domain sequence of the cyclic peptide includes a peptide tag, optionally where the peptide tag is an epitope tag (e.g., a FLAG-tag, a V5-tag, Myc-tag, HA-tag and/or NE-tag), a polyglutamate tag, a Strep-tag and/or a HIS tag.

In one embodiment, the cyclic peptide is selected from Table 1 or Table 3, and optionally has an extended loop 6 as the first loop domain sequence of the cyclic peptide that is at least 30 amino acids in length.

In certain embodiments, the stabilized cyclic peptide sequence is selected from Table 5.

In one embodiment, the first loop domain sequence of the cyclic peptide is at least 30 amino acids in length, optionally at least 35 amino acids in length, optionally at least 40 amino acids in length, optionally at least 45 amino acids in length, and optionally 50 or more amino acids in length.

In some embodiments, trypsin resistance of the stabilized cyclic peptide is assessed under conditions where the cyclic peptide is exposed to 10 μg trypsin protease digestion at 37° C. for between two and 24 hours.

Another aspect of the instant disclosure provides a method for treating or preventing a disease or disorder in a subject involving administering to the subject a stabilized cyclic peptide in an amount effective to treat or prevent a disease or disorder in a subject, where the stabilized cyclic peptide possesses two or more loop domain sequences, where a first loop domain sequence of the stabilized cyclic peptide is at least 30 amino acids in length and the length of a second loop domain sequence of the stabilized cyclic peptide has been extended by at least three amino acids to improve the trypsin resistance of the stabilized cyclic peptide, thereby treating or preventing a disease or disorder in the subject. A further aspect of the instant disclosure provides a method for designing a multi-loop-expanded cyclotide possessing at least one loop domain sequence in excess of 25 amino acids in length that involves identifying a base cyclotide sequence having at least two loop domain sequences, where each loop domain sequence is of 25 amino acid residues or less in length; extending a first of the at least two loop domain sequences from an initial length (L₁ᵢ) to and extended length (L₁ₑ), where length L₁ₑ exceeds 25 amino acids, thereby forming a first extended loop domain sequence; and extending the length of a second loop domain sequence of the base cyclotide sequence possessing an original second loop domain sequence length of L₂ᵢ by between about 0.1·[(L₁ₑ−L₁ᵢ)/L₁ᵢ]·L₂ᵢ and about 100·[(L₁ₑ−L₁ᵢ)/L₁ᵢ]·L₂ᵢ amino acid residues, where the length of the extension is at least one amino acid, thereby designing a multi-loop-expanded cyclotide.

In one embodiment, the length of the second loop domain sequence of the base cyclotide sequence possessing an original second loop domain sequence length of L₂ᵢ is extended by between 0.5·[(L₁ₑ−L₁ᵢ)/L₁ᵢ]·L₂ᵢ and 1.5·[(L₁ₑ−L₁ᵢ)/L₁ᵢ]·L₂ᵢ amino acid residues.

In another embodiment, the base cyclotide is a MCoTI-I cyclotide.

Optionally, extending the length of the second loop domain sequence is performed by duplication, inverted duplication, or both, of the original second loop domain sequence.

In certain embodiments, the base cyclotide includes at least three loop domain sequences.

In one embodiment, three or more of the at least three loop domain sequences are extended.

Optionally, loop domain sequence extension for each loop domain sequence other than the first loop domain sequence is performed by duplication and/or inverted duplication of the original sequence of the same loop domain sequence.

In certain embodiments, the first loop domain sequence of the base cyclotide is the longest loop domain sequence of the base cyclotide.

In another embodiment, the first loop domain sequence of the base cyclotide is loop 6 of the base cyclotide.

In an additional embodiment, the second loop domain sequence of the cyclotide is extended by an amount that is about the original length of the original second loop domain sequence of the cyclotide, about twice the original length of the original second loop domain sequence of the cyclotide, about three times the original length of the original second loop domain sequence of the cyclotide, about four times the original length of the original second loop domain sequence of the cyclotide or about five times the original length of the original second loop domain sequence of the cyclotide.

In a related embodiment, each loop domain sequence of the cyclotide other than the first loop domain sequence of the cyclotide is extended by an amount that is about the original length of the original loop domain sequence now extended of the cyclotide, about twice the original length of the original loop domain sequence now extended of the cyclotide, about three times the original length of the original loop domain sequence now extended of the cyclotide, about four times the original length of the original loop domain sequence now extended of the cyclotide or about five times the original length of the original loop domain sequence now extended of the cyclotide.

In some embodiments, the loop domain sequences that are extended, other than the first loop domain sequence, are extended in length by about the same proportion relative to the corresponding base sequences of the loop domain sequences other than the first loop domain sequence now extended.

In one embodiment, the first loop domain sequence of the cyclotide includes a peptide exogenous to the base cyclotide sequence, e.g., a therapeutic peptide, optionally a polypeptide drug of 22-50 or more amino acids in length, an antibody molecule or fragment, optionally a monoclonal antibody, single domain antibodies such as camelid or cartilaginous fish antibody, scFv, antibody fragment such as Fv, Fab, Fab′ and F(ab′)₂ fragments, and other fragments, and/or a small molecule, optionally a small molecule attached to the cyclic peptide via a non-canonical amino acid and/or linker. Optionally, the therapeutic peptide is selected from Table 4 or FIG. 26 or 27.

In certain embodiments, the base cyclotide is selected from Table 1, Table 2 or Table 3.

In another embodiment, the multi-loop-expanded cyclotide sequence is selected from Table 5.

Another aspect of the instant disclosure provides a method for treating or preventing a disease or disorder in a subject involving administering to the subject a multi-loop-expanded cyclotide sequence prepared by a method of the instant disclosure in an amount effective to treat or prevent a disease or disorder in a subject, thereby treating or preventing a disease or disorder in the subject.

In one embodiment, the disease or disorder is a GPCR-related disease or disorder, a hormone-related disease or disorder, and/or a microbial infection and/or microbial infection-related disease or disorder.

Another aspect of the instant disclosure provides a composition possessing a cyclotide sequence of Table 5.

An additional aspect of the instant disclosure provides a pharmaceutical composition that includes a cyclotide sequence of Table 5 and a pharmaceutically acceptable carrier.

A further aspect of the instant disclosure provides a method for designing a cyclotide composition possessing at least 10 loop domain sequences and two linker sequences, involving identifying a first base cyclotide sequence and a second base cyclotide sequence, where each base cyclotide sequence possesses at least six loop domain sequences; severing the longest loops of each of the first base cyclotide sequence and the second base cyclotide sequence and removing between 0 and 7 amino acid residues from each end of the severed loop sequences, thereby creating (a) an N-terminal free end of the first base cyclotide sequence and a C-terminal free end of the first base cyclotide sequence and (b) an N-terminal free end of the second base cyclotide sequence and a C-terminal free end of the second base cyclotide sequence; joining the C-terminal free end of the first base cyclotide sequence to the N-terminus of a first linker sequence and joining the C-terminus of the first linker sequence to the N-terminal free end of the second base cyclotide sequence; and joining the C-terminal free end of the second base cyclotide sequence to the N-terminus of a second linker sequence and joining the C-terminus of the second linker sequence to the N-terminal free end of the first base cyclotide sequence, thereby designing a cyclotide composition possessing at least 10 loop domain sequences and two linker sequences.

In one embodiment, the first linker sequence, the second linker sequence, or both linker sequences include a peptide derived from a source exogenous to the base cyclic peptide sequence, optionally a therapeutic peptide, optionally a polypeptide drug of 22-50 or more amino acids in length, an antibody molecule or fragment, optionally a monoclonal antibody, single domain antibodies such as camelid or cartilaginous fish antibody, scFv, antibody fragment such as Fv, Fab, Fab′ and F(ab′)₂ fragments, and other fragments, and/or a small molecule, optionally a small molecule attached to the cyclic peptide via a non-canonical amino acid and/or linker, and/or an epitope tag (e.g., a FLAG-tag, a V5-tag, Myc-tag, HA-tag and/or NE-tag), a polyglutamate tag, a Strep-tag and/or a HIS tag. Optionally, the therapeutic peptide is selected from Table 4.

In another embodiment, the first linker sequence, the second linker sequence, or both linker sequences are at least 25 amino acid residues in length.

In certain embodiments, the second base cyclotide sequence is the reverse sequence of the first base cyclotide sequence.

In some embodiments, each loop domain sequence of the second base cyclotide sequence is the reverse sequence of the corresponding loop domain sequence of the first base cyclotide sequence.

Another aspect of the instant disclosure provides a method for treating or preventing a disease or disorder in a subject involving administering to the subject a cyclotide composition possessing at least 10 loop domain sequences and two linker sequences in an amount effective to treat or prevent a disease or disorder in a subject, thereby treating or preventing a disease or disorder in the subject.

In certain embodiments, the disease or disorder is a GPCR-related disease or disorder, a hormone-related disease or disorder, and/or a microbial infection or microbial infection-related disease or disorder.

In another embodiment, a cyclotide composition possessing at least 10 loop domain sequences and two linker sequences designed by a method of the disclosure is provided.

A further aspect of the instant disclosure provides a composition that includes a sequence of Table 5.

An additional aspect of the instant disclosure provides a pharmaceutical composition that includes a sequence of Table 5 and a pharmaceutically acceptable carrier.

In another aspect, the disclosure provides a method for designing a cyclotide composition possessing at least 15 loop domain sequences and three linker sequences, involving identifying a first base cyclotide sequence, a second base cyclotide sequence and a third base cyclotide sequence, where each base cyclotide sequence includes at least six loop domain sequences; severing the longest loops of each of the first base cyclotide sequence, the second base cyclotide sequence, and the third base cyclotide sequence and removing between 0 and 7 amino acid residues from each end of the severed loop sequences, thereby creating (a) an N-terminal free end of the first base cyclotide sequence and a C-terminal free end of the first base cyclotide sequence, (b) an N-terminal free end of the second base cyclotide sequence and a C-terminal free end of the second base cyclotide sequence and (c) an N-terminal free end of the third base cyclotide sequence and a C-terminal free end of the third base cyclotide sequence; joining the C-terminal free end of the first base cyclotide sequence to the N-terminus of a first linker sequence and joining the C-terminus of the first linker sequence to the N-terminal free end of the second base cyclotide sequence; joining the C-terminal free end of the second base cyclotide sequence to the N-terminus of a second linker sequence and joining the C-terminus of the second linker sequence to the N-terminal free end of the third base cyclotide sequence; and joining the C-terminal free end of the third base cyclotide sequence to the N-terminus of a third linker sequence and joining the C-terminus of the third linker sequence to the N-terminal free end of the first base cyclotide sequence, thereby designing a cyclotide composition possessing at least 15 loop domain sequences and three linker sequences.

In certain embodiments, the first linker sequence, the second linker sequence, the third linker sequence, or any combination thereof includes a peptide derived from a source exogenous to the base cyclic peptide sequence, optionally a therapeutic peptide, optionally a polypeptide drug of 22-50 or more amino acids in length, an antibody molecule or fragment, optionally a monoclonal antibody, single domain antibodies such as camelid or cartilaginous fish antibody, scFv, antibody fragment such as Fv, Fab, Fab′ and F(ab′)₂ fragments, and other fragments, and/or a small molecule, optionally a small molecule attached to the cyclic peptide via a non-canonical amino acid and/or linker, and/or an epitope tag (e.g., a FLAG-tag, a V5-tag, Myc-tag, HA-tag and/or NE-tag), a polyglutamate tag, a Strep-tag and/or a HIS tag.

In one embodiment, the therapeutic peptide is selected from Table 4.

Optionally, the first linker sequence, the second linker sequence, the third linker sequence, or any combination thereof are each at least 25 amino acid residues in length.

In certain embodiments, the second and/or third base cyclotide sequence is the reverse sequence of the first base cyclotide sequence and/or the first and/or third base cyclotide sequence is the reverse sequence of the second base cyclotide sequence.

A further aspect of the instant disclosure provides a method for treating or preventing a disease or disorder in a subject involving administering to the subject a cyclotide composition possessing at least 15 loop domain sequences and three linker sequences in an amount effective to treat or prevent a disease or disorder in a subject, thereby treating or preventing a disease or disorder in the subject.

In one embodiment, the disease or disorder is a GPCR-related disease or disorder, a hormone-related disease or disorder, and/or a microbial infection or microbial infection-related disease or disorder.

In an additional embodiment, a cyclotide composition possessing at least 15 loop domain sequences and three linker sequences designed by a method of the disclosure is provided.

Another aspect of the disclosure provides a method for designing a cyclotide composition possessing at least 20 loop domain sequences and four linker sequences, involving identifying a first base cyclotide sequence, a second base cyclotide sequence, a third base cyclotide sequence and a fourth base cyclotide sequence, where each base cyclotide sequence includes at least six loop domain sequences; severing the longest loops of each of the first base cyclotide sequence, the second base cyclotide sequence, the third base cyclotide sequence and the fourth base cyclotide sequence and removing between 0 and 7 amino acid residues from each end of the severed loop sequences, thereby creating (a) an N-terminal free end of the first base cyclotide sequence and a C-terminal free end of the first base cyclotide sequence, (b) an N-terminal free end of the second base cyclotide sequence and a C-terminal free end of the second base cyclotide sequence, (c) an N-terminal free end of the third base cyclotide sequence and a C-terminal free end of the third base cyclotide sequence and (d) an N-terminal free end of the third base cyclotide sequence and a C-terminal free end of the third base cyclotide sequence; joining the C-terminal free end of the first base cyclotide sequence to the N-terminus of a first linker sequence and joining the C-terminus of the first linker sequence to the N-terminal free end of the second base cyclotide sequence; joining the C-terminal free end of the second base cyclotide sequence to the N-terminus of a second linker sequence and joining the C-terminus of the second linker sequence to the N-terminal free end of the third base cyclotide sequence; joining the C-terminal free end of the third base cyclotide sequence to the N-terminus of a third linker sequence and joining the C-terminus of the third linker sequence to the N-terminal free end of the fourth base cyclotide sequence; and joining the C-terminal free end of the fourth base cyclotide sequence to the N-terminus of a fourth linker sequence and joining the C-terminus of the fourth linker sequence to the N-terminal free end of the first base cyclotide sequence, thereby designing a cyclotide composition possessing at least 20 loop domain sequences and four linker sequences.

In one embodiment, the first linker sequence, the second linker sequence, the third linker sequence, the fourth linker sequence, or any combination thereof includes a peptide derived from a source exogenous to the base cyclic peptide sequence, optionally a therapeutic peptide, optionally a polypeptide drug of 22-50 or more amino acids in length, an antibody molecule or fragment, optionally a monoclonal antibody, single domain antibodies such as camelid or cartilaginous fish antibody, scFv, antibody fragment such as Fv, Fab, Fab′ and F(ab′)₂ fragments, and other fragments, and/or a small molecule, optionally a small molecule attached to the cyclic peptide via a non-canonical amino acid and/or linker, and/or an epitope tag (e.g., a FLAG-tag, a V5-tag, Myc-tag, HA-tag and/or NE-tag), a polyglutamate tag, a Strep-tag and/or a HIS tag.

Optionally, the first linker sequence, the second linker sequence, the third linker sequence, the fourth linker sequence, or any combination thereof are each at least 25 amino acid residues in length.

In certain embodiments, the first, second and/or third base cyclotide sequence is the reverse sequence of the fourth base cyclotide sequence; the second, third and/or fourth base cyclotide sequence is the reverse sequence of the first base cyclotide sequence; the first, third and/or fourth base cyclotide sequence is the reverse sequence of the second base cyclotide sequence; and/or the first, second and/or fourth base cyclotide sequence is the reverse sequence of the third base cyclotide sequence.

An additional aspect of the disclosure provides a method for treating or preventing a disease or disorder in a subject involving administering to the subject a cyclotide composition possessing at least 20 loop domain sequences and four linker sequences in an amount effective to treat or prevent a disease or disorder in a subject, thereby treating or preventing a disease or disorder in the subject.

In one embodiment, the instant disclosure provides a cyclotide composition possessing at least 20 loop domain sequences and four linker sequences designed by a method of the disclosure.

A further aspect of the instant disclosure provides a method for identifying the presence of a protease-stabilized peptide composition in a solution involving preparing a tagged peptide capable of forming a protease-stabilized structure; subjecting the tagged peptide to one or more proteases under conditions that allow for protease activity; purifying tagged peptides, thereby generating a purified tagged peptide sample; labeling purified tagged peptide sample with fluorescent moieties that bind to one or more amino acids; and quantifying the level of fluorescence in the purified tagged peptide sample, as compared to an appropriate control, where the presence of an increased level of fluorescence in the purified tagged peptide sample identifies the presence and/or increased level of a protease-stabilized peptide composition in the purified tagged peptide sample, thereby identifying the presence of a protease-stabilized peptide composition in a solution.

In one embodiment, the protease-stabilized peptide composition is a cyclotide.

Optionally, the protease-stabilized peptide composition is trypsin-stabilized.

In certain embodiments, the one or more proteases include trypsin.

In some embodiments, the method is performed in 96-well or 384-well plate format.

In another embodiment, the tagged peptide is tagged with an epitope tag (e.g., a FLAG-tag, a V5-tag, Myc-tag, HA-tag and/or NE-tag), a polyglutamate tag, a Strep-tag and/or a HIS tag.

Another aspect of the disclosure provides a method for making a loop-expanded cyclic peptide possessing two or more loop domain sequences and at least one Cys-Cys linkage, the method involving extending the length of a first loop domain sequence and a second loop domain sequence of a base cyclic peptide sequence in proportion to one another, thereby forming a loop-expanded cyclic peptide, where the relative position of the Cys-Cys linkage is maintained within the loop-expanded cyclic peptide, as compared to the base cyclic peptide sequence, thereby making a loop-expanded cyclic peptide possessing two or more loop domain sequences.

In one embodiment, the loop-expanded cyclic peptide possesses four or more loop domain sequences and at least two Cys-Cys linkages, where the relative positions of the Cys-Cys linkages are maintained within the loop-expanded cyclic peptide as compared to the base cyclic peptide sequence, optionally where all loops of the base cyclic peptide sequence are extended in proportion to one another to form the loop-expanded cyclic peptide.

In another embodiment, the loop-expanded cyclic peptide possesses six or more loop domain sequences and at least three Cys-Cys linkages, where the relative positions of the Cys-Cys linkages are maintained within the loop-expanded cyclic peptide as compared to the base cyclic peptide sequence, optionally where all loops of the base cyclic peptide sequence are extended in proportion to one another to form the loop-expanded cyclic peptide.

In an additional embodiment, the loop-expanded cyclic peptide is trypsin resistant.

Another aspect of the invention provides a loop-expanded cyclic peptide possessing two or more loop domain sequences and at least one Cys-Cys linkage, the loop-expanded cyclic peptide formed by extending the length of a first loop domain sequence and a second loop domain sequence of a base cyclic peptide sequence in proportion to one another, thereby forming the loop-expanded cyclic peptide, where the relative position of the Cys-Cys linkage is maintained within the loop-expanded cyclic peptide, as compared to the base cyclic peptide sequence.

A further aspect of the invention provides a loop-expanded cyclic peptide possessing four or more loop domain sequences and at least two Cys-Cys linkages, the loop-expanded cyclic peptide formed by extending the length of the four or more loop domain sequences of a base cyclic peptide sequence in proportion to one another, thereby forming the loop-expanded cyclic peptide, where the relative positions of the Cys-Cys linkages are maintained within the loop-expanded cyclic peptide, as compared to the base cyclic peptide sequence.

An additional aspect of the invention provides a loop-expanded cyclic peptide possessing six or more loop domain sequences and at least three Cys-Cys linkages, the loop-expanded cyclic peptide formed by extending the length of the six or more loop domain sequences of a base cyclic peptide sequence in proportion to one another, thereby forming the loop-expanded cyclic peptide, where the relative positions of the Cys-Cys linkages are maintained within the loop-expanded cyclic peptide, as compared to the base cyclic peptide sequence.

In certain embodiments, the loop-expanded cyclic peptide is trypsin resistant.

A further aspect of the disclosure provides a cyclic peptide that includes: (i) a cyclotide amino acid sequence of Table 2 or a corresponding modified cyclic peptide amino acid sequence that is at least about 95% identical to a cyclotide amino acid sequence of Table 2; (ii) a first insert sequence having an amino acid sequence inserted into the cyclotide or cyclic peptide amino acid sequence of (i) between two amino acid residues of the (corresponding) loop 6 amino acid sequence of the cyclotide shown in FIG. 25, where, following insertion of the first insert sequence into the loop 6 amino acid sequence shown in FIG. 25, the loop 6 amino acid sequence containing the first insert sequence is at least 23 amino acid residues in length; and (iii) a stabilizing insertion of a second insert sequence of at least three amino acids that is inserted between amino acid residues of any one of the (corresponding) loops 1-5 of the cyclotide amino acid sequence of Table 2, wherein the (engineered) cyclic peptide sequence that is produced, excluding the first insert sequence, is at least 80% identical to the cyclotide amino acid sequence of Table 2 or to the corresponding modified cyclic peptide amino acid sequence that is at least about 95% identical to the cyclotide amino acid sequence of Table 2.

In one embodiment, the cyclic peptide further includes a third insert sequence of at least three amino acids that is inserted at any one of the (corresponding) loops 1-5 of said cyclotide amino acid sequence of Table 2, exclusive of the loop of (iii) that contains the second insert sequence.

In an additional embodiment, the cyclotide amino acid sequence of (i) is a cyclotide amino acid sequence of Table 2.

In certain embodiments, the first insert sequence comprises a sequence selected from FIGS. 26 and 27. Optionally, the first insert sequence is a sequence selected from FIGS. 26 and 27. Optionally, the first insert sequence is glucagon, glucagon-like peptide 1 (GLP-1), amylin, adrenomedullin or pramlintide.

An additional embodiment of the disclosure provides a cyclic peptide as describe above, where, within the cyclic peptide, the sequence of the loop including the second insert sequence is GPGKKIILLQQRR (SEQ ID NO: 363), GRRRRDDSSDD (SEQ ID NO: 364), GPGGGAA (SEQ ID NO: 365), GII (SEQ ID NO: 366) and/or GRRGGNNGGYY (SEQ ID NO: 367).

An additional aspect of the disclosure provides the cyclic peptide of SEQ ID NO: 36 or SEQ ID NO: 362.

### Definitions

By “agent” is meant any small compound, antibody, nucleic acid molecule, or polypeptide, or fragments thereof.

By “alteration” is meant a change (increase or decrease) in the expression levels or activity of a gene or polypeptide as detected by standard art known methods such as those described herein. As used herein, an alteration includes a 10% change in expression levels or activity, preferably a 25% change, more preferably a 40% change, and most preferably a 50% or greater change in expression levels or activity.

By “ameliorate” is meant to decrease, suppress, attenuate, diminish, arrest, or stabilize the development or progression of a disease.

In general, the meaning of the term “amino acid” or “amino acid residue” is known in the art and is used herein accordingly. Thereby, it is of note that when an “amino acid” is a component of a peptide/protein the term “amino acid” is used herein in the same sense as “amino acid residue”. Particularly, an “amino acid” or “amino acid residue” as referred to herein is envisaged to be a naturally-occurring amino acid, optionally a naturally-occurring L-amino acid. However, albeit generally less prevalent, an “amino acid” or “amino acid residue” in context of this invention may also be a D-amino acid or a non-naturally-occurring (i.e. a synthetic) amino acid, like, for example, norleucine, β-alanine, or selenocysteine.

The term “acidic amino acid(s)” as used herein is intended to mean an amino acid selected from the group comprising Asp, Asn, Glu, and Gln; the term “basic amino acid(s)” as used herein is intended to mean an amino acid selected from the group comprising Arg, Lys and His; the term “aliphatic amino acid(s)” as used herein is intended to mean any amino acid selected from the group comprising Gly, Ala, Ser, Thr, Val, Leu, Ile, Asp, Asn, Glu, Gln, Arg, Lys, Cys and Met, and the term “polar amino acid(s)” as used herein is intended to mean any amino acid selected from the group comprising Cys, Met, Ser, Tyr, Gln, Asn and Trp.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

A “cyclotide”, as used herein, is a head-to-tail cyclized peptide that includes at least one Cys-Cys disulfide linkage that thereby establishes distinct “loop” domains on either side of the Cysteine involved in the Cys-Cys disulfide linkage. In certain aspects, a naturally-occurring cyclotide chain (or a cyclotide chain sequence derived therefrom) includes six conserved cysteine residues capable of forming three disulfide bonds arranged in a cyclic cysteine-knot (CCK) motif, which thereby forms six distinct “loop” domains from the inter-cysteine sequences of the cyclotide. The inter-cysteine sequences of a cyclotide can tolerate a wide range of residue substitutions. In certain aspects, the term “cyclotide” used herein refers to cyclotides as described in WO 2013/093045 (Gruber and Gruendemann), WO 2013/162760 (Camarero et al.), WO 2011/005598 (Camarero), Craik (1999, J Mol Biol, 294: 1327-1336) and/or Clark (2006, Biochem J, 394: 85-93).

A “cyclotide loop domain sequence”, as used herein, refers to a sequence present in the region between given Cys-Cys intramolecular linkages of a cyclotide. In certain embodiments, i.e., within cyclotides comprising six cyclotide loop domain sequences (established by such cyclotides comprising six cysteine residues that are involved in intra-molecular disulfide bonding), each loop sequence comprises between 1 and 60 amino acid residues. In certain embodiments, exemplary lengths of the cyclotide loop domain sequences of naturally-occurring cyclotides are: a loop 1 cyclotide loop domain sequence of three to six amino acid residues in length; a loop 2 cyclotide loop domain sequence of four to eight amino acid residues in length; a loop 3 cyclotide loop domain sequence of three to ten amino acid residues in length; a loop 4 cyclotide loop domain sequence of one amino acid residue in length; a loop 5 cyclotide loop domain sequence of four to eight amino acid residues in length; and a loop 6 cyclotide loop domain sequence of five to thirteen amino acid residues in length.

It will be understood that for the various cyclotides to be used in the context of the present invention a certain flexibility and variability in the primary sequence, i.e., the amino acid sequence backbone, is possible, as long as the overall secondary and tertiary structure of the respective peptides, which is defined by at least some fixed amino acid residues and by their spatial arrangement, is ensured.

If not otherwise specified, the term “cyclotide(s)” when used herein is envisaged also to encompass “cyclotide mutant(s)/variant(s)”. Non-limiting examples of mutant/variant/modified cyclotides according to this invention are provided in the Tables herein and also include cyclotides consisting of a head-to-tail cyclized form of an amino acid sequence as defined elsewhere herein.

As to the mutants/variants of the cyclotides, it is, for example, envisaged that one or more amino acids of said peptides are replaced by another one or more naturally-occurring or synthetic amino acids. In this context, it is often desirable that this/these amino acid exchange(s) is/are conservative amino acid exchange(s), i.e., that the replacement amino acid belongs to the same category of amino acids as the amino acid to be replaced. For example, an acidic amino acid may be replaced by another acidic amino acid, a basic amino acid may be replaced by another basic amino acid, an aliphatic amino acid may be replaced by another aliphatic amino acid, and/or a polar amino acid may be replaced by another polar amino acid.

It is particularly envisaged that the amino acid exchanges which lead to mutants/variants of the disclosed cyclotides are such that the pattern of polarity and charge within the tertiary structure of the resulting mutant/variant still (substantially) mimics/corresponds to the three-dimensional structure of the respective cyclotide (optionally, e.g., the structure of a multi-loop-expanded cyclotide, as described elsewhere herein).

With respect to mutants/variants of the cyclotides disclosed herein, it is also contemplated that one or more of the (e.g., up to six) Cys residues, in particular the herein defined Cys residues involved in intramolecular linkages, may also be replaced by (an)other amino acid(s), as long as the replacement still leads to an individual intramolecular linkage, like that of a disulfide bond, within the cyclopeptide, i.e., to a correct mimicry of the native cyclotide structure. Such amino acid may, inter alia, be a non-naturally-occurring amino acid, like a non-naturally-occurring amino acid having an —SH group able to form a disulfide bond, though in certain aspects of the invention the Cys residues of the intramolecular linkages of a cyclotide of the invention are naturally-occurring amino acids, in most embodiments Cys itself.

It will also be acknowledged by those of ordinary skill in the art that one or several of the amino acids forming the cyclotide and/or cyclotide-derived compositions described herein to be employed according to the present invention may be modified. In accordance therewith, any amino acid as used/defined herein may also represent its modified form. For example, an alanine residue as used herein may comprise a modified alanine residue. Such modifications may, among others, be a methylation or acylation, or the like, whereby such modification or modified amino acid is preferred as long as the thus modified amino acid and more particularly the cyclotide containing said thus modified amino acid is still functionally active as defined herein. Respective assays for determining whether such a cyclotide, i.e., a cyclotide comprising one or several modified amino acids, fulfills this requirement, are known to one of ordinary skill in the art, and may also be described herein, particularly in the Examples.

The invention also provides the use of derivatives of the disclosed compositions, such as salts with physiologic organic and inorganic acids like HCl, H₂SO₄, H₃PO, malic acid, fumaric acid, citronic acid, tartaric acid, and acetic acid.

“Detect” refers to identifying the presence, absence, or amount of the polypeptide, nucleic acid (e.g., DNA, RNA, rRNA, etc.) and/or other composition/substance/moiety to be detected.

The term “domain” refers to a portion of a protein that is physically or functionally distinguished from other portions of the protein or peptide. In general, a domain can be a single, stable three-dimensional structure, regardless of size. The tertiary structure of a typical domain is stable in solution and remains the same whether such a member is isolated or covalently fused to other domains. A domain generally has a particular tertiary structure formed by the spatial relationships of secondary structure elements, such as beta-sheets, alpha helices, and unstructured loops. In domains of the cyclotide family, disulfide bridges determine the boundaries of loop domains.

By “effective amount” is meant the amount of an agent required to ameliorate the symptoms of a disease relative to those in an untreated patient. The effective amount of active agent(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

The term “epitope” includes any polypeptide determinant capable of specific binding to a binding partner, e.g., an antibody or antigen-binding portion thereof. In certain embodiments, epitope determinants include chemically active surface groupings of molecules such as amino acids, sugar side chains, phosphoryl, or sulfonyl, and, in certain embodiments, may have specific three dimensional structural characteristics, and/or specific charge characteristics. In various embodiments, an epitope may be a linear or sequential epitope, i.e., a linear sequence of amino acids, of the primary structure of the antigen. Alternatively, in other embodiments, an epitope may be a conformational epitope having a specific three-dimensional shape when the polypeptide encompassing the epitope assumes its secondary structure. For example, the conformational epitope may comprise non-linear, i.e., non-sequential, amino acids of the antigen. In a particular embodiment, an epitope is a region of an antigen that is bound by an antibody or antigen-binding portion thereof. In certain embodiments, an antibody or antigen-binding portion thereof is said to specifically bind an antigen when it preferentially recognizes its target antigen in a complex mixture of proteins and/or macromolecules.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 5, 7, 10, 20, 30, 40, 50, 60, 70, 80, 90, 100, 200, 300, 400, 500, 600, 700, 800, 900, or 1000 or more nucleotides or amino acids.

By “gene” is meant a locus (or region) of DNA that encodes a functional RNA or protein product, and is the molecular unit of heredity.

As used herein, the term “isolated” means separated from constituents, cellular and otherwise, in which the polynucleotide, peptide, polypeptide, protein, antibody, or fragments thereof, are normally associated with in nature. As is apparent to those of ordinary skill in the art, a non-naturally occurring polynucleotide, peptide, polypeptide, protein, antibody, or fragments thereof, does not require “isolation” to distinguish it from its naturally occurring counterpart. In addition, a “concentrated”, “separated” or “diluted” polynucleotide, peptide, polypeptide, protein, antibody, or fragments thereof, is distinguishable from its naturally occurring counterpart in that the concentration or number of molecules per volume is greater than (“concentrated”) or less than (“separated”) that of its naturally occurring counterpart.

By “marker” is meant any protein or polynucleotide having an alteration in expression level or activity that is associated with a disease or disorder.

By “modulate” is meant to alter (increase or decrease). Such alterations are detected by standard art known methods such as those described herein.

“Non-naturally occurring” as applied to a protein means that the protein contains at least one amino acid that is different from the corresponding wildtype or native protein. Non-natural sequences can be determined by performing BLAST search using, e.g., the lowest smallest sum probability where the comparison window is the length of the sequence of interest (the queried) and when compared to the non-redundant (“nr”) database of Genbank using BLAST 2.0. The BLAST 2.0 algorithm, which is described in Altschul et al. (1990) J. Mol. Biol. 215:403-410, respectively. Software for performing BLAST analyses is publicly available through the National Center for Biotechnology Information.

By “homologous amino acid sequence” is meant an amino acid sequence that is shared by one or more peptide sequences, such as proteins. For example, a homologous sequence can be an amino acid sequence that is shared by two or more proteins that are related but different proteins, such as different members of a protein family, different protein epitopes, different protein isoforms or completely evolutionarily divergent proteins, such as a cytokine and its corresponding receptors. Homologous sequences can also include conserved sequence regions shared by more than one polypeptide sequence. Homology does not need to be perfect homology (e.g., 100%), as partially homologous sequences are also contemplated by the instant invention (e.g., 99%, 98%, 97%, 96%, 95%, 94%, 93%, 97%, 91%, 90%, 89%, 88%, 87%, 86%, 85%, 84%, 83%, 82%, 81%, 80% etc.). Indeed, design and use of the cyclic peptides of the instant disclosure contemplates the possibility of using cyclic peptides (e.g., base cyclotides) possessing sequences that are, e.g., only 99%, 98%, 97%, 96%, 95%, 94%, 93%, 92%, 91%, 90%, 89%, 88%, 87%, 86% 85%, 84%, 83%, 82%, 81%, 80% etc. homologous of (identical to) those cyclic peptide sequences specifically recited herein.

Sequence identity may be determined by sequence comparison and alignment algorithms known in the art, such as the above-referenced BLAST algorithm. To determine the percent identity of two amino acid sequences (or two nucleic acid sequences), the sequences are aligned for comparison purposes (e.g., gaps can be introduced in the first sequence or second sequence for optimal alignment). The amino acid residues (or nucleic acid residues) at corresponding amino acid positions (or nucleic acid positions) are then compared. When a position in the first sequence is occupied by the same residue as the corresponding position in the second sequence, then the molecules are identical at that position. The percent identity between the two sequences is a function of the number of identical positions shared by the sequences (i.e., % homology=of identical positions/total # of positions)×100), optionally penalizing the score for the number of gaps introduced and/or length of gaps introduced.

By “nucleic acid” is meant biopolymers, or large biomolecules, essential for all known forms of life. Nucleic acids, which include DNA (deoxyribonucleic acid) and RNA (ribonucleic acid), are made from monomers known as nucleotides. Each nucleotide has three components: a 5-carbon sugar, a phosphate group, and a nitrogenous base. If the sugar is deoxyribose, the polymer is DNA. If the sugar is ribose, the polymer is RNA. Together with proteins, nucleic acids are the most important biological macromolecules; each are found in abundance in all living things, where they function in encoding, transmitting and expressing genetic information—in other words, information is conveyed through the nucleic acid sequence, or the order of nucleotides within a DNA or RNA molecule. Strings of nucleotides strung together in a specific sequence are the mechanism for storing and transmitting hereditary, or genetic information via protein synthesis. Nucleic acids include but are not limited to: deoxyribonucleic acid (DNA), ribonucleic acid (RNA), double-stranded DNA (dsDNA), single-stranded DNA (ssDNA), messenger RNA (mRNA), ribosomal RNA (rRNA), transfer RNA (tRNA), micro RNA (miRNA), and small interfering RNA (siRNA).

By “nucleic acid sequence” is meant a succession of letters that indicate the order of nucleotides within a DNA (using GACT) or RNA (GACU) molecule. By convention, sequences are usually presented from the 5′ end to the 3′ end. For DNA, the sense strand is used. Because nucleic acids are normally linear (unbranched) polymers, specifying the sequence is equivalent to defining the covalent structure of the entire molecule. For this reason, the nucleic acid sequence is also termed the primary structure. The sequence has capacity to represent information. Biological DNA represents the information which directs the functions of a living thing. In that context, the term genetic sequence is often used. Sequences can be read from the biological raw material through DNA sequencing methods. Nucleic acids also have a secondary structure and tertiary structure. Primary structure is sometimes referred to as primary sequence.

The terms “polypeptide”, “peptide”, “amino acid sequence” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified, for example, disulfide bond formation, glycosylation, lipidation, acetylation, phosphorylation, or any other manipulation, such as conjugation with a labeling component. As used herein, the term “amino acid” refers to natural and/or unnatural or synthetic amino acids, including but not limited to glycine and both the D or L optical isomers, and amino acid analogs and peptidomimetics. Standard single or three letter codes are used to designate amino acids.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

By “reduces” is meant a negative alteration of at least 10%, 25%, 50%, 75%, or 100%.

By “reference” is meant a standard or control condition.

A “reference sequence” is a defined sequence used as a basis for sequence comparison or a gene expression comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 6 amino acids, optionally at least about 10 amino acids, optionally at least about 16 amino acids, optionally at least about 20 amino acids, optionally at least about 25 amino acids, optionally about 35 amino acids, optionally about 50 amino acids, or optionally about 100 amino acids. For nucleic acids, the length of the reference nucleic acid sequence will optionally be at least about 18 nucleotides, optionally at least about 30 nucleotides, optionally at least about 40 nucleotides, optionally at least about 60 nucleotides, optionally at least about 75 nucleotides, optionally at least about 100 nucleotides, optionally at least about 300 or optionally at least about 500 or more nucleotides, or any integer thereabout or there between.

As used herein, “obtaining” as in “obtaining an agent” includes synthesizing, purchasing, or otherwise acquiring the agent.

By “subject” is meant a mammal, including, but not limited to, a human or non-human mammal, such as a bovine, equine, canine, ovine, or feline.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

As used herein, the terms “treat,” “treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

A “therapeutically effective amount” is an amount sufficient to effect beneficial or desired results, including clinical results. An effective amount can be administered in one or more administrations.

Any compositions or methods provided herein can be combined with one or more of any of the other compositions and methods provided herein.

Other features and advantages of the invention will be apparent to those skilled in the art from the following detailed description and claims.

### BRIEF DESCRIPTION OF THE DRAWINGS

FIGS. 1A and 1B depict the three-dimensional structures of two representative cyclic peptides. FIG. 1A shows the structures of the cyclic peptides kalata B1 and MCoTI-II. FIG. 1B depicts a close-up schematic of the MCoTI cyclic peptide with examples of loop 6 inserts shown.

FIGS. 2A to 2D depict protein gels demonstrating the successful confirmation of cyclization of native and FLAG-epitope-containing cyclotides, as well as demonstration of new cyclotide designs of the disclosure. The gels of FIGS. 2A and 2B compare lysates with trypsin treated empty vector (pYES), cyclic peptide (pYES-MCoTI) and FLAG-tagged cyclotide (pYES-MCoTI FLAG). Both native and FLAG-tagged versions of the cyclotides were cyclized.

FIG. 2B shows greater detail regarding the yeast expression vectors containing intein-flanked cyclotide variant sequences (yeast (S. cerevisiae) Nostoc puntiforme (Npu) split intein cyclization). FIG. 2C demonstrates the higher insert capacity (exceeding 22 amino acids) observed for the cyclotide designs of the disclosure, specifically demonstrating the stability of V2 expanded engineered cyclotides of the disclosure. FIG. 2D shows loop expansion cyclotide designs of the disclosure and associated human serum stability.

FIG. 3 depicts a schematic of a process for measuring the stability/extent of cyclization and/or cyclotide content of a sample, as identified by increased fluorescence (relative to the amount of fluorescence observed for a corresponding linear peptide(s), with purification occurring via an epitope tag, and fluorescently labeled amino acid residues—i.e., fluorescently labeled lysine residues—employed for peptide design). Notably, the process is highly scalable/adaptable to high-throughput implementation.

FIG. 4 depicts the results of protein gel electrophoresis for detection of epitope-tagged cyclotides (with washes performed at 10 mM and 40 mM imidazole, respectively), as would be performed in the scheme of FIG. 3 above. Combination with trypsin digests is also performed for cleaner/more pure samples, and trypsin digestion shows stability of cyclotides. [NOTE: imidazole is spelled incorrectly twice on the figure—please fix prior to filing.)

FIG. 5 depicts protein gel analysis of cyclotides exposed to trypsin digestion, with empty vector (pYES), cyclic peptide (pYES-MCoTI) and FLAG-tagged cyclotide (pYES-MCoTI FLAG) compared under the indicated conditions. MCoTI cyclotides possessing FLAG epitope tag inserts were identified as resistant to trypsin digestion to the same extent as a native MCoTI cyclotide (without insert).

FIGS. 6A and 6B depict the results of experiments assessing cyclotide production in E. coli. FIG. 6A demonstrates SDS-PAGE analysis of the expression levels and in vivo cleavage of precursor proteins for generating cyclotides using plasmids grown in E. coli. FIG. 6B is a diagram depicting the expression/purification scheme for using E. coli to produce cyclotides.

FIG. 7 depicts the results of protein gel electrophoresis demonstrating the enrichment of tagged precursor using chitin beads (as in the process of FIG. 6B). Here, the cyclotide precursor was generated and enriched from BL21 and Origami 2 strains of E. coli.

FIG. 8 depicts a diagram of a native and/or “base” cyclotide (solid lines), with “X” marking the site of peptide insertion in loop 6 that has been contemplated in the art, but with architecture for the expanded cyclotide loops of the current disclosure layered upon this “base” cyclotide structure (dotted lines).

FIG. 9 depicts protein gels of trypsin-exposed cyclotides of the current disclosure possessing doubled loop lengths (referred to as “2× cyclotides”). Empty vector (pYES), cyclic peptide (pYES-MCoTI) and FLAG-tagged cyclotide (pYES-MCoTI FLAG) structures are all compared, both with and without trypsin exposure at 0 and 24 hours. “2× cyclotides” exhibited similar levels of trypsin resistance as non-doubled cyclotides over the 24 h trypsin exposure period.

FIG. 10 depicts a scheme for the design of polygonal cyclotides. As described herein, in some embodiments, polygonal cyclotides comprise cyclotides in which two or more cyclotide subunits are connected by a flexible linker. FIG. 10 depicts a polygonal cyclotide (e.g., a “P-2” cyclotide structure) having two cyclotides connected by a pair of flexible linkers. Inclusion of exogenous sequences (e.g., epitopes, peptide drugs and/or other polypeptide inserts) within the flexible linker(s) is expressly contemplated.

FIG. 11 depicts the results of protein gel electrophoresis comparing levels of trypsin digestion resistance between a natural cyclotide and a polygonal (P-2) cyclotide. The P-2 cyclotide structure was also found to be resistant to trypsin digestion.

FIG. 12 depicts different types of cyclotide designs featured in the current disclosure, including a natural cyclotide (left), a (proportionately) multi-loop-expanded cyclotide (middle), and a polygonal (e.g., P-2 as presently shown) cyclotide.

FIGS. 13A and 13B depict schemes for expression and cyclization of cyclotides using microorganisms such as bacteria and yeast. FIG. 13A depicts a general scheme for expression and cyclization of cyclotides in vivo using bacteria (Camarero et al. Chembiochem. 8: 1363-66).

FIG. 13B depicts a partial scheme for in vivo expression of Kalata B1 (“KB1”) cyclotides in bacteria and cyclization in bacterial cytoplasm in vitro (Kimura et al. Angew Chem Int Ed Engl 45: 973-76).

FIG. 14 depicts a graph showing the quantitation of empty vector, cyclotide and FLAG-tagged cyclotide using trypsin agarose beads. Four elutions were performed, at 200 per elution. Protein content was quantified using fluorescent labeling of lysines. For such assays, BSA was employed as a standard, while 9.20 μg MCoTI-I and 0.820 μg MCoTI-I-L6FLAG was present.

FIGS. 15A, 15B, and 15C are additional depictions of intein reaction mechanisms. FIG. 15A depicts the intein trans-splicing mechanism. FIG. 15B depicts products resulting from different intein reactions. Mutation of the last asparagine (ASN) and first cysteine (CYS) to alanine (ALA) renders most inteins N- and C-terminal cleaving, respectively. FIG. 15C depicts an exemplary NpuDanE intein and mini-MtuRecA intein, and the structural alignment therof. Conserved catalytic residues for NpuDnaE and mini-MtuRecA inteins are highlighted in differing shading.

FIG. 16 depicts a mechanism of intein-mediated protein ligation, using an intein bound to a chitin bead. In this exemplary method of producing a novel cyclotide, a GyrA intein is employed. A target gene is first cloned at a multi-cloning site (MCS) within a vector comprising an intein tag sequence, located upstream or downstream of the intein tag sequence, to produce a target protein-intein fusion protein. The expressed fusion protein is loaded on a chitin resin. After washing the resin, the target protein can be cleaved and eluted using a thiol agent (e.g. DTT). When the target protein is eluted by the thiol agent, the C-terminus of the target protein can be activated and be susceptible to thiol attack from a peptide containing an N-terminal Cys residue, such that the target protein and the peptide can be fused. A subsequent S—N acyl shift forms a standard peptide bond between the target protein and the attacking Cys-presenting polypeptide.

FIG. 17 shows a high throughput cyclic peptide pipeline designed for testing of therapeutic peptide inserts, optionally at every possible location within loop 6 of the expanded cyclic peptide structures of the disclosure.

FIG. 18 shows high throughput screening of glucagon-harboring cyclotide peptides of the disclosure, with a number of engineered cyclotides of the disclosure (particularly “V2” and “V3” designs set off by arrows) identified to exhibit receptor binding between engineered cyclotides and GPCR.

FIG. 19 demonstrates the successful production of cyclotide using an in vitro process for cyclotide formation.

FIG. 20 shows initial use of MALDI MS and NMR attempts to confirm cyclotide formation. Such studies are ongoing.

FIG. 21 shows cell-free synthesis of azide-labeled cyclotides.

FIG. 22 shows high throughput assessment of stability and/or cyclization, using azide-linked “click” chemistry approaches.

FIG. 23 shows an attempted enrichment of IntC and precursor with his-tag.

FIG. 24 shows an exemplary evaluation of TSP-1 mimetics on human red blood cells and human serum, with the graph particularly showing the percentage of peptide remaining over a 24 h incubation period in human serum. All data are represented as S.D. (n=3) and were obtained from www.bioscirep.org/content/35/6/e00270.

FIG. 25 presents a list of base cyclotide sequences, showing loop sequences for each such cyclotide sequence (excluding the cysteine residues that define the limits of each loop).

FIG. 26 presents a list of expressly contemplated candidate peptides for insertion, e.g., into loop regions. It is contemplated that in certain embodiments, insert sequences of the cyclic peptides of the instant disclosure may comprise one or more sequences selected from FIG. 26 and/or FIG. 27 below, including, e.g., multiple copies (optionally in tandem) of one or more insert sequences selected from FIGS. 26 and/or 27.

FIG. 27 presents a list of expressly contemplated antimicrobial candidate peptides for insertion, e.g., into loop regions.

FIG. 28 shows the trypsin digest/protease resistance of a 2× engineered cyclotide design that harbored a glucagon insert sequence. Native cyclotide and the engineered design carrying glucagon (glucagon is a 29 amino acid long peptide) each exhibited resistance towards trypsin up to 18 hrs with no change in levels, whereas a linear glucagon peptide was degraded.

FIG. 29 shows a Western blot that demonstrated production of a MCoTI-II precursor in cell free conditions, visualized using anti-chitin binding domain antibody.

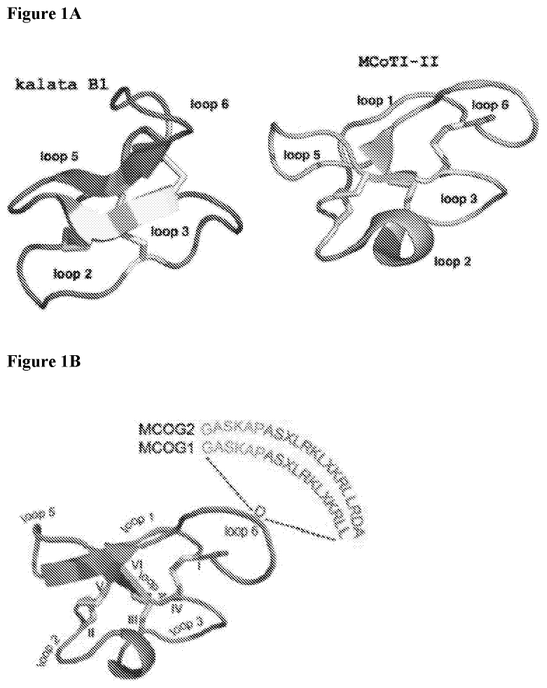

### DETAILED DESCRIPTION OF THE INVENTION

The present invention is based, at least in part, upon the identification of methods for producing cyclic peptides (e.g., cyclotides) capable of harboring exogenous polypeptide sequences of significant length (e.g., about 22 amino acids to 50+ amino acids in length, or more) while retaining a cyclic structure and advantageous properties associated with cyclic peptide structures (e.g., cyclotide structures), such as structural stability, protease stability and/or resistance, resistance to low pH and/or to denaturing chemicals. In certain aspects, proportionate lengthening of two or more “loop” domains of a cyclic peptide structure (e.g., a naturally occurring cyclotide structure) is performed, to extend the available space within the modified/extended cyclic peptide structure for insertion of an exogenous polypeptide. In some aspects, the cyclic peptide compositions produced by such methods are provided, optionally as framework polypeptides that allow for production and/or therapeutic delivery of a relevant peptide, e.g., a peptide drug molecule (e.g., a polypeptide drug of 22-50 or more amino acids in length), a small molecule(s) (optionally via use of non-canonical amino acids and/or linkers for attachment, as described below), and/or antibody molecules or fragments (including, e.g., monoclonal antibodies, single domain antibodies such as camelids and cartilaginous fish antibodies, scFvs, antibody fragments such as Fv, Fab, Fab′ and F(ab′)₂ fragments, and other fragments). Other aspects of the invention provide methods for high-throughput detection of the extent of cyclization/relative amount of cyclotide content (vs., e.g., content of linear peptides) in a polypeptide-containing solution. The parameters of the invention are set forth in additional detail below.

Naturally-occurring cysteine-knot microproteins (“cyclotides”) are small peptides, originally identified in various plant species and typically consisting of about 30-40 amino acids, which can be found as either cyclic or linear forms, where the cyclic form has no free N- or C-terminal amino or carboxyl end (WO 2014/057284). Such naturally-occurring cyclotides have a defined structure based on three intramolecular disulfide bonds and a small triple stranded β-sheet (Craik et al. Toxicon 39: 43-60). The cyclic proteins exhibit conserved cysteine residues defining a structure referred to as a “cysteine knot”. The natural cyclotide family includes both cyclic molecules and their linear derivatives, as well as linear molecules which have undergone cyclization. These molecules are useful as molecular framework structures that possess enhanced stability as compared to less structured peptides (Colgrave and Craik. Biochemistry 43: 5965-5975).

Cyclotides are remarkably stable due to the presence of the cysteine knot, possess a small size that makes them readily accessible to chemical synthesis, and are also highly tolerant of sequence variations. Cyclotides are therefore appealing for use as scaffolds for delivery of therapeutics. The cyclotide scaffold is found in almost 30 different protein families, among which conotoxins, spider toxins, squash inhibitors, agouti-related proteins and plant cyclotides are the most populated families. Within the squash inhibitor family of cyclotides, both cyclic and linear cyclotides have been identified from Momordica cochinchinensis: the cyclic trypsin inhibitors (MCoTI)-I and -II and their linear counterpart MCoTI-III (Hernandez et al. Biochemistry 39: 5722-30). Cyclic peptides have been identified to display improved stability, better resistance to proteases, and reduced flexibility when compared to their linear counterparts, thereby resulting in enhanced biological activities. Insert capacities of canonical cyclotides still capable of functioning as cyclotides after insertion of the additional material have been identified as limited to approximately 20-22 amino acid residues in length (D'Souza et al. Biochemistry 55: 396-405).

### Cyclotide Biology

Cyclotides are small disulfide-rich proteins with a cyclic backbone (thus named cyclo-peptides). They may contain six conserved cystine residues arranged in a cystine knot topology in which two disulfide bonds and their connecting backbone segments form an embedded ring in the structure that is penetrated by a third disulfide bond. Currently known cyclotides have a range of biological activities including anti-HIV and neurotensin inhibition, uterotonic activity, anti-microbial activity and insecticidal activity. Without wishing to be bound by theory, the antibacterial activities may be the result of membrane disruption by the hydrophobic cyclotides. Cyclotides have been found in a variety of tropical plants from the Rubiaceae and Violaceae families.

Cyclotides are exceptionally stable due to the circular protein backbone and knotted arrangement of disulfide bonds. These molecules are exceptionally stable to enzymatic degradation. Because of this stability, they represent useful templates in pharmaceutical applications as described herein. A large proportion of the new cyclotides have been discovered based on their structural properties rather than biological activities. These cyclotides are relatively hydrophobic and can be readily identified from crude plant extracts by their characteristically late elution on RP-HPLC.

In addition to Rubiaceae and Violaceae families, macrocyclic peptides have recently been discovered in the Cucurbitaceae family, noting discovery of the trypsin inhibitors MCoTI-I and MCoTI-II. These 34 residue macrocyclic peptides have no sequence homology to the previously characterized cyclotides, with the exception of the six cysteine residues. However, they contain a cystine knot motif and have a similar size. The MCoTI peptides were originally isolated based on their trypsin inhibitory activity and are homologous to linear cystine knot peptides from the squash family of trypsin inhibitors such as EETI-II and CMTI.

### Cyclotide Structural Topology

The core structural motif of a naturally occurring cyclotide and/or a variant or a derivative of a native cyclotide, called the cyclic cystine knot (CCK), is characterized by a cystine knot embedded in a macrocyclic backbone. The cystine knot involves two intracysteine backbone segments connected by disulfide bonds, CysI-CysIV and CysII-CysV, which form a ring that is penetrated by a third disulfide bond, CysIII-CysVI. The conserved structural characteristics of the cyclotides also include a beta-hairpin, which is generally part of a triple-stranded beta-sheet. In some embodiments, the third strand may be distorted from ideal beta geometry and contain a beta-bulge.

Cyclotides may possess a number of structural features, including Moebius strips, knots, and cystine knots. Moebius strips are a geometric shape with only one surface. They are a strip which is twisted halfway around and attached to itself. In some embodiments, a cis-Pro peptide bond in loop 5 can be thought of as providing a twist in the conceptual ribbon of the peptide backbone, leading to the circular backbone being regarded as a Moebius strip. When this cis-Pro is not present, all backbone peptide bonds are in the trans arrangement, making the backbone bracelet-like. Due to the existence of the cyclic backbone, it is debatable whether cyclotides may be regarded as true knots. Other cystine knotted peptides are topologically simple and are able to be unfolded, however, cyclotides are not topologically simple and may not be unfolded. The cystine knot structural motif is present in peptides and proteins from a variety of species, including fungi, okants, marine molluscs, insects and spiders. There are three classes of cystine knots: Growth Factor Cystine Knot (GFCK), Inhibitor Cystine Knot (ICK) and the Cyclic Cystine Knot (CCK). The cystine knot comprises an embedded ring formed by two disulfide bonds and their connecting backbone segments which is threaded by a third disulfide bond. It may be associated with a nearby beta-sheet structure and in some embodiments is a highly efficient motif for structure stabilization. Because of this stability, it makes an ideal framework for molecular engineering applications. Known peptides containing the cystine knot may be 26-48 residues long and may include various types of agents for treating and/or preventing disease. In some embodiments, the stability of peptide toxins containing the cystine knot motif, their unique structural scaffold, and range of bioactivities may be harnessed for drug design as well as molecular engineering applications.

### Known Cyclotide Sequences

Cybase (cyclic peptide database; www.cybase.org.au) is an online repository of known cyclotide sequences. Cybase contains the sequences of over 800 highly stable cyclic peptides. Naturally occurring cyclotide sequences exhibit limited flexibility and diversity, and possess structural limitations including: 6 cysteine residues (three Cys-Cys disulfide linkages), 6 loops, limited amino acid length, and limited amino acid sequence variability. It is contemplated that the processes described herein for enhancing cyclotide insert lengths can be applied to any cyclotide sequence presented in Cybase. In certain aspects, the cyclotide-improving methods of the current disclosure are applied to one or more of a select number of known cyclotide sequence(s), as set forth in Table 1.

TABLE 1
Select List of Base Cyclotide Sequences

| | |
|---|---|
| MCoTI with CFN | C F N G S G S D G G V C P K I L Q R C R R D S D C P G A C I C |
| junction (peptide bond | R G N G Y C GS (SEQ ID NO: 1) |
| formation between | |
| terminals) | |
| MCoT | G S G S D G G V C P K I L Q R C R R D S D C P G A C I C R G |
| | N G Y C G S (SEQ ID NO: 2) |
| MCOTI I | GGVCPKILQRCRRDSDCPGACICRGNGYCGSGSD (SEQ ID |
| | NO: 3) |
| MCOTI II | GGVCPKILKKCRRDSDCPGACICRGNGYCGSGSD (SEQ ID |
| | NO: 4) |
| Mcoti-III | ERACPRILKKCRRDSDCPGACICRGNGYCG (SEQ ID NO: 5) |
| C15 | GGVCNNNATPSLKVCRRDSDCPGACICRGNGYCGSGSD |
| | (SEQ ID NO: 6) |
| C7 | GGVCGMDLFEESPYCRRDSDCPGACICRGNGYCGSGSD |
| | (SEQ ID NO: 7) |
| C8 | GGVCWLRDEHPFKNCRRDSDCPGACICRSNGYCGSGSD |
| | (SEQ ID NO: 8) |
| C9 | GGVCTYWYLYHTKGCRRDSDCPGACICRGNGYCGSGSD |
| | (SEQ ID NO: 9) |
| C10 | GGVCNLDVANWTVWCRRDSDCPGACICRCNCYCGSGSD |
| | (SEQ ID NO: 10) |
| C11 | GGVCRHSYSQIPLWCRRDSDCPGACICRGNGYCGSGSD |
| | (SEQ ID NO: 11) |
| C12 | GGVCLELAKAYFQMCRRDSDCPGACICRGNGYCGSGSD |
| | (SEQ ID NO: 12) |
| C13 | GGVCQQMHFRVMVHCRRDSDCPGACICRGNGYCGSGSD |
| | (SEQ ID NO: 13) |
| C14 | GGVCTHWRWRSTIWCRRDSDCPGACICRGNGYCGSGSD |
| | (SEQ ID NO: 14) |
| C16 | GGVCFVTDHWEHAPCRRDSDCPGACICRGNGYCGSGSD |
| | (SEQ ID NO: 15) |
| C17 | GGVCFDHHSHYIRRCRRDSDCPGACICRGNGYCGSGSD |
| | (SEQ ID NO: 16) |
| C18 | GGVCQWWLHMINAVCRRDSDCPGACICRGNGYCGSGSD |
| | (SEQ ID NO: 17) |
| C19 | GGVCPFLPTEWWNSCRRDSDCPGACICRGNGYCGSGSD |
| | (SEQ ID NO: 18) |
| C20 | GGVCVRKWWYTESICRRDSDCPGACICRGNGYCGSGSD |
| | (SEQ ID NO: 19) |
| C21 | GGVCYDDETPPHETQHCRRDSDCPGACICRGNGYCGSGSD |
| | (SEQ ID NO: 20) |
| C22 | GGVCQRRKWYWKESIQCRRDSDCPGACICRGNGYCGSGSD |
| | (SEQ ID NO: 21) |
| C23 | GGVCQYTKPFVKGPHHCRRDSDCPGACICRGNGYCGSGSD |
| | (SEQ ID NO: 22) |
| C24 | GGVCSKKRKMSSVVHPCRRDSDCPGACICRGNGYCGSGSD |
| | (SEQ ID NO: 23) |
| C25 | GGVCEVYVWNGELKAWCRRDSDCPGACICRGNGYCGSGSD |
| | (SEQ ID NO: 24) |
| C26 | GGVCRFQQGKWWEPHQCRRDSDCPGACICRGNGYCGSGSD |
| | (SEQ ID NO: 25) |
| C27 | GGVCHMQHPWSAFAWYCHRDSDCPGACICRGNGYCGSGSD |
| | (SEQ ID NO: 26) |
| C28 | GGVCESDPFTEEFMHHCRRDSDCPGACICRGNGYCGSGSD |
| | (SEQ ID NO: 27) |
| C29 | GGVCHKHGYDPVYVWSCRRDSDCPGACICRGNGYYGSGSD |
| | (SEQ ID NO: 28) |

The cyclotide sequences of Table 1 (above) include certain cyclotides described in WO 2014/057284 as particularly effective at crossing the blood-brain barrier (BBB). The methods of the instant disclosure are contemplated to improve the delivery capacity of such cyclotides (any of which can be employed as a base cyclotide sequence in the current methods), optionally while retaining the translocating properties of such cylcotides, including the ability to cross the BBB.

An additional list of base cyclotide sequences expressly contemplated for use in the methods and/or as components of the compositions of the instant disclosure include the following:

TABLE 2
Additional Base Cyclotide Sequences

kalata_B1
(SEQ ID NO: 37)
GLPVCGETCVGGTCNTPGCTCSWPVCTRN
cycloviolacin_O1
(SEQ ID NO: 38)
GIPCAESCVYIPCTVTALLGCSCSNRVCYN
kalata_B2
(SEQ ID NO: 39)
GLPVCGETCFGGTCNTPGCSCTWPICTRD
palicourein
(SEQ ID NO: 40)
GDPTFCGETCRVIPVCTYSAALGCTCDDRSDGLCKRN
vhr1
(SEQ ID NO: 41)
GIPCAESCVWIPCTVTALLGCSCSNKVCYN
tricyclon_A
(SEQ ID NO: 42)
GGTIFDCGESCFLGTCYTKGCSCGEWKLCYGTN
circulin_A
(SEQ ID NO: 43)
GIPCGESCVWIPCISAALGCSCKNKVCYRN
cycloviolacin_O2
(SEQ ID NO: 44)
GIPCGESCVWIPCISSAIGCSCKSKVCYRN
kalata_B6
(SEQ ID NO: 45)
GLPTCGETCFGGTCNTPGCSCSSWPICTRN
kalata_B3
(SEQ ID NO: 46)
GLPTCGETCFGGTCNTPGCTCDPWPICTRD
kalata_B7
(SEQ ID NO: 47)
GLPVCGETCTLGTCYTQGCTCSWPICKRN
cycloviolacin_O8
(SEQ ID NO: 48)
GTLPCGESCVWIPCISSVVGCSCKSKVCYKN
cycloviolacin_O11
(SEQ ID NO: 49)
GTLPCGESCVWIPCISAVVGCSCKSKVCYKN
kalata_B4
(SEQ ID NO: 50)
GLPVCGETCVGGTCNTPGCTCSWPVCTRD
vodo_M
(SEQ ID NO: 51)
GAPICGESCFTGKCYTVQCSCSWPVCTRN
cyclopsychotride_A
(SEQ ID NO: 52)
SIPCGESCVFIPCTVTALLGCSCKSKVCYKN
cycloviolacin_H1
(SEQ ID NO: 53)
GIPCGESCVYIPCLTSAIGCSCKSKVCYRN
cycloviolacin_O9
(SEQ ID NO: 54)
GIPCGESCVWIPCLTSAVGCSCKSKVCYRN
vico_A
(SEQ ID NO: 55)
GSIPCAESCVYIPCFTGIAGCSCKNKVCYYN
vitri_A
(SEQ ID NO: 56)
GIPCGESCVWIPCITSAIGCSCKSKVCYRN
kalata_S
(SEQ ID NO: 57)
GLPVCGETCVGGTCNTPGCSCSWPVCTRN
cycloviolacin_O12
(SEQ ID NO: 58)
GLPICGETCVGGTCNTPGCSCSWPVCTRN
vodo_N
(SEQ ID NO: 59)
GLPVCGETCTLGKCYTAGCSCSWPVCYRN
vico_B
(SEQ ID NO: 60)
GSIPCAESCVYIPCITGIAGCSCKNKVCYYN
Hypa A
(SEQ ID NO: 61)
GIPCAESCVYIPCTITALLGCSCKNKVCYN
circulin_B
(SEQ ID NO: 62)
GVIPCGESCVFIPCISTLLGCSCKNKVCYRN
circulin_C
(SEQ ID NO: 63)
GIPCGESCVFIPCITSVAGCSCKSKVCYRN
circulin_D
(SEQ ID NO: 64)
KIPCGESCVWIPCVTSIFNCKCENKVCYHD
circulin_E
(SEQ ID NO: 65)
KIPCGESCVWIPCLTSVFNCKCENKVCYHD
circulin_F
(SEQ ID NO: 66)
AIPCGESCVWIPCISAAIGCSCKNKVCYR
cycloviolacin_O4
(SEQ ID NO: 67)
GIPCGESCVWIPCISSAIGCSCKNKVCYRN
cycloviolacin_O3
(SEQ ID NO: 68)
GIPCGESCVWIPCLTSAIGCSCKSKVCYRN
cycloviolacin_O5
(SEQ ID NO: 69)
GTPCGESCVWIPCISSAVGCSCKNKVCYKN
cycloviolacin_O6
(SEQ ID NO: 70)
GTLPCGESCVWIPCISAAVGCSCKSKVCYKN
cycloviolacin_O7
(SEQ ID NO: 71)
SIPCGESCVWIPCTITALAGCKCKSKVCYN
cycloviolacin_O10
(SEQ ID NO: 72)
GIPCGESCVYIPCLTSAVGCSCKSKVCYRN
kalata_B5
(SEQ ID NO: 73)
GTPCGESCVYIPCISGVIGCSCTDKVCYLN
vary_peptide_B
(SEQ ID NO: 74)
GLPVCGETCFGGTCNTPGCSCDPWPMCSRN
vary_peptide_C
(SEQ ID NO: 75)
GVPICGETCVGGTCNTPGCSCSWPVCTRN
vary_peptide_D
(SEQ ID NO: 76)
GLPICGETCVGGSCNTPGCSCSWPVCTRN
vary_peptide_F
(SEQ ID NO: 77)
GVPICGETCTLGTCYTAGCSCSWPVCTRN
vary_peptide_G
(SEQ ID NO: 78)
GVPVCGETCFGGTCNTPGCSCDPWPVCSRN
vary_peptide_H
(SEQ ID NO: 79)
GLPVCGETCFGGTCNTPGCSCETWPVCSRN
cycloviolin_A
(SEQ ID NO: 80)
GVIPCGESCVFIPCISAAIGCSCKNKVCYRN
cycloviolin_B
(SEQ ID NO: 81)
GTACGESCYVLPCFTVGCTCTSSQCFKN
cycloviolin_C
(SEQ ID NO: 82)
GIPCGESCVFIPCLTTVAGCSCKNKVCYRN
cycloviolin_D
(SEQ ID NO: 83)
GFPCGESCVFIPCISAAIGCSCKNKVCYRN
violapeptide_1
(SEQ ID NO: 84)
GLPVCGETCVGGTCNTPGCSCSRPVCTXN
vhl-1
(SEQ ID NO: 85)
SISCGESCAMISFCFTEVIGCSCKNKVCYLN
hcf-1
(SEQ ID NO: 86)
GIPCGESCHYIPCVTSAIGCSCRNRSCMRN
htf-1
(SEQ ID NO: 87)
GIPCGDSCHYIPCVTSTIGCSCTNGSCMRN
vh1-2
(SEQ ID NO: 88)
GLPVCGETCFTGTCYTNGCTCDPWPVCTRN
cycloviolacin_H3
(SEQ ID NO: 89)
GLPVCGETCFGGTCNTPGCICDPWPVCTRN
cycloviolacin_H2
(SEQ ID NO: 90)
SAIACGESCVYIPCFIPGCSCRNRVCYLN
Hyfl_A
(SEQ ID NO: 91)
SISCGESCVYIPCTVTALVGCTCKDKVCYLN
Hyfl_B
(SEQ ID NO: 92)
GSPIQCAETCFIGKCYTEELGCTCTAFLCMKN
Hyfl_C
(SEQ ID NO: 93)
GSPRQCAETCFIGKCYTEELGCTCTAFLCMKN
Hyfl_D
(SEQ ID NO: 94)
GSVPCGESCVYIPCFTGIAGCSCKSKVCYYN
Hyfl_E
(SEQ ID NO: 95)
GEIPCGESCVYLPCFLPNCYCRNHVCYLN
Hyfl_F
(SEQ ID NO: 96)
SISCGETCTTFNCWIPNCKCNHHDKVCYWN
Hyfl_I
(SEQ ID NO: 97)
GIPCGESCVFIPCISGVIGCSCKSKVCYRN
Hyfl_J
(SEQ ID NO: 98)
GIACGESCAYFGCWIPGCSCRNKVCYFN
Hyfl_K
(SEQ ID NO: 99)
GTPCGESCVYIPCFTAVVGCTCKDKVCYLN
Hyfl_L
(SEQ ID NO: 100)
GTPCAESCVYLPCFTGVIGCTCKDKVCYLN
Hyfl_M
(SEQ ID NO: 101)
GNIPCGESCIFFPCFNPGCSCKDNLCYYN
tricyclon_B
(SEQ ID NO: 102)
GGTIFDCGESCFLGTCYTKGCSCGEWKLCYGEN
kalata_B8
(SEQ ID NO: 103)
GSVLNCGETCLLGTCYTTGCTCNKYRVCTKD
cycloviolacin_H4
(SEQ ID NO: 104)
GIPCAESCVWIPCTVTALLGCSCSNNVCYN
cycloviolacin_O13
(SEQ ID NO: 105)
GIPCGESCVWIPCISAAIGCSCKSKVCYRN
violacin_A
(SEQ ID NO: 106)
SAISCGETCFKFKCYTPRCSCSYPVCK
cycloviolacin_O14
(SEQ ID NO: 107)
GSIPACGESCFKGKCYTPGCSCSKYPLCAKN
cycloviolacin_O15
(SEQ ID NO: 108)
GLVPCGETCFTGKCYTPGCSCSYPICKKN
cycloviolacin_O16
(SEQ ID NO: 109)
GLPCGETCFTGKCYTPGCSCSYPICKKIN
cycloviolacin_O17
(SEQ ID NO: 110)
GIPCGESCVWIPCISAAIGCSCKNKVCYRN
cycloviolacin_O18
(SEQ ID NO: 111)
GIPCGESCVYIPCTVTALAGCKCKSKVCYN
cycloviolacin_O19
(SEQ ID NO: 112)
GTLPCGESCVWIPCISSVVGCSCKSKVCYKD
cycloviolacin_O20
(SEQ ID NO: 113)
GIPCGESCVWIPCLTSAIGCSCKSKVCYRD
cycloviolacin_O21
(SEQ ID NO: 114)
GLPVCGETCVTGSCYTPGCTCSWPVCTRN
cycloviolacin_O22
(SEQ ID NO: 115)
GLPICGETCVGGTCNTPGCTCSWPVCTRN
cycloviolacin_O23
(SEQ ID NO: 116)
GLPTCGETCFGGTCNTPGCTCDSSWPICTHN
cycloviolacin_O24
(SEQ ID NO: 117)
GLPTCGETCFGGTCNTPGCTCDPWPVCTHN
cycloviolacin_O25
(SEQ ID NO: 118)
DIFCGETCAFIPCITHVPGTCSCKSKVCYFN
kalata_B9
(SEQ ID NO: 119)
GSVFNCGETCVLGTCYTPGCTCNTYRVCTKD
kalata_B9_linear
(SEQ ID NO: 120)
GSVFNCGETCVLGTCYTPGCTCNTYRVCTKD
kalata_B10
(SEQ ID NO: 121)
GLPTCGETCFGGTCNTPGCSCSSWPICTRD
kalata_B10_linear
(SEQ ID NO: 122)
GLPTCGETCFGGTCNTPGCSCSSWPICTRD
kalata_B11
(SEQ ID NO: 123)
GLPVCGETCFGGTCNTPGCSCTDPICTRD
kalata_B12
(SEQ ID NO: 124)
GSLCGDTCFVLGCNDSSCSCNYPICVKD
kalata_B13
(SEQ ID NO: 125)
GLPVCGETCFGGTCNTPGCACDPWPVCTRD
kalata_B14
(SEQ ID NO: 126)
GLPVCGESCFGGTCNTPGCACDPWPVCTRD
kalata_B15
(SEQ ID NO: 127)
GLPVCGESCFGGSCYTPGCSCTWPICTRD
kalata_B16
(SEQ ID NO: 128)
GIPCAESCVYIPCTITALLGCKCQDKVCYD
kalata_B17
(SEQ ID NO: 129)
GIPCAESCVYIPCTITALLGCKCKDQVCYN
kalata_B18
(SEQ ID NO: 130)
GVPCAESCVYIPCISTVLGCSCSNQVCYRN
PS-1
(SEQ ID NO: 131)
GFIPCGETCIWDKTCHAAGCSCSVANICVRN
CD-1
(SEQ ID NO: 132)
GADGFCGESCYVIPCISYLVGCSCDTIEKVCKRN
cycloviolacin_Y1
(SEQ ID NO: 133)
GGTIFDCGETCFLGTCYTPGCSCGNYGFCYGTN
cycloviolacin_Y2
(SEQ ID NO: 134)
GGTIFDCGESCFLGTCYTAGCSCGNWGLCYGTN
cycloviolacin_Y3
(SEQ ID NO: 135)
GGTIFDCGETCFLGTCYTAGCSCGNWGLCYGTN
cycloviolacin_Y4
(SEQ ID NO: 136)
GVPCGESCVFIPCITGVIGCSCSSNVCYLN
cycloviolacin_Y5
(SEQ ID NO: 137)
GIPCAESCVWIPCTVTALVGCSCSDKVCYN
vibi_A
(SEQ ID NO: 138)
GLPVCGETCFGGTCNTPGCSCSYPICTRN
vibi_B
(SEQ ID NO: 139)
GLPVCGETCFGGTCNTPGCTCSYPICTRN
vibi_C
(SEQ ID NO: 140)
GLPVCGETCAFGSCYTPGCSCSWPVCTRN
vibi_D
(SEQ ID NO: 141)
GLPVCGETCFGGRCNTPGCTCSYPICTRN
vibi_E
(SEQ ID NO: 142)
GIPCAESCVWIPCTVTALIGCGCSNKVCYN
vibi_F
(SEQ ID NO: 143)
GTIPCGESCVFIPCLTSALGCSCKSKVCYKN
vibi_G
(SEQ ID NO: 144)
GTFPCGESCVFIPCLTSAIGCSCKSKVCYKN
vibi_H
(SEQ ID NO: 145)
GLLPCAESCVYIPCLTTVIGCSCKSKVCYKN
vibi_I
(SEQ ID NO: 146)
GIPCGESCVWIPCLTSTVGCSCKSKVCYRN
vibi_J
(SEQ ID NO: 147)
GTFPCGESCVWIPCISKVIGCACKSKVCYKN
vibi_K
(SEQ ID NO: 148)
GIPCGESCVWIPCLTSAVGCPCKSKVCYRN
Viba_2
(SEQ ID NO: 149)
GIPCGESCVYLPCFTAPLGCSCSSKVCYRN
Viba_5
(SEQ ID NO: 150)
GIPCGESCVWIPCLTATIGCSCKSKVCYRN
Viba_10
(SEQ ID NO: 151)
GIPCAESCVYLPCVTIVIGCSCKDKVCYN
Viba_12
(SEQ ID NO: 152)
GIPCAESCVWIPCTVTALLGCSCKDKVCYN
Viba_14
(SEQ ID NO: 153)
GRLCGERCVIERTRAWCRTVGCICSLHTLECVRN
Viba_17
(SEQ ID NO: 154)
GLPVCGETCVGGTCNTPGCGCSWPVCTRN
Viba_15
(SEQ ID NO: 155)
GLPVCGETCVGGTCNTPGCACSWPVCTRN
Mra4
(SEQ ID NO: 156)
GSIPCGESCVYIPCISSLLGCSCKSKVCYKN
Mra5
(SEQ ID NO: 157)
GIPCAESCVYIPCLTSAIGCSCKSKVCYRN
Mra13
(SEQ ID NO: 158)
GIPCGESCVYLPCFTTIIGCKCQGKVCYH
Mra14a
(SEQ ID NO: 159)
GSIPCGESCVFIPCISSVVGCSCKNKVCYKN
Mra14b
(SEQ ID NO: 160)
GTIPCGESCVFIPCLTSAIGCSCKSKVCYKN
Mra17
(SEQ ID NO: 161)
GSIPCGESCVYIPCISSLLGCSCESKVCYKN
Mra29
(SEQ ID NO: 162)
GSIPCGESCVFIPCISSIVGCSCKSKVCYKN
Mra30
(SEQ ID NO: 163)
GIPCGESCVFIPCLTSAIGCSCKSKVCYRN
Mra22
(SEQ ID NO: 164)
GVPCGESCVWIPCLTSIVGCSCKNNVCTLN
Mra23
(SEQ ID NO: 165)
GVIPCGESCVFIPCISSVLGCSCKNKVCYRN
Mra24
(SEQ ID NO: 166)
GHPTCGETCLLGTCYTPGCTCKRPVCYKN
Mra25
(SEQ ID NO: 167)
GSAILCGESCTLGECYTPGCTCSWPICTKN
Mra26
(SEQ ID NO: 168)
GHPICGETCVGNKCYTPGCTCTWPVCYRN
Mra30
(SEQ ID NO: 169)
GSIPCGEGCVFIPCISSIVGCSCKSKVCYKN
Viba_1
(SEQ ID NO: 170)
GIPCGEGCVYLPCFTAPLGCSCSSKVCYRN
Viba_3
(SEQ ID NO: 171)
GIPCGESCVWIPCLTAAIGCSCSSKVCYRN
Viba_4
(SEQ ID NO: 172)
GVPCGESCVWIPCLTSAIGCSCKSSVCYRN
Viba_6
(SEQ ID NO: 173)
GIPCGESCVLIPCISSVIGCSCKSKVCYRN
Viba_7
(SEQ ID NO: 174)
GVIPCGESCVFIPCISSVIGCSCKSKVCYRN
Viba_8
(SEQ ID NO: 175)
GAGCIETCYTFPCISEMINCSCKNSRCQKN
Viba_9
(SEQ ID NO: 176)
GIPCGESCVWIPCISSAIGCSCKNKVCYRK
Viba_11
(SEQ ID NO: 177)
GIPCGESCVWIPCISGAIGCSCKSKVCYRN
Viba_13
(SEQ ID NO: 178)
TIPCAESCVWIPCTVTALLGCSCKDKVCYN
Viba_16
(SEQ ID NO: 179)
GLPICGETCTLGTCYTVGCTCSWPICTRN
Cter_A
(SEQ ID NO: 180)
GVIPCGESCVFIPCISTVIGCSCKNKVCYRN
Cter_B
(SEQ ID NO: 181)
GVPCAESCVWIPCTVTALLGCSCKDKVCYLN
hcf-1_variant
(SEQ ID NO: 182)
GIPCGESCHIPCVTSAIGCSCRNRSCMRN
Vp1-1
(SEQ ID NO: 183)
GSQSCGESCVLIPCISGVIGCSCSSMICYFN
Vpf-1
(SEQ ID NO: 184)
GIPCGESCVFIPCLTAAIGCSCRSKVCYRN
cO31
(SEQ ID NO: 185)
GLPVCGETCVGGTCNTPGCSCSIPVCTRN
cO28
(SEQ ID NO: 186)
GLPVCGETCVGGTCNTPGCSCSWPVCFRD
cO32
(SEQ ID NO: 187)
GAPVCGETCFGGTCNTPGCTCDPWPVCTND
cO33
(SEQ ID NO: 188)
GLPVCGETCVGGTCNTPYCTCSWPVCTRD
cO34
(SEQ ID NO: 189)
GLPVCGETCVGGTCNTEYCTCSWPVCTRD
cO35
(SEQ ID NO: 190)
GLPVCGETCVGGTCNTPYCFCSWPVCTRD
cO29
(SEQ ID NO: 191)
GIPCGESCVWIPCISGAIGCSCKSKVCYKN
cO30
(SEQ ID NO: 192)
GIPCGESCVWIPCISSAIGCSCKNKVCFKN
cO26
(SEQ ID NO: 193)
GSIPACGESCFRGKCYTPGCSCSKYPLCAKD
cO27
(SEQ ID NO: 194)
GSIPACGESCFKGWCYTPGCSCSKYPLCAKD
Globa_F
(SEQ ID NO: 195)
GSFPCGESCVFIPCISAIAGCSCKNKVCYKN
Globa_A
(SEQ ID NO: 196)
GIPCGESCVFIPCITAAIGCSCKTKVCYRN
Globa_B
(SEQ ID NO: 197)
GVIPCGESCVFIPCISAVLGCSCKSKVCYRN
Globa_D
(SEQ ID NO: 198)
GIPCGETCVFMPCISGPMGCSCKHMVCYRN
Globa_E
(SEQ ID NO: 199)
GSAFGCGETCVKGKCNTPGCVCSWPVCKKN
Globa_C
(SEQ ID NO: 200)
APCGESCVYIPCLLTAPIGCSCSNIVCYRN
Glopa_D
(SEQ ID NO: 201)
GVPCGESCVWVPCTVTALMGCSCVREVCRKD
Glopa_E
(SEQ ID NO: 202)
GIPCAESCVWIPCTVTKMLGCSCKDKVCYN
Glopa_A
(SEQ ID NO: 203)
GGSIPCIETCVWTGCFLVPGCSCKSDKKCYLN
Glopa_B
(SEQ ID NO: 204)
GGSVPCIETCVWTGCFLVPGCSCKSDKKCYLN
Glopa_C
(SEQ ID NO: 205)
GDIPLCGETCFEGGNCRIPGCTCVWPFCSKN
cO36
(SEQ ID NO: 206)
GLPTCGETCFGGTCNTPGCTCDPFPVCTHD
cycloviolacin_T1
(SEQ ID NO: 207)
GIPVCGETCVGGTCNTPGCSCSWPVCTRN
psyle_A
(SEQ ID NO: 208)
GIACGESCVFLGCFIPGCSCKSKVCYFN
psyle_B
(SEQ ID NO: 209)
GIPCGETCVAFGCWIPGCSCKDKLCYYD
psyle_C
(SEQ ID NO: 210)
KLCGETCFKFKCYTPGCSCSYPFCK
psyle_D
(SEQ ID NO: 211)
GIPCGESCVFIPCTVTALLGCSCQNKVCYRD
psyle_E
(SEQ ID NO: 212)
GVIPCGESCVFIPCISSVLGCSCKNKVCYRD
psyle_F
(SEQ ID NO: 213)
GVIPCGESCVFIPCITAAVGCSCKNKVCYRD
vaby_A
(SEQ ID NO: 214)
GLPVCGETCAGGTCNTPGCSCSWPICTRN
vaby_B
(SEQ ID NO: 215)
GLPVCGETCAGGTCNTPGCSCTWPICTRN
vaby_C
(SEQ ID NO: 216)
GLPVCGETCAGGRCNTPGCSCSWPVCTRN
vaby_D
(SEQ ID NO: 217)
GLPVCGETCFGGTCNTPGCTCDPWPVCTRN
vaby_E
(SEQ ID NO: 218)
GLPVCGETCFGGTCNTPGCSCDPWPVCTRN
kalata_B19
(SEQ ID NO: 219)
GFPCGESCVYVPCLTAAIGCSCSNKVCYKN
Oak6_cyclotide_2
(SEQ ID NO: 220)
GLPICGETCFGGTCNTPGCICDPWPVCTRD
Oak7_cyclotide
(SEQ ID NO: 221)
GSHCGETCFFFGCYKPGCSCDELRQCYKN
Oak8_cyclotide
(SEQ ID NO: 222)
GVPCGESCVFIPCLTAVVGCSCSNKVCYLN
Oak6_cyclotide_1
(SEQ ID NO: 223)
GLPVCGETCFGGTCNTPGCACDPWPVCTRN
Cter_C
(SEQ ID NO: 224)
GVPCAESCVWIPCTVTALLGCSCKDKVCYLD
Cter_D
(SEQ ID NO: 225)
GIPCAESCVWIPCTVTALLGCSCKDKVCYLN
Cter_E
(SEQ ID NO: 226)
GIPCAESCVWIPCTVTALLGCSCKDKVCYLD
Cter_F
(SEQ ID NO: 227)
GIPCGESCVFIPCISSVVGCSCKSKVCYLD
Cter_G
(SEQ ID NO: 228)
GLPCGESCVFIPCITTVVGCSCKNKVCYNN
Cter_H
(SEQ ID NO: 229)
GLPCGESCVFIPCITTVVGCSCKNKVCYND
Cter_I
(SEQ ID NO: 230)
GTVPCGESCVFIPCITGIAGCSCKNKVCYIN
Cter_J
(SEQ ID NO: 231)
GTVPCGESCVFIPCITGIAGCSCKNKVCYID
Cter_K
(SEQ ID NO: 232)
HEPCGESCVFIPCITTVVGCSCKNKVCYN
Cter_L
(SEQ ID NO: 233)
HEPCGESCVFIPCITTVVGCSCKNKVCYD
Cter_M
(SEQ ID NO: 234)
GLPTCGETCTLGTCYVPDCSCSWPICMKN
Cter_N
(SEQ ID NO: 235)
GSAFCGETCVLGTCYTPDCSCTALVCLKN
Cter_O
(SEQ ID NO: 236)
GIPCGESCVFIPCITGIAGCSCKSKVCYRN
Cter_P
(SEQ ID NO: 237)
GIPCGESCVFIPCITAAIGCSCKSKVCYRN
Cter_Q
(SEQ ID NO: 238)
GIPCGESCVFIPCISTVIGCSCKNKVCYRN
Cter_R
(SEQ ID NO: 239)
GIPCGESCVFIPCTVTALLGCSCKDKVCYKN
vitri_B
(SEQ ID NO: 240)
GVPICGESCVGGTCNTPGCSCSWPVCTTN
vitri_C
(SEQ ID NO: 241)
GLPICGETCVGGTCNTPGCFCTWPVCTRN
vitri_D
(SEQ ID NO: 242)
GLPVCGETCFTGSCYTPGCSCNWPVCNRN
vitri_E
(SEQ ID NO: 243)
GLPVCGETCVGGTCNTPGCSCSWPVCFRN
vitri_F
(SEQ ID NO: 244)
GLTPCGESCVWIPCISSVVGCACKSKVCYKD
hedyotide_B1
(SEQ ID NO: 245)
GTRCGETCFVLPCWSAKFGCYCQKGFCYRN
Parigidin-brl
(SEQ ID NO: 246)
GGSVPCGESCVFIPCITSLAGCSCKNKVCYYD
hedyotide_B2
(SEQ ID NO: 247)
GIQCGESCVWIPCISSAWGCSCKNKICSS
viphi_A
(SEQ ID NO: 248)
GSIPCGESCVFIPCISSVIGCACKSKVCYKN
viphi_B
(SEQ ID NO: 249)
GLPVCGETCTIGTCYTAGCTCSWPICTRN
viphi_C
(SEQ ID NO: 250)
GVPCGESCVYIPCITSVIGCSCSSKVCYIN
viphi_D
(SEQ ID NO: 251)
GIPCGESCVFIPCISSVIGCSCSSKVCYRN
viphi_E
(SEQ ID NO: 252)
GSIPCGESCVFIPCISAVIGCSCSNKVCYKN
viphi_F
(SEQ ID NO: 253)
GSIPCGESCVFIPCISAIIGCSCSSKVCYKN
viphi_G
(SEQ ID NO: 254)
GSIPCGESCVFIPCISAIIGCSCSNKVCYKN
viphi_H
(SEQ ID NO: 255)
GIPCAESCVWIPCTVTAIVGCSCSWGVCYN
cliotide_T8
(SEQ ID NO: 256)
GIPCGESCVFIPCISSVVGCSCKSKVCYNN
cliotide_T9
(SEQ ID NO: 257)
GIPCGESCVFIPCLTTVVGCSCKNKVCYNN
cliotide_T2
(SEQ ID NO: 258)
GEFLKCGESCVQGECYTPGCSCDWPICKKN
cliotide_T12
(SEQ ID NO: 259)
GIPCGESCVFIPCITGAIGCSCKSKVCYRD
Panitide_L1
(SEQ ID NO: 260)
QLPICGETCVLGTCYTPGCRCQYPICVR
Panitide_L2
(SEQ ID NO: 261)
QLPICGETCVLGRCYTPNCRCQYPICVR
Panitide_L4
(SEQ ID NO: 262)
QAFCGETCLLGTCYTPGCRCTAGICLK
Panitide_L6
(SEQ ID NO: 263)
QLPICGETCVLGTCYTPGCSCAYPICVR
Panitide_L3
(SEQ ID NO: 264)
QAFCGETCLLGKCYTPGCSCHTGICLK
Panitide_L5
(SEQ ID NO: 265)
QLPICGETCVLGTCYTPGCSCAYPICAR
Panitide_L7
(SEQ ID NO: 266)
QAFCGETCVLGTCYTPGCSCNFGICLK
Panitide_L8
(SEQ ID NO: 267)
QDCGETCVLGTCYTPGCSCSAYPLCV
vigno_1
(SEQ ID NO: 268)
GLPLCGETCAGGTCNTPGCSCSWPVCVRN
vigno_2
(SEQ ID NO: 269)
GSSPLCGETCAGGTCNTPGCSCSWPVCVRD
vigno_3
(SEQ ID NO: 270)
GLPLCGETCVGGTCNTPGCSCSWPVCTRN
vigno_4
(SEQ ID NO: 271)
GLPLCGETCVGGTCNTPACSCSWPVCTRN
vigno_5
(SEQ ID NO: 272)
GLPLCGETCVGGTCNTPGCSCGWPVCVRN
vigno_6
(SEQ ID NO: 273)
GIPCGESCVWIPCISSAIGCSCKGSKVCYRN
vigno_7
(SEQ ID NO: 274)
GTLPCGESCVWIPCISSVVGCSCKNKVCYKN
vigno_8
(SEQ ID NO: 275)
GIPCGESCVWIPCITSAVGCSCKSKVCYRN
vigno_9
(SEQ ID NO: 276)
GIPCGESCVWIPCISSALGCSCKSKVCYRN
vigno_10
(SEQ ID NO: 277)
GTIPCGESCVWIPCISSVVGCSCKSKVCYKD
caripe_1
(SEQ ID NO: 278)
GVIPCGESCVFIPCISTVIGCSCKDKVCYRN
caripe_2
(SEQ ID NO: 279)
GIPCGESCVFIRCTITALLGCSCSNNVCYKN
caripe_4
(SEQ ID NO: 280)
LICSSTCLRIPCLSPRCTCRHHICYLN
caripe_6
(SEQ ID NO: 281)
GAICTGTCFRNPCLSRRCTCRHYICYLN
chacur_1
(SEQ ID NO: 282)
GLPVCGETCVGGTCNTPGCTCSWPICTRN
psybra_1
(SEQ ID NO: 283)
GLPICGETCTLGTCNTPGCTCSWPICTKN
paltet_1
(SEQ ID NO: 284)
GLPICGETCFTGTCNTPGCTCSYPVCTRN
psypoe_1
(SEQ ID NO: 285)
GSVICGETCFTTVCNTPGCYCGAYXCTRN
caripe_7
(SEQ ID NO: 286)
GIPCGESCVFIPCTVTALLGCSCKNKVCYRN
caripe_8
(SEQ ID NO: 287)
GVIPCGESCVFIPCITAAIGCSCKKKVCYRN
chassatide_C18
(SEQ ID NO: 288)
GIPCGESCVFIPCISALLGCSCSNKVCYNN
chassatide_C16
(SEQ ID NO: 289)
GVPCAESCVYIPCTITALFGCSCKDKVCYNN
chassatide_C15
(SEQ ID NO: 290)
GIPCAESCVYIPCTITALLGCSCKDKVCYKN
chassatide_C13
(SEQ ID NO: 291)
GFPCAESCVYIPCTVTALLGCSCRNRVCYRN
chassatide_C17
(SEQ ID NO: 292)
IPCGESCVYIPCISAVLGCSCQNKVCYR
chassatide_C14
(SEQ ID NO: 293)
GIPCAESCVYIPCTITALFGCSCKDKVCYNN
chassatide_C8
(SEQ ID NO: 294)
AIPCGESCVWIPCISTVIGCSCSNKVCYR
chassatide_C7
(SEQ ID NO: 295)
IPCGESCVWIPCITAIAGCSCKNKVCYT
chassatide_C4
(SEQ ID NO: 296)
GASCGETCFTGICFTAGCSCNPWPTCTRN
chassatide_C2
(SEQ ID NO: 297)
GIPCAESCVWIPCTITALMGCSCKNNVCYNN
chassatide_C1
(SEQ ID NO: 298)
GDACGETCFTGICFTAGCSCNPWPTCTRN
chassatide_C3
(SEQ ID NO: 299)
GIPCGESCVWIPCISSALGCSCKNKVCYRN
chassatide_C5
(SEQ ID NO: 300)
GVIPCGESCVFIPCISSVVGCSCKNKVCYRN
chassatide_C6
(SEQ ID NO: 301)
GVIPCGESCVFIPCISSVIGCSCKNKVCYRN
chassatide_C9
(SEQ ID NO: 302)
GIPCGESCVFIPCVTTVIGCSCKDKVCYNN
chassatide_C10
(SEQ ID NO: 303)
GEYCGESCYLIPCFTPGCYCVSRQCVNKN
chassatide_C11
(SEQ ID NO: 304)
IPCGESCVWIPCISGMFGCSCKDKVCYS
chassatide_C12
(SEQ ID NO: 305)
EYCGESCYLIPCFTPGCYCVSRQCVNKN
Phyb_A
(SEQ ID NO: 306)
GIGCGESCVWIPCVSAAIGCSCSNKICYRN
Phyb_D
(SEQ ID NO: 307)
GIPCGESCMWIPCISAAIGCSCTNHVCYKN
Phyb_E
(SEQ ID NO: 308)
GIPCGESCVWIPCISGVQGCSCSNKICYRN
Phyb_F
(SEQ ID NO: 309)
GIPCGGSCVWIPCISGVQGCSCSNKICYRN
Phyb_G
(SEQ ID NO: 310)
GIPCGESCAWIPCISAVQGCSCRNKICYRN
Phyb_H
(SEQ ID NO: 311)
GLPCGESCIWIECISGAIGCSCRNKVCYRN
Phyb_I
(SEQ ID NO: 312)
GIPCGESCIWIPCTTTALLGCSCSNKVCYKN
Phyb_J
(SEQ ID NO: 313)
SYTCGESCLWIPCTVTAAFGCYCSNKVCVKD
Phyb_K
(SEQ ID NO: 314)
STDCGEPCVYIPCTITALLGCSCLNKVCVRP
Phyb_L
(SEQ ID NO: 315)
QSISCAETCVWIPCATSLIGCSCVNSICTYTN
Phyb_B
(SEQ ID NO: 316)
GVPCGESCVWMYCISAAMGCSCRNKVCYRN
Phyb_C
(SEQ ID NO: 317)
GIPCGESCVWMYCITATMGCSCRNKVCYKN
vocC
(SEQ ID NO: 318)
GLPVCGETCVGGTCNTPGCSCSWPVCIRN
vitri_peptide_1
(SEQ ID NO: 319)
GLIPCGESCVWIPCISSVIGCSCKSKVCYKN
vitri_peptide_2
(SEQ ID NO: 320)
GSIPCGESCVWIPCISGIAGCSCSNKVCYLN
vitri_peptide_3
(SEQ ID NO: 321)
GSWPCGESCVYIPCITSIAGCECSKNVCYKN
vitri_peptide_4
(SEQ ID NO: 322)
GTPCGESCIYVPCISAVFGCWCQSKVCYKD
vitri_peptide_8
(SEQ ID NO: 323)
PTPCGETCIWISCVTAAIGCYCHESICYR
vitri_peptide_9a/53
(SEQ ID NO: 324)
GTIFDCGETCLLGKCYTPGCSCGSWALCYGQN
vitri_peptide_14
(SEQ ID NO: 325)
GSSCGETCEVFSCFITRCACIDGLCYRN
vitri_peptide_18a
(SEQ ID NO: 326)
GVPICGETCFQGTCNTPGCTCKWPICERN
vitri_peptide_20
(SEQ ID NO: 327)
GDLVPCGESCVYIPCLTTVLGCSCSENVCYRN
vitri_peptide_21
(SEQ ID NO: 328)
GGPLDCQETCTLSDRCYTKGCTCNWPICYKN
vitri_peptide_22a
(SEQ ID NO: 329)
GAPVCGETCFTGLCYSSGCSCIYPVCNRN
vitri_peptide_94b
(SEQ ID NO: 330)
GVAVCGETCTLGTCYTPGCSCDWPICKRN
vitri_peptide_23
(SEQ ID NO: 331)
GLPTCGETCTLGTCYTPGCTCSWPLCTKN
vitri_peptide_24/28
(SEQ ID NO: 332)
GEPVCGDSCVFFGCDDEGCTCGPWSLCYRN
vitri_peptide_27a
(SEQ ID NO: 333)
GAFTPCGETCLTGECHTEGCSCVGQTFCVKK
vitri_peptide_29
(SEQ ID NO: 334)
GVPSSDCLETCFGGKCNAHRCTCSQWPLCAKN
vitri_peptide_30
(SEQ ID NO: 335)
GFACGETCIFTSCFITGCTCNSSLCFRN
vitri_peptide_36/37
(SEQ ID NO: 336)
GGTIFSCGESCFQGTCYTKGCACGDWKLCYGEN
vitri_peptide_38
(SEQ ID NO: 337)
GDTCYETCFTGFCFIGGCKCDFPVCVKN
vitri_peptide_39
(SEQ ID NO: 338)
GAPICGESCFTGTCYTVQCSCSWPVCTRN
vitri_peptide_39_linear
(SEQ ID NO: 339)
GAPICGESCFTGTCYTVQCSCSWPVCTRN
vitri_peptide_24a
(SEQ ID NO: 340)
GGTIFNCGESCFQGTCYTKGCACGDWKLCYGEN
vitri_peptide_50
(SEQ ID NO: 341)
GDIPCGESCVYIPCITGVLGCSCSHNVCYYN
vitri_peptide_18b
(SEQ ID NO: 342)
GSVFNCGETCVFGTCFTSGCSCVYRVCSKD
Cter_35
(SEQ ID NO: 343)
GAFCGETCVLGTCYTPGCSCAPVICLNN
mech_2
(SEQ ID NO: 344)
GIPTCGETCTLGKCNTPKCTCNWPICYKD
mech_3
(SEQ ID NO: 345)
GIPTCGETCTLGKCNTPKCTCNWPICYKN
mech_4
(SEQ ID NO: 346)
GSIPCGESCVYIPCISSIIGCSCKSKVCYKD
mela_7
(SEQ ID NO: 347)
GIPTCGETCFKGKCYTPGCSCSYPICKKN
caripe_10
(SEQ ID NO: 348)
GVIPCGESCVFIPCFSTVIGCSCKNKVCYRN
caripe_11
(SEQ ID NO: 349)
GVIPCGESCVFIPCISTVIGCSCKKKVCYRN
caripe_12
(SEQ ID NO: 350)
GVIPCGESCVFIPCFSSVIGCSCKNKVCYRN
caripe_13
(SEQ ID NO: 351)
GIPCGESCVFIPCFTSVFGCSCKDKVCYRN

Any one and/or multiples of base cyclotide sequences can be used within the methods of the current disclosure and/or as components of the compositions within the scope of the current disclosure. Additional exemplary base cyclotide sequences of the disclosure for use in the methods herein are presented in Table 3 below.

TABLE 3
Additional Base Cyclotide Sequences

| | |
|---|---|
| 2x (peptide bond formation between terminals) | C F N G G S S G G S S H H H H H H H H D |
| can be with or without CFN | D G G G G V V C G P G K K I I L L Q Q R R |
| | C G R R R R D D S S D D C G P G G G A A |
| | C G I I C G R R G G N N G G Y Y C G G S |
| | (SEQ ID NO: 29) |
| 3x (peptide bond formation between terminals) | C F N G G G S S S G G G S S S H H H H H H |
| can be with or without CFN | H H D D D G G G G G G V V V C G G P G G |
| | K K K I I I L L L Q Q Q R R R C R R R R R |
| | R D D D S S S D D D C G G P G G A A A C |
| | G G I I I G C G R R R G G G N N N G G G Y |
| | Y Y G G C G S (SEQ ID NO: 30) |
| 4x (peptide bond formation between terminals) | C F N G A G E S G A G E S C G G G G S P G |
| can be with or without CFN | G K K K I I I L L L Q Q Q R R R G A G E S |
| | C G G G G S R R R R R R D D D S S S D D |
| | D G A G E S C G G G G S P G G G G G A A |
| | A G A G E S C G G G G S I I I G A G E S C |
| | G G G G S R R R G G G N N N G G G Y Y Y |
| | G A G E S C G G G G S G G G G S G G G S |
| | S S G G G S S S H H H H H H H H G G G G |
| | G G V V V G S (SEQ ID NO: 31) |

Defensins are also expressly contemplated for use in the methods and/or as components of the compositions of the instant disclosure, with exemplary defensin sequences include the following:

RTD-1:
(SEQ ID NO: 352)
RCICTRGFCRCLCRRGVC
RTD-2:
(SEQ ID NO: 353)
GVCRCLCRRGVCRCLCRR
RTD-3:
(SEQ ID NO: 354)
GFCRCICTRGFCRCICTR
RTD-1a:
(SEQ ID NO: 355)
MRTFALLTAIVILLLVALHAQAEARQARADEAAAQQQPGTDDQGMAHSFTW
PENAALPLSESAKGLRCICTRGFCRLL
RTD-lb:
(SEQ ID NO: 356)
MRTFALLTAIVILLLVALHAQAEARQARADEAAAQQQPGADDQGMAHSFTR
PENAALPLSESARGLRCLCRRGVCQLL
BTD-a:
(SEQ ID NO: 357)
RCVCTRGF
BTD-b:
(SEQ ID NO: 358)
RCVCRRGVC
BTD-c:
(SEQ ID NO: 359)
RCICLLGIC
BTD-d:
(SEQ ID NO: 360)
RCFCRRGVC

It is expressly contemplated that the methods of the current disclosure can be applied to any natural and/or “base” cyclotide sequence, as well as to any natural and/or “base” defensin sequence.

### Cyclotide Geometry

In certain embodiments, the present invention provides methods and compositions for producing improved cyclotides. In particular, the methods of the present invention allow for the design and generation of cyclotides with modified loop architecture for increased insert capacity. Furthermore, modified loops with greater insert capacity possess improved drug delivery attributes.

Cyclic peptides are extremely stable under harsh conditions such as high acidity gastrointestinal conditions and when exposed to proteases that degrade exposed C- or N-termini of linear peptides. It would be useful to exploit this stability to use cyclic peptides to deliver therapeutic agents orally. However, many of the known stable cyclic peptides are highly conserved and small, such as cyclotides, which has heretofore limited their use as vehicles for therapeutics due to the loss of structure upon introduction of the heterologous therapeutic. In certain aspects, the current disclosure provides processes and peptides capable of expanding the size of natural cyclic peptides such that a wide range of small to medium therapeutics can be encoded within the cyclic loops of such cyclic peptides. Further encoding of specific proteases within such cyclic peptide structures can also allow for the selective release of linear peptides.

In some embodiments, loop size of multiple and/or all loops of a “base” cyclotide sequence is doubled in the resultant cyclotide of the disclosure, as compared to the loop sizes of the “base” cyclotide. In some embodiments, doubling the loop size allows for insertion of longer sequences in loop 6 by maintaining relative proportionality of loops allowing for proper disulfide bridge formation. In some embodiments, amino acid composition is also maintained within the loops during loop expansion, optionally via expansion using duplication of “base” cyclotide loop sequences.

Within the current disclosure, a number of parameters were identified and considered relevant in designing a cyclotide library comprising various different types of cyclotide structures (e.g., “2×”, “3×”, “4×”, “P-2”, “P-3”, “P-4”, etc., as described further herein). Cysteine residues and proper disulfide bond formation were considered along with the number of cysteines, their variability and location. Loop proportionality was newly considered herein during cyclotide design. The amino acid composition (including variability and flexibility) of the loops themselves were also considered. The geometry of desired cyclotides has also been considered, such as proportionally expanding the loops versus generating polygonal structures with repeating cyclotide units. “P-2” and “2×” series cyclotides as described herein are novel structures possessing an increased carrier (insert) capacity of up to 53 amino acids or longer in length. The resulting structures as set forth herein possess significantly high stability in human plasma, consistent with a cyclic peptide structure being maintained even after performing such large-scale expansion of canonical/base cyclotide sequences.

### Multi-Loop-Expanded Cyclotide Structures

Some aspects of the current disclosure identify proportionate scaling of naturally occurring peptides as a remarkably effective approach for improving the insert capacity of naturally-occurring and/or “base” cyclotide sequences. Such approaches provide 2×, 3×, 4×, etc. variants that topologically resemble naturally-occurring cyclotides but possess greater capacity for including encoded linear peptides within one or more of their loops.

While the currently exemplified approaches relate to loop expansion performed upon canonical cyclotide structures (those possessing six cysteine residues, having three disulfide linkages and six loop regions as defined by the placement of the Cys-Cys linkages), it is also contemplated that the current loop expansion approaches set forth herein can also be applied to cyclic peptides possessing as few as two Cys-Cys linkages (i.e., thereby resulting in a cyclic peptide structure possessing four loops) and/or that the currently described approaches could be applied to cyclic peptide structures possessing four or more Cys-Cys linkages (i.e., which would thereby create a cyclic peptide structure possessing eight or more loops). Indeed, it is presently contemplated that cyclic peptides possessing more than 3 bonds can be engineered and are expected to function as cyclotides as described herein as known in the art. Engineering of such cyclic peptides (including those with four or more Cys-Cys linkages) with desired folding patterns is contemplated as making insertion of peptides, e.g., therapeutic peptides (optionally a polypeptide drug of 22-50 or more amino acids in length, an antibody molecule or fragment, optionally a monoclonal antibody, single domain antibodies such as camelid or cartilaginous fish antibody, scFv, antibody fragment such as Fv, Fab, Fab′ and F(ab)₂ fragments, and other fragments, and/or a small molecule, optionally a small molecule attached to the cyclic peptide via a non-canonical amino acid and/or linker) easier and more controlled. It is also noted that while the exemplified multi-loop-expanded cyclotide structures are proportionately expanded on all loops, it is expressly contemplated that retention of cyclotide properties (e.g., protease resistance, advantages for delivery vehicle development, etc.) could also be achieved via proportionate expansion of as few as two of six loops of a cyclotide sequence. Thus, in certain embodiments, two loops of a base cyclotide are proportionately extended (e.g., one containing an insert peptide and another extended in proportion to the length of extension performed upon the insert-containing loop), or optionally three or more loops of a base cyclotide are proportionately expanded, or optionally four or more loops of a base cyclotide are proportionately expanded, or optionally five or more loops of a base cyclotide are proportionately expanded, thereby resulting in a multi-loop-expanded cyclotide of the current disclosure, which retains at least one cyclotide attribute (e.g., protease resistance, heat stability, etc.).

One exemplary means by which an individual loop sequence of a cyclotide of the instant disclosure can be extended is via residue-by-residue duplication of adjacent amino acids of the base loop sequence. For example, in implementing such an approach upon a base loop 2 cyclotide sequence of RRDSD of MCoTI-II, duplication of each residue in series results in an expanded loop 2 sequence of RRRRDDSSDD, which can optionally also be flanked by an additional glycine (G) residue in certain exemplary “2×” expanded MCoTI-II structures (e.g., where loop 2 sequence is ultimately GRRRRDDSSDD, with the additional G residue included, e.g., for steric reasons). In the exemplified such embodiments, the (stabilizing) expanded loop 2 sequence is considered to include a total insert sequence of five or six amino acid residues (depending upon whether a G residue has also been inserted)—thus, the insert sequence within such embodiments can be interspersed between a number of native amino acid residues of loop 2, rather than requiring insertion of such an insert sequence as a linear sequence between two and only two adjacent residues of the native (base) cyclic peptide loop sequence. In related embodiments, stabilizing insertion sequences of any of loops 1-5 of a cyclotide are assessed to contain an insert sequence of a total length that is simply represented by taking the total length of the loop after inclusion of the insert-containing sequence into the loop and subtracting the original total length of the corresponding “base” loop sequence (lacking the insert sequence/pre-insertion). Alternatively, in a subset of embodiments, an insert sequence is required to be a linear (e.g., exogenous) sequence that is inserted between two and only two adjacent amino acid residues of the “base” loop sequence (pre-insertion).

### Polygonal Cyclotide Structures

Certain aspects of the current disclosure provide for linking of cyclic peptides in a polygonal series, e.g., where each vertex is occupied by a cyclotide and the edges of the polygon can optionally be encoded with linear peptides. The polygonal series of the current disclosure starts with two cyclotides that are joined by linking arms but that are still topologically a circular peptide; and it is further contemplated that the polygonal series can be extended to a triangle (3 cyclotides), a square (4 cyclotides), and so on. This greatly expanded encoding capacity for peptides can be used to deliver high value linear peptides that would otherwise be easily degraded, such as defensins, which could be used to regulate and tailor microbiome populations.

Certain currently envisioned uses for the polygonal cyclotides of the disclosure include scaffold molecules for delivery of peptides, e.g., therapeutic peptides (optionally a polypeptide drug of 22-50 or more amino acids in length), an antibody molecule or fragment, optionally a monoclonal antibody, single domain antibodies such as camelid or cartilaginous fish antibody, scFv, antibody fragment such as F′-v Fab, Fab′ and F(ab′)₂ fragments and other fragments, and/or a small molecule, optionally a small molecule attached to the cyclic peptide via a non-canonical amino acid and/or linker, in a highly stable, optionally orally administered form, the encoding of bi-functional molecules with two or more linear peptides, and the selective release of linear anti-microbial peptides in the proximity of microbial pathogens by encoding proteases on either side of the linear peptide, and the replacement of naturally produced defensins as a therapy.

### Linear Representation of Cyclic Peptides

As will be recognized by the skilled artisan, for purpose of listing specific cyclic peptide sequences, such cyclic peptide sequences must necessarily be presented in linear fashion. Where such peptide sequences are indeed indicated as cyclic peptides, these linear sequences should be considered to be continuous sequences, subject to the prospect of, in certain embodiments, encompassing disruptions of the continuous nature of such sequences as noted in the section immediately below.

### Non-Cyclic “Cyclic” Peptides

While the instant disclosure exemplifies certain methods and compositions upon peptide sequences that are entirely cyclic (i.e., having a continuous amino acid sequence), as a skilled artisan would recognize, the instant methods and compositions can be applied to peptides that are technically non-cyclic (due, e.g. to one or more disruptions in one or more loop sequences), yet such technically non-cyclic peptides retain characteristics of the a corresponding cyclic peptide from which such a technically non-cyclic peptide derives. Thus, in certain embodiments, the skilled artisan would also understand that the engineered cyclic peptides of the instant disclosure, and corresponding methods, allow for the removal of one or more internal peptide bonds as may be engineered or form naturally in the engineered cyclic peptide, without significantly disrupting the stability and/or activity of the engineered cyclotide (e.g., without significantly disrupting the trypsin resistance of the engineered cyclotide). Alpha-defensin is contemplated as an example of such a technically non-cyclic peptide that possesses sufficiently cyclic peptide characteristics to still qualify in certain embodiments as a cyclic peptide. In certain embodiments, such technically non-cyclic peptides can be referred to as “substantially cyclic” peptides, and are characterized as having, e.g., a cleavage and/or gap within one or more loop sequences that disrupts the otherwise continuous sequence of the substantially cyclic peptide (e.g., a substantially cyclic peptide that otherwise maintains (or substantially maintains) the structure of a corresponding cyclic peptide from which it derives and/or retains (or substantially retains) one or more functions (e.g., stability, activity, etc.) of a corresponding cyclic peptide from which it derives. Thus, in certain embodiments, the term “cyclic peptide” also encompasses such “substantially cyclic” peptides that are technically non-cyclic due to, e.g., cleavage/disruption of a loop sequence. Alternatively, in some embodiments, the term “cyclic peptide” refers to a peptide sequence configuration that has no such disruptions of the linear (though continuous/circular) peptide sequence.

### Charge Bias in Cyclic Peptides

It is expressly contemplated that the methods and compositions of the instant disclosure can be implemented while designing cyclic peptide sequences that alter the native charge distribution of a base cyclotide sequence, e.g., to achieve a weighting of charges, e.g., in loops 1 and/or 2, which can, in certain instances, result in enhanced cyclic peptide cell penetration properties. Such approaches are set forth in additional detail in Huang et al. (Frontiers in Pharmacology 6 (Article 17): 1-7), which is expressly incorporated herein by reference in its entirety. In certain embodiments, loop 1 and/or loop 2 of a base cyclotide sequence is not only extended via any method of extending such loop sequences, but charged residues (e.g., arginines, lysines, etc) can be included within such loops (optionally substituted for native residues of such loops), to create a charge bias within such loops. Without wishing to be bound by theory, where loops 1 and/or 2 are biased towards highly charged residues, while residues of, e.g., loop 6 are relatively non-charged, opposing faces of the engineered cyclotide are rendered respectively hydrophilic (charged) and hydrophobic (non-charged), which can impart enhanced cell penetration properties to such charge-biased cyclotide structures.

### Cyclotide Expression and Purification

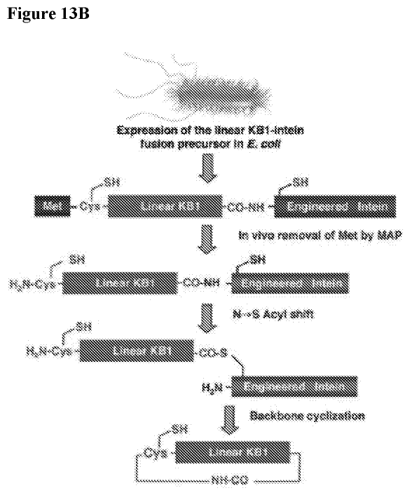

Cyclotide expression and purification can be performed via art-recognized methods. Exemplary approaches for expressing and purifying cyclotides from bacteria are presented in FIGS. 13A and 13B. FIG. 13A specifically depicts a bacterial system for expressing and cyclizing cyclotides in E. coli in vivo (Camarero et al. Chembiochem. 8: 1363-66). FIG. 13B depicts an approach for expressing and cyclizing the cyclotide in bacterial cytoplasm in vitro. Yeast and bacterial cell free droplet systems can also be employed for cyclotide production (Kimura et al. Angew Chem Int Ed Engl 45: 973-76).

### Cyclic Peptide Cyclization Systems

A variety of systems can be used to produce cyclic peptides, including the above-referenced expression systems (e.g., in yeast and/or bacteria). In vitro cyclic peptide synthesis is available and is expected to provide particular advantages for certain embodiments of the disclosure. In particular, in vitro production of cyclotides can be performed as described for cyclotide Kalata B1 in Kimura et al. (Angew. Chem. Int. Ed. 45: 973-976). In the Kimura et al. approach, a precursor is expressed in E. coli, and the precursor is enriched via use of an epitope tag. This precursor is then subjected to in vitro reactions for cyclization and disulfide bond formation.

In certain embodiments, it is expressly contemplated that all peptide synthesis (including, e.g., precursor peptides) can be performed in in vitro systems, including, e.g., in cell-free extracts. One advantage of such in vitro systems is “[t]he ability to site-specifically incorporate non-canonical amino acids (ncAAs) into proteins” (Gan et al. Biotechnol. Bioeng. 114: 1074-1086). Inclusion of ncAAs, such as p-azido-phenylalanine (pAzF), can provide non-natural moieties within primary peptide sequences, to which attachment of other moieties (e.g., small molecules, optionally therapeutic small molecule payloads protected by the surrounding cyclic peptide of the disclosure). For azide-presenting ncAAs, as is known in the art, an azide labelled cyclic peptide can be purified out of a reaction by using an alkyne labelled bead (via a click chemistry reaction). Such advantages of in vitro protein synthesis systems (e.g., acting as a lexicographer at the level of primary sequence to achieve residue-by-residue specificity of incorporation of non-natural amino acids, thereby providing non-natural moieties for attachment of payload molecules, linkers (including scaffolding linkers), etc.).

### Cleavable Moieties in Cyclic Peptides (Including Diagnostic Use)

Cyclic peptides of the disclosure can be designed to protect a payload (such as a therapeutic peptide, a small molecule, an antibody or antibody fragment, etc.), yet it is also contemplated that expanded loop sequences of the cyclic peptides of the disclosure can also include cleavable moieties, thereby enabling release under appropriate conditions for cleavage of such cleavable moieties in the vicinity of such cyclic peptides. Cleavage of such cleavable moieties can occur in a manner as contemplated in the art, including, e.g., protease cleavage, gamma ray induced cleavage, UV-induced cleavage, etc.

In one embodiment, it is expressly contemplated that a cyclic peptide including a cleavable moiety and optionally a tag (e.g., a DNA tag), an epitope or other readily detectable moiety is made and administered to a subject (e.g., in oral form, as the cyclic peptide of the disclosure is optionally resistant to enzymes of the GI tract), with cleavage of the cleavable moiety then occurring in the subject, resulting in release of the readily detectable moiety, ultimately allowing for detection of the released detectable moiety. In certain embodiments, such a cleavable moiety is a recognition site for, e.g., a liver enzyme, with the detectable moiety optionally released into the urine of a subject, in which the detectable moiety can then be detected. Proteases secreted by a pathogen can release antimicrobial peptides from the cyclic peptide scaffold resulting in self-killing of the bug. Proteases present in human blood can be used to release peptides from the scaffold and release into blood.

### Cyclic Peptide Development Process

In certain aspects of the disclosure, variant cyclic peptides are designed and produced, optionally using high-throughput genetic assembly. Testing for stability and functionality of cyclic peptide variants can then be performed via high throughput screening.

### Therapeutic Peptides

In certain embodiments, the present disclosure provides methods of drug delivery via use of cyclic peptides possessing modified loop domains. Drugs delivered using the processes and/or compositions of the present disclosure can include: peptide drugs, protein drugs, antigens, enzymes, hormones, nucleoproteins, glycoproteins, lipoproteins, polypeptides, angiogenic agents, anticoagulants, fibrinolytic agents, growth factors and antibodies. Candidate therapeutic peptides for delivery within the cyclotide structures provided herein include the following:

TABLE 4
Candidate Therapeutic Peptides (for insertion into cyclotide loop regions,
optionally for insertion into the longest loop (i.e., Loop 6 in canonical cyclotides))

| | |
|---|---|
| Glucagon | His-Ser-Gln-Gly-Thr-Phe-Thr-Ser-Asp-Tyr-Ser-Lys-Tyr- |
| | Leu-Asp-Ser-Arg-Arg-Ala-Gln-Asp-Phe-Val-Gln-Trp- |
| | Leu-Met-Asn-Thr(SEQ ID NO. 32) |
| Antimicrobial peptides SMAP-29 | R G L R R L G R K I A H G V K K Y G P T V L R I I R |
| (have release designs with protease | I A G(SEQ ID NO. 33) |
| sites on either side) | |
| Glucagon-like peptide-1 | DEFERHAEGTFTSDVSSYLEGQAAKEFIAWLVKGR; |
| | HAEGTFTSDVSSYLEGQAAKEFIAWLVKGR (GLP-1 |
| | (7-36) sequence)(SEQ ID NO 361) |

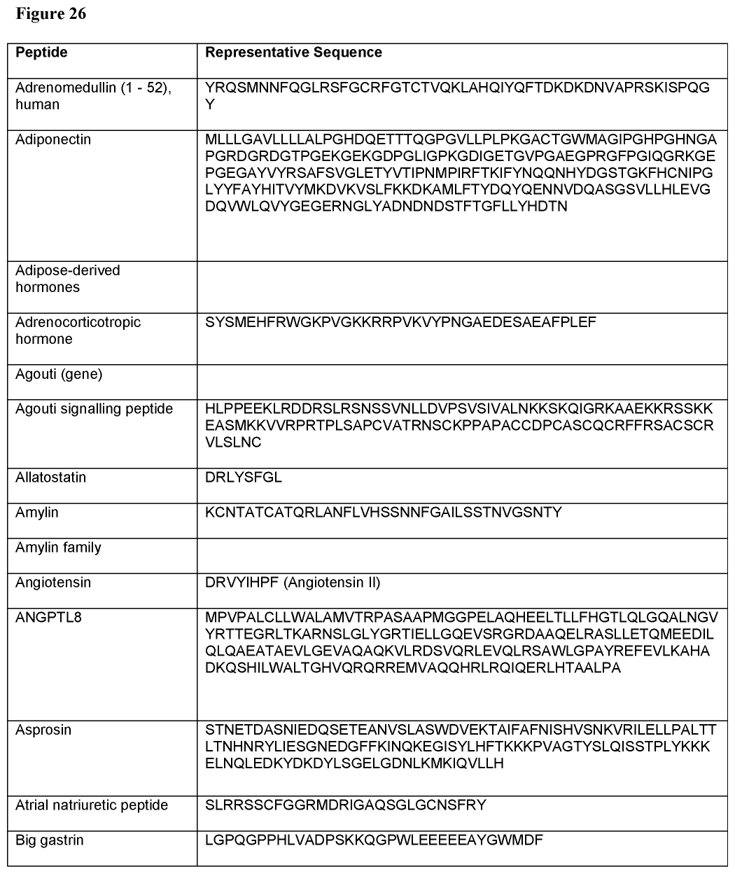

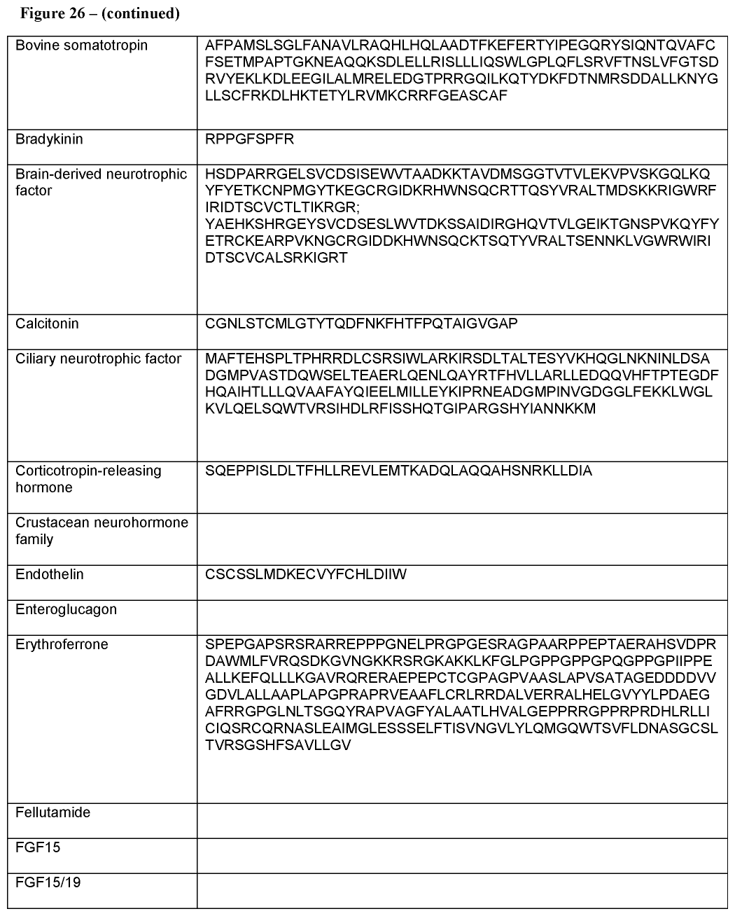

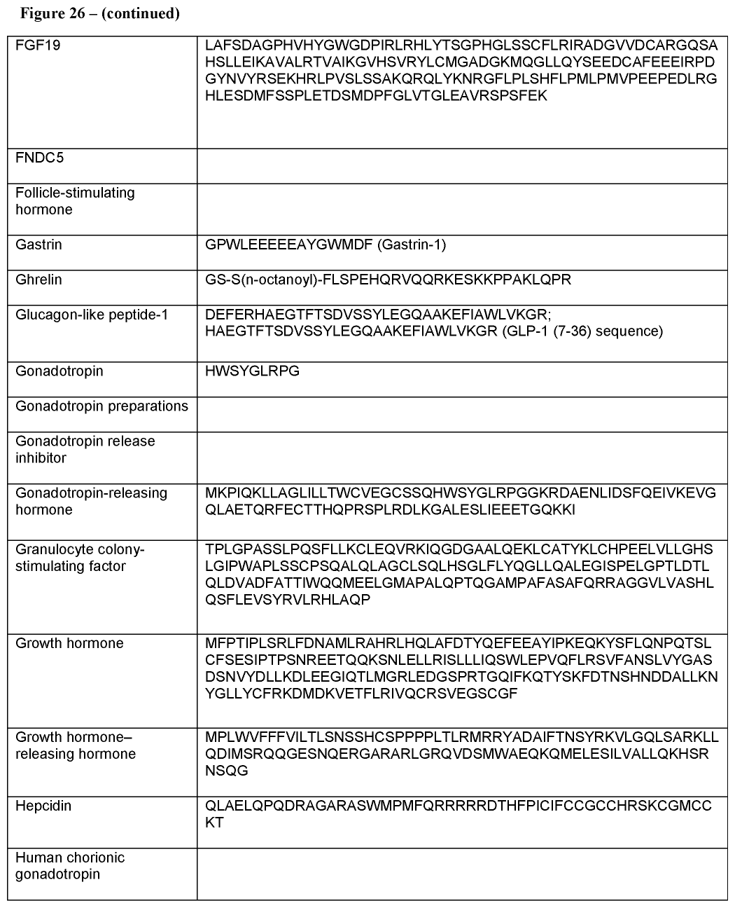

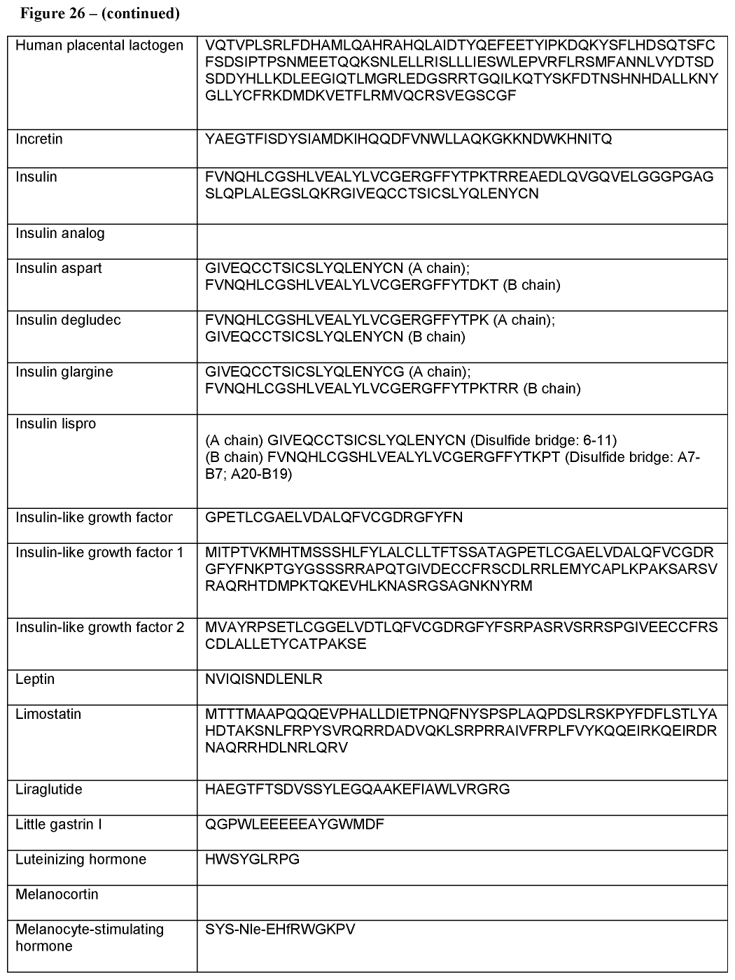

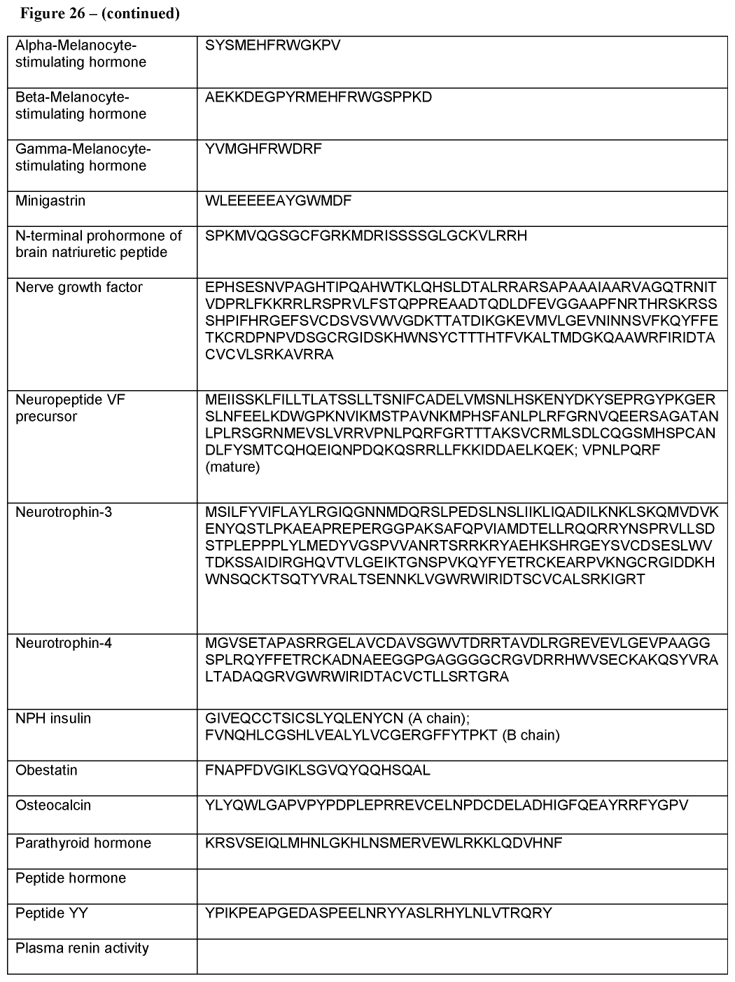

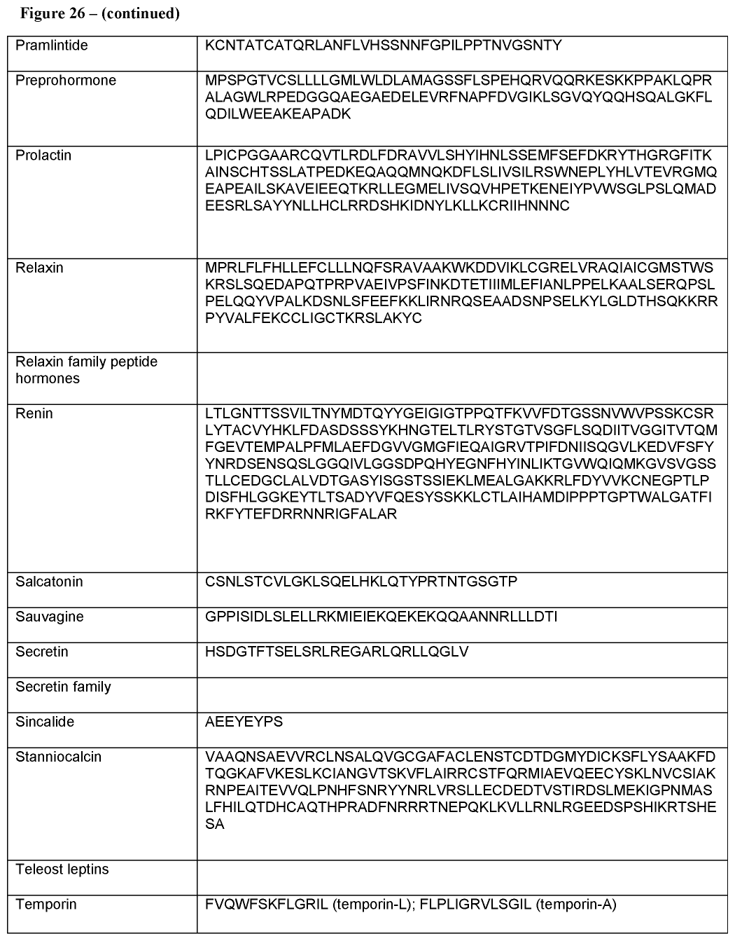

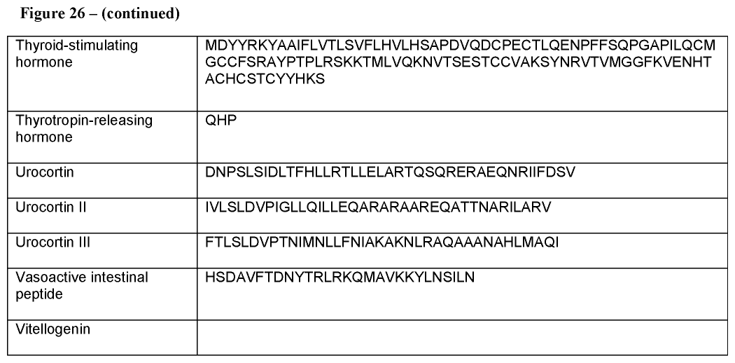

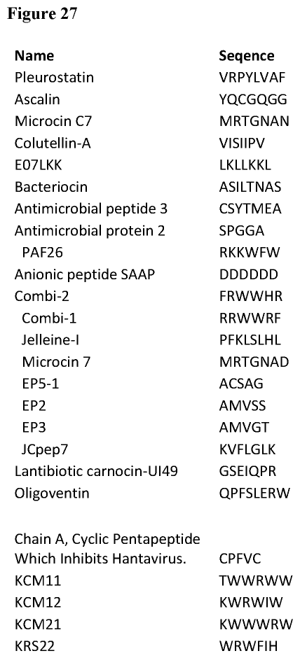

Additional examples of candidate peptides for insertion, e.g., into loop regions, are presented in FIGS. 26 and 27.

Other examples of drugs that may be delivered using the presently disclosed processes and/or compositions may include: human growth hormone, methionine-human growth hormone; des-phenylalanine human growth hormone, alpha-, beta- or gamma-interferon, erythropoietin, glucagon, calcitonin, heparin, interleukin-1, interleukin-2, Factor VIII, Factor IX, luteinizing hormone, relaxin, follicle-stimulating hormone, atrial natriuretic factor, filgrastim epidermal growth factors (EGFs), platelet-derived growth factor (PDGFs), insulin-like growth factors (IG Fs), fibroblast-growth factors (FGFs), transforming-growth factors (TGFs), interleukins (ILs), colony stimulating factors (CSFs, MCFs, GCSFs, GMCSFs), Interferons (IFNs), endothelial growth factors (VEGF, EGFs), erythropoietins (EPOs), angiopoietins (ANGs), placenta-derived growth factors (PIGFs), and hypoxia induced transcriptional regulators (HIFs). Such delivery methods and/or compositions of the disclosure can also include administration and/or co-administration of anti-infective agents such as antibiotics, antimicrobial agents, antiviral, antibacterial, antiparasitic, antifungal substances and combinations thereof, antiallergenics, androgenic steroids, decongestants, hypnotics, steroidal anti-inflammatory agents, anti-cholinergics, sympathomimetics, sedatives, miotics, psychic energizers, tranquilizers, vaccines, estrogens, progestational agents, humoral agents, prostaglandins, analgesics, antispasmodics, antimalarials, antihistamines, cardioactive agents, nonsteroidal anti-inflammatory agents, antiparkinsonian agents, antihypertensive agents, β-adrenergic blocking agents, nutritional agents, cancer therapeutics, benzophenanthridine alkaloids small molecule drugs, desensitizing materials, polynucleotides, polysaccharides, steroids, analgesics, local anesthetics, antibiotic agents, chemotherapeutic agents, immunosuppressive agents, anti-inflammatory agents, antiproliferative agents, antimitotic agents and ocular drugs, and metabolites, analogs, derivatives, fragments, and purified, isolated, recombinant and chemically synthesized versions of these species.

### Delivery of Polypeptides/Cyclotides

In certain embodiments the present invention relates to a method for treating a subject having a disease or disorder, or at risk of developing a disease or disorder, for which administration of a polypeptide is contemplated to provide prophylactic and/or therapeutic effect. In such embodiments, a polypeptide of the disclosure can act as a novel therapeutic agent for controlling the disease or disorder. The method comprises administering a pharmaceutical composition as disclosed herein to the patient (e.g., human), such that a therapeutic effect of such administration is observed. Because of their high stability, the cyclotide agents of the current disclosure, when therapeutic polypeptides are included, can be administered via a number of delivery routes to cells and tissues of a subject, to prophylactic and/or therapeutic advantage.

Therapeutic use of the cyclotide and/or cyclotide-derived agents of the instant disclosure can involve use of formulations of cyclotide agents, optionally comprising multiple different insert polypeptide sequences, within a single cyclotide structure or within a library of different cyclotide structures. For example, two or more, three or more, four or more, five or more, etc. of the presently described agents can be combined to produce a formulation that, e.g., targets multiple different targets associated with a disease or disorder.

Thus, the cyclotide agents of the instant disclosure, individually, or in combination or in conjunction with other drugs, can be used to treat, inhibit, reduce, or prevent a disease or disorder in a subject. For example, the cyclotide molecules can be administered to a subject or can be administered to appropriate cells evident to those skilled in the art, individually or in combination with one or more drugs under conditions suitable for the treatment.

The cyclotide agents of the instant disclosure also can be used in combination with other known treatments to treat, inhibit, reduce, or prevent a disease or disorder in a subject or organism. For example, the described molecules could be used in combination with one or more known compounds, treatments, or procedures to treat, inhibit, reduce, or prevent a disease or disorder in a subject or organism as are known in the art.

A cyclotide agent of the invention can optionally be conjugated or unconjugated to another moiety (e.g. a non-peptide moiety such as a marker and/or nucleic acid), or an organic compound (e.g., a dye, cholesterol, or the like). Modifying cyclotide agents in this way may improve cellular uptake or enhance cellular targeting activities of the resulting cyclotide agent derivative, as compared to the corresponding unconjugated cyclotide agent, are optionally useful for tracing the cyclotide agent derivative in the cell, or can further improve the stability of the cyclotide agent derivative, as compared to the corresponding unconjugated cyclotide agent.

A cyclic peptide of the invention can optionally present one or more conjugated peptides that are not components of the linear circular sequence of a cyclic peptide. For example, one or more residues of, e.g., loop 6 of certain cyclic peptides of the invention can optionally be conjugated to one or more linear peptide sequences. In certain embodiments, such conjugated linear peptides are positioned as a series of conjugated linear peptides (optionally, the conjugated linear peptides can be of identical sequence with one another, or can possess distinct sequences). Such conjugated linear peptides may also be conjugated with, e.g., spacing of one or more unconjugated amino acid residues interspersing such conjugated amino acid residues.

### Therapeutic Targets

Exemplary therapeutic targets for the cyclic peptides of the instant disclosure include G protein coupled receptors (GPCRs; e.g., via delivery of native and/or altered agonists, antagonists and/or derivatives thereof, within cyclic peptides), hormone receptors (e.g., via delivery of native and/or altered hormone peptides and/or derivatives thereof, within cyclic peptides) and microbial infections (e.g., via delivery of peptides within cyclic peptides of the instant disclosure, where the delivered peptides are capable of affecting microbial infection—e.g., via disruption of microbial infection, growth and/or other microbial processes).

Exemplary diseases and disorders associated with GPCRs include cardiovascular disorders, gastrointestinal and liver diseases, inflammatory diseases, metabolic diseases, hematological disorders, respiratory diseases, neurological disorders, urological disorders and cancer disorders. Specific diseases and disorders associated with individual GPCRs include the following:

| | Metabolic & |
|---|---|
| GPCR | Cardiovascular Indications |
| GPR12 | Obesity, Cognitive Impairments |
| GPR21 | Obesity, Diabetes |
| GPR22 | Cardiovascular Diseases, Anxiety |
| GPR25 | Arterial Stiffness |
| GPR37L1 | Hypertension |
| GPR39 | Diabetes |
| GPR50 | Metabolic Disorders |
| GPR61 | Eating Disorders |
| GPR82 | Appetite, Body Weight |
| GPR101 | Appetite and Eating Disorders |
| GPR132 | Atherosclerosis |
| GPR146 | Dyslipidemia, Diabetes |
| GPR171 | Eating Disorders |
| GPR176 | Atherosclerosis |
| SREB1/GPR27 | Diabetes, Schizophrenia |
| | CNS |
| GPCR | Indications |
| GPR17 | Myelin Disorders, Multiple Sclerosis |
| GPR31 | Anxiety Disorders |
| GPR37 | Parkinson’s Disease |
| GPR52 | Schizophrenia |
| GPR63 | Autism |
| GPR78 | Bipolar Disorder, Schizophrenia |
| GPR139 | Motor Disorders |
| GPR151 | Cognition, Mood Disorders, Pain |
| GPR153 | Schizophrenia |
| MAS1 | Cognitive Impairments |
| MRGE | Pain |
| OPN4 | Circadian Rhythm, Sleep Disorders |
| SREB2/GPR85 | Schizophrenia, Obesity |
| SREB3/GPR173 | Schizophrenia, Obesity |
| | Oncology |
| GPCR | Indications |
| GPR19 | Melanoma, Lung Cancer |
| GPR20 | Gastro-Intestinal Stromal Tumors, |
| | Acute Myeloid Leukemia |
| GPR65 | Renal Cell Carcinoma, Ovarian Cancer, |
| | Inflammation |
| GPR68 | Ovarian Cancer, Prostate Cancer, Osteoporosis |
| GPR80 | Hepatocellular Carcinoma |
| GPR87 | Squamous Cell Carcinoma |
| GPR150 | Ovarian Cancer |
| GPR161 | Breast Cancer, Congenital |
| | Cataracts & Birth Defects |
| GPR174 | Regulatory T-Cell Modulation |
| LGR4 | Cancer Stem Cells, Bone Diseases |
| LGR5 | Cancer Stem Cells, Esophageal Adenocarcinoma |
| P2Y8 | Leukemias, Lymphomas |
| | Miscellaneous |
| GPCR | indications |
| GPR15 | HIV Enteropathy, Rheumatoid Arthritis |
| GPR32 | Acute Inflammatory Responses |
| GPR83 | Autoimmune Diseases, PTSD |
| GPR183 | Osteoporosis and EBV |
| CCRL2 | Rheumatoid Arthritis |
| LGR6 | Hair Follicle Stem Cells, Wound Repair |

GPCRs with Unknown Indications

| | |
|---|---|
| GPR45 | GPR182 |
| GPR135 | MRGF |
| GPR141 | OPN5 |
| GPR162 | |

Exemplary diseases and disorders associated with hormones and hormone receptors include the following endocrine system diseases and disorders:
Glucose homeostasis disorders, e.g., Diabetes mellitus (Type 1 Diabetes, Type 2 Diabetes, Gestational Diabetes and/or Mature Onset Diabetes of the Young), Hypoglycemia (Idiopathic hypoglycemia and/or Insulinoma) and Glucagonoma;
Thyroid disorders, e.g., Goiter, Hyperthyroidism (e.g., Graves-Basedow disease, Toxic multinodular goitre), Hypothyroidism, Thyroiditis (e.g., Hashimoto's thyroiditis), Thyroid cancer, and Thyroid hormone resistance;
Calcium homeostasis disorders and Metabolic bone disease, e.g., Parathyroid gland disorders (Primary hyperparathyroidism, Secondary hyperparathyroidism, Tertiary hyperparathyroidism, Hypoparathyroidism (e.g., Pseudohypoparathyroidism)), Osteoporosis, Osteitis deformans (Paget's disease of bone), and Rickets and osteomalacia; Pituitary gland disorders, e.g., Posterior pituitary (e.g., Diabetes insipidus), Anterior pituitary (e.g., Hypopituitarism (or Panhypopituitarism), Pituitary tumors (e.g.,
Pituitary adenomas, Prolactinoma (or Hyperprolactinemia), Acromegaly, gigantism, and Cushing's disease), and Sex hormone disorders, e.g., Disorders of sex development or intersex disorders (e.g., Hermaphroditism, Gonadal dysgenesis, Androgen insensitivity syndromes), Hypogonadism (Gonadotropin deficiency), e.g., Inherited (genetic and chromosomal) disorders (e.g., Kallmann syndrome, Klinefelter syndrome, Turner syndrome), Acquired disorders (e.g., Ovarian failure (also known as Premature Menopause) and Testicular failure), Disorders of Puberty (e.g., Delayed puberty, Precocious puberty), and Menstrual function or fertility disorders (e.g., Amenorrhea, Polycystic ovary syndrome);
Tumours of the endocrine glands not mentioned above, e.g., Multiple endocrine neoplasia (e.g., MEN type 1, MEN type 2a, MEN type 2b) and Carcinoid syndrome;
Autoimmune polyendocrine syndromes; and
Incidentaloma—an unexpected finding on diagnostic imaging, often of endocrine glands.

Exemplary microbial infections and/or associated diseases and disorders include the following infective microbes: Acinetobacter baumannii, Actinomyces israelii, Actinomyces gerencseriae and Propionibacterium propionicus, Trypanosoma brucei, HIV (Human immunodeficiency virus), Entamoeba histolytica, Anaplasma species, Angiostrongylus, Anisakis, Bacillus anthracis, Arcanobacterium haemolyticum, Junin virus, Ascaris lumbricoides, Aspergillus species, Astroviridae family, Babesia species, Bacillus cereus, multiple bacteria, List of bacterial vaginosis microbiota, Bacteroides species, Balantidium coli, Bartonella, Baylisascaris species, BK virus, Piedraia hortae, Blastocystis species, Blastomyces dermatitidis, Machupo virus, Clostridium botulinum; Note: Botulism is not an infection by Clostridium botulinum but caused by the intake of botulinum toxin, Sabiá virus, Brucella species, the bacterial family Enterobacteriaceae, usually Burkholderia cepacia and other Burkholderia species, Mycobacterium ulcerans, Caliciviridae family, Campylobacter species, usually Candida albicans and other Candida species, Intestinal disease by Capillaria philippinensis, hepatic disease by Capillaria hepatica and pulmonary disease by Capillaria aerophila, Bartonella bacilliformis, Bartonella henselae, usually Group A Streptococcus and Staphylococcus, Trypanosoma cruzi, Haemophilus ducreyi, Varicella zoster virus (VZV), Alphavirus, Chlamydia trachomatis, Chlamydophila pneumoniae, Vibrio cholerae, usually Fonsecaea pedrosoi, Batrachochytrium dendrabatidis, Clonorchis sinensis, Clostridium difficile, Coccidioides immitis and Coccidioides posadasii, Colorado tick fever virus (CTFV), usually rhinoviruses and coronaviruses, PRNP, Crimean-Congo hemorrhagic fever virus, Cryptococcus neoformans, Cryptosporidium species, usually Ancylostoma braziliense; multiple other parasites, Cyclospora cayetanensis, Taenia solium, Cytomegalovirus, Dengue viruses (DEN-1, DEN-2, DEN-3 and DEN-4)—Flaviviruses, Green algae Desmodesmus armatus, Dientamoeba fragilis, Corynebacterium diphtheriae, Diphyllobothrium, Dracunculus medinensis, Ebolavirus (EBOV), Echinococcus species, Ehrlichia species, Enterobius vermicularis, Enterococcus species, Enterovirus species, Rickettsia prowazekii, Parvovirus B19, Human herpesvirus 6 (HHV-6) and Human herpesvirus 7 (HHV-7), Fasciola hepatica and Fasciola gigantica, Fasciolopsis buski, PRNP, Filarioidea superfamily, Clostridium perfringens, multiple, Fusobacterium species, usually Clostridium perfringens; other Clostridium species, Geotrichum candidum, PRNP, Giardia lamblia, Burkholderia mallei, Gnathostoma spinigerum and Gnathostoma hispidum, Neisseria gonorrhoeae, Klebsiella granulomatis, Streptococcus pyogenes, Streptococcus agalactiae, Haemophilus influenzae, Enteroviruses, mainly Coxsackie A virus and Enterovirus 71 (EV71), Sin Nombre virus, Heartland virus, Helicobacter pylori, Escherichia coli O157:H7, 0111 and 0104:H4, Bunyaviridae family, Hepatitis A virus, Hepatitis B virus, Hepatitis C virus, Hepatitis D Virus, Hepatitis E virus, Herpes simplex virus 1 and 2 (HSV-1 and HSV-2), Histoplasma capsulatum, Ancylostoma duodenale and Necator americanus, Human bocavirus (HBoV), Ehrlichia ewingii, Anaplasma phagocytophilum, Human metapneumovirus (hMPV), Ehrlichia chaffeensis, Human papillomavirus (HPV), Human parainfluenza viruses (HPIV), Hymenolepis nana and Hymenolepis diminuta, Epstein-Barr virus (EBV), Orthomyxoviridae family, Isospora belli, unknown; evidence supports that it is infectious, multiple, Kingella kingae, PRNP, Lassa virus, Legionella pneumophila, Legionella pneumophila, Leishmania species, Mycobacterium leprae and Mycobacterium lepromatosis, Leptospira species, Listeria monocytogenes, Borrelia burgdorferi, Borrelia garinii, and Borrelia afzelii, Wuchereria bancrofti and Brugia malayi, Lymphocytic choriomeningitis virus (LCMV), Plasmodium species, Marburg virus, Measles virus, Middle East respiratory syndrome coronavirus, Burkholderia pseudomallei, multiple, Neisseria meningitidis, usually Metagonimus yokagawai, Microsporidia phylum, Molluscum contagiosum virus (MCV), Monkeypox virus, Mumps virus, Rickettsia typhi, Mycoplasma pneumoniae, numerous species of bacteria (Actinomycetoma) and fungi (Eumycetoma), parasitic dipterous fly larvae, most commonly Chlamydia trachomatis and Neisseria gonorrhoeae, (New) Variant Creutzfeldt-Jakob disease (vCJD, nvCJD), PRNP, usually Nocardia asteroides and other Nocardia species, Onchocerca volvulus, Opisthorchis viverrini and Opisthorchis felineus, Paracoccidioides brasiliensis, usually Paragonimus westermani and other Paragonimus species, Pasteurella species, Pediculus humanus capitis, Pediculus humanus corporis, Phthirus pubis, multiple, Bordetella pertussis, Yersinia pestis, Streptococcus pneumoniae, Pneumocystis jirovecii, multiple, Poliovirus, Prevotella species, usually Naegleria fowleri, JC virus, Chlamydophila psittaci, Coxiella burnetii, Rabies virus, Borrelia hermsii, Borrelia recurrentis, and other Borrelia species, Respiratory syncytial virus (RSV), Rhinosporidium seeberi, Rhinovirus, Rickettsia species, Rickettsia akari, Rift Valley fever virus, Rickettsia rickettsii, Rotavirus, Rubella virus, Salmonella species, SARS coronavirus, Sarcoptes scabiei, Schistosoma species, multiple, Shigella species, Varicella zoster virus (VZV), Variola major or Variola minor, Sporothrix schenckii, Staphylococcus species, Staphylococcus species, Strongyloides stercoralis, Measles virus, Treponema pallidum, Taenia species, Clostridium tetani, usually Trichophyton species, usually Trichophyton tonsurans, usually Trichophyton species, usually Epidermophyton floccosum, Trichophyton rubrum, and Trichophyton mentagrophytes, Trichophyton rubrum, usually Hortaea werneckii, usually Trichophyton species, usually Trichophyton species, Malassezia species, Toxocara canis or Toxocara cati, Toxocara canis or Toxocara cati, Chlamydia trachomatis, Toxoplasma gondii, Trichinella spiralis, Trichomonas vaginalis, Trichuris trichiura, usually Mycobacterium tuberculosis, Francisella tularensis, Salmonella enterica subsp. enterica, serovar typhi, Rickettsia, Ureaplasma urealyticum, Coccidioides immitis or Coccidioides posadasii, Venezuelan equine encephalitis virus, Guanarito virus, Vibrio vulnificus, Vibrio parahaemolyticus, multiple viruses, West Nile virus, Trichosporon beigelii, Yersinia pseudotuberculosis, Yersinia enterocolitica, Yellow fever virus and Mucorales order (Mucormycosis) and Entomophthorales order (Entomophthoramycosis).

### Pharmaceutical Compositions

In certain embodiments, the present disclosure provides for a pharmaceutical composition comprising the cyclotide agent of the present disclosure. The cyclotide agent sample can be suitably formulated and introduced into a subject and/or the environment of a cell by any means that allows for a sufficient portion of the sample exert an effect in the subject or cell, if it is to occur. Many formulations for peptides are known in the art and can be used. For example, the cyclotide agent of the instant disclosure can be formulated in buffer solutions such as phosphate buffered saline solutions.

Such compositions typically include the cyclotide molecule and a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions.

A pharmaceutical composition is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in a selected solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques. The materials can also be obtained commercially from Alza Corporation and Nova Pharmaceuticals, Inc. Liposomal suspensions can also be used as pharmaceutically acceptable carriers. These can be prepared according to methods known to those skilled in the art, for example, as described in U.S. Pat. No. 4,522,811.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For a compound used in the method of the disclosure, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of a cyclotide or other peptide molecule (i.e., an effective dosage) depends on the cyclotide or peptide selected. For instance, single dose amounts of a cyclotide (or, e.g., a construct(s) encoding for such cyclotide) in the range of approximately 1 pg to 1000 mg may be administered; in some embodiments, 10, 30, 100, or 1000 pg, or 10, 30, 100, or 1000 ng, or 10, 30, 100, or 1000 μg, or 10, 30, 100, or 1000 mg may be administered. In some embodiments, 1-5 g of the compositions can be administered. The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a nucleic acid (e.g., encoding a cyclotide), cyclotide, protein, polypeptide, or antibody can include a single treatment or, preferably, can include a series of treatments.

It can be appreciated that the method of introducing cyclotide into a subject and/or to the environment of a cell, will depend on the subject and/or type of cell and the make up of its environment. For example, cyclotide formulations can be administered to animals such as by intravenous, intramuscular, or intraperitoneal injection, or orally or by inhalation or other methods as are known in the art. When the formulation is suitable for administration into animals such as mammals and more specifically humans, the formulation is also pharmaceutically acceptable. Pharmaceutically acceptable formulations for administering polypeptides are known and can be used. It will be appreciated that the cyclotide formulations of the invention are well-suited for oral and inhalational administration, though are administrable to a subject via any art-known route of administration.

Suitable amounts of a cyclotide agent are introduced to a subject and these amounts can be empirically determined using standard methods.

The method can be carried out by addition of the cyclotide agent compositions to an extracellular matrix in which cells can live provided that the cyclotide agent composition is formulated so that a sufficient amount of the cyclotide agent can emerge from the matrix to exert its effect. For example, the method is amenable for use with cells present in a liquid such as a liquid culture or cell growth media, in tissue explants, or in whole organisms, including animals, such as mammals and especially humans.

The cyclotide agent can be formulated as a pharmaceutical composition which comprises a pharmacologically effective amount of a cyclotide-contained polypeptide agent and pharmaceutically acceptable carrier. A pharmacologically or therapeutically effective amount refers to that amount of a cyclotide and/or cyclotide-contained agent effective to produce the intended pharmacological, therapeutic or preventive result. The phrases “pharmacologically effective amount” and “therapeutically effective amount” or simply “effective amount” refer to that amount of a cyclotide and/or cyclotide-contained agent effective to produce the intended pharmacological, therapeutic or preventive result. For example, if a given clinical treatment is considered effective when there is at least a 20% reduction in a measurable parameter associated with a disease or disorder, a therapeutically effective amount of a drug for the treatment of that disease or disorder is the amount necessary to effect at least a 20% reduction in that parameter.

Suitably formulated pharmaceutical compositions of this disclosure can be administered by means known in the art such as by parenteral routes, including intravenous, intramuscular, intraperitoneal and subcutaneous administration. In some embodiments, the pharmaceutical compositions are administered by intravenous or intraparenteral infusion or injection.

In general, a suitable dosage unit of cyclotide will be in the range of 0.001 to 0.25 milligrams per kilogram body weight of the recipient per day, or in the range of 0.01 to 20 micrograms per kilogram body weight per day, or in the range of 0.001 to 5 micrograms per kilogram of body weight per day, or in the range of 1 to 500 nanograms per kilogram of body weight per day, or in the range of 0.01 to 10 micrograms per kilogram body weight per day, or in the range of 0.10 to 5 micrograms per kilogram body weight per day, or in the range of 0.1 to 2.5 micrograms per kilogram body weight per day. A pharmaceutical composition comprising the cyclotide can be administered once daily. However, the therapeutic agent may also be dosed in dosage units containing two, three, four, five, six or more sub-doses administered at appropriate intervals throughout the day. In that case, the cyclotide contained in each sub-dose is correspondingly smaller in order to achieve the total daily dosage unit. The dosage unit can also be compounded for a single dose over several days, e.g., using a conventional sustained release formulation which provides sustained and consistent release of the cyclotide over a several day period. Sustained release formulations are well known in the art. In this embodiment, the dosage unit contains a corresponding multiple of the daily dose. Regardless of the formulation, the pharmaceutical composition of such embodiments contains cyclotide in a quantity sufficient to exert an effect in the animal or human being treated. The composition can be compounded in such a way that the sum of the multiple units of cyclotide together contain a sufficient dose.

Data can be obtained from cell culture assays and animal studies to formulate a suitable dosage range for humans. The dosage of compositions of the disclosure lies within a range of circulating concentrations that include the ED₅₀ (as determined by known methods) with little or no toxicity. The dosage may vary within this range depending upon the dosage form employed and the route of administration utilized. For a compound used in the method of the disclosure, in certain instances, the therapeutically effective dose can be estimated initially from cell culture assays. A dose may be formulated in animal models to achieve a circulating plasma concentration range of the compound that includes the IC₅₀ (i.e., the concentration of the test compound which achieves a half-maximal inhibition of symptoms) as determined in cell culture. Such information can be used to more accurately determine useful doses in humans. Levels of cyclotide in plasma may be measured by standard methods, for example, by high performance liquid chromatography.

The pharmaceutical compositions can be included in a kit, container, pack, or dispenser together with instructions for administration.

### Methods of Treatment

The present disclosure provides for both prophylactic and therapeutic methods of treating a subject at risk of (or susceptible to) a disease or disorder.

In certain aspects, the disclosure provides a method for preventing in a subject, a disease or disorder as described herein, by administering to the subject a therapeutic agent (e.g., a cyclotide molecule comprising an insert polypeptide, or vector or transgene encoding same). Subjects at risk for the disease can be identified by, for example, one or a combination of diagnostic or prognostic assays known in the art. Administration of a prophylactic agent can occur prior to the detection of, e.g., a disease or disorder in a subject, or the manifestation of symptoms characteristic of the disease or disorder, such that the disease or disorder is prevented or, alternatively, delayed in its progression.

Another aspect of the disclosure pertains to methods of treating subjects therapeutically, i.e., altering the onset of symptoms of the disease or disorder. These methods can be performed in vitro (e.g., by culturing the cell with the cyclotide agent) or, alternatively, in vivo (e.g., by administering the cyclotide agent to a subject).

The practice of the present invention employs, unless otherwise indicated, conventional techniques of chemistry, molecular biology, microbiology, recombinant DNA, genetics, immunology, cell biology, cell culture and transgenic biology, which are within the skill of the art. See, e.g., Maniatis et al., 1982, Molecular Cloning (Cold Spring Harbor Laboratory Press, Cold Spring Harbor, N.Y.); Sambrook et al., 1989, Molecular Cloning, 2nd Ed. (Cold Spring Harbor Laboratory Press, Cold Spring Harbor, N.Y.); Sambrook and Russell, 2001, Molecular Cloning, 3rd Ed. (Cold Spring Harbor Laboratory Press, Cold Spring Harbor, N.Y.); Ausubel et al., 1992), Current Protocols in Molecular Biology (John Wiley & Sons, including periodic updates); Glover, 1985, DNA Cloning (IRL Press, Oxford); Anand, 1992; Guthrie and Fink, 1991; Harlow and Lane, 1988, Antibodies, (Cold Spring Harbor Laboratory Press, Cold Spring Harbor, N.Y.); Jakoby and Pastan, 1979; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Riott, Essential Immunology, 6th Edition, Blackwell Scientific Publications, Oxford, 1988; Hogan et al., Manipulating the Mouse Embryo, (Cold Spring Harbor Laboratory Press, Cold Spring Harbor, N.Y., 1986); Westerfield, M., The zebrafish book. A guide for the laboratory use of zebrafish (Danio rerio), (4th Ed., Univ. of Oregon Press, Eugene, 2000).

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

This invention is further illustrated by the following examples which should not be construed as limiting.

### EXAMPLES

### Example 1: Cyclization of Native and FLAG Epitope Tagged Cyclotide

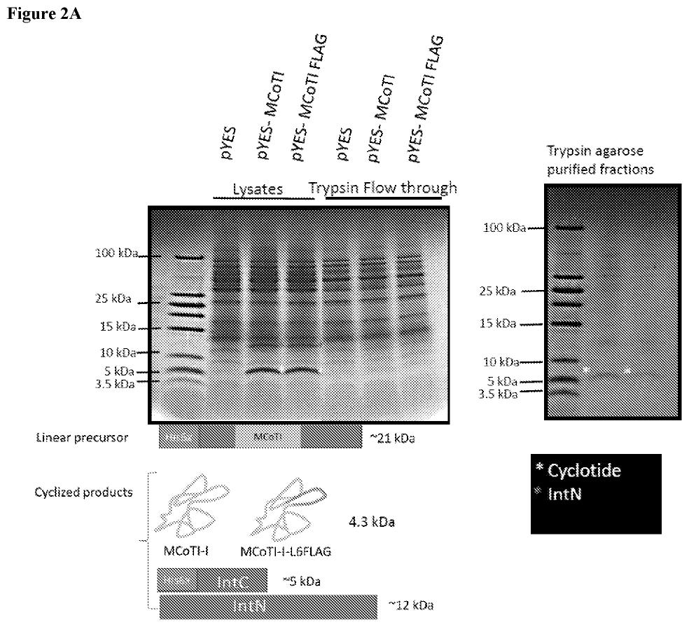

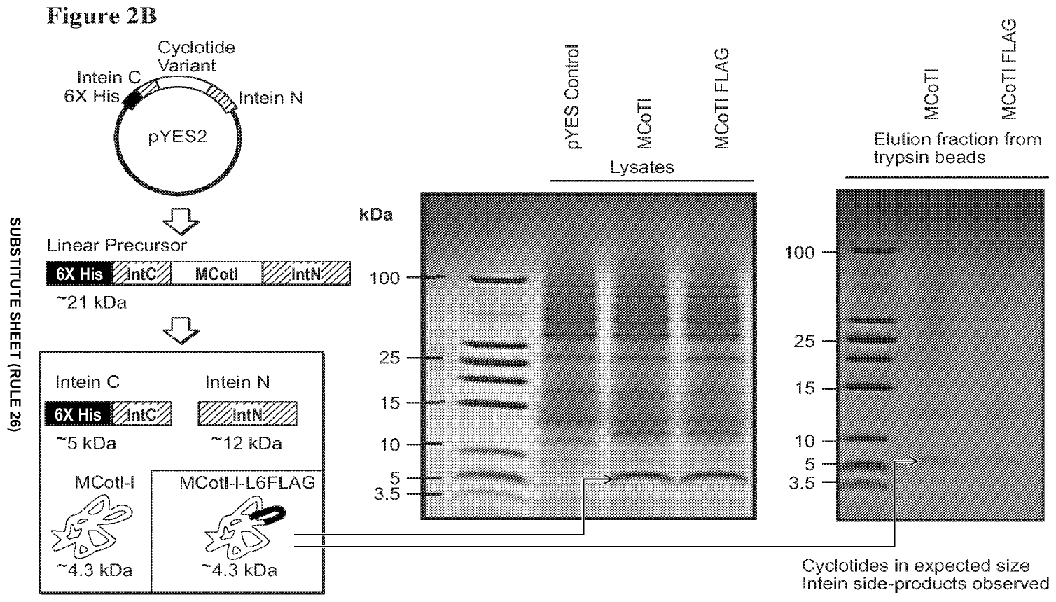

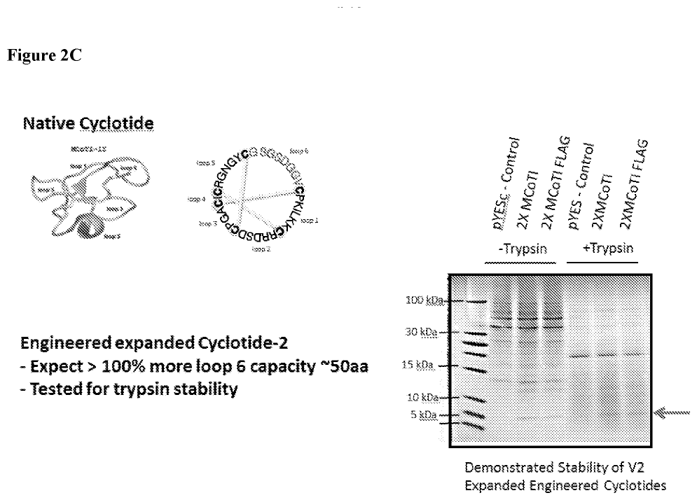

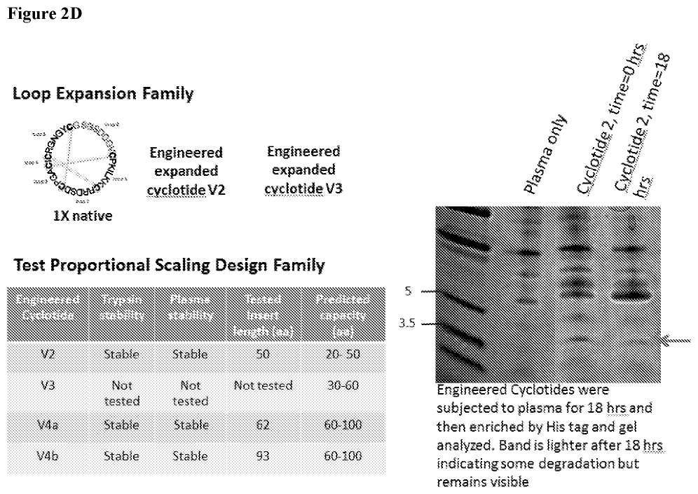

This example demonstrates the assessment of stability and cyclization of cyclotides. Native and FLAG epitope-tagged cyclotides were expressed in yeast. Sodium dodecyl sulfate polyacrylamide gel electrophoresis (SDS-PAGE) was performed on the lysates and flow throughs of empty vector (pYES), native cyclotide (pYES-MCoTI) and FLAG epitope-tagged cyclotide (pYES-MCoTI FLAG), as depicted in FIGS. 2A and 2B. FIGS. 2A and 2B depict the stained SDS-PAGE gel, as well as a breakdown of the structural organization of the different cyclized products located therein. For comparison purposes, the right-hand panel depicts trypsin agarose purified fractions of the cyclotide, aligned for cyclotide size with lysates on the SDS-PAGE gel. It was thereby demonstrated that FLAG-tagged and native cyclotides exhibited similar levels of resistance to trypsin digestion. As shown in FIG. 2C, higher insert capacity (exceeding 22 amino acids) was observed for the cyclotide designs of the disclosure, specifically demonstrating the stability of V2 expanded engineered cyclotides of the disclosure. As observed in FIG. 2D, loop expanded cyclotide designs of the disclosure were not only stable in trypsin but also stable in human serum.

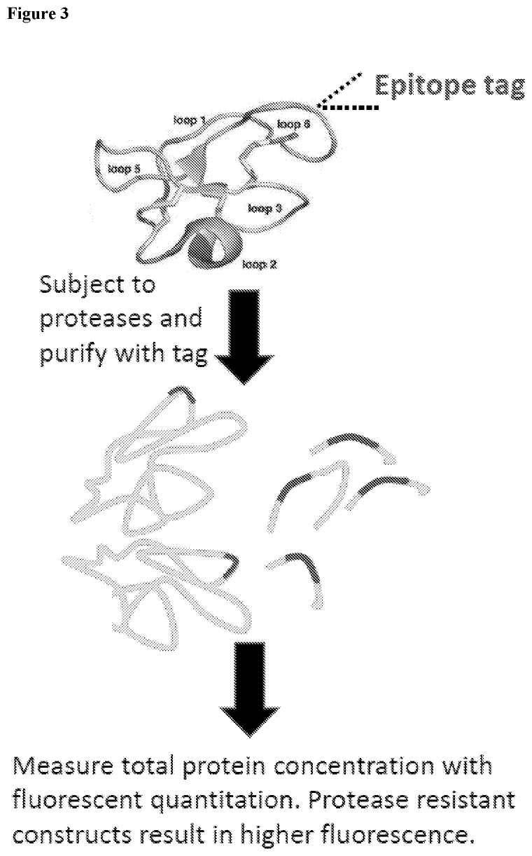

To further assess stability and/or extent of cyclization, a high throughput process for preferential detection of cyclotide sequences (here, harboring an epitope tag insert) was developed, as summarized in FIG. 3. The high throughput process was developed to enable automation of cyclization assays. Using HPLC to determine the stability and/or extent of cyclization of a cyclic peptide is time consuming (requiring approximately 30 minutes per sample), and is therefore not readily adaptable to a high throughput process. In contrast, the process outlined in FIG. 3 and described herein significantly reduces the time required to design, generate and purify cyclotides. Several cyclotides can be analyzed in bulk and the analysis time can be significantly reduced. In the exemplary process of FIG. 3, a parental cyclotide harboring a loop 6 insert (here, an epitope tag) was first designed for testing. Epitope tagged cyclotides particularly allowed for enrichment/purification of cyclotides, for various applications. Variants of the cyclotide can also be generated for analysis. Conditions under which cyclotide stability can be tested include exposure to heat, varying pH (i.e., elevated acidity) and protease digestion. Lysine residues of cyclotides and variants were fluorescently labeled. The variants and parent/“base” sequences were exposed to protease digestion to test stability. The samples were purified via binding of their epitope tags, and elutions were quantitated using a fluorescent protein quantitation protocol in plate format. Total protein concentration was measured via fluorescent quantitation. Properly cyclized protease-resistant constructs resulted in higher fluorescence signals when tested in the presence of protease (e.g., trypsin), due to the higher protein content of the epitope-containing, bound material, as depicted in FIG. 3. Development of such a high throughput stability assay for cyclotides harboring epitope tags allows for high throughput “bulk” analysis of stable cyclotide structures, such as those obtained from libraries and/or multi-loop-expanded or other candidate cyclotide sequence described herein. Analysis time can be significantly reduced using this strategy, especially as compared to traditional HPLC approaches.

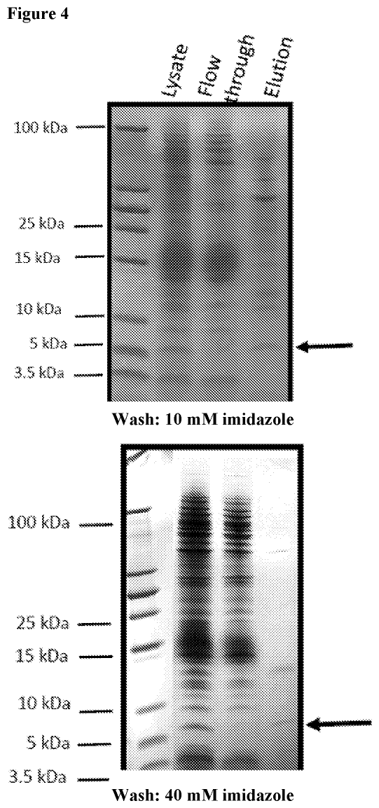

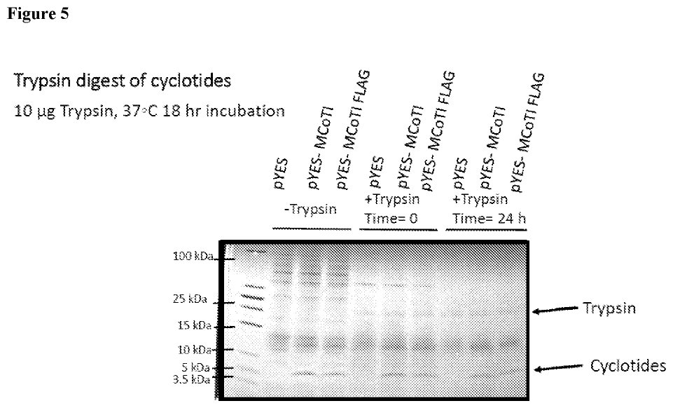

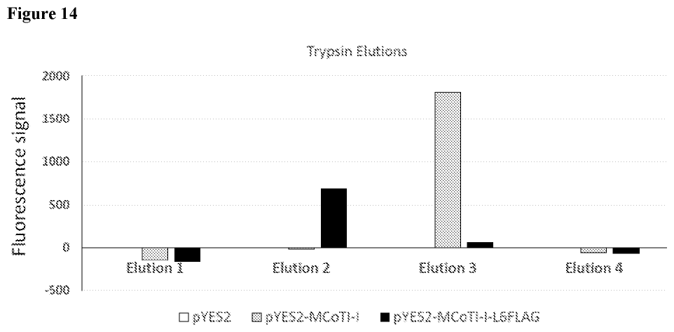

Specific testing of this novel high-throughput analysis technique involved designing and testing cyclotides that harbor a 6×His tag in loop 6. Cyclotide production was established in yeast (S. cerevisiae). Cyclotides were successfully enriched and purified by optimizing the purification with stringent washes of 10 mM and 40 mM imidazole to eliminate contaminants, as shown in FIG. 4. Protease conditions were tested on cyclotides and variants. Samples were subjected to 10 μg of trypsin at 37° C. for up to 24 hours. As FIG. 5 demonstrates, FLAG-tagged and native cyclotides were confirmed as resistant to trypsin digestion. In addition, the functionality of such cyclotides was verified and MALDI-MS analysis of these cyclotides' structure was established. Purification and quantitation of cyclotides was demonstrated using trypsin agarose beads. Four elutions were collected in 200 μL fractions. Protein content of bound/eluted material was quantified based upon detection of fluorescently labeled lysines within bound/eluted sequences. (Due to reductive amination of primary amines in cyclized proteins, the N-terminal primary amine was unavailable for fluorescent labeling in cyclotides, rendering fluorescent labeling of lysines advantageous for such assays.) Assaying for fluorescent signal, as depicted in FIG. 14, FLAG-tagged and native cyclotides were purified in such experiments in amounts of 0.820 μg and 9.20 μg, respectively.

### Example 2: Cyclotide Production in E. coli

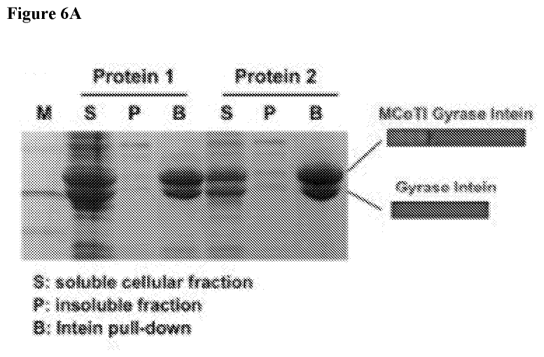

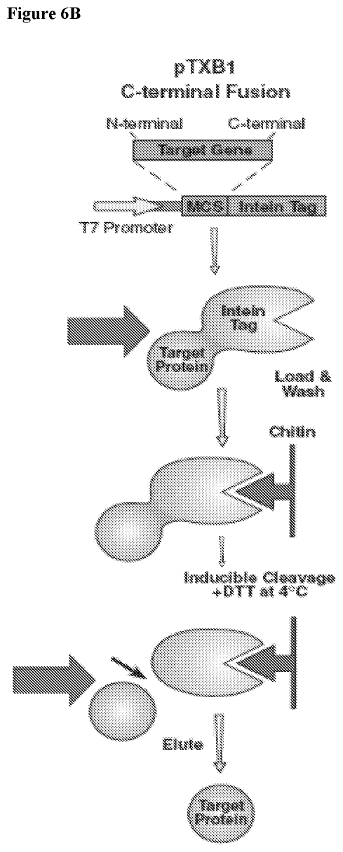

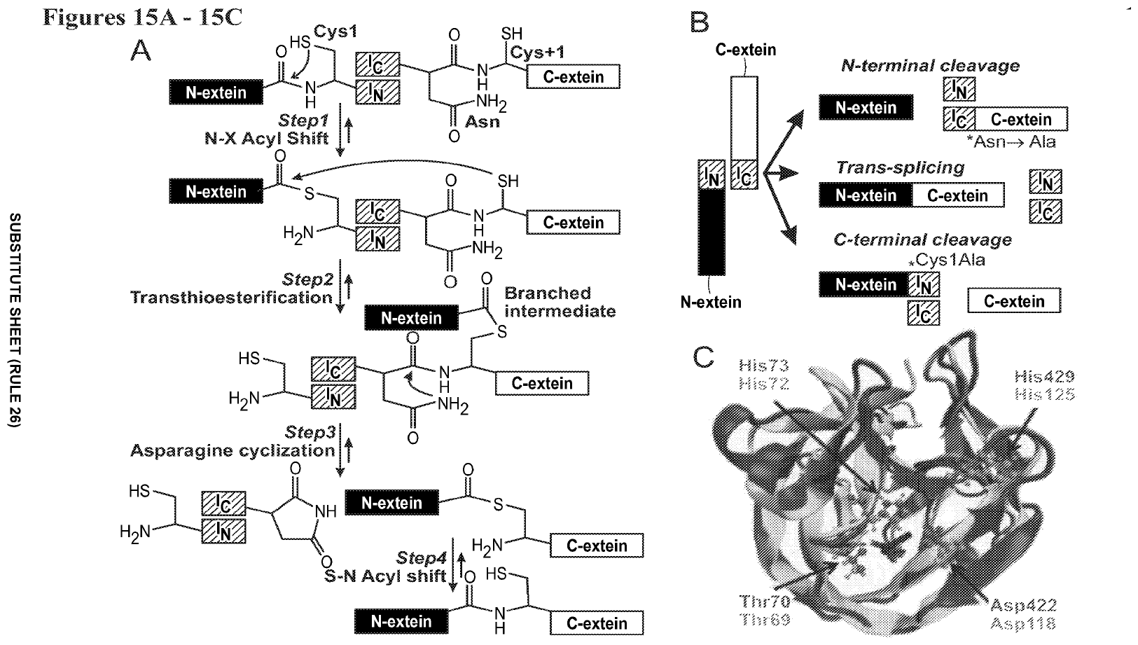

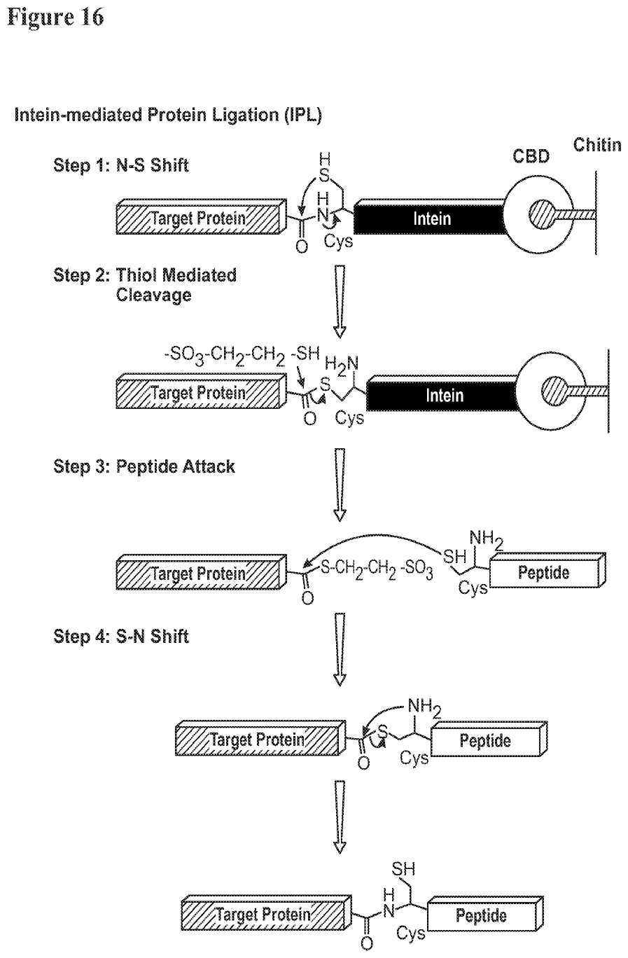

E. coli was tested as a production source for cyclotide generation. Cyclotide and/or candidate cyclotide sequences are designed and cloned into E. coli plasmids, optionally fused with a cleavable intein tag/chitin binding domain. Mechanisms of intein-mediated protein ligation from a chitin bead are depicted in FIG. 16. Further depictions of intein reaction mechanisms are shown in FIGS. 15A to 15C. After expression, purified fusion protein(s) can be washed and loaded onto a column of chitin beads. After sufficient time for chitin binding, the column-bound fusion protein is subjected to cleavage, to purify the protein of interest (i.e., a cyclotide). The cyclotide can then be cyclized in vitro, as shown in FIG. 6B.

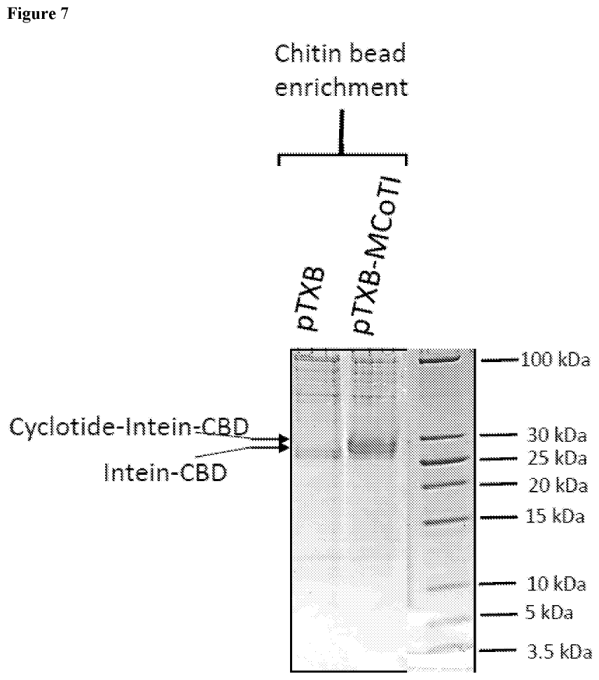

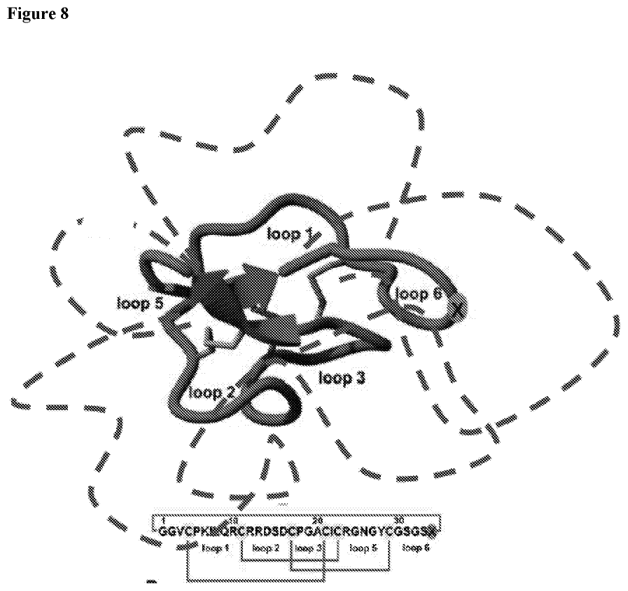

Using this approach, cyclotides were designed and cloned into the E. coli pTXB plasmid (New England Biolabs), for expression as polypeptides fused to a Gyrase (GyrA) Intein tag/chitin binding domain. E. coli BL21 and Origami 2 strains were used for expression. As shown in FIG. 6A, SDS-PAGE analysis demonstrated successful expression and in vivo cleavage of precursor (MCoTI) proteins from the Gyrase intein. After expression in E. coli, the cyclotide-intein fusion protein was loaded onto a column with chitin, which was then washed. FIG. 7 demonstrates that successful chitin bead enrichment was obtained for cyclotide-intein fusion polypeptides (pTXB-MCoTI), and not for the pTXB vector alone (empty plasmid), thereby also demonstrating that chitin beads bound to the chitin biding domain (CBD) of such intein-fused polypeptides. Following inducible cleavage and DTT exposure at 4° C., the cyclotide was then purified from the column, while the chitin bound intein tag remained attached. The cyclotide was then cyclized in vitro.

The following sequence was cloned into the Ptxb1 plasmid NEB, using NdeI and SpeI cloning sites:

(SEQ ID NO: 34)
ATATCATATGTGTGGTAGTGGCAGCGATGGAGGGGTGTGTCCGAAGATCCT
AAAGAAGTGCAGGAGAGACAGCGACTGCCCCGGCGCTTGCATCTGTCGTGG
CAATGGTTATTGCATCACGGGAGATGCACTAGTTATAT

The above DNA sequence encodes for the following polypeptide:

(cyclic)
(SEQ ID NO: 35)
CGSGSDGGVCPKILKKCRRDSDCPGACICRGNGY.

### Example 3: Design and Generation of Novel Cyclotide Structures Possessing Increased Insert Capacity

Prior to the current disclosure, the effective insert capacity of native cyclotides within loop 6 has been limited to about 20-22 amino acids—insertion of longer sequences into native cyclotides has been specifically observed to render such sequences non-cyclotide-like in their attributes/functionality. Without wishing to be bound by theory, such longer sequence insertions have been believed to cause instability in the cyclotide structure. This instability renders the loop 6-extended cyclotide structure no longer resistant to protease, acid, or heat stress, in contrast to the native cyclotide structure and/or a cyclotide structure harboring a shorter insert length. In certain aspects, the present disclosure has identified approaches for designing novel cyclotide sequences possessing increased insert capacity, optionally with polypeptide insert capacities ranging up to 50 amino acids or more in length. A cyclic peptide database (Cybase) containing the sequences of over 800 highly stable cyclic peptides has been used to design novel cyclotides. Naturally occurring cyclotide sequences exhibit limited flexibility/diversity, and possess structural limitations including: 6 cysteine residues (three Cys-Cys disulfide linkages), 6 loops, limited amino acid length, and limited amino acid sequence variability. Historically, the insert size maximum for a cyclotide has been considered to be approximately 20-22 amino acids, which is further noted as a length that has been rarely achieved in practice. Given the apparent rigidity of the natural cyclotide structure, it has seemed highly unlikely that mutating a few amino acids of any individual cyclotide would produce a “traditional” cyclotide structure more capable of harboring substantially large insertions within the structure.

Rather than rely upon, e.g., a limited series of point mutations in an attempt to achieve greater cyclotide insert capacity, it has been newly identified herein that there are effective ways to increase insert capacity of cyclotide structure(s) while still retaining cyclotide properties (i.e., protease resistance, compact structure, heat stability, acid stability, delivery properties, etc.), which require introduction of more extensive sequence alterations than simple point mutations. As described herein, doubling the size of all loop regions of a cyclotide has been newly identified as an approach that allows for insertion of remarkably longer insert sequences within loop 6 of a cyclotide structure, while retaining the functional properties of a cyclotide (protease stability, etc.). Without wishing to be bound by theory, this approach appears to provide a cyclotide structure with dramatically increased insert capacity because relative proportionality of loop sizes are maintained in the resultant “multi-loop-expanded” cyclotide structure, which allows for proper disulfide bridge formation to be maintained. Notably, amino acid composition also appears to be relevant to performing such loop extensions. Thus, doubling of loop sizes while maintaining cyclotide character and allowing for extended insert length has initially been achieved via repeating of native sequences within each loop. However, it is also contemplated that suitable extension of multiple loops of a cyclotide can also be achieved using extension sequences that simply continue to provide an appropriate structure for each loop (e.g., use of variant sequences, even possibly extensive variants, of the native cyclotide sequences that are being extended in each loop).

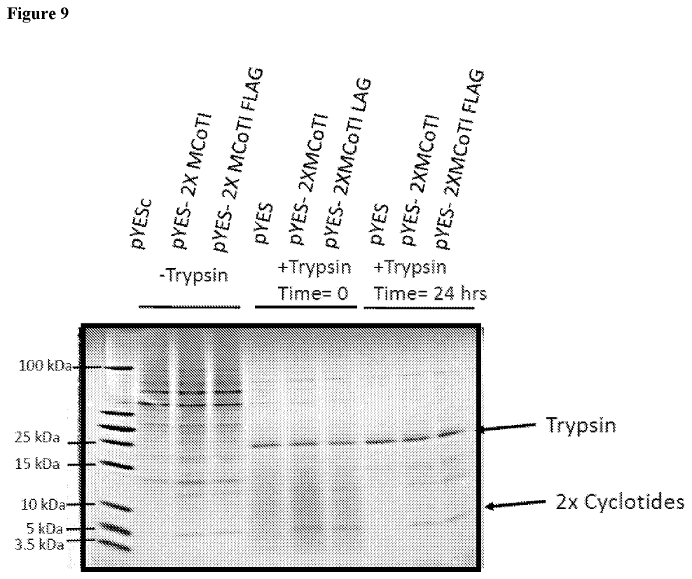

MCoTI cyclotides possessing doubled loop lengths (“2×” cyclotides) were designed and tested for protease resistance. FLAG-tagged and native 2× cyclotides were expressed in yeast and purified (pYES-2×MCoTI FLAG and pYES-2×MCoTI). The cyclotides and empty yeast vector were then subjected to 10 μg trypsin protease digestion at 37° C. for up to 24 hours. As shown in FIG. 9, the 2× cyclotides were identified as resistant to trypsin digestion.

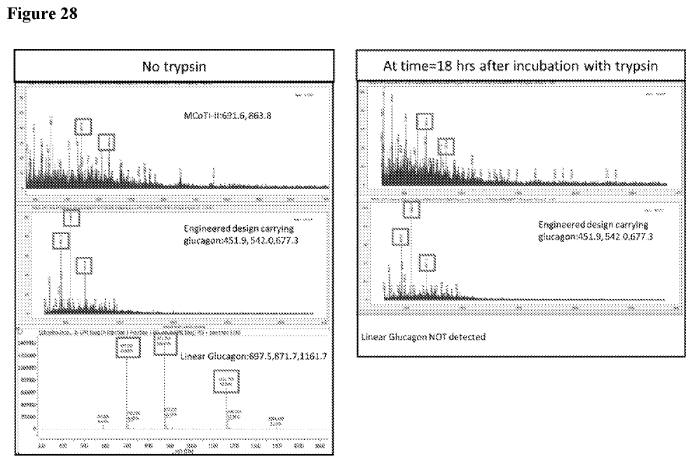

An additional, engineered “2×” cyclotide structure possessing a glucagon insert sequence in loop 6 was confirmed as stable when exposed to trypsin protease. As shown in FIG. 28, a 2× engineered MCoTI-II-glucagon cyclotide having the following sequence:

(SEQ ID NO: 362)
CGGSSGGSSHREIRHESQGTFTSDYSKYLDSRRAQDFVQWLMNTGGDDGG
GGVVCGPGKKIILLQQRRCGRRRRDDSSDDCGPGGGAACGIICGRRGGNN
GGYY

was confirmed as significantly more trypsin resistant than, e.g., a linear glucagon peptide, over a time course of up to 18 hours with no change in levels identified.

TABLE 5
Multi-Loop-Expanded Cyclotide Sequences, Exemplary

| | |
|---|---|
| P-2 (His tag can be replaced with other tags | C F N G S G S G G G S S G G G H H H H H |
| and this region would also encode | H G G G S S G S G C Y G N G R C I C A G P |
| therapeutics) can be with or without CFN | C D S D R R C R Q L I K P C V G G D S G G |
| | G G G G S H H H H H H S G G G D G G V C |
| | P K I L Q R C R R D S D C P G A C I C R G |
| | N G Y C G S (SEQ ID NO: 36) |

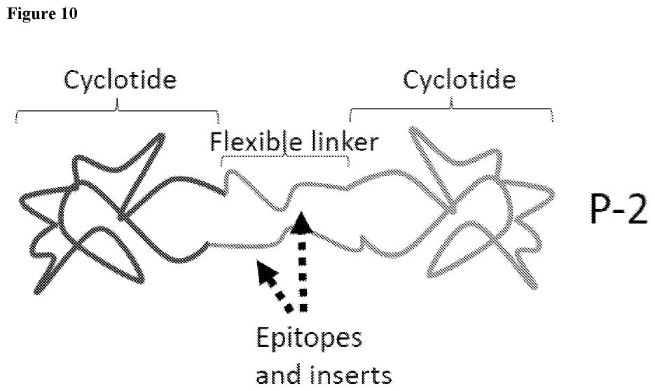

Structures that retain cyclotide character while incorporating two or more distinct base cyclotides, referred to herein as “polygonal cyclotides,” were also designed and contemplated to provided increased peptide insert capacity. As shown in FIG. 10, an exemplary “P-2” polygonal cyclotide was designed that includes two base cyclotide structures connected as a mirror image of each other via a pair of flexible linkers. Each of these flexible linkers optionally may include epitopes and inserts, optionally of lengths exceeding those of traditional cyclotide structures (e.g., 25 or more amino acids in length, 30 or more amino acids in length, etc.), while retaining cyclotide properties.

To assess whether an exemplary “P-2” cyclotide would retain cyclotide properties such as protease resistance, an exemplary polygonal cyclotide (P-2) possessing the following sequence and a natural cyclotide (MCoTI) were both expressed and subjected to trypsin protease digestion for 8 hours at 37° C.

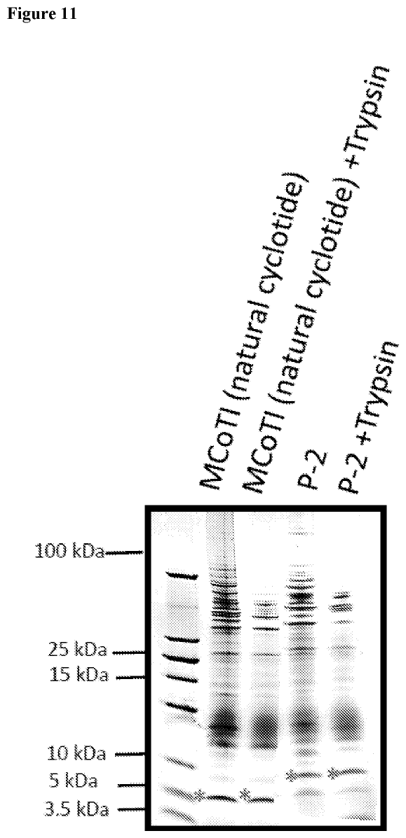

As shown in FIG. 11, SDS-PAGE gel analysis of peptides subjected to trypsin digestion revealed that the exemplary “P-2” polygonal cyclotide robustly retained the cyclotide property of trypsin resistance, to similar extent as the base MCoTI natural cyclotide over a 24 h period. Based upon the sizes of linkers used within this “P-2” polygonal cyclotide, the peptide insert capacity of this “P-2” polygonal cyclotide appears to be at least 50-60 amino acid residues, thereby providing an attractive cyclotide framework for introduction of dramatically extended peptide inserts that would retain cyclotide characteristics (e.g., protease resistance, etc.) relevant to use of such structures as a delivery vehicle for such peptide inserts, even upon introduction of such extended length peptide inserts into the cyclotide structure(s).

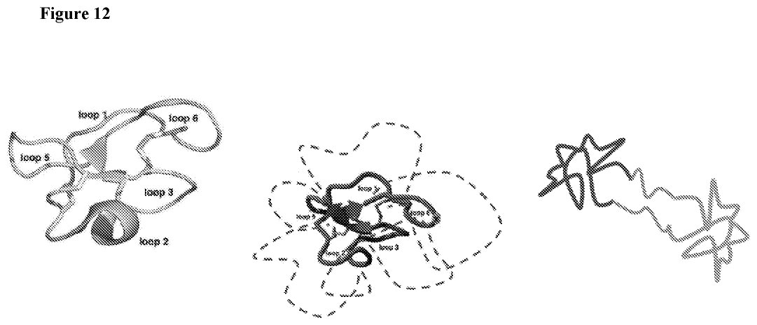

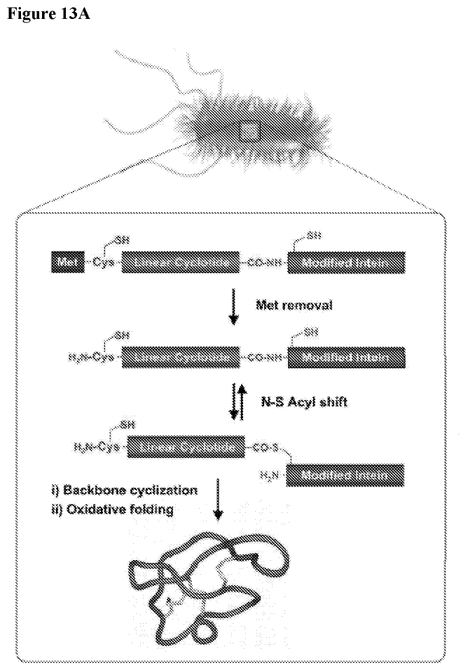

FIG. 12 highlights the different cyclotide structures as described herein, including the 2× and P-2 (polygonal) cyclotides.

As shown in FIG. 12, contemplated polygonal cyclotides are not limited to those possessing only two base cyclotide structures, but can include three, four, or even more base cyclotide structures, joined together via linkers, which still retain cyclotide properties, such as enhanced protease resistance relative to linear peptides.

Accordingly, polygonal cyclotides have thereby been produced possessing an insert capacity of at least 50-60 amino acids within a loop (e.g., within a flexible linker), while retaining the protease resistant properties of a canonical cyclotide structure.

### Example 4: High Throughput Cyclic Peptide Pipeline Development

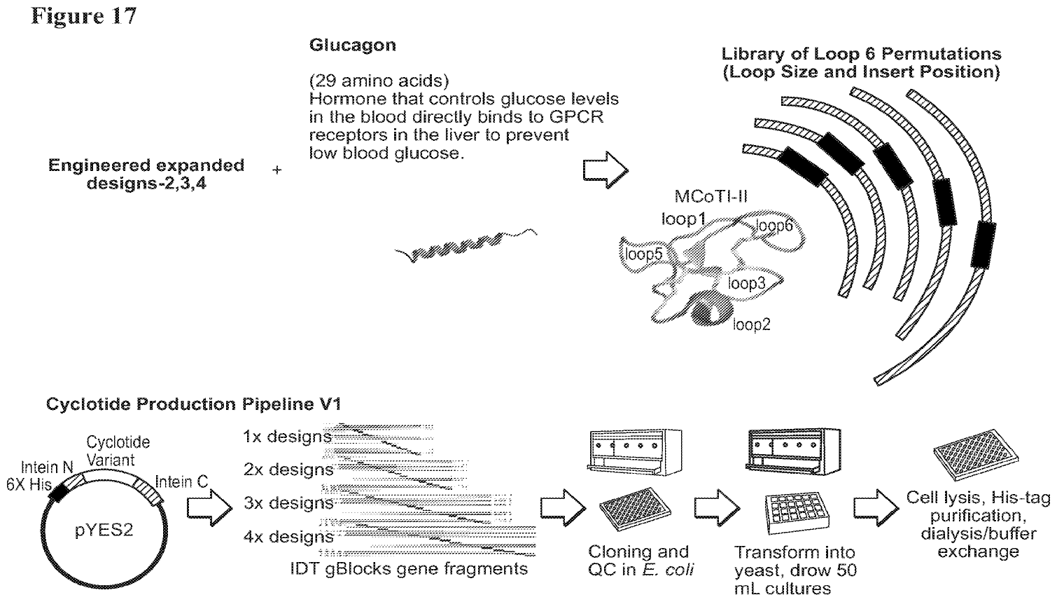

The engineered cyclic peptides of the instant disclosure expand the scope of what is feasible to perform using cyclic peptides, especially in the delivery space. In particular, the cyclic peptides of the disclosure provide an expanded “design space” for cyclic peptide use, which, in turn, necessitates development of an efficient way to screen for cyclic peptides of the disclosure that retain advantageous cyclic peptide properties (e.g., stability, enzyme, heat and/or acid resistance, etc.) while also exhibiting desired insert and/or payload-specific properties. As shown in FIG. 17, a high throughput cyclic peptide pipeline has been designed for testing of therapeutic peptide inserts, optionally at every possible location within loop 6 of the expanded cyclic peptide structures of the disclosure. The exemplary screening process involves production of 2×, 3× and 4× (MCoTI-II-base cyclotide) expanded cyclic peptide structures and insertion of glucagon (having a length of 29 amino acid residues) as the payload peptide sequence of expanded loop 6 of such cyclic peptide sequences. Many different insertion sites for glucagon within the expanded loop 6 sequence are tested, with expression of all sites shown performed using the yeast expression vector pYES2, which is cloned in E. coli and expressed in 50 mL yeast cultures. A high throughput 96 well plate format is then used for cell lysis, His-tag purification of expressed cyclic peptides and dialysis/buffer exchange. Once purified, the cyclic peptides are tested for stability and/or activity, such that optimal cyclic peptides are ultimately selected for further use and development.

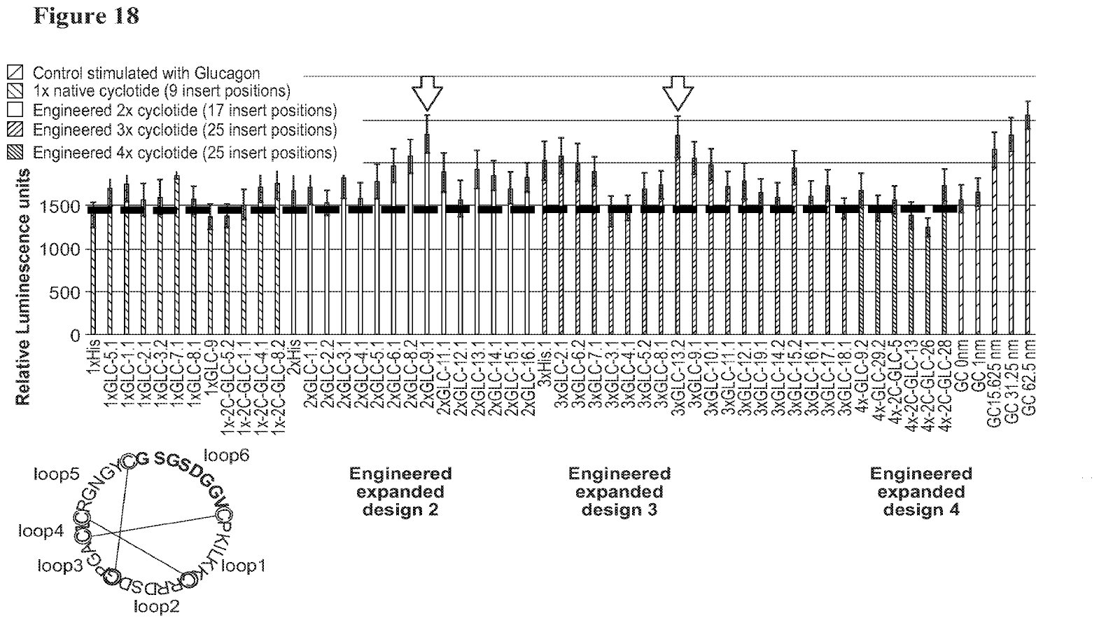

The above-described cyclic peptide screening methods were applied to 56 cyclotide designs across “V2”, “V3” and “V4” formats (2×, 3× and 4× loop expanded cyclotides, respectively), with such cyclotides harboring glucagon (a hormone that controls glucose levels in the blood and directly binds to GPCR in the liver to prevent low blood glucose). As shown in FIG. 18, a number of engineered cyclotides of the disclosure (particularly “V2” and “V3” designs set off by arrows) exhibited receptor binding between engineered cyclotides and GPCR. When certain “V2” glucagon cyclotides were assayed for trypsin resistance, trypsin resistance was confirmed, with SDS-PAGE analysis having identified the presence of new structures that were confirmed for mass by MALDI-MS (data not shown). An EC50 for a selected “V2” glucagon-harboring cyclotide was also calculated (data not shown).

A similar high throughput approach was performed in an attempt to identify cylcotides of the disclosure harboring antimicrobial peptides that were active, though an initial construct appeared not to achieve appropriate levels of release of the inserted antimicrobial peptide (data not shown).

### Example 5: In Vitro Cyclic Peptide Production

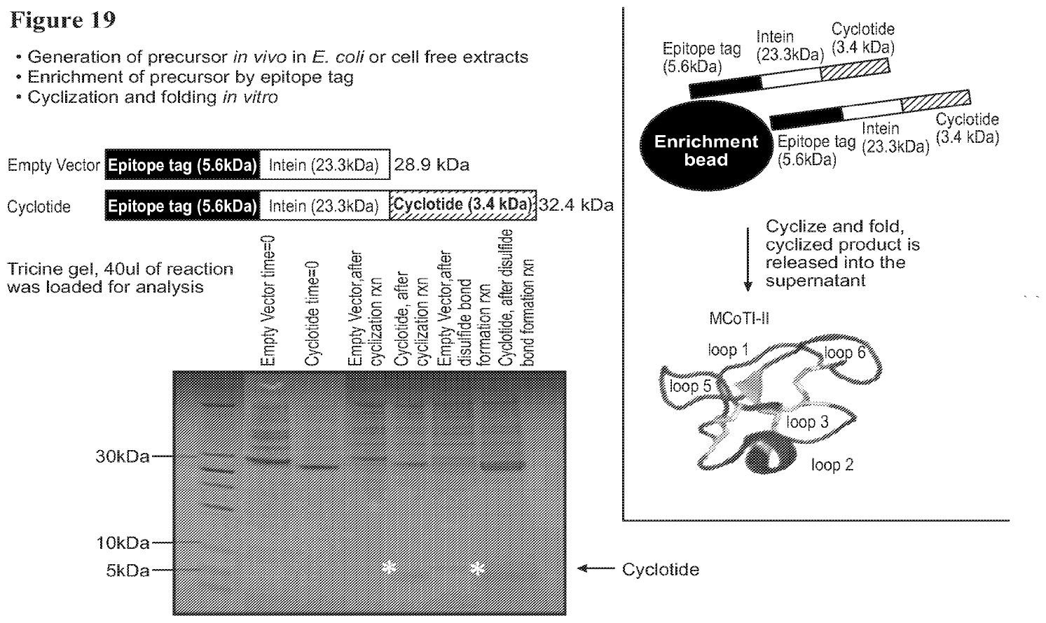

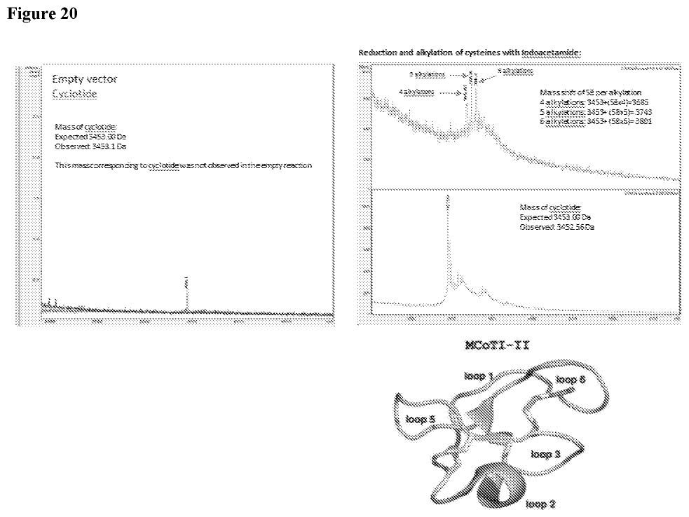

In certain embodiments, the engineered cyclic peptides of the disclosure can be produced in vitro, optionally allowing for improved high throughput production, as well as incorporation of ncAAs, linkers, etc., as described in detail above. One such approach for high throughput in vitro production is shown in FIG. 19, which demonstrates successful production of cyclotide in an in vitro process. In the process, precursor peptides (empty vector or cyclotide-containing) were produced in either E. coli or in cell-free extracts, precursor peptides were then isolated by epitope tag pull-down (shown at right in FIG. 19), and cyclization and folding were performed in vitro.

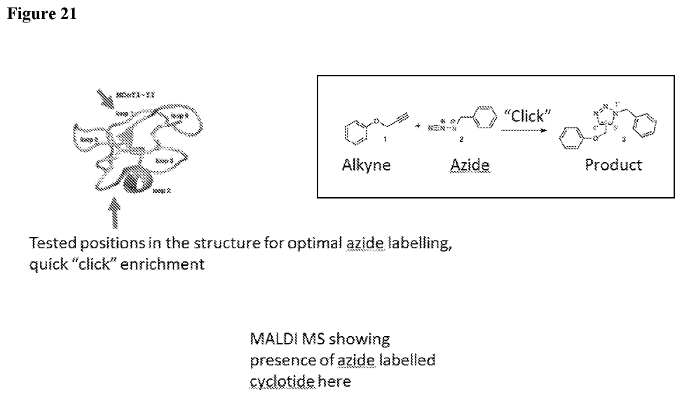

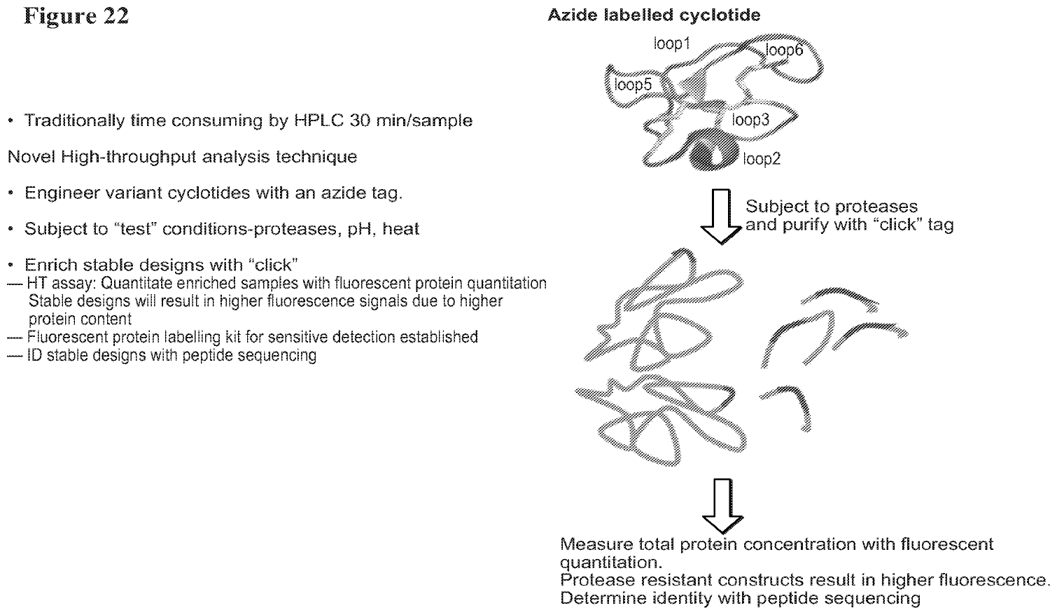

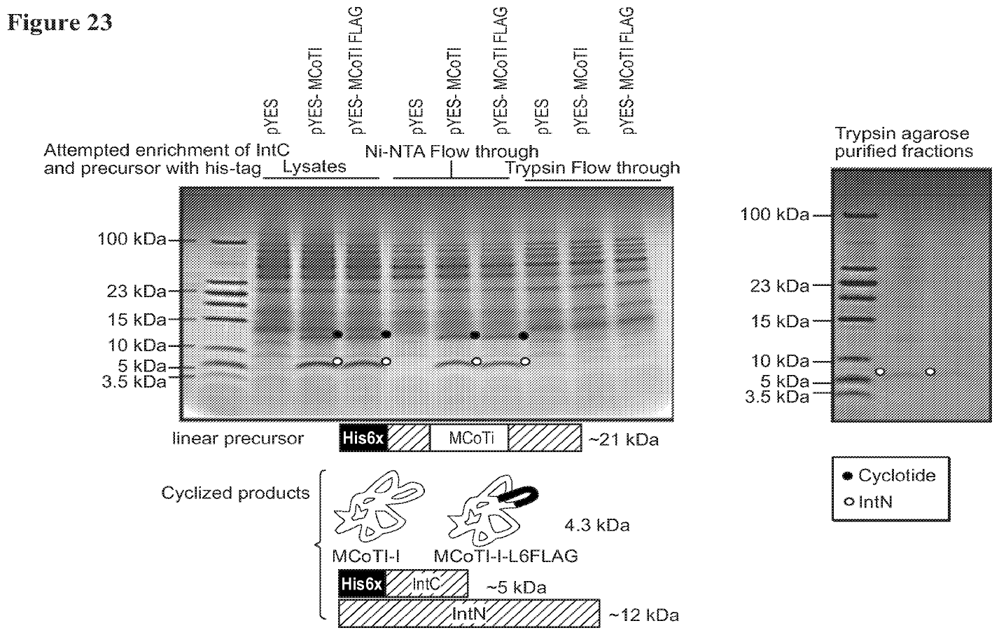

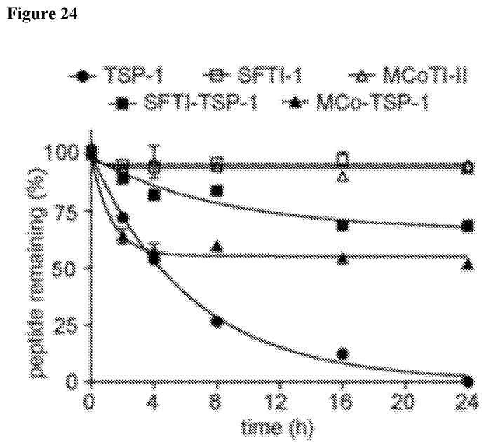

Cell-free synthesis of cyclic peptides of the disclosure was also identified to be automatable and plate-compatible in additional experiments, allowing for high-throughput production. As shown in FIG. 21, cell free synthesis of azide labeled cyclotides is successfully performed, allowing for azide labeling and use of “click” chemistry for enrichment, etc. Use of azide labelling allows for much quicker assessment of stability and/or cyclization, and therefore enrichment of stable cyclic peptide designs, as shown in FIG. 22.

Thus, cyclic peptide production was established in yeast and cyclic peptide stability was verified by assessment of resistance to both trypsin protease and by assessment of stability in human serum. In addition, an automated pipeline for cyclotide production was developed, and stable epitope-tagged cyclic peptide constructs were designed and created possessing 100% to 500% greater insert capacity in loop 6 of exemplified cyclotides than were previously available for cyclotides. Further, 128 member cyclotide libraries were created and tested as described above, for both glucagon-harboring cyclotides and SMAP 29 (sheep myeloid antimicrobial peptide 29)-harboring cyclotides. All such production and screening was identified as adaptable to high throughput approaches.

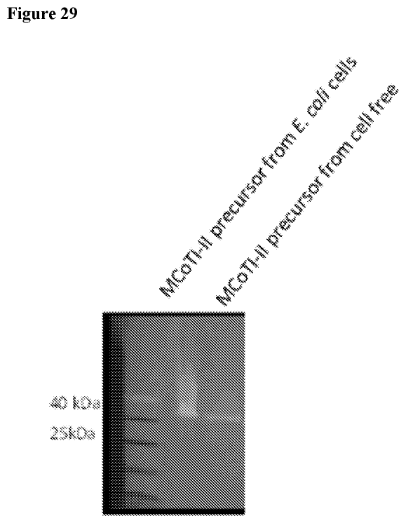

A final exemplary protocol for the cell free production of precursor is provided by the following: a cell free reaction was set up with BL21 cell free S30 extract (DTT free), minimum of 750 μL final volume (to cover all assays below) as follows in a DNAse/RNAse free 2 mL tube or 96-well plate as follows: 50 μL SS reagent; 50 μL MM reagent; 25 μL SM reagent; 300 DNA template (250 ng/μL); 10 μL RNase inhibitor; 50 μL 0.9 μM urea; 15 μL T7 polymerase (5 mg/ml); 250 μL extract. With a final volume of 750 the mixture was incubated at 30° C. (not 37° C.) in a PCR machine for 1 h. Cell free production was confirmed by Western blot, as shown in FIG. 29.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A method for stabilizing a cyclic peptide possessing two or more loop domain sequences, wherein a first loop domain sequence of the cyclic peptide is at least 25 amino acids in length, the method comprising:
extending the length of a second loop domain sequence of the cyclic peptide by at least three amino acids, wherein the extending of the second loop domain sequence of the cyclic peptide improves the trypsin resistance of the cyclic peptide,
thereby stabilizing the cyclic peptide possessing two or more loop domain sequences.

2. The method of claim 1, wherein the cyclic peptide is a cyclotide, optionally wherein the cyclotide is a MCoTI-I cyclotide having an extended loop 6 as the first loop domain sequence of the cyclotide that is at least 25 amino acids in length.

3. The method of claim 1, wherein the at least three amino acid extension of the second loop domain sequence is a duplication of an at least three amino acid sequence found within the same second loop domain sequence.

4. The method of claim 1, wherein the at least three amino acid extension of the second loop domain sequence is an inverted duplication of an at least three amino acid sequence found within the same second loop domain sequence.

5. The method of claim 1, wherein the cyclic peptide comprises at least three loop domain sequences, optionally wherein three or more of the at least three loop domain sequences are extended, optionally wherein:
(A) loop domain sequence extension for each loop domain sequence other than the first loop domain sequence is performed by duplication and/or inverted duplication of the original sequence of the same loop domain sequence;
(B) at least two loop domain sequences of the cyclic peptide other than the first loop domain sequence of the cyclic peptide are extended by duplication, inverted duplication, or both, of the original sequence of the same loop domain sequence now extended;
(C) two or more loop domain sequences of the cyclic peptide other than the first loop domain sequence of the cyclic peptide are extended, wherein each of the two or more loop domain sequences of the cyclic peptide other than the first loop domain sequence of the cyclic peptide is extended by an amount selected from the group consisting of about the original length of the original loop domain sequence now extended of the cyclic peptide, about twice the original length of the original loop domain sequence now extended of the cyclic peptide, about three times the original length of the original loop domain sequence now extended of the cyclic peptide, about four times the original length of the original loop domain sequence now extended of the cyclic peptide and about five times the original length of the original loop domain sequence now extended of the cyclic peptide;
(D) two or more loop domain sequences of the cyclic peptide other than the first loop domain sequence of the cyclic peptide are extended, wherein each of the two or more loop domain sequences of the cyclic peptide other than the first loop domain sequence of the cyclic peptide is extended by about the original length of the original loop domain sequence now extended of the cyclic peptide;
(E) two or more loop domain sequences of the cyclic peptide other than the first loop domain sequence of the cyclic peptide are extended, wherein each of the two or more loop domain sequences of the cyclic peptide other than the first loop domain sequence of the cyclic peptide is extended by about twice the original length of the original loop domain sequence now extended of the cyclic peptide;
(F) two or more loop domain sequences of the cyclic peptide other than the first loop domain sequence of the cyclic peptide are extended, wherein each of the two or more loop domain sequences of the cyclic peptide other than the first loop domain sequence of the cyclic peptide is extended by about three times the original length of the original loop domain sequence now extended of the cyclic peptide;
(G) two or more loop domain sequences of the cyclic peptide other than the first loop domain sequence of the cyclic peptide are extended, wherein each of the two or more loop domain sequences of the cyclic peptide other than the first loop domain sequence of the cyclic peptide is extended by about four times the original length of the original loop domain sequence now extended of the cyclic peptide; and/or
(H) two or more loop domain sequences of the cyclic peptide other than the first loop domain sequence of the cyclic peptide are extended, wherein each of the two or more loop domain sequences of the cyclic peptide other than the first loop domain sequence of the cyclic peptide is extended by about five times the original length of the original loop domain sequence now extended of the cyclic peptide.

6-7. (canceled)

8. The method of claim 1, wherein the second loop domain sequence of the cyclic peptide is extended by an amount selected from the group consisting of about the original length of the original second loop domain sequence of the cyclic peptide, about twice the original length of the original second loop domain sequence of the cyclic peptide, about three times the original length of the original second loop domain sequence of the cyclic peptide, about four times the original length of the original second loop domain sequence of the cyclic peptide and about five times the original length of the original second loop domain sequence of the cyclic peptide, optionally wherein each loop domain sequence of the cyclic peptide other than the first loop domain sequence of the cyclic peptide is extended by an amount selected from the group consisting of about the original length of the original loop domain sequence now extended of the cyclic peptide, about twice the original length of the original loop domain sequence now extended of the cyclic peptide, about three times the original length of the original loop domain sequence now extended of the cyclic peptide, about four times the original length of the original loop domain sequence now extended of the cyclic peptide and about five times the original length of the original loop domain sequence now extended of the cyclic peptide.

9-16. (canceled)

17. The method of claim 1, wherein the first loop domain sequence of the cyclic peptide comprises a peptide derived from a source exogenous to the base cyclic peptide sequence, optionally a therapeutic peptide, optionally a polypeptide drug of 22-50 or more amino acids in length, an antibody molecule or fragment, optionally a monoclonal antibody, single domain antibodies such as camelid or cartilaginous fish antibody, scFv, antibody fragment such as Fv, Fab, Fab′ and F(ab′)₂ fragments, and other fragments, and/or a small molecule, optionally a small molecule attached to the cyclic peptide via a non-canonical amino acid and/or linker, optionally wherein the therapeutic peptide is selected from Table 4.

18. (canceled)

19. The method of claim 1, wherein the first loop domain sequence of the cyclic peptide comprises a peptide tag, optionally wherein the peptide tag is an epitope tag (e.g., a FLAG-tag, a V5-tag, Myc-tag, HA-tag and/or NE-tag), a polyglutamate tag, a Strep-tag and/or a HIS tag.

20. The method of claim 1, wherein the cyclic peptide is selected from Table 1 or Table 3, optionally having an extended loop 6 as the first loop domain sequence of the cyclic peptide that is at least 30 amino acids in length.

21. The method of claim 1, wherein the stabilized cyclic peptide sequence is selected from Table 5.

22. The method of claim 1, wherein the first loop domain sequence of the cyclic peptide is at least 30 amino acids in length, optionally at least 35 amino acids in length, optionally at least 40 amino acids in length, optionally at least 45 amino acids in length, and optionally 50 or more amino acids in length.

23. The method of claim 1, wherein:
trypsin resistance of the stabilized cyclic peptide is assessed under the following conditions: the cyclic peptide is exposed to 10 μg trypsin protease digestion at 37° C. for between two and 24 hours; and/or
the loops of the cyclic peptide are expanded to maintain position of Cys-Cys linkages within the cyclic peptide structure, optionally wherein the cyclic peptide structure possesses three Cys-Cys linkages, optionally wherein the three Cys-Cys linkages form in the following order: first Cys covalently binds fourth Cys; second Cys covalently binds fifth Cys; and third Cys covalently binds sixth Cys, optionally wherein a chaperone molecule aids Cys-Cys bond formation.

24. (canceled)

25. A method selected from the group consisting of:
(A) a method for treating or preventing a disease or disorder in a subject comprising: administering to the subject a stabilized cyclic peptide in an amount effective to treat or prevent a disease or disorder in a subject, wherein the stabilized cyclic peptide possesses two or more loop domain sequences, wherein a first loop domain sequence of the stabilized cyclic peptide is at least 30 amino acids in length and the length of a second loop domain sequence of the stabilized cyclic peptide has been extended by at least three amino acids to improve the trypsin resistance of the stabilized cyclic peptide, thereby treating or preventing a disease or disorder in the subject;
(B) a method for designing a multi-loop-expanded cyclic peptide possessing at least one loop domain sequence in excess of 25 amino acids in length comprising: identifying a base cyclic peptide sequence comprising at least two loop domain sequences, wherein each loop domain sequence is of 25 amino acid residues or less in length; extending a first of the at least two loop domain sequences from an initial length (L₁ᵢ) to and extended length (L₁ₑ), wherein length L₁ₑ exceeds 25 amino acids, thereby forming a first extended loop domain sequence; and extending the length of a second loop domain sequence of the base cyclic peptide sequence possessing an original second loop domain sequence length of L₂ᵢ by between about 0.1·[(L₁ₑ−L₁ᵢ)/L₁ᵢ]·L₂ᵢ and about 100·[(L₁ₑ−L₁ᵢ)/L₁ᵢ]·L₂ᵢ amino acid residues, wherein the length of the extension is at least one amino acid, thereby designing a multi-loop-expanded cyclic peptide;
(C) a method for designing a cyclotide composition comprising at least 10 loop domain sequences and two linker sequences, comprising: identifying a first base cyclotide sequence and a second base cyclotide sequence, wherein each base cyclotide sequence comprises at least six loop domain sequences; severing the longest loops of each of the first base cyclotide sequence and the second base cyclotide sequence and removing between 0 and 7 amino acid residues from each end of the severed loop sequences, thereby creating (a) an N-terminal free end of the first base cyclotide sequence and a C-terminal free end of the first base cyclotide sequence and (b) an N-terminal free end of the second base cyclotide sequence and a C-terminal free end of the second base cyclotide sequence; joining the C-terminal free end of the first base cyclotide sequence to the N-terminus of a first linker sequence and joining the C-terminus of the first linker sequence to the N-terminal free end of the second base cyclotide sequence; and joining the C-terminal free end of the second base cyclotide sequence to the N-terminus of a second linker sequence and joining the C-terminus of the second linker sequence to the N-terminal free end of the first base cyclotide sequence, thereby designing a cyclotide composition comprising at least 10 loop domain sequences and two linker sequences;
(D) a method for treating or preventing a disease or disorder in a subject comprising:
administering to the subject a cyclotide composition comprising at least 10 loop domain sequences and two linker sequences in an amount effective to treat or prevent a disease or disorder in a subject, thereby treating or preventing a disease or disorder in the subject;
(E) a method for designing a cyclotide composition comprising at least 15 loop domain sequences and three linker sequences, comprising: identifying a first base cyclotide sequence, a second base cyclotide sequence and a third base cyclotide sequence, wherein each base cyclotide sequence comprises at least six loop domain sequences; severing the longest loops of each of the first base cyclotide sequence, the second base cyclotide sequence, and the third base cyclotide sequence and removing between 0 and 7 amino acid residues from each end of the severed loop sequences, thereby creating (a) an N-terminal free end of the first base cyclotide sequence and a C-terminal free end of the first base cyclotide sequence, (b) an N-terminal free end of the second base cyclotide sequence and a C-terminal free end of the second base cyclotide sequence and (c) an N-terminal free end of the third base cyclotide sequence and a C-terminal free end of the third base cyclotide sequence; joining the C-terminal free end of the first base cyclotide sequence to the N-terminus of a first linker sequence and joining the C-terminus of the first linker sequence to the N-terminal free end of the second base cyclotide sequence; joining the C-terminal free end of the second base cyclotide sequence to the N-terminus of a second linker sequence and joining the C-terminus of the second linker sequence to the N-terminal free end of the third base cyclotide sequence; and joining the C-terminal free end of the third base cyclotide sequence to the N-terminus of a third linker sequence and joining the C-terminus of the third linker sequence to the N-terminal free end of the first base cyclotide sequence, thereby designing a cyclotide composition comprising at least 15 loop domain sequences and three linker sequences;
(F) a method for treating or preventing a disease or disorder in a subject comprising: administering to the subject a cyclotide composition comprising at least 15 loop domain sequences and three linker sequences in an amount effective to treat or prevent a disease or disorder in a subject, thereby treating or preventing a disease or disorder in the subject (G) a method for designing a cyclotide composition comprising at least 20 loop domain sequences and four linker sequences, comprising: identifying a first base cyclotide sequence, a second base cyclotide sequence, a third base cyclotide sequence and a fourth base cyclotide sequence, wherein each base cyclotide sequence comprises at least six loop domain sequences; severing the longest loops of each of the first base cyclotide sequence, the second base cyclotide sequence, the third base cyclotide sequence and the fourth base cyclotide sequence and removing between 0 and 7 amino acid residues from each end of the severed loop sequences, thereby creating (a) an N-terminal free end of the first base cyclotide sequence and a C-terminal free end of the first base cyclotide sequence, (b) an N-terminal free end of the second base cyclotide sequence and a C-terminal free end of the second base cyclotide sequence, (c) an N-terminal free end of the third base cyclotide sequence and a C-terminal free end of the third base cyclotide sequence and (d) an N-terminal free end of the third base cyclotide sequence and a C-terminal free end of the third base cyclotide sequence; joining the C-terminal free end of the first base cyclotide sequence to the N-terminus of a first linker sequence and joining the C-terminus of the first linker sequence to the N-terminal free end of the second base cyclotide sequence; joining the C-terminal free end of the second base cyclotide sequence to the N-terminus of a second linker sequence and joining the C-terminus of the second linker sequence to the N-terminal free end of the third base cyclotide sequence; joining the C-terminal free end of the third base cyclotide sequence to the N-terminus of a third linker sequence and joining the C-terminus of the third linker sequence to the N-terminal free end of the fourth base cyclotide sequence; and joining the C-terminal free end of the fourth base cyclotide sequence to the N-terminus of a fourth linker sequence and joining the C-terminus of the fourth linker sequence to the N-terminal free end of the first base cyclotide sequence, thereby designing a cyclotide composition comprising at least 20 loop domain sequences and four linker sequences;
(H) a method for treating or preventing a disease or disorder in a subject comprising: administering to the subject a cyclotide composition comprising at least 20 loop domain sequences and four linker sequences in an amount effective to treat or prevent a disease or disorder in a subject, thereby treating or preventing a disease or disorder in the subject;
(I) a method for identifying the presence of a protease-stabilized peptide composition in a solution comprising: preparing a tagged peptide capable of forming a protease-stabilized structure; subjecting the tagged peptide to one or more proteases under conditions that allow for protease activity; purifying tagged peptides, thereby generating a purified tagged peptide sample; labeling purified tagged peptide sample with fluorescent moieties that bind to one or more amino acids; and quantifying the level of fluorescence in the purified tagged peptide sample, as compared to an appropriate control, wherein the presence of an increased level of fluorescence in the purified tagged peptide sample identifies the presence and/or increased level of a protease-stabilized peptide composition in the purified tagged peptide sample, thereby identifying the presence of a protease-stabilized peptide composition in a solution; and
(J) a method for making a loop-expanded cyclic peptide possessing two or more loop domain sequences and at least one Cys-Cys linkage, the method comprising: extending the length of a first loop domain sequence and a second loop domain sequence of a base cyclic peptide sequence in proportion to one another, thereby forming a loop-expanded cyclic peptide, wherein the relative position of the Cys-Cys linkage is maintained within the loop-expanded cyclic peptide, as compared to the base cyclic peptide sequence, thereby making a loop-expanded cyclic peptide possessing two or more loop domain sequences.

26. (canceled)

27. The method of claim 25, wherein:
(i) the length of the second loop domain sequence of the base cyclic peptide sequence possessing an original second loop domain sequence length of L₂ᵢ is extended by between 0.5·[(L₁ₑ−L₁ᵢ)/L₁ᵢ]·L₂ᵢ and 1.5·[(L₁ₑ−L₁ᵢ)/L₁ᵢ]·L₂ᵢ amino acid residues;
(ii) the base cyclic peptide is a cyclotide, optionally a MCoTI-I cyclotide;
(iii) extending the length of the second loop domain sequence is performed by duplication, inverted duplication, or both, of the original second loop domain sequence, optionally wherein loop domain sequence extension for each loop domain sequence other than the first loop domain sequence is performed by duplication and/or inverted duplication of the original sequence of the same loop domain sequence;
(iv) the base cyclic peptide comprises at least three loop domain sequences, optionally wherein three or more of the at least three loop domain sequences are extended, optionally wherein the loop domain sequences that are extended, other than the first loop domain sequence, are extended in length by about the same proportion relative to the corresponding base sequences of the loop domain sequences other than the first loop domain sequence now extended;
(v) the first loop domain sequence of the base cyclic peptide is the longest loop domain sequence of the base cyclic peptide;
(vi) the first loop domain sequence of the base cyclic peptide is loop 6 of the base cyclic peptide;
(vii) the second loop domain sequence of the cyclic peptide is extended by an amount selected from the group consisting of about the original length of the original second loop domain sequence of the cyclic peptide, about twice the original length of the original second loop domain sequence of the cyclic peptide, about three times the original length of the original second loop domain sequence of the cyclic peptide, about four times the original length of the original second loop domain sequence of the cyclic peptide and about five times the original length of the original second loop domain sequence of the cyclic peptide, optionally wherein each loop domain sequence of the cyclic peptide other than the first loop domain sequence of the cyclic peptide is extended by an amount selected from the group consisting of about the original length of the original loop domain sequence now extended of the cyclic peptide, about twice the original length of the original loop domain sequence now extended of the cyclic peptide, about three times the original length of the original loop domain sequence now extended of the cyclic peptide, about four times the original length of the original loop domain sequence now extended of the cyclic peptide and about five times the original length of the original loop domain sequence now extended of the cyclic peptide;
(viii) the first loop domain sequence of the cyclic peptide comprises a peptide derived from a source exogenous to the base cyclic peptide sequence, optionally a therapeutic peptide, optionally a polypeptide drug of 22-50 or more amino acids in length, an antibody molecule or fragment, optionally a monoclonal antibody, single domain antibodies such as camelid or cartilaginous fish antibody, scFv, antibody fragment such as Fv, Fab, Fab′ and F(ab′)2 fragments, and other fragments, and/or a small molecule, optionally a small molecule attached to the cyclic peptide via a non-canonical amino acid and/or linker, optionally wherein the therapeutic peptide is selected from Table 4;
(ix) the base cyclic peptide is selected from Table 1 or Table 3;
(x) the multi-loop-expanded cyclic peptide sequence is selected from Table 5;
(xi) the loops of the cyclic peptide are expanded to maintain position of Cys-Cys linkages within the cyclic peptide structure, optionally wherein the cyclic peptide structure possesses three Cys-Cys linkages, optionally wherein the three Cys-Cys linkages form in the following order: first Cys covalently binds fourth Cys; second Cys covalently binds fifth Cys; and third Cys covalently binds sixth Cys, optionally wherein a chaperone molecule aids Cys-Cys bond formation;
(xii) the disease or disorder is selected from the group consisting of: a GPCR-related disease or disorder, a hormone-related disease or disorder, and a microbial infection and/or microbial infection-related disease or disorder;
(xiii) each loop domain sequence of the second base cyclotide sequence is the reverse sequence of the corresponding loop domain sequence of the first base cyclotide sequence;
(xiv) the first linker sequence, the second linker sequence, or both linker sequences are at least 25 amino acid residues in length;
(xv) the therapeutic peptide is selected from Table 4;
(xvi) the second base cyclotide sequence is the reverse sequence of the first base cyclotide sequence;
(xvii) the first linker sequence, the second linker sequence, the third linker sequence, or any combination thereof comprises a peptide derived from a source exogenous to the base cyclic peptide sequence, optionally a therapeutic peptide, optionally a polypeptide drug of 22-50 or more amino acids in length, an antibody molecule or fragment, optionally a monoclonal antibody, single domain antibodies such as camelid or cartilaginous fish antibody, scFv, antibody fragment such as Fv, Fab, Fab′ and F(ab′)₂ fragments, and other fragments, and/or a small molecule, optionally a small molecule attached to the cyclic peptide via a non-canonical amino acid and/or linker, and/or an epitope tag (e.g., a FLAG-tag, a V5-tag, Myc-tag, HA-tag and/or NE-tag), a polyglutamate tag, a Strep-tag and/or a HIS tag;
(xviii) the first linker sequence, the second linker sequence, the third linker sequence, or any combination thereof are each at least 25 amino acid residues in length;
(xix) the second and/or third base cyclotide sequence is the reverse sequence of the first base cyclotide sequence and/or the first and/or third base cyclotide sequence is the reverse sequence of the second base cyclotide sequence;
(xx) the first linker sequence, the second linker sequence, the third linker sequence, the fourth linker sequence, or any combination thereof comprises a peptide derived from a source exogenous to the base cyclic peptide sequence, optionally a therapeutic peptide, optionally a polypeptide drug of 22-50 or more amino acids in length, an antibody molecule or fragment, optionally a monoclonal antibody, single domain antibodies such as camelid or cartilaginous fish antibody, scFv, antibody fragment such as Fv, Fab, Fab′ and F(ab′)₂ fragments and other fragments, and/or a small molecule, optionally a small molecule attached to the cyclic peptide via a non-canonical amino acid and/or linker, and/or an epitope tag (e.g., a FLAG-tag, a V5-tag, Myc-tag, HA-tag and/or NE-tag), a polyglutamate tag, a Strep-tag and/or a HIS tag;
(xxi) the first linker sequence, the second linker sequence, the third linker sequence, the fourth linker sequence, or any combination thereof are each at least 25 amino acid residues in length;
(xxii) the first, second and/or third base cyclotide sequence is the reverse sequence of the fourth base cyclotide sequence; the second, third and/or fourth base cyclotide sequence is the reverse sequence of the first base cyclotide sequence; the first, third and/or fourth base cyclotide sequence is the reverse sequence of the second base cyclotide sequence; and/or the first, second and/or fourth base cyclotide sequence is the reverse sequence of the third base cyclotide sequence;
(xxiii) the protease-stabilized peptide composition is a cyclotide;
(xxiv) the protease-stabilized peptide composition is trypsin-stabilized;
(xxv) the one or more proteases comprise trypsin;
(xxvi) the method is performed in 96-well or 384-well plate format;
(xxvii) the tagged peptide is tagged with an epitope tag (e.g., a FLAG-tag, a V5-tag, Myc-tag, HA-tag and/or NE-tag), a polyglutamate tag, a Strep-tag and/or a HIS tag;
(xxviii) the loop-expanded cyclic peptide possesses four or more loop domain sequences and at least two Cys-Cys linkages, wherein the relative positions of the Cys-Cys linkages are maintained within the loop-expanded cyclic peptide as compared to the base cyclic peptide sequence, optionally wherein all loops of the base cyclic peptide sequence are extended in proportion to one another to form the loop-expanded cyclic peptide;
(xxix) the loop-expanded cyclic peptide possesses six or more loop domain sequences and at least three Cys-Cys linkages, wherein the relative positions of the Cys-Cys linkages are maintained within the loop-expanded cyclic peptide as compared to the base cyclic peptide sequence, optionally wherein all loops of the base cyclic peptide sequence are extended in proportion to one another to form the loop-expanded cyclic peptide; and/or
(xxx) the loop-expanded cyclic peptide is trypsin resistant.

28-42. (canceled)

43. A method for treating or preventing a disease or disorder in a subject comprising:
administering to the subject a multi-loop-expanded cyclic peptide sequence prepared by the method of claim 25 in an amount effective to treat or prevent a disease or disorder in a subject,
thereby treating or preventing a disease or disorder in the subject.

44. (canceled)

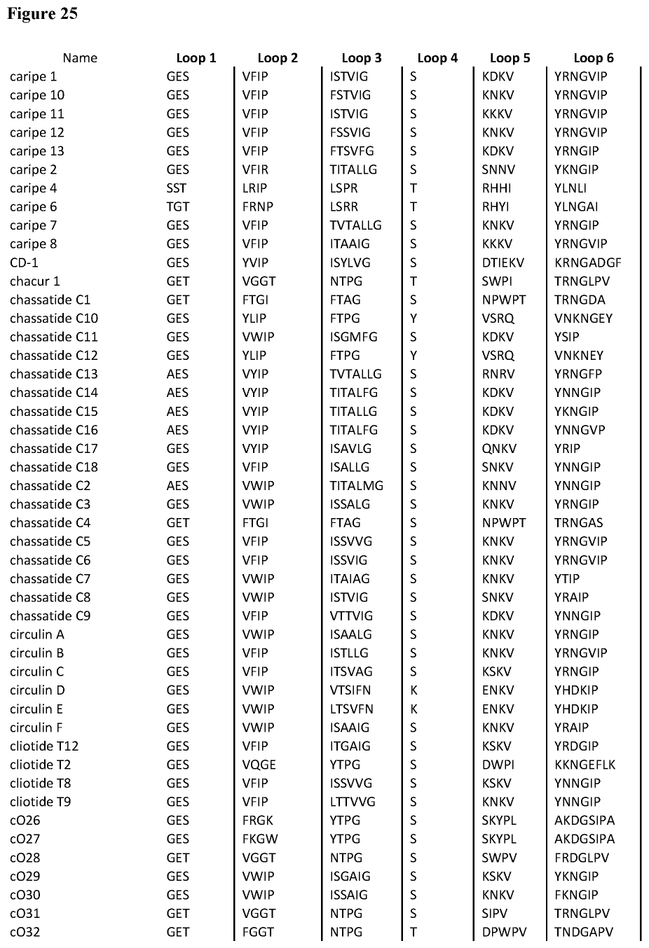

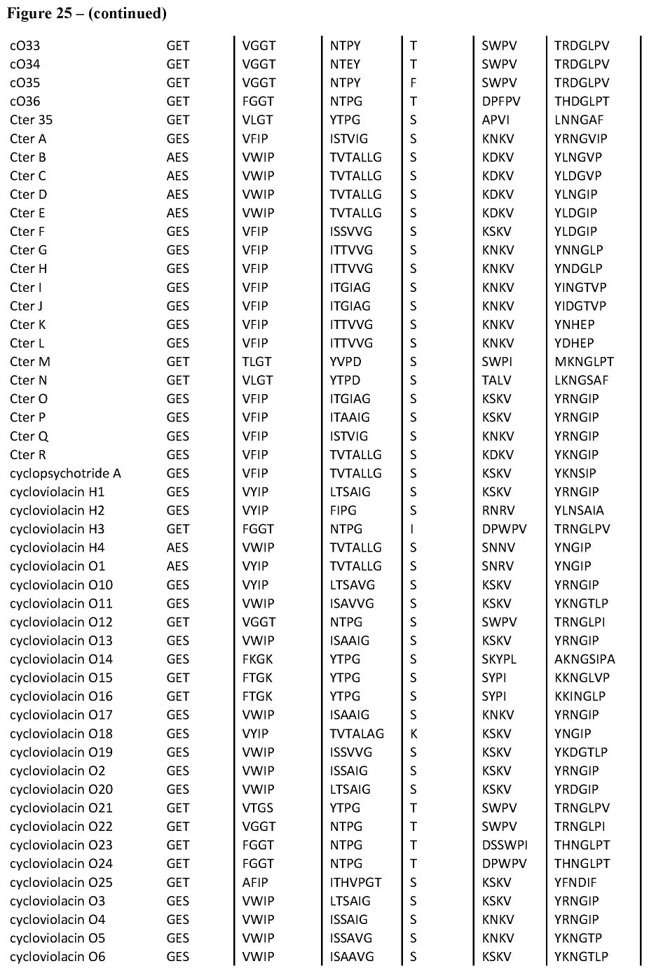

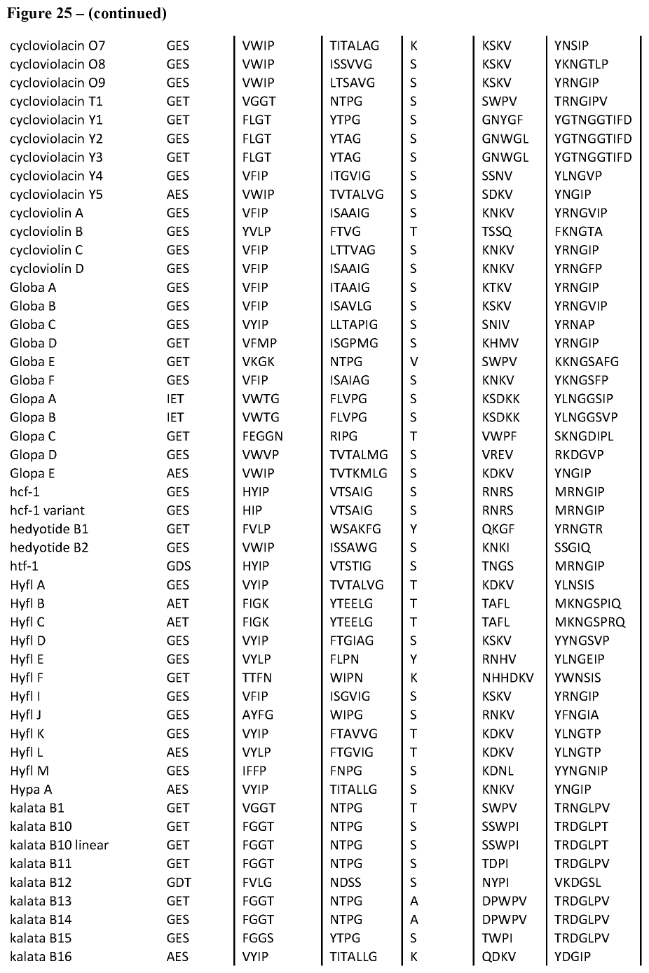

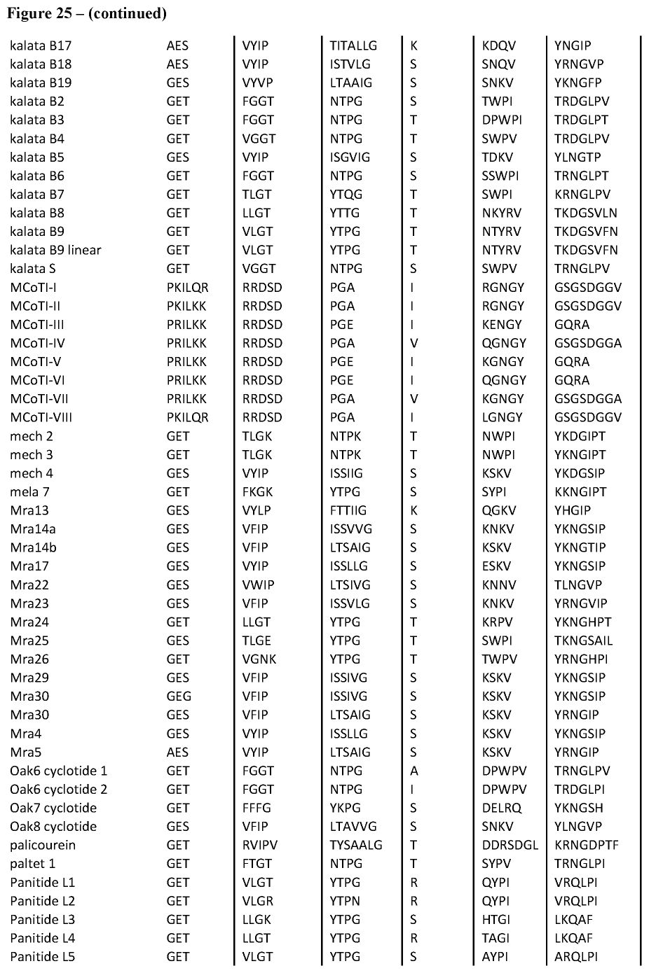

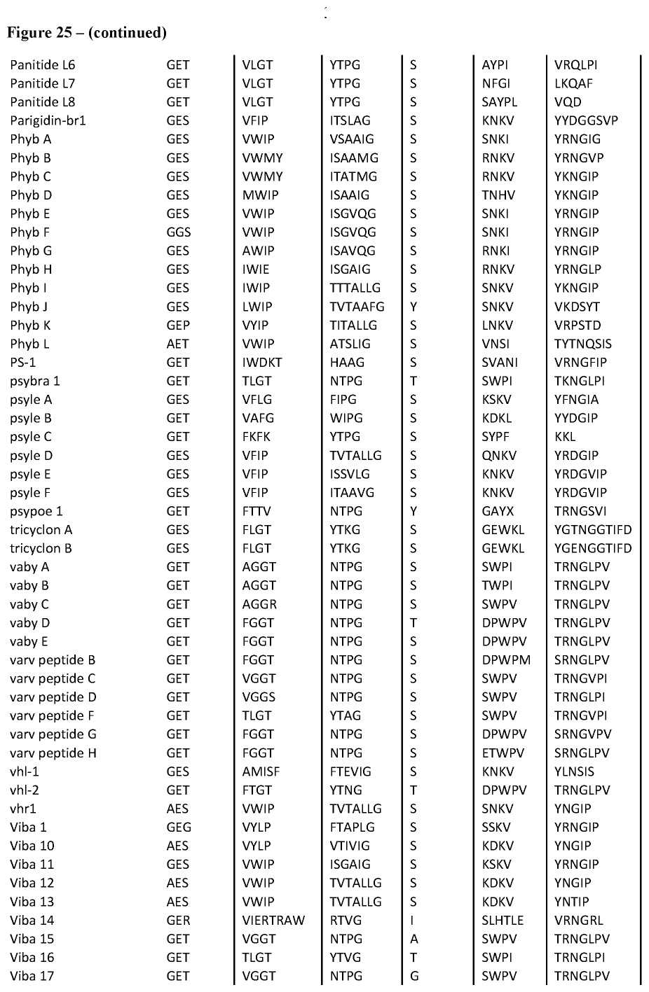

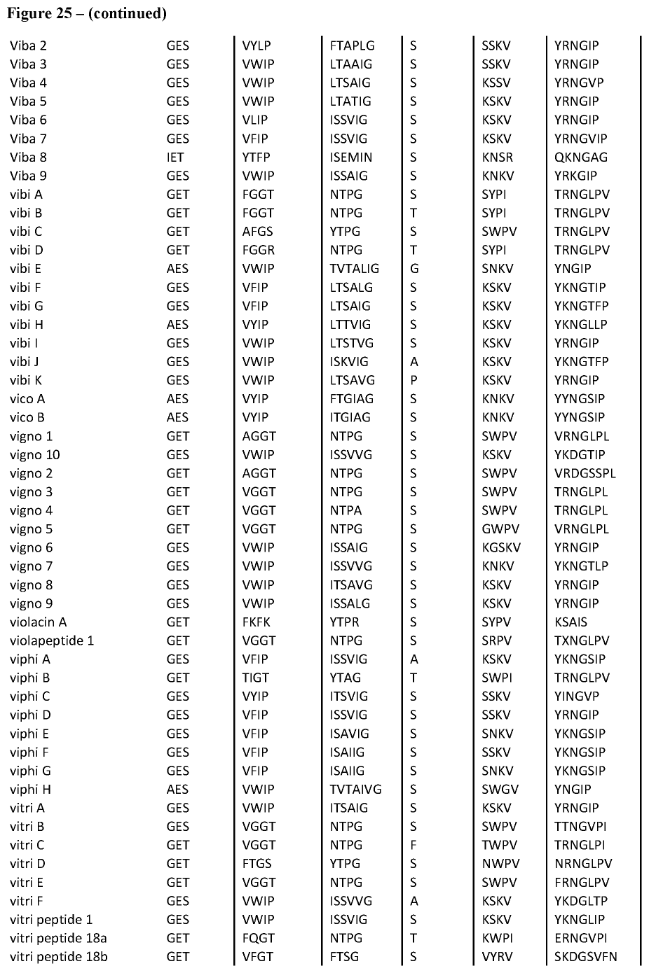

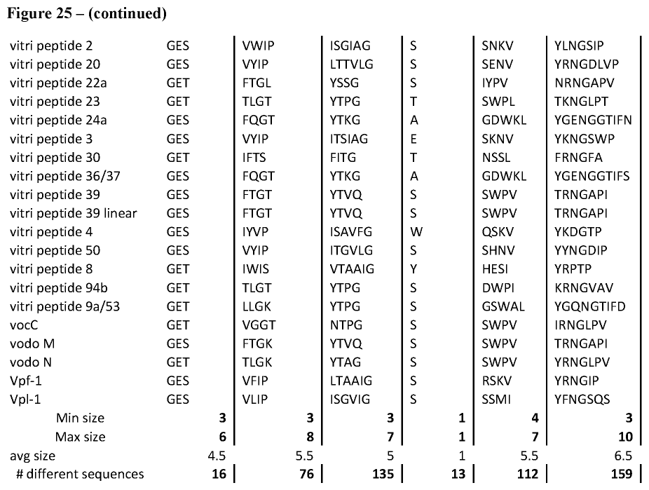

45. A composition comprising a composition selected from the group consisting of:
(A) a cyclic peptide sequence of Table 5;
(B) a pharmaceutical composition comprising a cyclic peptide sequence of Table 5 and a pharmaceutically acceptable carrier;
(C) a P-2, P-3 and/or P-4 format cyclic polypeptide;
(D) a pharmaceutical composition comprising a P-2, P-3 and/or P-4 format cyclic polypeptide and a pharmaceutically acceptable carrier;
(E) a loop-expanded cyclic peptide possessing two or more loop domain sequences and at least one Cys-Cys linkage, the loop-expanded cyclic peptide formed by extending the length of a first loop domain sequence and a second loop domain sequence of a base cyclic peptide sequence in proportion to one another, thereby forming the loop-expanded cyclic peptide, wherein the relative position of the Cys-Cys linkage is maintained within the loop-expanded cyclic peptide, as compared to the base cyclic peptide sequence;
(F) a loop-expanded cyclic peptide possessing four or more loop domain sequences and at least two Cys-Cys linkages, the loop-expanded cyclic peptide formed by extending the length of the four or more loop domain sequences of a base cyclic peptide sequence in proportion to one another, thereby forming the loop-expanded cyclic peptide, wherein the relative positions of the Cys-Cys linkages are maintained within the loop-expanded cyclic peptide, as compared to the base cyclic peptide sequence;
(G) a loop-expanded cyclic peptide possessing six or more loop domain sequences and at least three Cys-Cys linkages, the loop-expanded cyclic peptide formed by extending the length of the six or more loop domain sequences of a base cyclic peptide sequence in proportion to one another, thereby forming the loop-expanded cyclic peptide, wherein the relative positions of the Cys-Cys linkages are maintained within the loop-expanded cyclic peptide, as compared to the base cyclic peptide sequence;
(H) a cyclic peptide comprising:
(i) a cyclotide amino acid sequence of Table 2 or a corresponding modified cyclic peptide amino acid sequence that is at least about 95% identical to said cyclotide amino acid sequence of Table 2;
(ii) a first insert sequence comprising an amino acid sequence inserted into said cyclotide amino acid sequence between two amino acid residues of the (corresponding) loop 6 amino acid sequence of said cyclotide shown in FIG. 25, wherein, following insertion of the first insert sequence into the loop 6 amino acid sequence shown in FIG. 25, the loop 6 amino acid sequence containing the first insert sequence is at least 23 amino acid residues in length; and
(iii) a stabilizing insertion of a second insert sequence of at least three amino acids that is inserted between amino acid residues of any one of the (corresponding) loops 1-5 of said cyclotide amino acid sequence of Table 2, wherein the cyclic peptide sequence, excluding the first insert sequence, is at least 80% identical to the cyclotide amino acid sequence of Table 2 or the corresponding modified cyclic peptide amino acid sequence that is at least about 95% identical to the cyclotide amino acid sequence of Table 2; and
(I) a cyclic peptide comprising SEQ ID NO: 36 or SEQ ID NO: 362.

46-54. (canceled)

55. A cyclotide composition comprising at least 10 loop domain sequences and two linker sequences designed by the method of claim 25.

56-64. (canceled)

65. A cyclotide composition comprising at least 15 loop domain sequences and three linker sequences designed by the method of claim 25.

66-72. (canceled)

73. A cyclotide composition comprising at least 20 loop domain sequences and four linker sequences designed by the method of claim 25.

74-86. (canceled)

87. The composition of claim 45, wherein:
(A) the loop-expanded cyclic peptide is trypsin resistant; and/or
(B) wherein for the cyclic peptide:
(a) the cyclic peptide further comprises a third insert sequence of at least three amino acids that is inserted at any one of the (corresponding) loops 1-5 of said cyclotide amino acid sequence of Table 2, exclusive of the loop of (iii) that contains the second insert sequence;
(b) the cyclotide amino acid sequence of (i) is 100% identical to said cyclotide amino acid sequence of Table 2;
(c) the first insert sequence comprises a sequence selected from FIGS. 26 and 27, optionally wherein the first insert sequence is selected from the group consisting of glucagon, glucagon-like peptide 1 (GLP-1), amylin, adrenomedullin and pramlintide; and/or
(d) the sequence of the loop comprising the second insert sequence is selected from the group consisting of GPGKKIILLQQRR (SEQ ID NO: 363), GRRRRDDSSDD (SEQ ID NO: 364), GPGGGAA (SEQ ID NO: 365), GII (SEQ ID NO: 366) and GRRGGNNGGYY (SEQ ID NO: 367).

88-93. (canceled)


SEQUENCE LISTING (1757 sequences)

SEQ ID NO: 1 - protein, 39 aa, Unknown
  Description of Unknown MCoTI with CFN junction sequence
CFNGSGSDGGVCPKILQRCRRDSDCPGACICRGNGYCGS

SEQ ID NO: 2 - protein, 36 aa, Unknown
  Description of Unknown MCoT sequence
GSGSDGGVCPKILQRCRRDSDCPGACICRGNGYCGS

SEQ ID NO: 3 - protein, 34 aa, Unknown
  Description of Unknown MCOTI I sequence
GGVCPKILQRCRRDSDCPGACICRGNGYCGSGSD

SEQ ID NO: 4 - protein, 34 aa, Unknown
  Description of Unknown MCOTI II sequence
GGVCPKILKKCRRDSDCPGACICRGNGYCGSGSD

SEQ ID NO: 5 - protein, 30 aa, Unknown
  Description of Unknown Mcoti-III sequence
ERACPRILKKCRRDSDCPGACICRGNGYCG

SEQ ID NO: 6 - protein, 38 aa, Unknown
  Description of Unknown C15 sequence
GGVCNNNATPSLKVCRRDSDCPGACICRGNGYCGSGSD

SEQ ID NO: 7 - protein, 38 aa, Unknown
  Description of Unknown C7 sequence
GGVCGMDLFEESPYCRRDSDCPGACICRGNGYCGSGSD

SEQ ID NO: 8 - protein, 38 aa, Unknown
  Description of Unknown C8 sequence
GGVCWLRDEHPFKNCRRDSDCPGACICRSNGYCGSGSD

SEQ ID NO: 9 - protein, 38 aa, Unknown
  Description of Unknown C9 sequence
GGVCTYWYLYHTKGCRRDSDCPGACICRGNGYCGSGSD

SEQ ID NO: 10 - protein, 38 aa, Unknown
  Description of Unknown C10 sequence
GGVCNLDVANWTVWCRRDSDCPGACICRCNCYCGSGSD

SEQ ID NO: 11 - protein, 38 aa, Unknown
  Description of Unknown C11 sequence
GGVCRHSYSQIPLWCRRDSDCPGACICRGNGYCGSGSD

SEQ ID NO: 12 - protein, 38 aa, Unknown
  Description of Unknown C12 sequence
GGVCLELAKAYFQMCRRDSDCPGACICRGNGYCGSGSD

SEQ ID NO: 13 - protein, 38 aa, Unknown
  Description of Unknown C13 sequence
GGVCQQMHFRVMVHCRRDSDCPGACICRGNGYCGSGSD

SEQ ID NO: 14 - protein, 38 aa, Unknown
  Description of Unknown C14 sequence
GGVCTHWRWRSTIWCRRDSDCPGACICRGNGYCGSGSD

SEQ ID NO: 15 - protein, 38 aa, Unknown
  Description of Unknown C16 sequence
GGVCFVTDHWEHAPCRRDSDCPGACICRGNGYCGSGSD

SEQ ID NO: 16 - protein, 38 aa, Unknown
  Description of Unknown C17 sequence
GGVCFDHHSHYIRRCRRDSDCPGACICRGNGYCGSGSD

SEQ ID NO: 17 - protein, 38 aa, Unknown
  Description of Unknown C18 sequence
GGVCQWWLHMINAVCRRDSDCPGACICRGNGYCGSGSD

SEQ ID NO: 18 - protein, 38 aa, Unknown
  Description of Unknown C19 sequence
GGVCPFLPTEWWNSCRRDSDCPGACICRGNGYCGSGSD

SEQ ID NO: 19 - protein, 38 aa, Unknown
  Description of Unknown C20 sequence
GGVCVRKWWYTESICRRDSDCPGACICRGNGYCGSGSD

SEQ ID NO: 20 - protein, 40 aa, Unknown
  Description of Unknown C21 sequence
GGVCYDDETPPHETQHCRRDSDCPGACICRGNGYCGSGSD

SEQ ID NO: 21 - protein, 40 aa, Unknown
  Description of Unknown C22 sequence
GGVCQRRKWYWKESIQCRRDSDCPGACICRGNGYCGSGSD

SEQ ID NO: 22 - protein, 40 aa, Unknown
  Description of Unknown C23 sequence
GGVCQYTKPFVKGPHHCRRDSDCPGACICRGNGYCGSGSD

SEQ ID NO: 23 - protein, 40 aa, Unknown
  Description of Unknown C24 sequence
GGVCSKKRKMSSVVHPCRRDSDCPGACICRGNGYCGSGSD

SEQ ID NO: 24 - protein, 40 aa, Unknown
  Description of Unknown C25 sequence
GGVCEVYVWNGELKAWCRRDSDCPGACICRGNGYCGSGSD

SEQ ID NO: 25 - protein, 40 aa, Unknown
  Description of Unknown C26 sequence
GGVCRFQQGKWWEPHQCRRDSDCPGACICRGNGYCGSGSD

SEQ ID NO: 26 - protein, 40 aa, Unknown
  Description of Unknown C27 sequence
GGVCHMQHPWSAFAWYCHRDSDCPGACICRGNGYCGSGSD

SEQ ID NO: 27 - protein, 40 aa, Unknown
  Description of Unknown C28 sequence
GGVCESDPFTEEFMHHCRRDSDCPGACICRGNGYCGSGSD

SEQ ID NO: 28 - protein, 40 aa, Unknown
  Description of Unknown C29 sequence
GGVCHKHGYDPVYVWSCRRDSDCPGACICRGNGYYGSGSD

SEQ ID NO: 29 - protein, 81 aa, Artificial Sequence
  Description of Artificial Sequence Synthetic polypeptide
CFNGGSSGGSSHHHHHHHHDDGGGGVVCGPGKKIILLQQRRCGRRRRDDSSDDCGPGGGAACGIICGRRGGNNGGYYCGGS

SEQ ID NO: 30 - protein, 110 aa, Artificial Sequence
  Description of Artificial Sequence Synthetic polypeptide
CFNGGGSSSGGGSSSHHHHHHHHDDDGGGGGGVVVCGGPGGKKKIIILLLQQQRRRCRRRRRRDDDSSSDDDCGGPGGAAACGGIIIGCGRRRGGGNNNGGGYYYGGCGS

SEQ ID NO: 31 - protein, 170 aa, Artificial Sequence
  Description of Artificial Sequence Synthetic polypeptide
CFNGAGESGAGESCGGGGSPGGKKKIIILLLQQQRRRGAGESCGGGGSRRRRRRDDDSSSDDDGAGESCGGGGSPGGGGGAAAGAGESCGGGGSIIIGAGESCGGGGSRRRGGGNNNGGGYYYGAGESCGGGGSGGGGSGGGSSSGGGSSSHHHHHHHHGGGGGGVVVGS

SEQ ID NO: 32 - protein, 29 aa, Unknown
  Description of Unknown Glucagon sequence
HSQGTFTSDYSKYLDSRRAQDFVQWLMNT

SEQ ID NO: 33 - protein, 29 aa, Unknown
  Description of Unknown Antimicrobial peptides SMAP-29 sequence
RGLRRLGRKIAHGVKKYGPTVLRIIRIAG

SEQ ID NO: 34 - DNA, 140 bp, Artificial Sequence
  Description of Artificial Sequence Synthetic polynucleotide
atatcatatgtgtggtagtggcagcgatggaggggtgtgtccgaagatcctaaagaagtgcaggagagacagcgactgccccggcgcttgcatctgtcgtggcaatggttattgcatcacgggagatgcactagttatat

SEQ ID NO: 35 - protein, 34 aa, Artificial Sequence
  Description of Artificial Sequence Synthetic polypeptide
CGSGSDGGVCPKILKKCRRDSDCPGACICRGNGY

SEQ ID NO: 36 - protein, 109 aa, Artificial Sequence
  Description of Artificial Sequence Synthetic polypeptide
CFNGSGSGGGSSGGGHHHHHHGGGSSGSGCYGNGRCICAGPCDSDRRCRQLIKPCVGGDSGGGGGGSHHHHHHSGGGDGGVCPKILQRCRRDSDCPGACICRGNGYCGS

SEQ ID NO: 37 - protein, 29 aa, Unknown
  Description of Unknown kalata_B1 sequence
GLPVCGETCVGGTCNTPGCTCSWPVCTRN

SEQ ID NO: 38 - protein, 30 aa, Unknown
  Description of Unknown cycloviolacin_O1 sequence
GIPCAESCVYIPCTVTALLGCSCSNRVCYN

SEQ ID NO: 39 - protein, 29 aa, Unknown
  Description of Unknown kalata_B2 sequence
GLPVCGETCFGGTCNTPGCSCTWPICTRD

SEQ ID NO: 40 - protein, 37 aa, Unknown
  Description of Unknown palicourein sequence
GDPTFCGETCRVIPVCTYSAALGCTCDDRSDGLCKRN

SEQ ID NO: 41 - protein, 30 aa, Unknown
  Description of Unknown vhr1 sequence
GIPCAESCVWIPCTVTALLGCSCSNKVCYN

SEQ ID NO: 42 - protein, 33 aa, Unknown
  Description of Unknown tricyclon_A sequence
GGTIFDCGESCFLGTCYTKGCSCGEWKLCYGTN

SEQ ID NO: 43 - protein, 30 aa, Unknown
  Description of Unknown circulin_A sequence
GIPCGESCVWIPCISAALGCSCKNKVCYRN

SEQ ID NO: 44 - protein, 30 aa, Unknown
  Description of Unknown cycloviolacin_O2 sequence
GIPCGESCVWIPCISSAIGCSCKSKVCYRN

SEQ ID NO: 45 - protein, 30 aa, Unknown
  Description of Unknown kalata_B6 sequence
GLPTCGETCFGGTCNTPGCSCSSWPICTRN

SEQ ID NO: 46 - protein, 30 aa, Unknown
  Description of Unknown kalata_B3 sequence
GLPTCGETCFGGTCNTPGCTCDPWPICTRD

SEQ ID NO: 47 - protein, 29 aa, Unknown
  Description of Unknown kalata_B7 sequence
GLPVCGETCTLGTCYTQGCTCSWPICKRN

SEQ ID NO: 48 - protein, 31 aa, Unknown
  Description of Unknown cycloviolacin_O8 sequence
GTLPCGESCVWIPCISSVVGCSCKSKVCYKN

SEQ ID NO: 49 - protein, 31 aa, Unknown
  Description of Unknown cycloviolacin_O11 sequence
GTLPCGESCVWIPCISAVVGCSCKSKVCYKN

SEQ ID NO: 50 - protein, 29 aa, Unknown
  Description of Unknown kalata_B4 sequence
GLPVCGETCVGGTCNTPGCTCSWPVCTRD

SEQ ID NO: 51 - protein, 29 aa, Unknown
  Description of Unknown vodo_M sequence
GAPICGESCFTGKCYTVQCSCSWPVCTRN

SEQ ID NO: 52 - protein, 31 aa, Unknown
  Description of Unknown cyclopsychotride_A sequence
SIPCGESCVFIPCTVTALLGCSCKSKVCYKN

SEQ ID NO: 53 - protein, 30 aa, Unknown
  Description of Unknown cycloviolacin_H1 sequence
GIPCGESCVYIPCLTSAIGCSCKSKVCYRN

SEQ ID NO: 54 - protein, 30 aa, Unknown
  Description of Unknown cycloviolacin_O9 sequence
GIPCGESCVWIPCLTSAVGCSCKSKVCYRN

SEQ ID NO: 55 - protein, 31 aa, Unknown
  Description of Unknown vico_A sequence
GSIPCAESCVYIPCFTGIAGCSCKNKVCYYN

SEQ ID NO: 56 - protein, 30 aa, Unknown
  Description of Unknown vitri_A sequence
GIPCGESCVWIPCITSAIGCSCKSKVCYRN

SEQ ID NO: 57 - protein, 29 aa, Unknown
  Description of Unknown kalata_S sequence
GLPVCGETCVGGTCNTPGCSCSWPVCTRN

SEQ ID NO: 58 - protein, 29 aa, Unknown
  Description of Unknown cycloviolacin_O12 sequence
GLPICGETCVGGTCNTPGCSCSWPVCTRN

SEQ ID NO: 59 - protein, 29 aa, Unknown
  Description of Unknown vodo_N sequence
GLPVCGETCTLGKCYTAGCSCSWPVCYRN

SEQ ID NO: 60 - protein, 31 aa, Unknown
  Description of Unknown vico_B sequence
GSIPCAESCVYIPCITGIAGCSCKNKVCYYN

SEQ ID NO: 61 - protein, 30 aa, Unknown
  Description of Unknown Hypa_A sequence
GIPCAESCVYIPCTITALLGCSCKNKVCYN

SEQ ID NO: 62 - protein, 31 aa, Unknown
  Description of Unknown circulin_B sequence
GVIPCGESCVFIPCISTLLGCSCKNKVCYRN

SEQ ID NO: 63 - protein, 30 aa, Unknown
  Description of Unknown circulin_C sequence
GIPCGESCVFIPCITSVAGCSCKSKVCYRN

SEQ ID NO: 64 - protein, 30 aa, Unknown
  Description of Unknown circulin_D sequence
KIPCGESCVWIPCVTSIFNCKCENKVCYHD

SEQ ID NO: 65 - protein, 30 aa, Unknown
  Description of Unknown circulin_E sequence
KIPCGESCVWIPCLTSVFNCKCENKVCYHD

SEQ ID NO: 66 - protein, 29 aa, Unknown
  Description of Unknown circulin_F sequence
AIPCGESCVWIPCISAAIGCSCKNKVCYR

SEQ ID NO: 67 - protein, 30 aa, Unknown
  Description of Unknown cycloviolacin_O4 sequence
GIPCGESCVWIPCISSAIGCSCKNKVCYRN

SEQ ID NO: 68 - protein, 30 aa, Unknown
  Description of Unknown cycloviolacin_O3 sequence
GIPCGESCVWIPCLTSAIGCSCKSKVCYRN

SEQ ID NO: 69 - protein, 30 aa, Unknown
  Description of Unknown cycloviolacin_O5 sequence
GTPCGESCVWIPCISSAVGCSCKNKVCYKN

SEQ ID NO: 70 - protein, 31 aa, Unknown
  Description of Unknown cycloviolacin_O6 sequence
GTLPCGESCVWIPCISAAVGCSCKSKVCYKN

SEQ ID NO: 71 - protein, 30 aa, Unknown
  Description of Unknown cycloviolacin_O7 sequence
SIPCGESCVWIPCTITALAGCKCKSKVCYN

SEQ ID NO: 72 - protein, 30 aa, Unknown
  Description of Unknown cycloviolacin_O10 sequence
GIPCGESCVYIPCLTSAVGCSCKSKVCYRN

SEQ ID NO: 73 - protein, 30 aa, Unknown
  Description of Unknown kalata_B5 sequence
GTPCGESCVYIPCISGVIGCSCTDKVCYLN

SEQ ID NO: 74 - protein, 30 aa, Unknown
  Description of Unknown varv_peptide_B sequence
GLPVCGETCFGGTCNTPGCSCDPWPMCSRN

SEQ ID NO: 75 - protein, 29 aa, Unknown
  Description of Unknown varv_peptide_C sequence
GVPICGETCVGGTCNTPGCSCSWPVCTRN

SEQ ID NO: 76 - protein, 29 aa, Unknown
  Description of Unknown varv_peptide_D sequence
GLPICGETCVGGSCNTPGCSCSWPVCTRN

SEQ ID NO: 77 - protein, 29 aa, Unknown
  Description of Unknown varv_peptide_F sequence
GVPICGETCTLGTCYTAGCSCSWPVCTRN

SEQ ID NO: 78 - protein, 30 aa, Unknown
  Description of Unknown varv_peptide_G sequence
GVPVCGETCFGGTCNTPGCSCDPWPVCSRN

SEQ ID NO: 79 - protein, 30 aa, Unknown
  Description of Unknown varv_peptide_H sequence
GLPVCGETCFGGTCNTPGCSCETWPVCSRN

SEQ ID NO: 80 - protein, 31 aa, Unknown
  Description of Unknown cycloviolin_A sequence
GVIPCGESCVFIPCISAAIGCSCKNKVCYRN

SEQ ID NO: 81 - protein, 28 aa, Unknown
  Description of Unknown cycloviolin_B sequence
GTACGESCYVLPCFTVGCTCTSSQCFKN

SEQ ID NO: 82 - protein, 30 aa, Unknown
  Description of Unknown cycloviolin_C sequence
GIPCGESCVFIPCLTTVAGCSCKNKVCYRN

SEQ ID NO: 83 - protein, 30 aa, Unknown
  Description of Unknown cycloviolin_D sequence
GFPCGESCVFIPCISAAIGCSCKNKVCYRN

SEQ ID NO: 84 - protein, 29 aa, Unknown
  Description of Unknown violapeptide_1 sequence
  MOD_RES (28)..(28): Any amino acid
GLPVCGETCVGGTCNTPGCSCSRPVCTXN

SEQ ID NO: 85 - protein, 31 aa, Unknown
  Description of Unknown vhl-1 sequence
SISCGESCAMISFCFTEVIGCSCKNKVCYLN

SEQ ID NO: 86 - protein, 30 aa, Unknown
  Description of Unknown hcf-1 sequence
GIPCGESCHYIPCVTSAIGCSCRNRSCMRN

SEQ ID NO: 87 - protein, 30 aa, Unknown
  Description of Unknown htf-1 sequence
GIPCGDSCHYIPCVTSTIGCSCTNGSCMRN

SEQ ID NO: 88 - protein, 30 aa, Unknown
  Description of Unknown vhl-2 sequence
GLPVCGETCFTGTCYTNGCTCDPWPVCTRN

SEQ ID NO: 89 - protein, 30 aa, Unknown
  Description of Unknown cycloviolacin_H3 sequence
GLPVCGETCFGGTCNTPGCICDPWPVCTRN

SEQ ID NO: 90 - protein, 29 aa, Unknown
  Description of Unknown cycloviolacin_H2 sequence
SAIACGESCVYIPCFIPGCSCRNRVCYLN

SEQ ID NO: 91 - protein, 31 aa, Unknown
  Description of Unknown Hyfl_A sequence
SISCGESCVYIPCTVTALVGCTCKDKVCYLN

SEQ ID NO: 92 - protein, 32 aa, Unknown
  Description of Unknown Hyfl_B sequence
GSPIQCAETCFIGKCYTEELGCTCTAFLCMKN

SEQ ID NO: 93 - protein, 32 aa, Unknown
  Description of Unknown Hyfl_C sequence
GSPRQCAETCFIGKCYTEELGCTCTAFLCMKN

SEQ ID NO: 94 - protein, 31 aa, Unknown
  Description of Unknown Hyfl_D sequence
GSVPCGESCVYIPCFTGIAGCSCKSKVCYYN

SEQ ID NO: 95 - protein, 29 aa, Unknown
  Description of Unknown Hyfl_E sequence
GEIPCGESCVYLPCFLPNCYCRNHVCYLN

SEQ ID NO: 96 - protein, 30 aa, Unknown
  Description of Unknown Hyfl_F sequence
SISCGETCTTFNCWIPNCKCNHHDKVCYWN

SEQ ID NO: 97 - protein, 30 aa, Unknown
  Description of Unknown Hyfl_I sequence
GIPCGESCVFIPCISGVIGCSCKSKVCYRN

SEQ ID NO: 98 - protein, 28 aa, Unknown
  Description of Unknown Hyfl_J sequence
GIACGESCAYFGCWIPGCSCRNKVCYFN

SEQ ID NO: 99 - protein, 30 aa, Unknown
  Description of Unknown Hyfl_K sequence
GTPCGESCVYIPCFTAVVGCTCKDKVCYLN

SEQ ID NO: 100 - protein, 30 aa, Unknown
  Description of Unknown Hyfl_L sequence
GTPCAESCVYLPCFTGVIGCTCKDKVCYLN

SEQ ID NO: 101 - protein, 29 aa, Unknown
  Description of Unknown Hyfl_M sequence
GNIPCGESCIFFPCFNPGCSCKDNLCYYN

SEQ ID NO: 102 - protein, 33 aa, Unknown
  Description of Unknown tricyclon_B sequence
GGTIFDCGESCFLGTCYTKGCSCGEWKLCYGEN

SEQ ID NO: 103 - protein, 31 aa, Unknown
  Description of Unknown kalata_B8 sequence
GSVLNCGETCLLGTCYTTGCTCNKYRVCTKD

SEQ ID NO: 104 - protein, 30 aa, Unknown
  Description of Unknown cycloviolacin_H4 sequence
GIPCAESCVWIPCTVTALLGCSCSNNVCYN

SEQ ID NO: 105 - protein, 30 aa, Unknown
  Description of Unknown cycloviolacin_O13 sequence
GIPCGESCVWIPCISAAIGCSCKSKVCYRN

SEQ ID NO: 106 - protein, 27 aa, Unknown
  Description of Unknown violacin_A sequence
SAISCGETCFKFKCYTPRCSCSYPVCK

SEQ ID NO: 107 - protein, 31 aa, Unknown
  Description of Unknown cycloviolacin_O14 sequence
GSIPACGESCFKGKCYTPGCSCSKYPLCAKN

SEQ ID NO: 108 - protein, 29 aa, Unknown
  Description of Unknown cycloviolacin_O15 sequence
GLVPCGETCFTGKCYTPGCSCSYPICKKN

SEQ ID NO: 109 - protein, 29 aa, Unknown
  Description of Unknown cycloviolacin_O16 sequence
GLPCGETCFTGKCYTPGCSCSYPICKKIN

SEQ ID NO: 110 - protein, 30 aa, Unknown
  Description of Unknown cycloviolacin_O17 sequence
GIPCGESCVWIPCISAAIGCSCKNKVCYRN

SEQ ID NO: 111 - protein, 30 aa, Unknown
  Description of Unknown cycloviolacin_O18 sequence
GIPCGESCVYIPCTVTALAGCKCKSKVCYN

SEQ ID NO: 112 - protein, 31 aa, Unknown
  Description of Unknown cycloviolacin_O19 sequence
GTLPCGESCVWIPCISSVVGCSCKSKVCYKD

SEQ ID NO: 113 - protein, 30 aa, Unknown
  Description of Unknown cycloviolacin_O20 sequence
GIPCGESCVWIPCLTSAIGCSCKSKVCYRD

SEQ ID NO: 114 - protein, 29 aa, Unknown
  Description of Unknown cycloviolacin_O21 sequence
GLPVCGETCVTGSCYTPGCTCSWPVCTRN

SEQ ID NO: 115 - protein, 29 aa, Unknown
  Description of Unknown cycloviolacin_O22 sequence
GLPICGETCVGGTCNTPGCTCSWPVCTRN

SEQ ID NO: 116 - protein, 31 aa, Unknown
  Description of Unknown cycloviolacin_O23 sequence
GLPTCGETCFGGTCNTPGCTCDSSWPICTHN

SEQ ID NO: 117 - protein, 30 aa, Unknown
  Description of Unknown cycloviolacin_O24 sequence
GLPTCGETCFGGTCNTPGCTCDPWPVCTHN

SEQ ID NO: 118 - protein, 31 aa, Unknown
  Description of Unknown cycloviolacin_O25 sequence
DIFCGETCAFIPCITHVPGTCSCKSKVCYFN

SEQ ID NO: 119 - protein, 31 aa, Unknown
  Description of Unknown kalata_B9 sequence
GSVFNCGETCVLGTCYTPGCTCNTYRVCTKD

SEQ ID NO: 120 - protein, 31 aa, Unknown; identical to SEQ ID NO: 119
  Description of Unknown kalata_B9_linear sequence

SEQ ID NO: 121 - protein, 30 aa, Unknown
  Description of Unknown kalata_B10 sequence
GLPTCGETCFGGTCNTPGCSCSSWPICTRD

SEQ ID NO: 122 - protein, 30 aa, Unknown; identical to SEQ ID NO: 121
  Description of Unknown kalata_B10_linear sequence

SEQ ID NO: 123 - protein, 29 aa, Unknown
  Description of Unknown kalata_B11 sequence
GLPVCGETCFGGTCNTPGCSCTDPICTRD

SEQ ID NO: 124 - protein, 28 aa, Unknown
  Description of Unknown kalata_B12 sequence
GSLCGDTCFVLGCNDSSCSCNYPICVKD

SEQ ID NO: 125 - protein, 30 aa, Unknown
  Description of Unknown kalata_B13 sequence
GLPVCGETCFGGTCNTPGCACDPWPVCTRD

SEQ ID NO: 126 - protein, 30 aa, Unknown
  Description of Unknown kalata_B14 sequence
GLPVCGESCFGGTCNTPGCACDPWPVCTRD

SEQ ID NO: 127 - protein, 29 aa, Unknown
  Description of Unknown kalata_B15 sequence
GLPVCGESCFGGSCYTPGCSCTWPICTRD

SEQ ID NO: 128 - protein, 30 aa, Unknown
  Description of Unknown kalata_B16 sequence
GIPCAESCVYIPCTITALLGCKCQDKVCYD

SEQ ID NO: 129 - protein, 30 aa, Unknown
  Description of Unknown kalata_B17 sequence
GIPCAESCVYIPCTITALLGCKCKDQVCYN

SEQ ID NO: 130 - protein, 30 aa, Unknown
  Description of Unknown kalata_B18 sequence
GVPCAESCVYIPCISTVLGCSCSNQVCYRN

SEQ ID NO: 131 - protein, 31 aa, Unknown
  Description of Unknown PS-1 sequence
GFIPCGETCIWDKTCHAAGCSCSVANICVRN

SEQ ID NO: 132 - protein, 34 aa, Unknown
  Description of Unknown CD-1 sequence
GADGFCGESCYVIPCISYLVGCSCDTIEKVCKRN

SEQ ID NO: 133 - protein, 33 aa, Unknown
  Description of Unknown cycloviolacin_Y1 sequence
GGTIFDCGETCFLGTCYTPGCSCGNYGFCYGTN

SEQ ID NO: 134 - protein, 33 aa, Unknown
  Description of Unknown cycloviolacin_Y2 sequence
GGTIFDCGESCFLGTCYTAGCSCGNWGLCYGTN

SEQ ID NO: 135 - protein, 33 aa, Unknown
  Description of Unknown cycloviolacin_Y3 sequence
GGTIFDCGETCFLGTCYTAGCSCGNWGLCYGTN

SEQ ID NO: 136 - protein, 30 aa, Unknown
  Description of Unknown cycloviolacin_Y4 sequence
GVPCGESCVFIPCITGVIGCSCSSNVCYLN

SEQ ID NO: 137 - protein, 30 aa, Unknown
  Description of Unknown cycloviolacin_Y5 sequence
GIPCAESCVWIPCTVTALVGCSCSDKVCYN

SEQ ID NO: 138 - protein, 29 aa, Unknown
  Description of Unknown vibi_A sequence
GLPVCGETCFGGTCNTPGCSCSYPICTRN

SEQ ID NO: 139 - protein, 29 aa, Unknown
  Description of Unknown vibi_B sequence
GLPVCGETCFGGTCNTPGCTCSYPICTRN

SEQ ID NO: 140 - protein, 29 aa, Unknown
  Description of Unknown vibi_C sequence
GLPVCGETCAFGSCYTPGCSCSWPVCTRN

SEQ ID NO: 141 - protein, 29 aa, Unknown
  Description of Unknown vibi_D sequence
GLPVCGETCFGGRCNTPGCTCSYPICTRN

SEQ ID NO: 142 - protein, 30 aa, Unknown
  Description of Unknown vibi_E sequence
GIPCAESCVWIPCTVTALIGCGCSNKVCYN

SEQ ID NO: 143 - protein, 31 aa, Unknown
  Description of Unknown vibi_F sequence
GTIPCGESCVFIPCLTSALGCSCKSKVCYKN

SEQ ID NO: 144 - protein, 31 aa, Unknown
  Description of Unknown vibi_G sequence
GTFPCGESCVFIPCLTSAIGCSCKSKVCYKN

SEQ ID NO: 145 - protein, 31 aa, Unknown
  Description of Unknown vibi_H sequence
GLLPCAESCVYIPCLTTVIGCSCKSKVCYKN

SEQ ID NO: 146 - protein, 30 aa, Unknown
  Description of Unknown vibi_I sequence
GIPCGESCVWIPCLTSTVGCSCKSKVCYRN

SEQ ID NO: 147 - protein, 31 aa, Unknown
  Description of Unknown vibi_J sequence
GTFPCGESCVWIPCISKVIGCACKSKVCYKN

SEQ ID NO: 148 - protein, 30 aa, Unknown
  Description of Unknown vibi_K sequence
GIPCGESCVWIPCLTSAVGCPCKSKVCYRN

SEQ ID NO: 149 - protein, 30 aa, Unknown
  Description of Unknown Viba_2 sequence
GIPCGESCVYLPCFTAPLGCSCSSKVCYRN

SEQ ID NO: 150 - protein, 30 aa, Unknown
  Description of Unknown Viba_5 sequence
GIPCGESCVWIPCLTATIGCSCKSKVCYRN

SEQ ID NO: 151 - protein, 29 aa, Unknown
  Description of Unknown Viba_10 sequence
GIPCAESCVYLPCVTIVIGCSCKDKVCYN

SEQ ID NO: 152 - protein, 30 aa, Unknown
  Description of Unknown Viba_12 sequence
GIPCAESCVWIPCTVTALLGCSCKDKVCYN

SEQ ID NO: 153 - protein, 34 aa, Unknown
  Description of Unknown Viba_14 sequence
GRLCGERCVIERTRAWCRTVGCICSLHTLECVRN

SEQ ID NO: 154 - protein, 29 aa, Unknown
  Description of Unknown Viba_17 sequence
GLPVCGETCVGGTCNTPGCGCSWPVCTRN

SEQ ID NO: 155 - protein, 29 aa, Unknown
  Description of Unknown Viba_15 sequence
GLPVCGETCVGGTCNTPGCACSWPVCTRN

SEQ ID NO: 156 - protein, 31 aa, Unknown
  Description of Unknown Mra4 sequence
GSIPCGESCVYIPCISSLLGCSCKSKVCYKN

SEQ ID NO: 157 - protein, 30 aa, Unknown
  Description of Unknown Mra5 sequence
GIPCAESCVYIPCLTSAIGCSCKSKVCYRN

SEQ ID NO: 158 - protein, 29 aa, Unknown
  Description of Unknown Mra13 sequence
GIPCGESCVYLPCFTTIIGCKCQGKVCYH

SEQ ID NO: 159 - protein, 31 aa, Unknown
  Description of Unknown Mra14a sequence
GSIPCGESCVFIPCISSVVGCSCKNKVCYKN

SEQ ID NO: 160 - protein, 31 aa, Unknown
  Description of Unknown Mra14b sequence
GTIPCGESCVFIPCLTSAIGCSCKSKVCYKN

SEQ ID NO: 161 - protein, 31 aa, Unknown
  Description of Unknown Mra17 sequence
GSIPCGESCVYIPCISSLLGCSCESKVCYKN

SEQ ID NO: 162 - protein, 31 aa, Unknown
  Description of Unknown Mra29 sequence
GSIPCGESCVFIPCISSIVGCSCKSKVCYKN

SEQ ID NO: 163 - protein, 30 aa, Unknown
  Description of Unknown Mra30 sequence
GIPCGESCVFIPCLTSAIGCSCKSKVCYRN

SEQ ID NO: 164 - protein, 30 aa, Unknown
  Description of Unknown Mra22 sequence
GVPCGESCVWIPCLTSIVGCSCKNNVCTLN

SEQ ID NO: 165 - protein, 31 aa, Unknown
  Description of Unknown Mra23 sequence
GVIPCGESCVFIPCISSVLGCSCKNKVCYRN

SEQ ID NO: 166 - protein, 29 aa, Unknown
  Description of Unknown Mra24 sequence
GHPTCGETCLLGTCYTPGCTCKRPVCYKN

SEQ ID NO: 167 - protein, 30 aa, Unknown
  Description of Unknown Mra25 sequence
GSAILCGESCTLGECYTPGCTCSWPICTKN

SEQ ID NO: 168 - protein, 29 aa, Unknown
  Description of Unknown Mra26 sequence
GHPICGETCVGNKCYTPGCTCTWPVCYRN

SEQ ID NO: 169 - protein, 31 aa, Unknown
  Description of Unknown Mra30 sequence
GSIPCGEGCVFIPCISSIVGCSCKSKVCYKN

SEQ ID NO: 170 - protein, 30 aa, Unknown
  Description of Unknown Viba_1 sequence
GIPCGEGCVYLPCFTAPLGCSCSSKVCYRN

SEQ ID NO: 171 - protein, 30 aa, Unknown
  Description of Unknown Viba_3 sequence
GIPCGESCVWIPCLTAAIGCSCSSKVCYRN

SEQ ID NO: 172 - protein, 30 aa, Unknown
  Description of Unknown Viba_4 sequence
GVPCGESCVWIPCLTSAIGCSCKSSVCYRN

SEQ ID NO: 173 - protein, 30 aa, Unknown
  Description of Unknown Viba_6 sequence
GIPCGESCVLIPCISSVIGCSCKSKVCYRN

SEQ ID NO: 174 - protein, 31 aa, Unknown
  Description of Unknown Viba_7 sequence
GVIPCGESCVFIPCISSVIGCSCKSKVCYRN

SEQ ID NO: 175 - protein, 30 aa, Unknown
  Description of Unknown Viba_8 sequence
GAGCIETCYTFPCISEMINCSCKNSRCQKN

SEQ ID NO: 176 - protein, 30 aa, Unknown
  Description of Unknown Viba_9 sequence
GIPCGESCVWIPCISSAIGCSCKNKVCYRK

SEQ ID NO: 177 - protein, 30 aa, Unknown
  Description of Unknown Viba_11 sequence
GIPCGESCVWIPCISGAIGCSCKSKVCYRN

SEQ ID NO: 178 - protein, 30 aa, Unknown
  Description of Unknown Viba_13 sequence
TIPCAESCVWIPCTVTALLGCSCKDKVCYN

SEQ ID NO: 179 - protein, 29 aa, Unknown
  Description of Unknown Viba_16 sequence
GLPICGETCTLGTCYTVGCTCSWPICTRN

SEQ ID NO: 180 - protein, 31 aa, Unknown
  Description of Unknown Cter_A sequence
GVIPCGESCVFIPCISTVIGCSCKNKVCYRN

SEQ ID NO: 181 - protein, 31 aa, Unknown
  Description of Unknown Cter_B sequence
GVPCAESCVWIPCTVTALLGCSCKDKVCYLN

SEQ ID NO: 182 - protein, 29 aa, Unknown
  Description of Unknown hcf-1_variant sequence
GIPCGESCHIPCVTSAIGCSCRNRSCMRN

SEQ ID NO: 183 - protein, 31 aa, Unknown
  Description of Unknown Vpl-1 sequence
GSQSCGESCVLIPCISGVIGCSCSSMICYFN

SEQ ID NO: 184 - protein, 30 aa, Unknown
  Description of Unknown Vpf-1 sequence
GIPCGESCVFIPCLTAAIGCSCRSKVCYRN

SEQ ID NO: 185 - protein, 29 aa, Unknown
  Description of Unknown cO31 sequence
GLPVCGETCVGGTCNTPGCSCSIPVCTRN

SEQ ID NO: 186 - protein, 29 aa, Unknown
  Description of Unknown cO28 sequence
GLPVCGETCVGGTCNTPGCSCSWPVCFRD

SEQ ID NO: 187 - protein, 30 aa, Unknown
  Description of Unknown cO32 sequence
GAPVCGETCFGGTCNTPGCTCDPWPVCTND

SEQ ID NO: 188 - protein, 29 aa, Unknown
  Description of Unknown cO33 sequence
GLPVCGETCVGGTCNTPYCTCSWPVCTRD

SEQ ID NO: 189 - protein, 29 aa, Unknown
  Description of Unknown cO34 sequence
GLPVCGETCVGGTCNTEYCTCSWPVCTRD

SEQ ID NO: 190 - protein, 29 aa, Unknown
  Description of Unknown cO35 sequence
GLPVCGETCVGGTCNTPYCFCSWPVCTRD

SEQ ID NO: 191 - protein, 30 aa, Unknown
  Description of Unknown cO29 sequence
GIPCGESCVWIPCISGAIGCSCKSKVCYKN

SEQ ID NO: 192 - protein, 30 aa, Unknown
  Description of Unknown cO30 sequence
GIPCGESCVWIPCISSAIGCSCKNKVCFKN

SEQ ID NO: 193 - protein, 31 aa, Unknown
  Description of Unknown cO26 sequence
GSIPACGESCFRGKCYTPGCSCSKYPLCAKD

SEQ ID NO: 194 - protein, 31 aa, Unknown
  Description of Unknown cO27 sequence
GSIPACGESCFKGWCYTPGCSCSKYPLCAKD

SEQ ID NO: 195 - protein, 31 aa, Unknown
  Description of Unknown Globa_F sequence
GSFPCGESCVFIPCISAIAGCSCKNKVCYKN

SEQ ID NO: 196 - protein, 30 aa, Unknown
  Description of Unknown Globa_A sequence
GIPCGESCVFIPCITAAIGCSCKTKVCYRN

SEQ ID NO: 197 - protein, 31 aa, Unknown
  Description of Unknown Globa_B sequence
GVIPCGESCVFIPCISAVLGCSCKSKVCYRN

SEQ ID NO: 198 - protein, 30 aa, Unknown
  Description of Unknown Globa_D sequence
GIPCGETCVFMPCISGPMGCSCKHMVCYRN

SEQ ID NO: 199 - protein, 30 aa, Unknown
  Description of Unknown Globa_E sequence
GSAFGCGETCVKGKCNTPGCVCSWPVCKKN

SEQ ID NO: 200 - protein, 30 aa, Unknown
  Description of Unknown Globa_C sequence
APCGESCVYIPCLLTAPIGCSCSNIVCYRN

SEQ ID NO: 201 - protein, 31 aa, Unknown
  Description of Unknown Glopa_D sequence
GVPCGESCVWVPCTVTALMGCSCVREVCRKD

SEQ ID NO: 202 - protein, 30 aa, Unknown
  Description of Unknown Glopa_E sequence
GIPCAESCVWIPCTVTKMLGCSCKDKVCYN

SEQ ID NO: 203 - protein, 32 aa, Unknown
  Description of Unknown Glopa_A sequence
GGSIPCIETCVWTGCFLVPGCSCKSDKKCYLN

SEQ ID NO: 204 - protein, 32 aa, Unknown
  Description of Unknown Glopa_B sequence
GGSVPCIETCVWTGCFLVPGCSCKSDKKCYLN

SEQ ID NO: 205 - protein, 31 aa, Unknown
  Description of Unknown Glopa_C sequence
GDIPLCGETCFEGGNCRIPGCTCVWPFCSKN

SEQ ID NO: 206 - protein, 30 aa, Unknown
  Description of Unknown cO36 sequence
GLPTCGETCFGGTCNTPGCTCDPFPVCTHD

SEQ ID NO: 207 - protein, 29 aa, Unknown
  Description of Unknown cycloviolacin_T1 sequence
GIPVCGETCVGGTCNTPGCSCSWPVCTRN

SEQ ID NO: 208 - protein, 28 aa, Unknown
  Description of Unknown psyle_A sequence
GIACGESCVFLGCFIPGCSCKSKVCYFN

SEQ ID NO: 209 - protein, 28 aa, Unknown
  Description of Unknown psyle_B sequence
GIPCGETCVAFGCWIPGCSCKDKLCYYD

SEQ ID NO: 210 - protein, 25 aa, Unknown
  Description of Unknown psyle_C sequence
KLCGETCFKFKCYTPGCSCSYPFCK

SEQ ID NO: 211 - protein, 31 aa, Unknown
  Description of Unknown psyle_D sequence
GIPCGESCVFIPCTVTALLGCSCQNKVCYRD

SEQ ID NO: 212 - protein, 31 aa, Unknown
  Description of Unknown psyle_E sequence
GVIPCGESCVFIPCISSVLGCSCKNKVCYRD

SEQ ID NO: 213 - protein, 31 aa, Unknown
  Description of Unknown psyle_F sequence
GVIPCGESCVFIPCITAAVGCSCKNKVCYRD

SEQ ID NO: 214 - protein, 29 aa, Unknown
  Description of Unknown vaby_A sequence
GLPVCGETCAGGTCNTPGCSCSWPICTRN

SEQ ID NO: 215 - protein, 29 aa, Unknown
  Description of Unknown vaby_B sequence
GLPVCGETCAGGTCNTPGCSCTWPICTRN

SEQ ID NO: 216 - protein, 29 aa, Unknown
  Description of Unknown vaby_C sequence
GLPVCGETCAGGRCNTPGCSCSWPVCTRN

SEQ ID NO: 217 - protein, 30 aa, Unknown
  Description of Unknown vaby_D sequence
GLPVCGETCFGGTCNTPGCTCDPWPVCTRN

SEQ ID NO: 218 - protein, 30 aa, Unknown
  Description of Unknown vaby_E sequence
GLPVCGETCFGGTCNTPGCSCDPWPVCTRN

SEQ ID NO: 219 - protein, 30 aa, Unknown
  Description of Unknown kalata_B19 sequence
GFPCGESCVYVPCLTAAIGCSCSNKVCYKN

SEQ ID NO: 220 - protein, 30 aa, Unknown
  Description of Unknown Oak6_cyclotide_2 sequence
GLPICGETCFGGTCNTPGCICDPWPVCTRD

SEQ ID NO: 221 - protein, 29 aa, Unknown
  Description of Unknown Oak7_cyclotide sequence
GSHCGETCFFFGCYKPGCSCDELRQCYKN

SEQ ID NO: 222 - protein, 30 aa, Unknown
  Description of Unknown Oak8_cyclotide sequence
GVPCGESCVFIPCLTAVVGCSCSNKVCYLN

SEQ ID NO: 223 - protein, 30 aa, Unknown
  Description of Unknown Oak6_cyclotide_1 sequence
GLPVCGETCFGGTCNTPGCACDPWPVCTRN

SEQ ID NO: 224 - protein, 31 aa, Unknown
  Description of Unknown Cter_C sequence
GVPCAESCVWIPCTVTALLGCSCKDKVCYLD

SEQ ID NO: 225 - protein, 31 aa, Unknown
  Description of Unknown Cter_D sequence
GIPCAESCVWIPCTVTALLGCSCKDKVCYLN

SEQ ID NO: 226 - protein, 31 aa, Unknown
  Description of Unknown Cter_E sequence
GIPCAESCVWIPCTVTALLGCSCKDKVCYLD

SEQ ID NO: 227 - protein, 30 aa, Unknown
  Description of Unknown Cter_F sequence
GIPCGESCVFIPCISSVVGCSCKSKVCYLD

SEQ ID NO: 228 - protein, 30 aa, Unknown
  Description of Unknown Cter_G sequence
GLPCGESCVFIPCITTVVGCSCKNKVCYNN

SEQ ID NO: 229 - protein, 30 aa, Unknown
  Description of Unknown Cter_H sequence
GLPCGESCVFIPCITTVVGCSCKNKVCYND

SEQ ID NO: 230 - protein, 31 aa, Unknown
  Description of Unknown Cter_I sequence
GTVPCGESCVFIPCITGIAGCSCKNKVCYIN

SEQ ID NO: 231 - protein, 31 aa, Unknown
  Description of Unknown Cter_J sequence
GTVPCGESCVFIPCITGIAGCSCKNKVCYID

SEQ ID NO: 232 - protein, 29 aa, Unknown
  Description of Unknown Cter_K sequence
HEPCGESCVFIPCITTVVGCSCKNKVCYN

SEQ ID NO: 233 - protein, 29 aa, Unknown
  Description of Unknown Cter_L sequence
HEPCGESCVFIPCITTVVGCSCKNKVCYD

SEQ ID NO: 234 - protein, 29 aa, Unknown
  Description of Unknown Cter_M sequence
GLPTCGETCTLGTCYVPDCSCSWPICMKN

SEQ ID NO: 235 - protein, 29 aa, Unknown
  Description of Unknown Cter_N sequence
GSAFCGETCVLGTCYTPDCSCTALVCLKN

SEQ ID NO: 236 - protein, 30 aa, Unknown
  Description of Unknown Cter_O sequence
GIPCGESCVFIPCITGIAGCSCKSKVCYRN

SEQ ID NO: 237 - protein, 30 aa, Unknown
  Description of Unknown Cter_P sequence
GIPCGESCVFIPCITAAIGCSCKSKVCYRN

SEQ ID NO: 238 - protein, 30 aa, Unknown
  Description of Unknown Cter_Q sequence
GIPCGESCVFIPCISTVIGCSCKNKVCYRN

SEQ ID NO: 239 - protein, 31 aa, Unknown
  Description of Unknown Cter_R sequence
GIPCGESCVFIPCTVTALLGCSCKDKVCYKN

SEQ ID NO: 240 - protein, 29 aa, Unknown
  Description of Unknown vitri_B sequence
GVPICGESCVGGTCNTPGCSCSWPVCTTN

SEQ ID NO: 241 - protein, 29 aa, Unknown
  Description of Unknown vitri_C sequence
GLPICGETCVGGTCNTPGCFCTWPVCTRN

SEQ ID NO: 242 - protein, 29 aa, Unknown
  Description of Unknown vitri_D sequence
GLPVCGETCFTGSCYTPGCSCNWPVCNRN

SEQ ID NO: 243 - protein, 29 aa, Unknown
  Description of Unknown vitri_E sequence
GLPVCGETCVGGTCNTPGCSCSWPVCFRN

SEQ ID NO: 244 - protein, 31 aa, Unknown
  Description of Unknown vitri_F sequence
GLTPCGESCVWIPCISSVVGCACKSKVCYKD

SEQ ID NO: 245 - protein, 30 aa, Unknown
  Description of Unknown hedyotide_B1 sequence
GTRCGETCFVLPCWSAKFGCYCQKGFCYRN

SEQ ID NO: 246 - protein, 32 aa, Unknown
  Description of Unknown Parigidin-br1 sequence
GGSVPCGESCVFIPCITSLAGCSCKNKVCYYD

SEQ ID NO: 247 - protein, 29 aa, Unknown
  Description of Unknown hedyotide_B2 sequence
GIQCGESCVWIPCISSAWGCSCKNKICSS

SEQ ID NO: 248 - protein, 31 aa, Unknown
  Description of Unknown viphi_A sequence
GSIPCGESCVFIPCISSVIGCACKSKVCYKN

SEQ ID NO: 249 - protein, 29 aa, Unknown
  Description of Unknown viphi_B sequence
GLPVCGETCTIGTCYTAGCTCSWPICTRN

SEQ ID NO: 250 - protein, 30 aa, Unknown
  Description of Unknown viphi_C sequence
GVPCGESCVYIPCITSVIGCSCSSKVCYIN

SEQ ID NO: 251 - protein, 30 aa, Unknown
  Description of Unknown viphi_D sequence
GIPCGESCVFIPCISSVIGCSCSSKVCYRN

SEQ ID NO: 252 - protein, 31 aa, Unknown
  Description of Unknown viphi_E sequence
GSIPCGESCVFIPCISAVIGCSCSNKVCYKN

SEQ ID NO: 253 - protein, 31 aa, Unknown
  Description of Unknown viphi_F sequence
GSIPCGESCVFIPCISAIIGCSCSSKVCYKN

SEQ ID NO: 254 - protein, 31 aa, Unknown
  Description of Unknown viphi_G sequence
GSIPCGESCVFIPCISAIIGCSCSNKVCYKN

SEQ ID NO: 255 - protein, 30 aa, Unknown
  Description of Unknown viphi_H sequence
GIPCAESCVWIPCTVTAIVGCSCSWGVCYN

SEQ ID NO: 256 - protein, 30 aa, Unknown
  Description of Unknown cliotide_T8 sequence
GIPCGESCVFIPCISSVVGCSCKSKVCYNN

SEQ ID NO: 257 - protein, 30 aa, Unknown
  Description of Unknown cliotide_T9 sequence
GIPCGESCVFIPCLTTVVGCSCKNKVCYNN

SEQ ID NO: 258 - protein, 30 aa, Unknown
  Description of Unknown cliotide_T2 sequence
GEFLKCGESCVQGECYTPGCSCDWPICKKN

SEQ ID NO: 259 - protein, 30 aa, Unknown
  Description of Unknown cliotide_T12 sequence
GIPCGESCVFIPCITGAIGCSCKSKVCYRD

SEQ ID NO: 260 - protein, 28 aa, Unknown
  Description of Unknown Panitide_L1 sequence
QLPICGETCVLGTCYTPGCRCQYPICVR

SEQ ID NO: 261 - protein, 28 aa, Unknown
  Description of Unknown Panitide_L2 sequence
QLPICGETCVLGRCYTPNCRCQYPICVR

SEQ ID NO: 262 - protein, 27 aa, Unknown
  Description of Unknown Panitide_L4 sequence
QAFCGETCLLGTCYTPGCRCTAGICLK

SEQ ID NO: 263 - protein, 28 aa, Unknown
  Description of Unknown Panitide_L6 sequence
QLPICGETCVLGTCYTPGCSCAYPICVR

SEQ ID NO: 264 - protein, 27 aa, Unknown
  Description of Unknown Panitide_L3 sequence
QAFCGETCLLGKCYTPGCSCHTGICLK

SEQ ID NO: 265 - protein, 28 aa, Unknown
  Description of Unknown Panitide_L5 sequence
QLPICGETCVLGTCYTPGCSCAYPICAR

SEQ ID NO: 266 - protein, 27 aa, Unknown
  Description of Unknown Panitide_L7 sequence
QAFCGETCVLGTCYTPGCSCNFGICLK

SEQ ID NO: 267 - protein, 26 aa, Unknown
  Description of Unknown Panitide_L8 sequence
QDCGETCVLGTCYTPGCSCSAYPLCV

SEQ ID NO: 268 - protein, 29 aa, Unknown
  Description of Unknown vigno_1 sequence
GLPLCGETCAGGTCNTPGCSCSWPVCVRN

SEQ ID NO: 269 - protein, 30 aa, Unknown
  Description of Unknown vigno_2 sequence
GSSPLCGETCAGGTCNTPGCSCSWPVCVRD

SEQ ID NO: 270 - protein, 29 aa, Unknown
  Description of Unknown vigno_3 sequence
GLPLCGETCVGGTCNTPGCSCSWPVCTRN

SEQ ID NO: 271 - protein, 29 aa, Unknown
  Description of Unknown vigno_4 sequence
GLPLCGETCVGGTCNTPACSCSWPVCTRN

SEQ ID NO: 272 - protein, 29 aa, Unknown
  Description of Unknown vigno_5 sequence
GLPLCGETCVGGTCNTPGCSCGWPVCVRN

SEQ ID NO: 273 - protein, 31 aa, Unknown
  Description of Unknown vigno_6 sequence
GIPCGESCVWIPCISSAIGCSCKGSKVCYRN

SEQ ID NO: 274 - protein, 31 aa, Unknown
  Description of Unknown vigno_7 sequence
GTLPCGESCVWIPCISSVVGCSCKNKVCYKN

SEQ ID NO: 275 - protein, 30 aa, Unknown
  Description of Unknown vigno_8 sequence
GIPCGESCVWIPCITSAVGCSCKSKVCYRN

SEQ ID NO: 276 - protein, 30 aa, Unknown
  Description of Unknown vigno_9 sequence
GIPCGESCVWIPCISSALGCSCKSKVCYRN

SEQ ID NO: 277 - protein, 31 aa, Unknown
  Description of Unknown vigno_10 sequence
GTIPCGESCVWIPCISSVVGCSCKSKVCYKD

SEQ ID NO: 278 - protein, 31 aa, Unknown
  Description of Unknown caripe_1 sequence
GVIPCGESCVFIPCISTVIGCSCKDKVCYRN

SEQ ID NO: 279 - protein, 31 aa, Unknown
  Description of Unknown caripe_2 sequence
GIPCGESCVFIRCTITALLGCSCSNNVCYKN

SEQ ID NO: 280 - protein, 27 aa, Unknown
  Description of Unknown caripe_4 sequence
LICSSTCLRIPCLSPRCTCRHHICYLN

SEQ ID NO: 281 - protein, 28 aa, Unknown
  Description of Unknown caripe_6 sequence
GAICTGTCFRNPCLSRRCTCRHYICYLN

SEQ ID NO: 282 - protein, 29 aa, Unknown
  Description of Unknown chacur_1 sequence
GLPVCGETCVGGTCNTPGCTCSWPICTRN

SEQ ID NO: 283 - protein, 29 aa, Unknown
  Description of Unknown psybra_1 sequence
GLPICGETCTLGTCNTPGCTCSWPICTKN

SEQ ID NO: 284 - protein, 29 aa, Unknown
  Description of Unknown paltet_1 sequence
GLPICGETCFTGTCNTPGCTCSYPVCTRN

SEQ ID NO: 285 - protein, 29 aa, Unknown
  Description of Unknown psypoe_1 sequence
  MOD_RES (25)..(25): Any amino acid
GSVICGETCFTTVCNTPGCYCGAYXCTRN

SEQ ID NO: 286 - protein, 31 aa, Unknown
  Description of Unknown caripe_7 sequence
GIPCGESCVFIPCTVTALLGCSCKNKVCYRN

SEQ ID NO: 287 - protein, 31 aa, Unknown
  Description of Unknown caripe_8 sequence
GVIPCGESCVFIPCITAAIGCSCKKKVCYRN

SEQ ID NO: 288 - protein, 30 aa, Unknown
  Description of Unknown chassatide_C18 sequence
GIPCGESCVFIPCISALLGCSCSNKVCYNN

SEQ ID NO: 289 - protein, 31 aa, Unknown
  Description of Unknown chassatide_C16 sequence
GVPCAESCVYIPCTITALFGCSCKDKVCYNN

SEQ ID NO: 290 - protein, 31 aa, Unknown
  Description of Unknown chassatide_C15 sequence
GIPCAESCVYIPCTITALLGCSCKDKVCYKN

SEQ ID NO: 291 - protein, 31 aa, Unknown
  Description of Unknown chassatide_C13 sequence
GFPCAESCVYIPCTVTALLGCSCRNRVCYRN

SEQ ID NO: 292 - protein, 28 aa, Unknown
  Description of Unknown chassatide_C17 sequence
IPCGESCVYIPCISAVLGCSCQNKVCYR

SEQ ID NO: 293 - protein, 31 aa, Unknown
  Description of Unknown chassatide_C14 sequence
GIPCAESCVYIPCTITALFGCSCKDKVCYNN

SEQ ID NO: 294 - protein, 29 aa, Unknown
  Description of Unknown chassatide_C8 sequence
AIPCGESCVWIPCISTVIGCSCSNKVCYR

SEQ ID NO: 295 - protein, 28 aa, Unknown
  Description of Unknown chassatide_C7 sequence
IPCGESCVWIPCITAIAGCSCKNKVCYT

SEQ ID NO: 296 - protein, 29 aa, Unknown
  Description of Unknown chassatide_C4 sequence
GASCGETCFTGICFTAGCSCNPWPTCTRN

SEQ ID NO: 297 - protein, 31 aa, Unknown
  Description of Unknown chassatide_C2 sequence
GIPCAESCVWIPCTITALMGCSCKNNVCYNN

SEQ ID NO: 298 - protein, 29 aa, Unknown
  Description of Unknown chassatide_C1 sequence
GDACGETCFTGICFTAGCSCNPWPTCTRN

SEQ ID NO: 299 - protein, 30 aa, Unknown
  Description of Unknown chassatide_C3 sequence
GIPCGESCVWIPCISSALGCSCKNKVCYRN

SEQ ID NO: 300 - protein, 31 aa, Unknown
  Description of Unknown chassatide_C5 sequence
GVIPCGESCVFIPCISSVVGCSCKNKVCYRN

SEQ ID NO: 301 - protein, 31 aa, Unknown
  Description of Unknown chassatide_C6 sequence
GVIPCGESCVFIPCISSVIGCSCKNKVCYRN

SEQ ID NO: 302 - protein, 30 aa, Unknown
  Description of Unknown chassatide_C9 sequence
GIPCGESCVFIPCVTTVIGCSCKDKVCYNN

SEQ ID NO: 303 - protein, 29 aa, Unknown
  Description of Unknown chassatide_C10 sequence
GEYCGESCYLIPCFTPGCYCVSRQCVNKN

SEQ ID NO: 304 - protein, 28 aa, Unknown
  Description of Unknown chassatide_C11 sequence
IPCGESCVWIPCISGMFGCSCKDKVCYS

SEQ ID NO: 305 - protein, 28 aa, Unknown
  Description of Unknown chassatide_C12 sequence
EYCGESCYLIPCFTPGCYCVSRQCVNKN

SEQ ID NO: 306 - protein, 30 aa, Unknown
  Description of Unknown Phyb_A sequence
GIGCGESCVWIPCVSAAIGCSCSNKICYRN

SEQ ID NO: 307 - protein, 30 aa, Unknown
  Description of Unknown Phyb_D sequence
GIPCGESCMWIPCISAAIGCSCTNHVCYKN

SEQ ID NO: 308 - protein, 30 aa, Unknown
  Description of Unknown Phyb_E sequence
GIPCGESCVWIPCISGVQGCSCSNKICYRN

SEQ ID NO: 309 - protein, 30 aa, Unknown
  Description of Unknown Phyb_F sequence
GIPCGGSCVWIPCISGVQGCSCSNKICYRN

SEQ ID NO: 310 - protein, 30 aa, Unknown
  Description of Unknown Phyb_G sequence
GIPCGESCAWIPCISAVQGCSCRNKICYRN

SEQ ID NO: 311 - protein, 30 aa, Unknown
  Description of Unknown Phyb_H sequence
GLPCGESCIWIECISGAIGCSCRNKVCYRN

SEQ ID NO: 312 - protein, 31 aa, Unknown
  Description of Unknown Phyb_I sequence
GIPCGESCIWIPCTTTALLGCSCSNKVCYKN

SEQ ID NO: 313 - protein, 31 aa, Unknown
  Description of Unknown Phyb_J sequence
SYTCGESCLWIPCTVTAAFGCYCSNKVCVKD

SEQ ID NO: 314 - protein, 31 aa, Unknown
  Description of Unknown Phyb_K sequence
STDCGEPCVYIPCTITALLGCSCLNKVCVRP

SEQ ID NO: 315 - protein, 32 aa, Unknown
  Description of Unknown Phyb_L sequence
QSISCAETCVWIPCATSLIGCSCVNSICTYTN

SEQ ID NO: 316 - protein, 30 aa, Unknown
  Description of Unknown Phyb_B sequence
GVPCGESCVWMYCISAAMGCSCRNKVCYRN

SEQ ID NO: 317 - protein, 30 aa, Unknown
  Description of Unknown Phyb_C sequence
GIPCGESCVWMYCITATMGCSCRNKVCYKN

SEQ ID NO: 318 - protein, 29 aa, Unknown
  Description of Unknown vocC sequence
GLPVCGETCVGGTCNTPGCSCSWPVCIRN

SEQ ID NO: 319 - protein, 31 aa, Unknown
  Description of Unknown vitri_peptide_1 sequence
GLIPCGESCVWIPCISSVIGCSCKSKVCYKN

SEQ ID NO: 320 - protein, 31 aa, Unknown
  Description of Unknown vitri_peptide_2 sequence
GSIPCGESCVWIPCISGIAGCSCSNKVCYLN

SEQ ID NO: 321 - protein, 31 aa, Unknown
  Description of Unknown vitri_peptide_3 sequence
GSWPCGESCVYIPCITSIAGCECSKNVCYKN

SEQ ID NO: 322 - protein, 30 aa, Unknown
  Description of Unknown vitri_peptide_4 sequence
GTPCGESCIYVPCISAVFGCWCQSKVCYKD

SEQ ID NO: 323 - protein, 29 aa, Unknown
  Description of Unknown vitri_peptide_8 sequence
PTPCGETCIWISCVTAAIGCYCHESICYR

SEQ ID NO: 324 - protein, 32 aa, Unknown
  Description of Unknown vitri_peptide_9a/53 sequence
GTIFDCGETCLLGKCYTPGCSCGSWALCYGQN

SEQ ID NO: 325 - protein, 28 aa, Unknown
  Description of Unknown vitri_peptide_14 sequence
GSSCGETCEVFSCFITRCACIDGLCYRN

SEQ ID NO: 326 - protein, 29 aa, Unknown
  Description of Unknown vitri_peptide_18a sequence
GVPICGETCFQGTCNTPGCTCKWPICERN

SEQ ID NO: 327 - protein, 32 aa, Unknown
  Description of Unknown vitri_peptide_20 sequence
GDLVPCGESCVYIPCLTTVLGCSCSENVCYRN

SEQ ID NO: 328 - protein, 31 aa, Unknown
  Description of Unknown vitri_peptide_21 sequence
GGPLDCQETCTLSDRCYTKGCTCNWPICYKN

SEQ ID NO: 329 - protein, 29 aa, Unknown
  Description of Unknown vitri_peptide_22a sequence
GAPVCGETCFTGLCYSSGCSCIYPVCNRN

SEQ ID NO: 330 - protein, 29 aa, Unknown
  Description of Unknown vitri_peptide_94b sequence
GVAVCGETCTLGTCYTPGCSCDWPICKRN

SEQ ID NO: 331 - protein, 29 aa, Unknown
  Description of Unknown vitri_peptide_23 sequence
GLPTCGETCTLGTCYTPGCTCSWPLCTKN

SEQ ID NO: 332 - protein, 30 aa, Unknown
  Description of Unknown vitri_peptide_24/28 sequence
GEPVCGDSCVFFGCDDEGCTCGPWSLCYRN

SEQ ID NO: 333 - protein, 31 aa, Unknown
  Description of Unknown vitri_peptide_27a sequence
GAFTPCGETCLTGECHTEGCSCVGQTFCVKK

SEQ ID NO: 334 - protein, 32 aa, Unknown
  Description of Unknown vitri_peptide_29 sequence
GVPSSDCLETCFGGKCNAHRCTCSQWPLCAKN

SEQ ID NO: 335 - protein, 28 aa, Unknown
  Description of Unknown vitri_peptide_30 sequence
GFACGETCIFTSCFITGCTCNSSLCFRN

SEQ ID NO: 336 - protein, 33 aa, Unknown
  Description of Unknown vitri_peptide_36/37 sequence
GGTIFSCGESCFQGTCYTKGCACGDWKLCYGEN

SEQ ID NO: 337 - protein, 28 aa, Unknown
  Description of Unknown vitri_peptide_38 sequence
GDTCYETCFTGFCFIGGCKCDFPVCVKN

SEQ ID NO: 338 - protein, 29 aa, Unknown
  Description of Unknown vitri_peptide_39 sequence
GAPICGESCFTGTCYTVQCSCSWPVCTRN

SEQ ID NO: 339 - protein, 29 aa, Unknown
  Description of Unknown vitri_peptide_39_linear sequence
GAPICGESCFTGTCYTVQCSCSWPVCTRN

SEQ ID NO: 340 - protein, 33 aa, Unknown
  Description of Unknown vitri_peptide_24a sequence
GGTIFNCGESCFQGTCYTKGCACGDWKLCYGEN

SEQ ID NO: 341 - protein, 31 aa, Unknown
  Description of Unknown vitri_peptide_50 sequence
GDIPCGESCVYIPCITGVLGCSCSHNVCYYN

SEQ ID NO: 342 - protein, 30 aa, Unknown
  Description of Unknown vitri_peptide_18b sequence
GSVFNCGETCVFGTCFTSGCSCVYRVCSKD

SEQ ID NO: 343 - protein, 28 aa, Unknown
  Description of Unknown Cter_35 sequence
GAFCGETCVLGTCYTPGCSCAPVICLNN

SEQ ID NO: 344 - protein, 29 aa, Unknown
  Description of Unknown mech_2 sequence
GIPTCGETCTLGKCNTPKCTCNWPICYKD

SEQ ID NO: 345 - protein, 29 aa, Unknown
  Description of Unknown mech_3 sequence
GIPTCGETCTLGKCNTPKCTCNWPICYKN

SEQ ID NO: 346 - protein, 31 aa, Unknown
  Description of Unknown mech_4 sequence
GSIPCGESCVYIPCISSIIGCSCKSKVCYKD

SEQ ID NO: 347 - protein, 29 aa, Unknown
  Description of Unknown mela_7 sequence
GIPTCGETCFKGKCYTPGCSCSYPICKKN

SEQ ID NO: 348 - protein, 31 aa, Unknown
  Description of Unknown caripe_10 sequence
GVIPCGESCVFIPCFSTVIGCSCKNKVCYRN

SEQ ID NO: 349 - protein, 31 aa, Unknown
  Description of Unknown caripe_11 sequence
GVIPCGESCVFIPCISTVIGCSCKKKVCYRN

SEQ ID NO: 350 - protein, 31 aa, Unknown
  Description of Unknown caripe_12 sequence
GVIPCGESCVFIPCFSSVIGCSCKNKVCYRN

SEQ ID NO: 351 - protein, 30 aa, Unknown
  Description of Unknown caripe_13 sequence
GIPCGESCVFIPCFTSVFGCSCKDKVCYRN

SEQ ID NO: 352 - protein, 18 aa, Unknown
  Description of Unknown RTD-1 sequence
RCICTRGFCRCLCRRGVC

SEQ ID NO: 353 - protein, 18 aa, Unknown
  Description of Unknown RTD-2 sequence
GVCRCLCRRGVCRCLCRR

SEQ ID NO: 354 - protein, 18 aa, Unknown
  Description of Unknown RTD-3 sequence
GFCRCICTRGFCRCICTR

SEQ ID NO: 355 - protein, 76 aa, Unknown
  Description of Unknown RTD-1a sequence
MRTFALLTAMLLLVALHAQAEARQARADEAAAQQQPGTDDQGMAHSFTWPENAALPLSESAKGLRCICTRGFCRLL

SEQ ID NO: 356 - protein, 76 aa, Unknown
  Description of Unknown RTD-1b sequence
MRTFALLTAMLLLVALHAQAEARQARADEAAAQQQPGADDQGMAHSFTRPENAALPLSESARGLRCLCRRGVCQLL

SEQ ID NO: 357 - protein, 8 aa, Unknown
  Description of Unknown BTD-a sequence
RCVCTRGF

SEQ ID NO: 358 - protein, 9 aa, Unknown
  Description of Unknown BTD-b sequence
RCVCRRGVC

SEQ ID NO: 359 - protein, 9 aa, Unknown
  Description of Unknown BTD-c sequence
RCICLLGIC

SEQ ID NO: 360 - protein, 9 aa, Unknown
  Description of Unknown BTD-d sequence
RCFCRRGVC

SEQ ID NO: 361 - protein, 35 aa, Unknown
  Description of Unknown Glucagon-like peptide-1 sequence
DEFERHAEGTFTSDVSSYLEGQAAKEFIAWLVKGR

SEQ ID NO: 362 - protein, 103 aa, Artificial Sequence
  Description of Artificial Sequence Synthetic polypeptide
CGGSSGGSSHHHHHHSQGTFTSDYSKYLDSRRAQDFVQWLMNTGGDDGGGGVVCGPGKKIILLQQRRCGRRRRDDSSDDCGPGGGAACGIICGRRGGNNGGYY

SEQ ID NO: 363 - protein, 13 aa, Artificial Sequence
  Description of Artificial Sequence Synthetic peptide
GPGKKIILLQQRR

SEQ ID NO: 364 - protein, 11 aa, Artificial Sequence
  Description of Artificial Sequence Synthetic peptide
GRRRRDDSSDD

SEQ ID NO: 365 - protein, 7 aa, Artificial Sequence
  Description of Artificial Sequence Synthetic peptide
GPGGGAA

SEQ ID NO: 366 - protein, 3 aa, Artificial Sequence
  Description of Artificial Sequence Synthetic peptide
GII

SEQ ID NO: 367 - protein, 11 aa, Artificial Sequence
  Description of Artificial Sequence Synthetic peptide
GRRGGNNGGYY

SEQ ID NO: 368 - protein, 21 aa, Unknown
  Description of Unknown MCOG2 sequence
  MOD_RES (9)..(9): Any amino acid
  MOD_RES (14)..(14): Any amino acid
GASKAPASXLRKLXKRLLRDA

SEQ ID NO: 369 - protein, 18 aa, Unknown
  Description of Unknown MCOG1 sequence
  MOD_RES (9)..(9): Any amino acid
  MOD_RES (14)..(14): Any amino acid
GASKAPASXLRKLXKRLL

SEQ ID NO: 370 - protein, 6 aa, Artificial Sequence
  Description of Artificial Sequence Synthetic 6xHis tag
HHHHHH

SEQ ID NO: 371 - protein, 34 aa, Artificial Sequence
  Description of Artificial Sequence Synthetic polypeptide
  MOD_RES (34)..(34): Any amino acid
GGVCPKILQRCRRDSDCPGACICRGNGYCGSGCX

SEQ ID NO: 372 - protein, 6 aa, Unknown
  Description of Unknown MCoTI-I sequence
PKILQR

SEQ ID NO: 373 - protein, 6 aa, Unknown
  Description of Unknown MCoTI-II sequence
PKILKK

SEQ ID NO: 374 - protein, 6 aa, Unknown
  Description of Unknown MCoTI-III sequence
PRILKK

SEQ ID NO: 375 - protein, 6 aa, Unknown
  Description of Unknown MCoTI-IV sequence
PRILKK

SEQ ID NO: 376 - protein, 6 aa, Unknown
  Description of Unknown MCoTI-V sequence
PRILKK

SEQ ID NO: 377 - protein, 6 aa, Unknown
  Description of Unknown MCoTI-VI sequence
PRILKK

SEQ ID NO: 378 - protein, 6 aa, Unknown
  Description of Unknown MCoTI-VII sequence
PRILKK

SEQ ID NO: 379 - protein, 6 aa, Unknown
  Description of Unknown MCoTI-VIII sequence
PKILQR

SEQ ID NO: 380 - protein, 4 aa, Unknown
  Description of Unknown caripe 1 sequence
VFIP

SEQ ID NO: 381 - protein, 4 aa, Unknown
  Description of Unknown caripe 10 sequence
VFIP

SEQ ID NO: 382 - protein, 4 aa, Unknown
  Description of Unknown caripe 11 sequence
VFIP

SEQ ID NO: 383 - protein, 4 aa, Unknown
  Description of Unknown caripe 12 sequence
VFIP

SEQ ID NO: 384 - protein, 4 aa, Unknown
  Description of Unknown caripe 13 sequence
VFIP

SEQ ID NO: 385 - protein, 4 aa, Unknown
  Description of Unknown caripe 2 sequence
VFIR

SEQ ID NO: 386 - protein, 4 aa, Unknown
  Description of Unknown caripe 4 sequence
LRIP

SEQ ID NO: 387 - protein, 4 aa, Unknown
  Description of Unknown caripe 6 sequence
FRNP

SEQ ID NO: 388 - protein, 4 aa, Unknown
  Description of Unknown caripe 7 sequence
VFIP

SEQ ID NO: 389 - protein, 4 aa, Unknown
  Description of Unknown caripe 8 sequence
VFIP

SEQ ID NO: 390 - protein, 4 aa, Unknown
  Description of Unknown CD-1 sequence
YVIP

SEQ ID NO: 391 - protein, 4 aa, Unknown
  Description of Unknown chacur 1 sequence
VGGT

SEQ ID NO: 392 - protein, 4 aa, Unknown
  Description of Unknown chassatide C1 sequence
FTGI

SEQ ID NO: 393 - protein, 4 aa, Unknown
  Description of Unknown chassatide C10 sequence
YLIP

SEQ ID NO: 394 - protein, 4 aa, Unknown
  Description of Unknown chassatide C11 sequence
VWIP

SEQ ID NO: 395 - protein, 4 aa, Unknown
  Description of Unknown chassatide C12 sequence
YLIP

SEQ ID NO: 396 - protein, 4 aa, Unknown
  Description of Unknown chassatide C13 sequence
VYIP

SEQ ID NO: 397 - protein, 4 aa, Unknown
  Description of Unknown chassatide C14 sequence
VYIP

SEQ ID NO: 398 - protein, 4 aa, Unknown
  Description of Unknown chassatide C15 sequence
VYIP

SEQ ID NO: 399 - protein, 4 aa, Unknown
  Description of Unknown chassatide C16 sequence
VYIP

SEQ ID NO: 400 - protein, 4 aa, Unknown
  Description of Unknown chassatide C17 sequence
VYIP

SEQ ID NO: 401 - protein, 4 aa, Unknown
  Description of Unknown chassatide C18 sequence
VFIP

SEQ ID NO: 402 - protein, 4 aa, Unknown
  Description of Unknown chassatide C2 sequence
VWIP

SEQ ID NO: 403 - protein, 4 aa, Unknown
  Description of Unknown chassatide C3 sequence
VWIP

SEQ ID NO: 404 - protein, 4 aa, Unknown
  Description of Unknown chassatide C4 sequence
FTGI

SEQ ID NO: 405 - protein, 4 aa, Unknown
  Description of Unknown chassatide C5 sequence
VFIP

SEQ ID NO: 406 - protein, 4 aa, Unknown
  Description of Unknown chassatide C6 sequence
VFIP

SEQ ID NO: 407 - protein, 4 aa, Unknown
  Description of Unknown chassatide C7 sequence
VWIP

SEQ ID NO: 408 - protein, 4 aa, Unknown
  Description of Unknown chassatide C8 sequence
VWIP

SEQ ID NO: 409 - protein, 4 aa, Unknown
  Description of Unknown chassatide C9 sequence
VFIP

SEQ ID NO: 410 - protein, 4 aa, Unknown
  Description of Unknown circulin A sequence
VWIP

SEQ ID NO: 411 - protein, 4 aa, Unknown
  Description of Unknown circulin B sequence
VFIP

SEQ ID NO: 412 - protein, 4 aa, Unknown
  Description of Unknown circulin C sequence
VFIP

SEQ ID NO: 413 - protein, 4 aa, Unknown
  Description of Unknown circulin D sequence
VWIP

SEQ ID NO: 414 - protein, 4 aa, Unknown
  Description of Unknown circulin E sequence
VWIP

SEQ ID NO: 415 - protein, 4 aa, Unknown
  Description of Unknown circulin F sequence
VWIP

SEQ ID NO: 416 - protein, 4 aa, Unknown
  Description of Unknown cliotide T12 sequence
VFIP

SEQ ID NO: 417 - protein, 4 aa, Unknown
  Description of Unknown cliotide T2 sequence
VQGE

SEQ ID NO: 418 - protein, 4 aa, Unknown
  Description of Unknown cliotide T8 sequence
VFIP

SEQ ID NO: 419 - protein, 4 aa, Unknown
  Description of Unknown cliotide T9 sequence
VFIP

SEQ ID NO: 420 - protein, 4 aa, Unknown
  Description of Unknown cO26 sequence
FRGK

SEQ ID NO: 421 - protein, 4 aa, Unknown
  Description of Unknown cO27 sequence
FKGW

SEQ ID NO: 422 - protein, 4 aa, Unknown
  Description of Unknown cO28 sequence
VGGT

SEQ ID NO: 423 - protein, 4 aa, Unknown
  Description of Unknown cO29 sequence
VWIP

SEQ ID NO: 424 - protein, 4 aa, Unknown
  Description of Unknown cO30 sequence
VWIP

SEQ ID NO: 425 - protein, 4 aa, Unknown
  Description of Unknown cO31 sequence
VGGT

SEQ ID NO: 426 - protein, 4 aa, Unknown
  Description of Unknown cO32 sequence
FGGT

SEQ ID NO: 427 - protein, 4 aa, Unknown
  Description of Unknown cO33 sequence
VGGT

SEQ ID NO: 428 - protein, 4 aa, Unknown
  Description of Unknown cO34 sequence
VGGT

SEQ ID NO: 429 - protein, 4 aa, Unknown
  Description of Unknown cO35 sequence
VGGT

SEQ ID NO: 430 - protein, 4 aa, Unknown
  Description of Unknown cO36 sequence
FGGT

SEQ ID NO: 431 - protein, 4 aa, Unknown
  Description of Unknown Cter 35 sequence
VLGT

SEQ ID NO: 432 - protein, 4 aa, Unknown
  Description of Unknown Cter A sequence
VFIP

SEQ ID NO: 433 - protein, 4 aa, Unknown
  Description of Unknown Cter B sequence
VWIP

SEQ ID NO: 434 - protein, 4 aa, Unknown
  Description of Unknown Cter C sequence
VWIP

SEQ ID NO: 435 - protein, 4 aa, Unknown
  Description of Unknown Cter D sequence
VWIP

SEQ ID NO: 436 - protein, 4 aa, Unknown
  Description of Unknown Cter E sequence
VWIP

SEQ ID NO: 437 - protein, 4 aa, Unknown
  Description of Unknown Cter F sequence
VFIP

SEQ ID NO: 438 - protein, 4 aa, Unknown
  Description of Unknown Cter G sequence
VFIP

SEQ ID NO: 439 - protein, 4 aa, Unknown
  Description of Unknown Cter H sequence
VFIP

SEQ ID NO: 440 - protein, 4 aa, Unknown
  Description of Unknown Cter I sequence
VFIP

SEQ ID NO: 441 - protein, 4 aa, Unknown
  Description of Unknown Cter J sequence
VFIP

SEQ ID NO: 442 - protein, 4 aa, Unknown
  Description of Unknown Cter K sequence
VFIP

SEQ ID NO: 443 - protein, 4 aa, Unknown
  Description of Unknown Cter L sequence
VFIP

SEQ ID NO: 444 - protein, 4 aa, Unknown
  Description of Unknown Cter M sequence
TLGT

SEQ ID NO: 445 - protein, 4 aa, Unknown
  Description of Unknown Cter N sequence
VLGT

SEQ ID NO: 446 - protein, 4 aa, Unknown
  Description of Unknown Cter O sequence
VFIP

SEQ ID NO: 447 - protein, 4 aa, Unknown
  Description of Unknown Cter P sequence
VFIP

SEQ ID NO: 448 - protein, 4 aa, Unknown
  Description of Unknown Cter Q sequence
VFIP

SEQ ID NO: 449 - protein, 4 aa, Unknown
  Description of Unknown Cter R sequence
VFIP

SEQ ID NO: 450 - protein, 4 aa, Unknown
  Description of Unknown cyclopsychotride A sequence
VFIP

SEQ ID NO: 451 - protein, 4 aa, Unknown
  Description of Unknown cycloviolacin H1 sequence
VYIP

SEQ ID NO: 452 - protein, 4 aa, Unknown
  Description of Unknown cycloviolacin H2 sequence
VYIP

SEQ ID NO: 453 - protein, 4 aa, Unknown
  Description of Unknown cycloviolacin H3 sequence
FGGT

SEQ ID NO: 454 - protein, 4 aa, Unknown
  Description of Unknown cycloviolacin H4 sequence
VWIP

SEQ ID NO: 455 - protein, 4 aa, Unknown
  Description of Unknown cycloviolacin O1 sequence
VYIP

SEQ ID NO: 456 - protein, 4 aa, Unknown
  Description of Unknown cycloviolacin O10 sequence
VYIP

SEQ ID NO: 457 - protein, 4 aa, Unknown
  Description of Unknown cycloviolacin O11 sequence
VWIP

SEQ ID NO: 458 - protein, 4 aa, Unknown
  Description of Unknown cycloviolacin O12 sequence
VGGT

SEQ ID NO: 459 - protein, 4 aa, Unknown
  Description of Unknown cycloviolacin O13 sequence
VWIP

SEQ ID NO: 460 - protein, 4 aa, Unknown
  Description of Unknown cycloviolacin O14 sequence
FKGK

SEQ ID NO: 461 - protein, 4 aa, Unknown
  Description of Unknown cycloviolacin O15 sequence
FTGK

SEQ ID NO: 462 - protein, 4 aa, Unknown
  Description of Unknown cycloviolacin O16 sequence
FTGK

SEQ ID NO: 463 - protein, 4 aa, Unknown
  Description of Unknown cycloviolacin O17 sequence
VWIP

SEQ ID NO: 464 - protein, 4 aa, Unknown
  Description of Unknown cycloviolacin O18 sequence
VYIP

SEQ ID NO: 465 - protein, 4 aa, Unknown
  Description of Unknown cycloviolacin O19 sequence
VWIP

SEQ ID NO: 466 - protein, 4 aa, Unknown
  Description of Unknown cycloviolacin O2 sequence
VWIP

SEQ ID NO: 467 - protein, 4 aa, Unknown
  Description of Unknown cycloviolacin O20 sequence
VWIP

SEQ ID NO: 468 - protein, 4 aa, Unknown
  Description of Unknown cycloviolacin O21 sequence
VTGS

SEQ ID NO: 469 - protein, 4 aa, Unknown
  Description of Unknown cycloviolacin O22 sequence
VGGT

SEQ ID NO: 470 - protein, 4 aa, Unknown
  Description of Unknown cycloviolacin O23 sequence
FGGT

SEQ ID NO: 471 - protein, 4 aa, Unknown
  Description of Unknown cycloviolacin O24 sequence
FGGT

SEQ ID NO: 472 - protein, 4 aa, Unknown
  Description of Unknown cycloviolacin O25 sequence
AFIP

SEQ ID NO: 473 - protein, 4 aa, Unknown
  Description of Unknown cycloviolacin O3 sequence
VWIP

SEQ ID NO: 474 - protein, 4 aa, Unknown
  Description of Unknown cycloviolacin O4 sequence
VWIP

SEQ ID NO: 475 - protein, 4 aa, Unknown
  Description of Unknown cycloviolacin O5 sequence
VWIP

SEQ ID NO: 476 - protein, 4 aa, Unknown
  Description of Unknown cycloviolacin O6 sequence
VWIP

SEQ ID NO: 477 - protein, 4 aa, Unknown
  Description of Unknown cycloviolacin O7 sequence
VWIP

SEQ ID NO: 478 - protein, 4 aa, Unknown
  Description of Unknown cycloviolacin O8 sequence
VWIP

SEQ ID NO: 479 - protein, 4 aa, Unknown
  Description of Unknown cycloviolacin O9 sequence
VWIP

SEQ ID NO: 480 - protein, 4 aa, Unknown
  Description of Unknown cycloviolacin T1 sequence
VGGT

SEQ ID NO: 481 - protein, 4 aa, Unknown
  Description of Unknown cycloviolacin Y1 sequence
FLGT

SEQ ID NO: 482 - protein, 4 aa, Unknown
  Description of Unknown cycloviolacin Y2 sequence
FLGT

SEQ ID NO: 483 - protein, 4 aa, Unknown
  Description of Unknown cycloviolacin Y3 sequence
FLGT

SEQ ID NO: 484 - protein, 4 aa, Unknown
  Description of Unknown cycloviolacin Y4 sequence
VFIP

SEQ ID NO: 485 - protein, 4 aa, Unknown
  Description of Unknown cycloviolacin Y5 sequence
VWIP

SEQ ID NO: 486 - protein, 4 aa, Unknown
  Description of Unknown cycloviolin A sequence
VFIP

SEQ ID NO: 487 - protein, 4 aa, Unknown
  Description of Unknown cycloviolin B sequence
YVLP

SEQ ID NO: 488 - protein, 4 aa, Unknown
  Description of Unknown cycloviolin C sequence
VFIP

SEQ ID NO: 489 - protein, 4 aa, Unknown
  Description of Unknown cycloviolin D sequence
VFIP

SEQ ID NO: 490 - protein, 4 aa, Unknown
  Description of Unknown Globa A sequence
VFIP

SEQ ID NO: 491 - protein, 4 aa, Unknown
  Description of Unknown Globa B sequence
VFIP

SEQ ID NO: 492 - protein, 4 aa, Unknown
  Description of Unknown Globa C sequence
VYIP

SEQ ID NO: 493 - protein, 4 aa, Unknown
  Description of Unknown Globa D sequence
VFMP

SEQ ID NO: 494 - protein, 4 aa, Unknown
  Description of Unknown Globa E sequence
VKGK

SEQ ID NO: 495 - protein, 4 aa, Unknown
  Description of Unknown Globa F sequence
VFIP

SEQ ID NO: 496 - protein, 4 aa, Unknown
  Description of Unknown Glopa A sequence
VWTG

SEQ ID NO: 497 - protein, 4 aa, Unknown
  Description of Unknown Glopa B sequence
VWTG

SEQ ID NO: 498 - protein, 5 aa, Unknown
  Description of Unknown Glopa C sequence
FEGGN

SEQ ID NO: 499 - protein, 4 aa, Unknown
  Description of Unknown Glopa D sequence
VWVP

SEQ ID NO: 500 - protein, 4 aa, Unknown
  Description of Unknown Glopa E sequence
VWIP

SEQ ID NO: 501 - protein, 4 aa, Unknown
  Description of Unknown hcf-1 sequence
HYIP

SEQ ID NO: 502 - protein, 3 aa, Unknown
  Description of Unknown hcf-1 variant sequence
HIP

SEQ ID NO: 503 - protein, 4 aa, Unknown
  Description of Unknown hedyotide B1 sequence
FVLP

SEQ ID NO: 504 - protein, 4 aa, Unknown
  Description of Unknown hedyotide B2 sequence
VWIP

SEQ ID NO: 505 - protein, 4 aa, Unknown
  Description of Unknown htf-1 sequence
HYIP

SEQ ID NO: 506 - protein, 4 aa, Unknown
  Description of Unknown Hyfl A sequence
VYIP

SEQ ID NO: 507 - protein, 4 aa, Unknown
  Description of Unknown Hyfl B sequence
FIGK

SEQ ID NO: 508 - protein, 4 aa, Unknown
  Description of Unknown Hyfl C sequence
FIGK

SEQ ID NO: 509 - protein, 4 aa, Unknown
  Description of Unknown Hyfl D sequence
VYIP

SEQ ID NO: 510 - protein, 4 aa, Unknown
  Description of Unknown Hyfl E sequence
VYLP

SEQ ID NO: 511 - protein, 4 aa, Unknown
  Description of Unknown Hyfl F sequence
TTFN

SEQ ID NO: 512 - protein, 4 aa, Unknown
  Description of Unknown Hyfl I sequence
VFIP

SEQ ID NO: 513 - protein, 4 aa, Unknown
  Description of Unknown Hyfl J sequence
AYFG

SEQ ID NO: 514 - protein, 4 aa, Unknown
  Description of Unknown Hyfl K sequence
VYIP

SEQ ID NO: 515 - protein, 4 aa, Unknown
  Description of Unknown Hyfl L sequence
VYLP

SEQ ID NO: 516 - protein, 4 aa, Unknown
  Description of Unknown Hyfl M sequence
IFFP

SEQ ID NO: 517 - protein, 4 aa, Unknown
  Description of Unknown Hypa A sequence
VYIP

SEQ ID NO: 518 - protein, 4 aa, Unknown
  Description of Unknown kalata B1 sequence
VGGT

SEQ ID NO: 519 - protein, 4 aa, Unknown
  Description of Unknown kalata B10 sequence
FGGT

SEQ ID NO: 520 - protein, 4 aa, Unknown
  Description of Unknown kalata B10 linear sequence
FGGT

SEQ ID NO: 521 - protein, 4 aa, Unknown
  Description of Unknown kalata B11 sequence
FGGT

SEQ ID NO: 522 - protein, 4 aa, Unknown
  Description of Unknown kalata B12 sequence
FVLG

SEQ ID NO: 523 - protein, 4 aa, Unknown
  Description of Unknown kalata B13 sequence
FGGT

SEQ ID NO: 524 - protein, 4 aa, Unknown
  Description of Unknown kalata B14 sequence
FGGT

SEQ ID NO: 525 - protein, 4 aa, Unknown
  Description of Unknown kalata B15 sequence
FGGS

SEQ ID NO: 526 - protein, 4 aa, Unknown
  Description of Unknown kalata B16 sequence
VYIP

SEQ ID NO: 527 - protein, 4 aa, Unknown
  Description of Unknown kalata B17 sequence
VYIP

SEQ ID NO: 528 - protein, 4 aa, Unknown
  Description of Unknown kalata B18 sequence
VYIP

SEQ ID NO: 529 - protein, 4 aa, Unknown
  Description of Unknown kalata B19 sequence
VYVP

SEQ ID NO: 530 - protein, 4 aa, Unknown
  Description of Unknown kalata B2 sequence
FGGT

SEQ ID NO: 531 - protein, 4 aa, Unknown
  Description of Unknown kalata B3 sequence
FGGT

SEQ ID NO: 532 - protein, 4 aa, Unknown
  Description of Unknown kalata B4 sequence
VGGT

SEQ ID NO: 533 - protein, 4 aa, Unknown
  Description of Unknown kalata B5 sequence
VYIP

SEQ ID NO: 534 - protein, 4 aa, Unknown
  Description of Unknown kalata B6 sequence
FGGT

SEQ ID NO: 535 - protein, 4 aa, Unknown
  Description of Unknown kalata B7 sequence
TLGT

SEQ ID NO: 536 - protein, 4 aa, Unknown
  Description of Unknown kalata B8 sequence
LLGT

SEQ ID NO: 537 - protein, 4 aa, Unknown
  Description of Unknown kalata B9 sequence
VLGT

SEQ ID NO: 538 - protein, 4 aa, Unknown
  Description of Unknown kalata B9 linear sequence
VLGT

SEQ ID NO: 539 - protein, 4 aa, Unknown
  Description of Unknown kalata S sequence
VGGT

SEQ ID NO: 540 - protein, 5 aa, Unknown
  Description of Unknown MCoTI-I sequence
RRDSD

SEQ ID NO: 541 - protein, 5 aa, Unknown
  Description of Unknown MCoTI-II sequence
RRDSD

SEQ ID NO: 542 - protein, 5 aa, Unknown
  Description of Unknown MCoTI-III sequence
RRDSD

SEQ ID NO: 543 - protein, 5 aa, Unknown
  Description of Unknown MCoTI-IV sequence
RRDSD

SEQ ID NO: 544 - protein, 5 aa, Unknown
  Description of Unknown MCoTI-V sequence
RRDSD

SEQ ID NO: 545 - protein, 5 aa, Unknown
  Description of Unknown MCoTI-VI sequence
RRDSD

SEQ ID NO: 546 - protein, 5 aa, Unknown
  Description of Unknown MCoTI-VII sequence
RRDSD

SEQ ID NO: 547 - protein, 5 aa, Unknown
  Description of Unknown MCoTI-VIII sequence
RRDSD

SEQ ID NO: 548 - protein, 4 aa, Unknown
  Description of Unknown mech 2 sequence
TLGK

SEQ ID NO: 549 - protein, 4 aa, Unknown
  Description of Unknown mech 3 sequence
TLGK

SEQ ID NO: 550 - protein, 4 aa, Unknown
  Description of Unknown mech 4 sequence
VYIP

SEQ ID NO: 551 - protein, 4 aa, Unknown
  Description of Unknown mela 7 sequence
FKGK

SEQ ID NO: 552 - protein, 4 aa, Unknown
  Description of Unknown Mra13 sequence
VYLP

SEQ ID NO: 553 - protein, 4 aa, Unknown
  Description of Unknown Mra14a sequence
VFIP

SEQ ID NO: 554 - protein, 4 aa, Unknown
  Description of Unknown Mra14b sequence
VFIP

SEQ ID NO: 555 - protein, 4 aa, Unknown
  Description of Unknown Mra17 sequence
VYIP

SEQ ID NO: 556 - protein, 4 aa, Unknown
  Description of Unknown Mra22 sequence
VWIP

SEQ ID NO: 557 - protein, 4 aa, Unknown
  Description of Unknown Mra23 sequence
VFIP

SEQ ID NO: 558 - protein, 4 aa, Unknown
  Description of Unknown Mra24 sequence
LLGT

SEQ ID NO: 559 - protein, 4 aa, Unknown
  Description of Unknown Mra25 sequence
TLGE

SEQ ID NO: 560 - protein, 4 aa, Unknown
  Description of Unknown Mra26 sequence
VGNK

SEQ ID NO: 561 - protein, 4 aa, Unknown
  Description of Unknown Mra29 sequence
VFIP

SEQ ID NO: 562 - protein, 4 aa, Unknown
  Description of Unknown Mra30 sequence
VFIP

SEQ ID NO: 563 - protein, 4 aa, Unknown
  Description of Unknown Mra30 sequence
VFIP

SEQ ID NO: 564 - protein, 4 aa, Unknown
  Description of Unknown Mra4 sequence
VYIP

SEQ ID NO: 565 - protein, 4 aa, Unknown
  Description of Unknown Mra5 sequence
VYIP

SEQ ID NO: 566 - protein, 4 aa, Unknown
  Description of Unknown Oak6 cyclotide 1 sequence
FGGT

SEQ ID NO: 567 - protein, 4 aa, Unknown
  Description of Unknown Oak6 cyclotide 2 sequence
FGGT

SEQ ID NO: 568 - protein, 4 aa, Unknown
  Description of Unknown Oak7 cyclotide sequence
FFFG

SEQ ID NO: 569 - protein, 4 aa, Unknown
  Description of Unknown Oak8 cyclotide sequence
VFIP

SEQ ID NO: 570 - protein, 5 aa, Unknown
  Description of Unknown palicourein sequence
RVIPV

SEQ ID NO: 571 - protein, 4 aa, Unknown
  Description of Unknown paltet 1 sequence
FTGT

SEQ ID NO: 572 - protein, 4 aa, Unknown
  Description of Unknown Panitide L1 sequence
VLGT

SEQ ID NO: 573 - protein, 4 aa, Unknown
  Description of Unknown Panitide L2 sequence
VLGR

SEQ ID NO: 574 - protein, 4 aa, Unknown
  Description of Unknown Panitide L3 sequence
LLGK

SEQ ID NO: 575 - protein, 4 aa, Unknown
  Description of Unknown Panitide L4 sequence
LLGT

SEQ ID NO: 576 - protein, 4 aa, Unknown
  Description of Unknown Panitide L5 sequence
VLGT

SEQ ID NO: 577 - protein, 4 aa, Unknown
  Description of Unknown Panitide L6 sequence
VLGT

SEQ ID NO: 578 - protein, 4 aa, Unknown
  Description of Unknown Panitide L7 sequence
VLGT

SEQ ID NO: 579 - protein, 4 aa, Unknown
  Description of Unknown Panitide L8 sequence
VLGT

SEQ ID NO: 580 - protein, 4 aa, Unknown
  Description of Unknown Parigidin-br1 sequence
VFIP

SEQ ID NO: 581 - protein, 4 aa, Unknown
  Description of Unknown Phyb A sequence
VWIP

SEQ ID NO: 582 - protein, 4 aa, Unknown
  Description of Unknown Phyb B sequence
VWMY

SEQ ID NO: 583 - protein, 4 aa, Unknown
  Description of Unknown Phyb C sequence
VWMY

SEQ ID NO: 584 - protein, 4 aa, Unknown
  Description of Unknown Phyb D sequence
MWIP

SEQ ID NO: 585 - protein, 4 aa, Unknown
  Description of Unknown Phyb E sequence
VWIP

SEQ ID NO: 586 - protein, 4 aa, Unknown
  Description of Unknown Phyb F sequence
VWIP

SEQ ID NO: 587 - protein, 4 aa, Unknown
  Description of Unknown Phyb G sequence
AWIP

SEQ ID NO: 588 - protein, 4 aa, Unknown
  Description of Unknown Phyb H sequence
IWIE

SEQ ID NO: 589 - protein, 4 aa, Unknown
  Description of Unknown Phyb I sequence
IWIP

SEQ ID NO: 590 - protein, 4 aa, Unknown
  Description of Unknown Phyb J sequence
LWIP

SEQ ID NO: 591 - protein, 4 aa, Unknown
  Description of Unknown Phyb K sequence
VYIP

SEQ ID NO: 592 - protein, 4 aa, Unknown
  Description of Unknown Phyb L sequence
VWIP

SEQ ID NO: 593 - protein, 5 aa, Unknown
  Description of Unknown PS-1 sequence
IWDKT

SEQ ID NO: 594 - protein, 4 aa, Unknown
  Description of Unknown psybra 1 sequence
TLGT

SEQ ID NO: 595 - protein, 4 aa, Unknown
  Description of Unknown psyle A sequence
VFLG

SEQ ID NO: 596 - protein, 4 aa, Unknown
  Description of Unknown psyle B sequence
VAFG

SEQ ID NO: 597 - protein, 4 aa, Unknown
  Description of Unknown psyle C sequence
FKFK

SEQ ID NO: 598 - protein, 4 aa, Unknown
  Description of Unknown psyle D sequence
VFIP

SEQ ID NO: 599 - protein, 4 aa, Unknown
  Description of Unknown psyle E sequence
VFIP

SEQ ID NO: 600 - protein, 4 aa, Unknown
  Description of Unknown psyle F sequence
VFIP

SEQ ID NO: 601 - protein, 4 aa, Unknown
  Description of Unknown psypoe 1 sequence
FTTV

SEQ ID NO: 602 - protein, 4 aa, Unknown
  Description of Unknown tricyclon A sequence
FLGT

SEQ ID NO: 603 - protein, 4 aa, Unknown
  Description of Unknown tricyclon B sequence
FLGT

SEQ ID NO: 604 - protein, 4 aa, Unknown
  Description of Unknown vaby A sequence
AGGT

SEQ ID NO: 605 - protein, 4 aa, Unknown
  Description of Unknown vaby B sequence
AGGT

SEQ ID NO: 606 - protein, 4 aa, Unknown
  Description of Unknown vaby C sequence
AGGR

SEQ ID NO: 607 - protein, 4 aa, Unknown
  Description of Unknown vaby D sequence
FGGT

SEQ ID NO: 608 - protein, 4 aa, Unknown
  Description of Unknown vaby E sequence
FGGT

SEQ ID NO: 609 - protein, 4 aa, Unknown
  Description of Unknown varv peptide B sequence
FGGT

SEQ ID NO: 610 - protein, 4 aa, Unknown
  Description of Unknown varv peptide C sequence
VGGT

SEQ ID NO: 611 - protein, 4 aa, Unknown
  Description of Unknown varv peptide D sequence
VGGS

SEQ ID NO: 612 - protein, 4 aa, Unknown
  Description of Unknown varv peptide F sequence
TLGT

SEQ ID NO: 613 - protein, 4 aa, Unknown
  Description of Unknown varv peptide G sequence
FGGT

SEQ ID NO: 614 - protein, 4 aa, Unknown
  Description of Unknown varv peptide H sequence
FGGT

SEQ ID NO: 615 - protein, 5 aa, Unknown
  Description of Unknown vhl-1 sequence
AMISF

SEQ ID NO: 616 - protein, 4 aa, Unknown
  Description of Unknown vhl-2 sequence
FTGT

SEQ ID NO: 617 - protein, 4 aa, Unknown
  Description of Unknown vhr1 sequence
VWIP

SEQ ID NO: 618 - protein, 4 aa, Unknown
  Description of Unknown Viba 1 sequence
VYLP

SEQ ID NO: 619 - protein, 4 aa, Unknown
  Description of Unknown Viba 10 sequence
VYLP

SEQ ID NO: 620 - protein, 4 aa, Unknown
  Description of Unknown Viba 11 sequence
VWIP

SEQ ID NO: 621 - protein, 4 aa, Unknown
  Description of Unknown Viba 12 sequence
VWIP

SEQ ID NO: 622 - protein, 4 aa, Unknown
  Description of Unknown Viba 13 sequence
VWIP

SEQ ID NO: 623 - protein, 8 aa, Unknown
  Description of Unknown Viba 14 sequence
VIERTRAW

SEQ ID NO: 624 - protein, 4 aa, Unknown
  Description of Unknown Viba 15 sequence
VGGT

SEQ ID NO: 625 - protein, 4 aa, Unknown
  Description of Unknown Viba 16 sequence
TLGT

SEQ ID NO: 626 - protein, 4 aa, Unknown
  Description of Unknown Viba 17 sequence
VGGT

SEQ ID NO: 627 - protein, 4 aa, Unknown
  Description of Unknown Viba 2 sequence
VYLP

SEQ ID NO: 628 - protein, 4 aa, Unknown
  Description of Unknown Viba 3 sequence
VWIP

SEQ ID NO: 629 - protein, 4 aa, Unknown
  Description of Unknown Viba 4 sequence
VWIP

SEQ ID NO: 630 - protein, 4 aa, Unknown
  Description of Unknown Viba 5 sequence
VWIP

SEQ ID NO: 631 - protein, 4 aa, Unknown
  Description of Unknown Viba 6 sequence
VLIP

SEQ ID NO: 632 - protein, 4 aa, Unknown
  Description of Unknown Viba 7 sequence
VFIP

SEQ ID NO: 633 - protein, 4 aa, Unknown
  Description of Unknown Viba 8 sequence
YTFP

SEQ ID NO: 634 - protein, 4 aa, Unknown
  Description of Unknown Viba 9 sequence
VWIP

SEQ ID NO: 635 - protein, 4 aa, Unknown
  Description of Unknown vibi A sequence
FGGT

SEQ ID NO: 636 - protein, 4 aa, Unknown
  Description of Unknown vibi B sequence
FGGT

SEQ ID NO: 637 - protein, 4 aa, Unknown
  Description of Unknown vibi C sequence
AFGS

SEQ ID NO: 638 - protein, 4 aa, Unknown
  Description of Unknown vibi D sequence
FGGR

SEQ ID NO: 639 - protein, 4 aa, Unknown
  Description of Unknown vibi E sequence
VWIP

SEQ ID NO: 640 - protein, 4 aa, Unknown
  Description of Unknown vibi F sequence
VFIP

SEQ ID NO: 641 - protein, 4 aa, Unknown
  Description of Unknown vibi G sequence
VFIP

SEQ ID NO: 642 - protein, 4 aa, Unknown
  Description of Unknown vibi H sequence
VYIP

SEQ ID NO: 643 - protein, 4 aa, Unknown
  Description of Unknown vibi I sequence
VWIP

SEQ ID NO: 644 - protein, 4 aa, Unknown
  Description of Unknown vibi J sequence
VWIP

SEQ ID NO: 645 - protein, 4 aa, Unknown
  Description of Unknown vibi K sequence
VWIP

SEQ ID NO: 646 - protein, 4 aa, Unknown
  Description of Unknown vico A sequence
VYIP

SEQ ID NO: 647 - protein, 4 aa, Unknown
  Description of Unknown vico B sequence
VYIP

SEQ ID NO: 648 - protein, 4 aa, Unknown
  Description of Unknown vigno 1 sequence
AGGT

SEQ ID NO: 649 - protein, 4 aa, Unknown
  Description of Unknown vigno 10 sequence
VWIP

SEQ ID NO: 650 - protein, 4 aa, Unknown
  Description of Unknown vigno 2 sequence
AGGT

SEQ ID NO: 651 - protein, 4 aa, Unknown
  Description of Unknown vigno 3 sequence
VGGT

SEQ ID NO: 652 - protein, 4 aa, Unknown
  Description of Unknown vigno 4 sequence
VGGT

SEQ ID NO: 653 - protein, 4 aa, Unknown
  Description of Unknown vigno 5 sequence
VGGT

SEQ ID NO: 654 - protein, 4 aa, Unknown
  Description of Unknown vigno 6 sequence
VWIP

SEQ ID NO: 655 - protein, 4 aa, Unknown
  Description of Unknown vigno 7 sequence
VWIP

SEQ ID NO: 656 - protein, 4 aa, Unknown
  Description of Unknown vigno 8 sequence
VWIP

SEQ ID NO: 657 - protein, 4 aa, Unknown
  Description of Unknown vigno 9 sequence
VWIP

SEQ ID NO: 658 - protein, 4 aa, Unknown
  Description of Unknown violacin A sequence
FKFK

SEQ ID NO: 659 - protein, 4 aa, Unknown
  Description of Unknown violapeptide 1 sequence
VGGT

SEQ ID NO: 660 - protein, 4 aa, Unknown
  Description of Unknown viphi A sequence
VFIP

SEQ ID NO: 661 - protein, 4 aa, Unknown
  Description of Unknown viphi B sequence
TIGT

SEQ ID NO: 662 - protein, 4 aa, Unknown
  Description of Unknown viphi C sequence
VYIP

SEQ ID NO: 663 - protein, 4 aa, Unknown
  Description of Unknown viphi D sequence
VFIP

SEQ ID NO: 664 - protein, 4 aa, Unknown
  Description of Unknown viphi E sequence
VFIP

SEQ ID NO: 665 - protein, 4 aa, Unknown
  Description of Unknown viphi F sequence
VFIP

SEQ ID NO: 666 - protein, 4 aa, Unknown
  Description of Unknown viphi G sequence
VFIP

SEQ ID NO: 667 - protein, 4 aa, Unknown
  Description of Unknown viphi H sequence
VWIP

SEQ ID NO: 668 - protein, 4 aa, Unknown
  Description of Unknown vitri A sequence
VWIP

SEQ ID NO: 669 - protein, 4 aa, Unknown
  Description of Unknown vitri B sequence
VGGT

SEQ ID NO: 670 - protein, 4 aa, Unknown
  Description of Unknown vitri C sequence
VGGT

SEQ ID NO: 671 - protein, 4 aa, Unknown
  Description of Unknown vitri D sequence
FTGS

SEQ ID NO: 672 - protein, 4 aa, Unknown
  Description of Unknown vitri E sequence
VGGT

SEQ ID NO: 673 - protein, 4 aa, Unknown
  Description of Unknown vitri F sequence
VWIP

SEQ ID NO: 674 - protein, 4 aa, Unknown
  Description of Unknown vitri peptide 1 sequence
VWIP

SEQ ID NO: 675 - protein, 4 aa, Unknown
  Description of Unknown vitri peptide 18a sequence
FQGT

SEQ ID NO: 676 - protein, 4 aa, Unknown
  Description of Unknown vitri peptide 18b sequence
VFGT

SEQ ID NO: 677 - protein, 4 aa, Unknown
  Description of Unknown vitri peptide 2 sequence
VWIP

SEQ ID NO: 678 - protein, 4 aa, Unknown
  Description of Unknown vitri peptide 20 sequence
VYIP

SEQ ID NO: 679 - protein, 4 aa, Unknown
  Description of Unknown vitri peptide 22a sequence
FTGL

SEQ ID NO: 680 - protein, 4 aa, Unknown
  Description of Unknown vitri peptide 23 sequence
TLGT

SEQ ID NO: 681 - protein, 4 aa, Unknown
  Description of Unknown vitri peptide 24a sequence
FQGT

SEQ ID NO: 682 - protein, 4 aa, Unknown
  Description of Unknown vitri peptide 3 sequence
VYIP

SEQ ID NO: 683 - protein, 4 aa, Unknown
  Description of Unknown vitri peptide 30 sequence
IFTS

SEQ ID NO: 684 - protein, 4 aa, Unknown
  Description of Unknown vitri peptide 36/37 sequence
FQGT

SEQ ID NO: 685 - protein, 4 aa, Unknown
  Description of Unknown vitri peptide 39 sequence
FTGT

SEQ ID NO: 686 - protein, 4 aa, Unknown
  Description of Unknown vitri peptide 39 linear sequence
FTGT

SEQ ID NO: 687 - protein, 4 aa, Unknown
  Description of Unknown vitri peptide 4 sequence
IYVP

SEQ ID NO: 688 - protein, 4 aa, Unknown
  Description of Unknown vitri peptide 50 sequence
VYIP

SEQ ID NO: 689 - protein, 4 aa, Unknown
  Description of Unknown vitri peptide 8 sequence
IWIS

SEQ ID NO: 690 - protein, 4 aa, Unknown
  Description of Unknown vitri peptide 94b sequence
TLGT

SEQ ID NO: 691 - protein, 4 aa, Unknown
  Description of Unknown vitri peptide 9a/53 sequence
LLGK

SEQ ID NO: 692 - protein, 4 aa, Unknown
  Description of Unknown vocC sequence
VGGT

SEQ ID NO: 693 - protein, 4 aa, Unknown
  Description of Unknown vodo M sequence
FTGK

SEQ ID NO: 694 - protein, 4 aa, Unknown
  Description of Unknown vodo N sequence
TLGK

SEQ ID NO: 695 - protein, 4 aa, Unknown
  Description of Unknown Vpf-1 sequence
VFIP

SEQ ID NO: 696 - protein, 4 aa, Unknown
  Description of Unknown Vpl-1 sequence
VLIP

SEQ ID NO: 697 - protein, 6 aa, Unknown
  Description of Unknown caripe 1 sequence
ISTVIG

SEQ ID NO: 698 - protein, 6 aa, Unknown
  Description of Unknown caripe 10 sequence
FSTVIG

SEQ ID NO: 699 - protein, 6 aa, Unknown
  Description of Unknown caripe 11 sequence
ISTVIG

SEQ ID NO: 700 - protein, 6 aa, Unknown
  Description of Unknown caripe 12 sequence
FSSVIG

SEQ ID NO: 701 - protein, 6 aa, Unknown
  Description of Unknown caripe 13 sequence
FTSVFG

SEQ ID NO: 702 - protein, 7 aa, Unknown
  Description of Unknown caripe 2 sequence
TITALLG

SEQ ID NO: 703 - protein, 4 aa, Unknown
  Description of Unknown caripe 4 sequence
LSPR

SEQ ID NO: 704 - protein, 4 aa, Unknown
  Description of Unknown caripe 6 sequence
LSRR

SEQ ID NO: 705 - protein, 7 aa, Unknown
  Description of Unknown caripe 7 sequence
TVTALLG

SEQ ID NO: 706 - protein, 6 aa, Unknown
  Description of Unknown caripe 8 sequence
ITAAIG

SEQ ID NO: 707 - protein, 6 aa, Unknown
  Description of Unknown CD-1 sequence
ISYLVG

SEQ ID NO: 708 - protein, 4 aa, Unknown
  Description of Unknown chacur 1 sequence
NTPG

SEQ ID NO: 709 - protein, 4 aa, Unknown
  Description of Unknown chassatide C1 sequence
FTAG

SEQ ID NO: 710 - protein, 4 aa, Unknown
  Description of Unknown chassatide C10 sequence
FTPG

SEQ ID NO: 711 - protein, 6 aa, Unknown
  Description of Unknown chassatide C11 sequence
ISGMFG

SEQ ID NO: 712 - protein, 4 aa, Unknown
  Description of Unknown chassatide C12 sequence
FTPG

SEQ ID NO: 713 - protein, 7 aa, Unknown
  Description of Unknown chassatide C13 sequence
TVTALLG

SEQ ID NO: 714 - protein, 7 aa, Unknown
  Description of Unknown chassatide C14 sequence
TITALFG

SEQ ID NO: 715 - protein, 7 aa, Unknown
  Description of Unknown chassatide C15 sequence
TITALLG

SEQ ID NO: 716 - protein, 7 aa, Unknown
  Description of Unknown chassatide C16 sequence
TITALFG

SEQ ID NO: 717 - protein, 6 aa, Unknown
  Description of Unknown chassatide C17 sequence
ISAVLG

SEQ ID NO: 718 - protein, 6 aa, Unknown
  Description of Unknown chassatide C18 sequence
ISALLG

SEQ ID NO: 719 - protein, 7 aa, Unknown
  Description of Unknown chassatide C2 sequence
TITALMG

SEQ ID NO: 720 - protein, 6 aa, Unknown
  Description of Unknown chassatide C3 sequence
ISSALG

SEQ ID NO: 721 - protein, 4 aa, Unknown
  Description of Unknown chassatide C4 sequence
FTAG

SEQ ID NO: 722 - protein, 6 aa, Unknown
  Description of Unknown chassatide C5 sequence
ISSVVG

SEQ ID NO: 723 - protein, 6 aa, Unknown
  Description of Unknown chassatide C6 sequence
ISSVIG

SEQ ID NO: 724 - protein, 6 aa, Unknown
  Description of Unknown chassatide C7 sequence
ITAIAG

SEQ ID NO: 725 - protein, 6 aa, Unknown
  Description of Unknown chassatide C8 sequence
ISTVIG

SEQ ID NO: 726 - protein, 6 aa, Unknown
  Description of Unknown chassatide C9 sequence
VTTVIG

SEQ ID NO: 727 - protein, 6 aa, Unknown
  Description of Unknown circulin A sequence
ISAALG

SEQ ID NO: 728 - protein, 6 aa, Unknown
  Description of Unknown circulin B sequence
ISTLLG

SEQ ID NO: 729 - protein, 6 aa, Unknown
  Description of Unknown circulin C sequence
ITSVAG

SEQ ID NO: 730 - protein, 6 aa, Unknown
  Description of Unknown circulin D sequence
VTSIFN

SEQ ID NO: 731 - protein, 6 aa, Unknown
  Description of Unknown circulin E sequence
LTSVFN

SEQ ID NO: 732 - protein, 6 aa, Unknown
  Description of Unknown circulin F sequence
ISAAIG

SEQ ID NO: 733 - protein, 6 aa, Unknown
  Description of Unknown cliotide T12 sequence
ITGAIG

SEQ ID NO: 734 - protein, 4 aa, Unknown
  Description of Unknown cliotide T2 sequence
YTPG

SEQ ID NO: 735 - protein, 6 aa, Unknown
  Description of Unknown cliotide T8 sequence
ISSVVG

SEQ ID NO: 736 - protein, 6 aa, Unknown
  Description of Unknown cliotide T9 sequence
LTTVVG

SEQ ID NO: 737 - protein, 4 aa, Unknown
  Description of Unknown cO26 sequence
YTPG

SEQ ID NO: 738 - protein, 4 aa, Unknown
  Description of Unknown cO27 sequence
YTPG

SEQ ID NO: 739 - protein, 4 aa, Unknown
  Description of Unknown cO28 sequence
NTPG

SEQ ID NO: 740 - protein, 6 aa, Unknown
  Description of Unknown cO29 sequence
ISGAIG

SEQ ID NO: 741 - protein, 6 aa, Unknown
  Description of Unknown cO30 sequence
ISSAIG

SEQ ID NO: 742 - protein, 4 aa, Unknown
  Description of Unknown cO31 sequence
NTPG

SEQ ID NO: 743 - protein, 4 aa, Unknown
  Description of Unknown cO32 sequence
NTPG

SEQ ID NO: 744 - protein, 4 aa, Unknown
  Description of Unknown cO33 sequence
NTPY

SEQ ID NO: 745 - protein, 4 aa, Unknown
  Description of Unknown cO34 sequence
NTEY

SEQ ID NO: 746 - protein, 4 aa, Unknown
  Description of Unknown cO35 sequence
NTPY

SEQ ID NO: 747 - protein, 4 aa, Unknown
  Description of Unknown cO36 sequence
NTPG

SEQ ID NO: 748 - protein, 4 aa, Unknown
  Description of Unknown Cter 35 sequence
YTPG

SEQ ID NO: 749 - protein, 6 aa, Unknown
  Description of Unknown Cter A sequence
ISTVIG

SEQ ID NO: 750 - protein, 7 aa, Unknown
  Description of Unknown Cter B sequence
TVTALLG

SEQ ID NO: 751 - protein, 7 aa, Unknown
  Description of Unknown Cter C sequence
TVTALLG

SEQ ID NO: 752 - protein, 7 aa, Unknown
  Description of Unknown Cter D sequence
TVTALLG

SEQ ID NO: 753 - protein, 7 aa, Unknown
  Description of Unknown Cter E sequence
TVTALLG

SEQ ID NO: 754 - protein, 6 aa, Unknown
  Description of Unknown Cter F sequence
ISSVVG

SEQ ID NO: 755 - protein, 6 aa, Unknown
  Description of Unknown Cter G sequence
ITTVVG

SEQ ID NO: 756 - protein, 6 aa, Unknown
  Description of Unknown Cter H sequence
ITTVVG

SEQ ID NO: 757 - protein, 6 aa, Unknown
  Description of Unknown Cter I sequence
ITGIAG

SEQ ID NO: 758 - protein, 6 aa, Unknown
  Description of Unknown Cter J sequence
ITGIAG

SEQ ID NO: 759 - protein, 6 aa, Unknown
  Description of Unknown Cter K sequence
ITTVVG

SEQ ID NO: 760 - protein, 6 aa, Unknown
  Description of Unknown Cter L sequence
ITTVVG

SEQ ID NO: 761 - protein, 4 aa, Unknown
  Description of Unknown Cter M sequence
YVPD

SEQ ID NO: 762 - protein, 4 aa, Unknown
  Description of Unknown Cter N sequence
YTPD

SEQ ID NO: 763 - protein, 6 aa, Unknown
  Description of Unknown Cter O sequence
ITGIAG

SEQ ID NO: 764 - protein, 6 aa, Unknown
  Description of Unknown Cter P sequence
ITAAIG

SEQ ID NO: 765 - protein, 6 aa, Unknown
  Description of Unknown Cter Q sequence
ISTVIG

SEQ ID NO: 766 - protein, 7 aa, Unknown
  Description of Unknown Cter R sequence
TVTALLG

SEQ ID NO: 767 - protein, 7 aa, Unknown
  Description of Unknown cyclopsychotride A sequence
TVTALLG

SEQ ID NO: 768 - protein, 6 aa, Unknown
  Description of Unknown cycloviolacin H1 sequence
LTSAIG

SEQ ID NO: 769 - protein, 4 aa, Unknown
  Description of Unknown cycloviolacin H2 sequence
FIPG

SEQ ID NO: 770 - protein, 4 aa, Unknown
  Description of Unknown cycloviolacin H3 sequence
NTPG

SEQ ID NO: 771 - protein, 7 aa, Unknown
  Description of Unknown cycloviolacin H4 sequence
TVTALLG

SEQ ID NO: 772 - protein, 7 aa, Unknown
  Description of Unknown cycloviolacin O1 sequence
TVTALLG

SEQ ID NO: 773 - protein, 6 aa, Unknown
  Description of Unknown cycloviolacin O10 sequence
LTSAVG

SEQ ID NO: 774 - protein, 6 aa, Unknown
  Description of Unknown cycloviolacin O11 sequence
ISAVVG

SEQ ID NO: 775 - protein, 4 aa, Unknown
  Description of Unknown cycloviolacin O12 sequence
NTPG

SEQ ID NO: 776 - protein, 6 aa, Unknown
  Description of Unknown cycloviolacin O13 sequence
ISAAIG

SEQ ID NO: 777 - protein, 4 aa, Unknown
  Description of Unknown cycloviolacin O14 sequence
YTPG

SEQ ID NO: 778 - protein, 4 aa, Unknown
  Description of Unknown cycloviolacin O15 sequence
YTPG

SEQ ID NO: 779 - protein, 4 aa, Unknown
  Description of Unknown cycloviolacin O16 sequence
YTPG

SEQ ID NO: 780 - protein, 6 aa, Unknown
  Description of Unknown cycloviolacin O17 sequence
ISAAIG

SEQ ID NO: 781 - protein, 7 aa, Unknown
  Description of Unknown cycloviolacin O18 sequence
TVTALAG

SEQ ID NO: 782 - protein, 6 aa, Unknown
  Description of Unknown cycloviolacin O19 sequence
ISSVVG

SEQ ID NO: 783 - protein, 6 aa, Unknown
  Description of Unknown cycloviolacin O2 sequence
ISSAIG

SEQ ID NO: 784 - protein, 6 aa, Unknown
  Description of Unknown cycloviolacin O20 sequence
LTSAIG

SEQ ID NO: 785 - protein, 4 aa, Unknown
  Description of Unknown cycloviolacin O21 sequence
YTPG

SEQ ID NO: 786 - protein, 4 aa, Unknown
  Description of Unknown cycloviolacin O22 sequence
NTPG

SEQ ID NO: 787 - protein, 4 aa, Unknown
  Description of Unknown cycloviolacin O23 sequence
NTPG

SEQ ID NO: 788 - protein, 4 aa, Unknown
  Description of Unknown cycloviolacin O24 sequence
NTPG

SEQ ID NO: 789 - protein, 7 aa, Unknown
  Description of Unknown cycloviolacin O25 sequence
ITHVPGT

SEQ ID NO: 790 - protein, 6 aa, Unknown
  Description of Unknown cycloviolacin O3 sequence
LTSAIG

SEQ ID NO: 791 - protein, 6 aa, Unknown
  Description of Unknown cycloviolacin O4 sequence
ISSAIG

SEQ ID NO: 792 - protein, 6 aa, Unknown
  Description of Unknown cycloviolacin O5 sequence
ISSAVG

SEQ ID NO: 793 - protein, 6 aa, Unknown
  Description of Unknown cycloviolacin O6 sequence
ISAAVG

SEQ ID NO: 794 - protein, 7 aa, Unknown
  Description of Unknown cycloviolacin O7 sequence
TITALAG

SEQ ID NO: 795 - protein, 6 aa, Unknown
  Description of Unknown cycloviolacin O8 sequence
ISSVVG

SEQ ID NO: 796 - protein, 6 aa, Unknown
  Description of Unknown cycloviolacin O9 sequence
LTSAVG

SEQ ID NO: 797 - protein, 4 aa, Unknown
  Description of Unknown cycloviolacin T1 sequence
NTPG

SEQ ID NO: 798 - protein, 4 aa, Unknown
  Description of Unknown cycloviolacin Y1 sequence
YTPG

SEQ ID NO: 799 - protein, 4 aa, Unknown
  Description of Unknown cycloviolacin Y2 sequence
YTAG

SEQ ID NO: 800 - protein, 4 aa, Unknown
  Description of Unknown cycloviolacin Y3 sequence
YTAG

SEQ ID NO: 801 - protein, 6 aa, Unknown
  Description of Unknown cycloviolacin Y4 sequence
ITGVIG

SEQ ID NO: 802 - protein, 7 aa, Unknown
  Description of Unknown cycloviolacin Y5 sequence
TVTALVG

SEQ ID NO: 803 - protein, 6 aa, Unknown
  Description of Unknown cycloviolin A sequence
ISAAIG

SEQ ID NO: 804 - protein, 4 aa, Unknown
  Description of Unknown cycloviolin B sequence
FTVG

SEQ ID NO: 805 - protein, 6 aa, Unknown
  Description of Unknown cycloviolin C sequence
LTTVAG

SEQ ID NO: 806 - protein, 6 aa, Unknown
  Description of Unknown cycloviolin D sequence
ISAAIG

SEQ ID NO: 807 - protein, 6 aa, Unknown
  Description of Unknown Globa A sequence
ITAAIG

SEQ ID NO: 808 - protein, 6 aa, Unknown
  Description of Unknown Globa B sequence
ISAVLG

SEQ ID NO: 809 - protein, 7 aa, Unknown
  Description of Unknown Globa C sequence
LLTAPIG

SEQ ID NO: 810 - protein, 6 aa, Unknown
  Description of Unknown Globa D sequence
ISGPMG

SEQ ID NO: 811 - protein, 4 aa, Unknown
  Description of Unknown Globa E sequence
NTPG

SEQ ID NO: 812 - protein, 6 aa, Unknown
  Description of Unknown Globa F sequence
ISAIAG

SEQ ID NO: 813 - protein, 5 aa, Unknown
  Description of Unknown Glopa A sequence
FLVPG

SEQ ID NO: 814 - protein, 5 aa, Unknown
  Description of Unknown Glopa B sequence
FLVPG

SEQ ID NO: 815 - protein, 4 aa, Unknown
  Description of Unknown Glopa C sequence
RIPG

SEQ ID NO: 816 - protein, 7 aa, Unknown
  Description of Unknown Glopa D sequence
TVTALMG

SEQ ID NO: 817 - protein, 7 aa, Unknown
  Description of Unknown Glopa E sequence
TVTKMLG

SEQ ID NO: 818 - protein, 6 aa, Unknown
  Description of Unknown hcf-1 sequence
VTSAIG

SEQ ID NO: 819 - protein, 6 aa, Unknown
  Description of Unknown hcf-1 variant sequence
VTSAIG

SEQ ID NO: 820 - protein, 6 aa, Unknown
  Description of Unknown hedyotide B1 sequence
WSAKFG

SEQ ID NO: 821 - protein, 6 aa, Unknown
  Description of Unknown hedyotide B2 sequence
ISSAWG

SEQ ID NO: 822 - protein, 6 aa, Unknown
  Description of Unknown htf-1 sequence
VTSTIG

SEQ ID NO: 823 - protein, 7 aa, Unknown
  Description of Unknown Hyfl A sequence
TVTALVG

SEQ ID NO: 824 - protein, 6 aa, Unknown
  Description of Unknown Hyfl B sequence
YTEELG

SEQ ID NO: 825 - protein, 6 aa, Unknown
  Description of Unknown Hyfl C sequence
YTEELG

SEQ ID NO: 826 - protein, 6 aa, Unknown
  Description of Unknown Hyfl D sequence
FTGIAG

SEQ ID NO: 827 - protein, 4 aa, Unknown
  Description of Unknown Hyfl E sequence
FLPN

SEQ ID NO: 828 - protein, 4 aa, Unknown
  Description of Unknown Hyfl F sequence
WIPN

SEQ ID NO: 829 - protein, 6 aa, Unknown
  Description of Unknown Hyfl I sequence
ISGVIG

SEQ ID NO: 830 - protein, 4 aa, Unknown
  Description of Unknown Hyfl J sequence
WIPG

SEQ ID NO: 831 - protein, 6 aa, Unknown
  Description of Unknown Hyfl K sequence
FTAVVG

SEQ ID NO: 832 - protein, 6 aa, Unknown
  Description of Unknown Hyfl L sequence
FTGVIG

SEQ ID NO: 833 - protein, 4 aa, Unknown
  Description of Unknown Hyfl M sequence
FNPG

SEQ ID NO: 834 - protein, 7 aa, Unknown
  Description of Unknown Hypa A sequence
TITALLG

SEQ ID NO: 835 - protein, 4 aa, Unknown
  Description of Unknown kalata B1 sequence
NTPG

SEQ ID NO: 836 - protein, 4 aa, Unknown
  Description of Unknown kalata B10 sequence
NTPG

SEQ ID NO: 837 - protein, 4 aa, Unknown
  Description of Unknown kalata B10 linear sequence
NTPG

SEQ ID NO: 838 - protein, 4 aa, Unknown
  Description of Unknown kalata B11 sequence
NTPG

SEQ ID NO: 839 - protein, 4 aa, Unknown
  Description of Unknown kalata B12 sequence
NDSS

SEQ ID NO: 840 - protein, 4 aa, Unknown
  Description of Unknown kalata B13 sequence
NTPG

SEQ ID NO: 841 - protein, 4 aa, Unknown
  Description of Unknown kalata B14 sequence
NTPG

SEQ ID NO: 842 - protein, 4 aa, Unknown
  Description of Unknown kalata B15 sequence
YTPG

SEQ ID NO: 843 - protein, 7 aa, Unknown
  Description of Unknown kalata B16 sequence
TITALLG

SEQ ID NO: 844 - protein, 7 aa, Unknown
  Description of Unknown kalata B17 sequence
TITALLG

SEQ ID NO: 845 - protein, 6 aa, Unknown
  Description of Unknown kalata B18 sequence
ISTVLG

SEQ ID NO: 846 - protein, 6 aa, Unknown
  Description of Unknown kalata B19 sequence
LTAAIG

SEQ ID NO: 847 - protein, 4 aa, Unknown
  Description of Unknown kalata B2 sequence
NTPG

SEQ ID NO: 848 - protein, 4 aa, Unknown
  Description of Unknown kalata B3 sequence
NTPG

SEQ ID NO: 849 - protein, 4 aa, Unknown
  Description of Unknown kalata B4 sequence
NTPG

SEQ ID NO: 850 - protein, 6 aa, Unknown
  Description of Unknown kalata B5 sequence
ISGVIG

SEQ ID NO: 851 - protein, 4 aa, Unknown
  Description of Unknown kalata B6 sequence
NTPG

SEQ ID NO: 852 - protein, 4 aa, Unknown
  Description of Unknown kalata B7 sequence
YTQG

SEQ ID NO: 853 - protein, 4 aa, Unknown
  Description of Unknown kalata B8 sequence
YTTG

SEQ ID NO: 854 - protein, 4 aa, Unknown
  Description of Unknown kalata B9 sequence
YTPG

SEQ ID NO: 855 - protein, 4 aa, Unknown
  Description of Unknown kalata B9 linear sequence
YTPG

SEQ ID NO: 856 - protein, 4 aa, Unknown
  Description of Unknown kalata S sequence
NTPG

SEQ ID NO: 857 - protein, 3 aa, Unknown
  Description of Unknown MCoTI-I sequence
PGA

SEQ ID NO: 858 - protein, 3 aa, Unknown
  Description of Unknown MCoTI-II sequence
PGA

SEQ ID NO: 859 - protein, 3 aa, Unknown
  Description of Unknown MCoTI-III sequence
PGE

SEQ ID NO: 860 - protein, 3 aa, Unknown
  Description of Unknown MCoTI-IV sequence
PGA

SEQ ID NO: 861 - protein, 3 aa, Unknown
  Description of Unknown MCoTI-V sequence
PGE

SEQ ID NO: 862 - protein, 3 aa, Unknown
  Description of Unknown MCoTI-VI sequence
PGE

SEQ ID NO: 863 - protein, 3 aa, Unknown
  Description of Unknown MCoTI-VII sequence
PGA

SEQ ID NO: 864 - protein, 3 aa, Unknown
  Description of Unknown MCoTI-VIII sequence
PGA

SEQ ID NO: 865 - protein, 4 aa, Unknown
  Description of Unknown mech 2 sequence
NTPK

SEQ ID NO: 866 - protein, 4 aa, Unknown
  Description of Unknown mech 3 sequence
NTPK

SEQ ID NO: 867 - protein, 6 aa, Unknown
  Description of Unknown mech 4 sequence
ISSIIG

SEQ ID NO: 868 - protein, 4 aa, Unknown
  Description of Unknown mela 7 sequence
YTPG

SEQ ID NO: 869 - protein, 6 aa, Unknown
  Description of Unknown Mra13 sequence
FTTIIG

SEQ ID NO: 870 - protein, 6 aa, Unknown
  Description of Unknown Mra14a sequence
ISSVVG

SEQ ID NO: 871 - protein, 6 aa, Unknown
  Description of Unknown Mra14b sequence
LTSAIG

SEQ ID NO: 872 - protein, 6 aa, Unknown
  Description of Unknown Mra17 sequence
ISSLLG

SEQ ID NO: 873 - protein, 6 aa, Unknown
  Description of Unknown Mra22 sequence
LTSIVG

SEQ ID NO: 874 - protein, 6 aa, Unknown
  Description of Unknown Mra23 sequence
ISSVLG

SEQ ID NO: 875 - protein, 4 aa, Unknown
  Description of Unknown Mra24 sequence
YTPG

SEQ ID NO: 876 - protein, 4 aa, Unknown
  Description of Unknown Mra25 sequence
YTPG

SEQ ID NO: 877 - protein, 4 aa, Unknown
  Description of Unknown Mra26 sequence
YTPG

SEQ ID NO: 878 - protein, 6 aa, Unknown
  Description of Unknown Mra29 sequence
ISSIVG

SEQ ID NO: 879 - protein, 6 aa, Unknown
  Description of Unknown Mra30 sequence
ISSIVG

SEQ ID NO: 880 - protein, 6 aa, Unknown
  Description of Unknown Mra30 sequence
LTSAIG

SEQ ID NO: 881 - protein, 6 aa, Unknown
  Description of Unknown Mra4 sequence
ISSLLG

SEQ ID NO: 882 - protein, 6 aa, Unknown
  Description of Unknown Mra5 sequence
LTSAIG

SEQ ID NO: 883 - protein, 4 aa, Unknown
  Description of Unknown Oak6 cyclotide 1 sequence
NTPG

SEQ ID NO: 884 - protein, 4 aa, Unknown
  Description of Unknown Oak6 cyclotide 2 sequence
NTPG

SEQ ID NO: 885 - protein, 4 aa, Unknown
  Description of Unknown Oak7 cyclotide sequence
YKPG

SEQ ID NO: 886 - protein, 6 aa, Unknown
  Description of Unknown Oak8 cyclotide sequence
LTAVVG

SEQ ID NO: 887 - protein, 7 aa, Unknown
  Description of Unknown palicourein sequence
TYSAALG

SEQ ID NO: 888 - protein, 4 aa, Unknown
  Description of Unknown paltet 1 sequence
NTPG

SEQ ID NO: 889 - protein, 4 aa, Unknown
  Description of Unknown Panitide L1 sequence
YTPG

SEQ ID NO: 890 - protein, 4 aa, Unknown
  Description of Unknown Panitide L2 sequence
YTPN

SEQ ID NO: 891 - protein, 4 aa, Unknown
  Description of Unknown Panitide L3 sequence
YTPG

SEQ ID NO: 892 - protein, 4 aa, Unknown
  Description of Unknown Panitide L4 sequence
YTPG

SEQ ID NO: 893 - protein, 4 aa, Unknown
  Description of Unknown Panitide L5 sequence
YTPG

SEQ ID NO: 894 - protein, 4 aa, Unknown
  Description of Unknown Panitide L6 sequence
YTPG

SEQ ID NO: 895 - protein, 4 aa, Unknown
  Description of Unknown Panitide L7 sequence
YTPG

SEQ ID NO: 896 - protein, 4 aa, Unknown
  Description of Unknown Panitide L8 sequence
YTPG

SEQ ID NO: 897 - protein, 6 aa, Unknown
  Description of Unknown Parigidin-br1 sequence
ITSLAG

SEQ ID NO: 898 - protein, 6 aa, Unknown
  Description of Unknown Phyb A sequence
VSAAIG

SEQ ID NO: 899 - protein, 6 aa, Unknown
  Description of Unknown Phyb B sequence
ISAAMG

SEQ ID NO: 900 - protein, 6 aa, Unknown
  Description of Unknown Phyb C sequence
ITATMG

SEQ ID NO: 901 - protein, 6 aa, Unknown
  Description of Unknown Phyb D sequence
ISAAIG

SEQ ID NO: 902 - protein, 6 aa, Unknown
  Description of Unknown Phyb E sequence
ISGVQG

SEQ ID NO: 903 - protein, 6 aa, Unknown
  Description of Unknown Phyb F sequence
ISGVQG

SEQ ID NO: 904 - protein, 6 aa, Unknown
  Description of Unknown Phyb G sequence
ISAVQG

SEQ ID NO: 905 - protein, 6 aa, Unknown
  Description of Unknown Phyb H sequence
ISGAIG

SEQ ID NO: 906 - protein, 7 aa, Unknown
  Description of Unknown Phyb I sequence
TTTALLG

SEQ ID NO: 907 - protein, 7 aa, Unknown
  Description of Unknown Phyb J sequence
TVTAAFG

SEQ ID NO: 908 - protein, 7 aa, Unknown
  Description of Unknown Phyb K sequence
TITALLG

SEQ ID NO: 909 - protein, 6 aa, Unknown
  Description of Unknown Phyb L sequence
ATSLIG

SEQ ID NO: 910 - protein, 4 aa, Unknown
  Description of Unknown PS-1 sequence
HAAG

SEQ ID NO: 911 - protein, 4 aa, Unknown
  Description of Unknown psybra 1 sequence
NTPG

SEQ ID NO: 912 - protein, 4 aa, Unknown
  Description of Unknown psyle A sequence
FIPG

SEQ ID NO: 913 - protein, 4 aa, Unknown
  Description of Unknown psyle B sequence
WIPG

SEQ ID NO: 914 - protein, 4 aa, Unknown
  Description of Unknown psyle C sequence
YTPG

SEQ ID NO: 915 - protein, 7 aa, Unknown
  Description of Unknown psyle D sequence
TVTALLG

SEQ ID NO: 916 - protein, 6 aa, Unknown
  Description of Unknown psyle E sequence
ISSVLG

SEQ ID NO: 917 - protein, 6 aa, Unknown
  Description of Unknown psyle F sequence
ITAAVG

SEQ ID NO: 918 - protein, 4 aa, Unknown
  Description of Unknown psypoe 1 sequence
NTPG

SEQ ID NO: 919 - protein, 4 aa, Unknown
  Description of Unknown tricyclon A sequence
YTKG

SEQ ID NO: 920 - protein, 4 aa, Unknown
  Description of Unknown tricyclon B sequence
YTKG

SEQ ID NO: 921 - protein, 4 aa, Unknown
  Description of Unknown vaby A sequence
NTPG

SEQ ID NO: 922 - protein, 4 aa, Unknown
  Description of Unknown vaby B sequence
NTPG

SEQ ID NO: 923 - protein, 4 aa, Unknown
  Description of Unknown vaby C sequence
NTPG

SEQ ID NO: 924 - protein, 4 aa, Unknown
  Description of Unknown vaby D sequence
NTPG

SEQ ID NO: 925 - protein, 4 aa, Unknown
  Description of Unknown vaby E sequence
NTPG

SEQ ID NO: 926 - protein, 4 aa, Unknown
  Description of Unknown varv peptide B sequence
NTPG

SEQ ID NO: 927 - protein, 4 aa, Unknown
  Description of Unknown varv peptide C sequence
NTPG

SEQ ID NO: 928 - protein, 4 aa, Unknown
  Description of Unknown varv peptide D sequence
NTPG

SEQ ID NO: 929 - protein, 4 aa, Unknown
  Description of Unknown varv peptide F sequence
YTAG

SEQ ID NO: 930 - protein, 4 aa, Unknown
  Description of Unknown varv peptide G sequence
NTPG

SEQ ID NO: 931 - protein, 4 aa, Unknown
  Description of Unknown varv peptide H sequence
NTPG

SEQ ID NO: 932 - protein, 6 aa, Unknown
  Description of Unknown vhl-1 sequence
FTEVIG

SEQ ID NO: 933 - protein, 4 aa, Unknown
  Description of Unknown vhl-2 sequence
YTNG

SEQ ID NO: 934 - protein, 7 aa, Unknown
  Description of Unknown vhr1 sequence
TVTALLG

SEQ ID NO: 935 - protein, 6 aa, Unknown
  Description of Unknown Viba 1 sequence
FTAPLG

SEQ ID NO: 936 - protein, 6 aa, Unknown
  Description of Unknown Viba 10 sequence
VTIVIG

SEQ ID NO: 937 - protein, 6 aa, Unknown
  Description of Unknown Viba 11 sequence
ISGAIG

SEQ ID NO: 938 - protein, 7 aa, Unknown
  Description of Unknown Viba 12 sequence
TVTALLG

SEQ ID NO: 939 - protein, 7 aa, Unknown
  Description of Unknown Viba 13 sequence
TVTALLG

SEQ ID NO: 940 - protein, 4 aa, Unknown
  Description of Unknown Viba 14 sequence
RTVG

SEQ ID NO: 941 - protein, 4 aa, Unknown
  Description of Unknown Viba 15 sequence
NTPG

SEQ ID NO: 942 - protein, 4 aa, Unknown
  Description of Unknown Viba 16 sequence
YTVG

SEQ ID NO: 943 - protein, 4 aa, Unknown
  Description of Unknown Viba 17 sequence
NTPG

SEQ ID NO: 944 - protein, 6 aa, Unknown
  Description of Unknown Viba 2 sequence
FTAPLG

SEQ ID NO: 945 - protein, 6 aa, Unknown
  Description of Unknown Viba 3 sequence
LTAAIG

SEQ ID NO: 946 - protein, 6 aa, Unknown
  Description of Unknown Viba 4 sequence
LTSAIG

SEQ ID NO: 947 - protein, 6 aa, Unknown
  Description of Unknown Viba 5 sequence
LTATIG

SEQ ID NO: 948 - protein, 6 aa, Unknown
  Description of Unknown Viba 6 sequence
ISSVIG

SEQ ID NO: 949 - protein, 6 aa, Unknown
  Description of Unknown Viba 7 sequence
ISSVIG

SEQ ID NO: 950 - protein, 6 aa, Unknown
  Description of Unknown Viba 8 sequence
ISEMIN

SEQ ID NO: 951 - protein, 6 aa, Unknown
  Description of Unknown Viba 9 sequence
ISSAIG

SEQ ID NO: 952 - protein, 4 aa, Unknown
  Description of Unknown vibi A sequence
NTPG

SEQ ID NO: 953 - protein, 4 aa, Unknown
  Description of Unknown vibi B sequence
NTPG

SEQ ID NO: 954 - protein, 4 aa, Unknown
  Description of Unknown vibi C sequence
YTPG

SEQ ID NO: 955 - protein, 4 aa, Unknown
  Description of Unknown vibi D sequence
NTPG

SEQ ID NO: 956 - protein, 7 aa, Unknown
  Description of Unknown vibi E sequence
TVTALIG

SEQ ID NO: 957 - protein, 6 aa, Unknown
  Description of Unknown vibi F sequence
LTSALG

SEQ ID NO: 958 - protein, 6 aa, Unknown
  Description of Unknown vibi G sequence
LTSAIG

SEQ ID NO: 959 - protein, 6 aa, Unknown
  Description of Unknown vibi H sequence
LTTVIG

SEQ ID NO: 960 - protein, 6 aa, Unknown
  Description of Unknown vibi I sequence
LTSTVG

SEQ ID NO: 961 - protein, 6 aa, Unknown
  Description of Unknown vibi J sequence
ISKVIG

SEQ ID NO: 962 - protein, 6 aa, Unknown
  Description of Unknown vibi K sequence
LTSAVG

SEQ ID NO: 963 - protein, 6 aa, Unknown
  Description of Unknown vico A sequence
FTGIAG

SEQ ID NO: 964 - protein, 6 aa, Unknown
  Description of Unknown vico B sequence
ITGIAG

SEQ ID NO: 965 - protein, 4 aa, Unknown
  Description of Unknown vigno 1 sequence
NTPG

SEQ ID NO: 966 - protein, 6 aa, Unknown
  Description of Unknown vigno 10 sequence
ISSVVG

SEQ ID NO: 967 - protein, 4 aa, Unknown
  Description of Unknown vigno 2 sequence
NTPG

SEQ ID NO: 968 - protein, 4 aa, Unknown
  Description of Unknown vigno 3 sequence
NTPG

SEQ ID NO: 969 - protein, 4 aa, Unknown
  Description of Unknown vigno 4 sequence
NTPA

SEQ ID NO: 970 - protein, 4 aa, Unknown
  Description of Unknown vigno 5 sequence
NTPG

SEQ ID NO: 971 - protein, 6 aa, Unknown
  Description of Unknown vigno 6 sequence
ISSAIG

SEQ ID NO: 972 - protein, 6 aa, Unknown
  Description of Unknown vigno 7 sequence
ISSVVG

SEQ ID NO: 973 - protein, 6 aa, Unknown
  Description of Unknown vigno 8 sequence
ITSAVG

SEQ ID NO: 974 - protein, 6 aa, Unknown
  Description of Unknown vigno 9 sequence
ISSALG

SEQ ID NO: 975 - protein, 4 aa, Unknown
  Description of Unknown violacin A sequence
YTPR

SEQ ID NO: 976 - protein, 4 aa, Unknown
  Description of Unknown violapeptide 1 sequence
NTPG

SEQ ID NO: 977 - protein, 6 aa, Unknown
  Description of Unknown viphi A sequence
ISSVIG

SEQ ID NO: 978 - protein, 4 aa, Unknown
  Description of Unknown viphi B sequence
YTAG

SEQ ID NO: 979 - protein, 6 aa, Unknown
  Description of Unknown viphi C sequence
ITSVIG

SEQ ID NO: 980 - protein, 6 aa, Unknown
  Description of Unknown viphi D sequence
ISSVIG

SEQ ID NO: 981 - protein, 6 aa, Unknown
  Description of Unknown viphi E sequence
ISAVIG

SEQ ID NO: 982 - protein, 6 aa, Unknown
  Description of Unknown viphi F sequence
ISAIIG

SEQ ID NO: 983 - protein, 6 aa, Unknown
  Description of Unknown viphi G sequence
ISAIIG

SEQ ID NO: 984 - protein, 7 aa, Unknown
  Description of Unknown viphi H sequence
TVTAIVG

SEQ ID NO: 985 - protein, 6 aa, Unknown
  Description of Unknown vitri A sequence
ITSAIG

SEQ ID NO: 986 - protein, 4 aa, Unknown
  Description of Unknown vitri B sequence
NTPG

SEQ ID NO: 987 - protein, 4 aa, Unknown
  Description of Unknown vitri C sequence
NTPG

SEQ ID NO: 988 - protein, 4 aa, Unknown
  Description of Unknown vitri D sequence
YTPG

SEQ ID NO: 989 - protein, 4 aa, Unknown
  Description of Unknown vitri E sequence
NTPG

SEQ ID NO: 990 - protein, 6 aa, Unknown
  Description of Unknown vitri F sequence
ISSVVG

SEQ ID NO: 991 - protein, 6 aa, Unknown
  Description of Unknown vitri peptide 1 sequence
ISSVIG

SEQ ID NO: 992 - protein, 4 aa, Unknown
  Description of Unknown vitri peptide 18a sequence
NTPG

SEQ ID NO: 993 - protein, 4 aa, Unknown
  Description of Unknown vitri peptide 18b sequence
FTSG

SEQ ID NO: 994 - protein, 6 aa, Unknown
  Description of Unknown vitri peptide 2 sequence
ISGIAG

SEQ ID NO: 995 - protein, 6 aa, Unknown
  Description of Unknown vitri peptide 20 sequence
LTTVLG

SEQ ID NO: 996 - protein, 4 aa, Unknown
  Description of Unknown vitri peptide 22a sequence
YSSG

SEQ ID NO: 997 - protein, 4 aa, Unknown
  Description of Unknown vitri peptide 23 sequence
YTPG

SEQ ID NO: 998 - protein, 4 aa, Unknown
  Description of Unknown vitri peptide 24a sequence
YTKG

SEQ ID NO: 999 - protein, 6 aa, Unknown
  Description of Unknown vitri peptide 3 sequence
ITSIAG

SEQ ID NO: 1000 - protein, 4 aa, Unknown
  Description of Unknown vitri peptide 30 sequence
FITG

SEQ ID NO: 1001 - protein, 4 aa, Unknown
  Description of Unknown vitri peptide 36/37 sequence
YTKG

SEQ ID NO: 1002 - protein, 4 aa, Unknown
  Description of Unknown vitri peptide 39 sequence
YTVQ

SEQ ID NO: 1003 - protein, 4 aa, Unknown
  Description of Unknown vitri peptide 39 linear sequence
YTVQ

SEQ ID NO: 1004 - protein, 6 aa, Unknown
  Description of Unknown vitri peptide 4 sequence
ISAVFG

SEQ ID NO: 1005 - protein, 6 aa, Unknown
  Description of Unknown vitri peptide 50 sequence
ITGVLG

SEQ ID NO: 1006 - protein, 6 aa, Unknown
  Description of Unknown vitri peptide 8 sequence
VTAAIG

SEQ ID NO: 1007 - protein, 4 aa, Unknown
  Description of Unknown vitri peptide 94b sequence
YTPG

SEQ ID NO: 1008 - protein, 4 aa, Unknown
  Description of Unknown vitri peptide 9a/53 sequence
YTPG

SEQ ID NO: 1009 - protein, 4 aa, Unknown
  Description of Unknown vocC sequence
NTPG

SEQ ID NO: 1010 - protein, 4 aa, Unknown
  Description of Unknown vodo M sequence
YTVQ

SEQ ID NO: 1011 - protein, 4 aa, Unknown
  Description of Unknown vodo N sequence
YTAG

SEQ ID NO: 1012 - protein, 6 aa, Unknown
  Description of Unknown Vpf-1 sequence
LTAAIG

SEQ ID NO: 1013 - protein, 6 aa, Unknown
  Description of Unknown Vpl-1 sequence
ISGVIG

SEQ ID NO: 1014 - protein, 4 aa, Unknown
  Description of Unknown caripe 1 sequence
KDKV

SEQ ID NO: 1015 - protein, 4 aa, Unknown
  Description of Unknown caripe 10 sequence
KNKV

SEQ ID NO: 1016 - protein, 4 aa, Unknown
  Description of Unknown caripe 11 sequence
KKKV

SEQ ID NO: 1017 - protein, 4 aa, Unknown
  Description of Unknown caripe 12 sequence
KNKV

SEQ ID NO: 1018 - protein, 4 aa, Unknown
  Description of Unknown caripe 13 sequence
KDKV

SEQ ID NO: 1019 - protein, 4 aa, Unknown
  Description of Unknown caripe 2 sequence
SNNV

SEQ ID NO: 1020 - protein, 4 aa, Unknown
  Description of Unknown caripe 4 sequence
RHHI

SEQ ID NO: 1021 - protein, 4 aa, Unknown
  Description of Unknown caripe 6 sequence
RHYI

SEQ ID NO: 1022 - protein, 4 aa, Unknown
  Description of Unknown caripe 7 sequence
KNKV

SEQ ID NO: 1023 - protein, 4 aa, Unknown
  Description of Unknown caripe 8 sequence
KKKV

SEQ ID NO: 1024 - protein, 6 aa, Unknown
  Description of Unknown CD-1 sequence
DTIEKV

SEQ ID NO: 1025 - protein, 4 aa, Unknown
  Description of Unknown chacur 1 sequence
SWPI

SEQ ID NO: 1026 - protein, 5 aa, Unknown
  Description of Unknown chassatide C1 sequence
NPWPT

SEQ ID NO: 1027 - protein, 4 aa, Unknown
  Description of Unknown chassatide C10 sequence
VSRQ

SEQ ID NO: 1028 - protein, 4 aa, Unknown
  Description of Unknown chassatide C11 sequence
KDKV

SEQ ID NO: 1029 - protein, 4 aa, Unknown
  Description of Unknown chassatide C12 sequence
VSRQ

SEQ ID NO: 1030 - protein, 4 aa, Unknown
  Description of Unknown chassatide C13 sequence
RNRV

SEQ ID NO: 1031 - protein, 4 aa, Unknown
  Description of Unknown chassatide C14 sequence
KDKV

SEQ ID NO: 1032 - protein, 4 aa, Unknown
  Description of Unknown chassatide C15 sequence
KDKV

SEQ ID NO: 1033 - protein, 4 aa, Unknown
  Description of Unknown chassatide C16 sequence
KDKV

SEQ ID NO: 1034 - protein, 4 aa, Unknown
  Description of Unknown chassatide C17 sequence
QNKV

SEQ ID NO: 1035 - protein, 4 aa, Unknown
  Description of Unknown chassatide C18 sequence
SNKV

SEQ ID NO: 1036 - protein, 4 aa, Unknown
  Description of Unknown chassatide C2 sequence
KNNV

SEQ ID NO: 1037 - protein, 4 aa, Unknown
  Description of Unknown chassatide C3 sequence
KNKV

SEQ ID NO: 1038 - protein, 5 aa, Unknown
  Description of Unknown chassatide C4 sequence
NPWPT

SEQ ID NO: 1039 - protein, 4 aa, Unknown
  Description of Unknown chassatide C5 sequence
KNKV

SEQ ID NO: 1040 - protein, 4 aa, Unknown
  Description of Unknown chassatide C6 sequence
KNKV

SEQ ID NO: 1041 - protein, 4 aa, Unknown
  Description of Unknown chassatide C7 sequence
KNKV

SEQ ID NO: 1042 - protein, 4 aa, Unknown
  Description of Unknown chassatide C8 sequence
SNKV

SEQ ID NO: 1043 - protein, 4 aa, Unknown
  Description of Unknown chassatide C9 sequence
KDKV

SEQ ID NO: 1044 - protein, 4 aa, Unknown
  Description of Unknown circulin A sequence
KNKV

SEQ ID NO: 1045 - protein, 4 aa, Unknown
  Description of Unknown circulin B sequence
KNKV

SEQ ID NO: 1046 - protein, 4 aa, Unknown
  Description of Unknown circulin C sequence
KSKV

SEQ ID NO: 1047 - protein, 4 aa, Unknown
  Description of Unknown circulin D sequence
ENKV

SEQ ID NO: 1048 - protein, 4 aa, Unknown
  Description of Unknown circulin E sequence
ENKV

SEQ ID NO: 1049 - protein, 4 aa, Unknown
  Description of Unknown circulin F sequence
KNKV

SEQ ID NO: 1050 - protein, 4 aa, Unknown
  Description of Unknown cliotide T12 sequence
KSKV

SEQ ID NO: 1051 - protein, 4 aa, Unknown
  Description of Unknown cliotide T2 sequence
DWPI

SEQ ID NO: 1052 - protein, 4 aa, Unknown
  Description of Unknown cliotide T8 sequence
KSKV

SEQ ID NO: 1053 - protein, 4 aa, Unknown
  Description of Unknown cliotide T9 sequence
KNKV

SEQ ID NO: 1054 - protein, 5 aa, Unknown
  Description of Unknown cO26 sequence
SKYPL

SEQ ID NO: 1055 - protein, 5 aa, Unknown
  Description of Unknown cO27 sequence
SKYPL

SEQ ID NO: 1056 - protein, 4 aa, Unknown
  Description of Unknown cO28 sequence
SWPV

SEQ ID NO: 1057 - protein, 4 aa, Unknown
  Description of Unknown cO29 sequence
KSKV

SEQ ID NO: 1058 - protein, 4 aa, Unknown
  Description of Unknown cO30 sequence
KNKV

SEQ ID NO: 1059 - protein, 4 aa, Unknown
  Description of Unknown cO31 sequence
SIPV

SEQ ID NO: 1060 - protein, 5 aa, Unknown
  Description of Unknown cO32 sequence
DPWPV

SEQ ID NO: 1061 - protein, 4 aa, Unknown
  Description of Unknown cO33 sequence
SWPV

SEQ ID NO: 1062 - protein, 4 aa, Unknown
  Description of Unknown cO34 sequence
SWPV

SEQ ID NO: 1063 - protein, 4 aa, Unknown
  Description of Unknown cO35 sequence
SWPV

SEQ ID NO: 1064 - protein, 5 aa, Unknown
  Description of Unknown cO36 sequence
DPFPV

SEQ ID NO: 1065 - protein, 4 aa, Unknown
  Description of Unknown Cter 35 sequence
APVI

SEQ ID NO: 1066 - protein, 4 aa, Unknown
  Description of Unknown Cter A sequence
KNKV

SEQ ID NO: 1067 - protein, 4 aa, Unknown
  Description of Unknown Cter B sequence
KDKV

SEQ ID NO: 1068 - protein, 4 aa, Unknown
  Description of Unknown Cter C sequence
KDKV

SEQ ID NO: 1069 - protein, 4 aa, Unknown
  Description of Unknown Cter D sequence
KDKV

SEQ ID NO: 1070 - protein, 4 aa, Unknown
  Description of Unknown Cter E sequence
KDKV

SEQ ID NO: 1071 - protein, 4 aa, Unknown
  Description of Unknown Cter F sequence
KSKV

SEQ ID NO: 1072 - protein, 4 aa, Unknown
  Description of Unknown Cter G sequence
KNKV

SEQ ID NO: 1073 - protein, 4 aa, Unknown
  Description of Unknown Cter H sequence
KNKV

SEQ ID NO: 1074 - protein, 4 aa, Unknown
  Description of Unknown Cter I sequence
KNKV

SEQ ID NO: 1075 - protein, 4 aa, Unknown
  Description of Unknown Cter J sequence
KNKV

SEQ ID NO: 1076 - protein, 4 aa, Unknown
  Description of Unknown Cter K sequence
KNKV

SEQ ID NO: 1077 - protein, 4 aa, Unknown
  Description of Unknown Cter L sequence
KNKV

SEQ ID NO: 1078 - protein, 4 aa, Unknown
  Description of Unknown Cter M sequence
SWPI

SEQ ID NO: 1079 - protein, 4 aa, Unknown
  Description of Unknown Cter N sequence
TALV

SEQ ID NO: 1080 - protein, 4 aa, Unknown
  Description of Unknown Cter O sequence
KSKV

SEQ ID NO: 1081 - protein, 4 aa, Unknown
  Description of Unknown Cter P sequence
KSKV

SEQ ID NO: 1082 - protein, 4 aa, Unknown
  Description of Unknown Cter Q sequence
KNKV

SEQ ID NO: 1083 - protein, 4 aa, Unknown
  Description of Unknown Cter R sequence
KDKV

SEQ ID NO: 1084 - protein, 4 aa, Unknown
  Description of Unknown cyclopsychotride A sequence
KSKV

SEQ ID NO: 1085 - protein, 4 aa, Unknown
  Description of Unknown cycloviolacin H1 sequence
KSKV

SEQ ID NO: 1086 - protein, 4 aa, Unknown
  Description of Unknown cycloviolacin H2 sequence
RNRV

SEQ ID NO: 1087 - protein, 5 aa, Unknown
  Description of Unknown cycloviolacin H3 sequence
DPWPV

SEQ ID NO: 1088 - protein, 4 aa, Unknown
  Description of Unknown cycloviolacin H4 sequence
SNNV

SEQ ID NO: 1089 - protein, 4 aa, Unknown
  Description of Unknown cycloviolacin O1 sequence
SNRV

SEQ ID NO: 1090 - protein, 4 aa, Unknown
  Description of Unknown cycloviolacin O10 sequence
KSKV

SEQ ID NO: 1091 - protein, 4 aa, Unknown
  Description of Unknown cycloviolacin O11 sequence
KSKV

SEQ ID NO: 1092 - protein, 4 aa, Unknown
  Description of Unknown cycloviolacin O12 sequence
SWPV

SEQ ID NO: 1093 - protein, 4 aa, Unknown
  Description of Unknown cycloviolacin O13 sequence
KSKV

SEQ ID NO: 1094 - protein, 5 aa, Unknown
  Description of Unknown cycloviolacin O14 sequence
SKYPL

SEQ ID NO: 1095 - protein, 4 aa, Unknown
  Description of Unknown cycloviolacin O15 sequence
SYPI

SEQ ID NO: 1096 - protein, 4 aa, Unknown
  Description of Unknown cycloviolacin O16 sequence
SYPI

SEQ ID NO: 1097 - protein, 4 aa, Unknown
  Description of Unknown cycloviolacin O17 sequence
KNKV

SEQ ID NO: 1098 - protein, 4 aa, Unknown
  Description of Unknown cycloviolacin O18 sequence
KSKV

SEQ ID NO: 1099 - protein, 4 aa, Unknown
  Description of Unknown cycloviolacin O19 sequence
KSKV

SEQ ID NO: 1100 - protein, 4 aa, Unknown
  Description of Unknown cycloviolacin O2 sequence
KSKV

SEQ ID NO: 1101 - protein, 4 aa, Unknown
  Description of Unknown cycloviolacin O20 sequence
KSKV

SEQ ID NO: 1102 - protein, 4 aa, Unknown
  Description of Unknown cycloviolacin O21 sequence
SWPV

SEQ ID NO: 1103 - protein, 4 aa, Unknown
  Description of Unknown cycloviolacin O22 sequence
SWPV

SEQ ID NO: 1104 - protein, 6 aa, Unknown
  Description of Unknown cycloviolacin O23 sequence
DSSWPI

SEQ ID NO: 1105 - protein, 5 aa, Unknown
  Description of Unknown cycloviolacin O24 sequence
DPWPV

SEQ ID NO: 1106 - protein, 4 aa, Unknown
  Description of Unknown cycloviolacin O25 sequence
KSKV

SEQ ID NO: 1107 - protein, 4 aa, Unknown
  Description of Unknown cycloviolacin O3 sequence
KSKV

SEQ ID NO: 1108 - protein, 4 aa, Unknown
  Description of Unknown cycloviolacin O4 sequence
KNKV

SEQ ID NO: 1109 - protein, 4 aa, Unknown
  Description of Unknown cycloviolacin O5 sequence
KNKV

SEQ ID NO: 1110 - protein, 4 aa, Unknown
  Description of Unknown cycloviolacin O6 sequence
KSKV

SEQ ID NO: 1111 - protein, 4 aa, Unknown
  Description of Unknown cycloviolacin O7 sequence
KSKV

SEQ ID NO: 1112 - protein, 4 aa, Unknown
  Description of Unknown cycloviolacin O8 sequence
KSKV

SEQ ID NO: 1113 - protein, 4 aa, Unknown
  Description of Unknown cycloviolacin O9 sequence
KSKV

SEQ ID NO: 1114 - protein, 4 aa, Unknown
  Description of Unknown cycloviolacin T1 sequence
SWPV

SEQ ID NO: 1115 - protein, 5 aa, Unknown
  Description of Unknown cycloviolacin Y1 sequence
GNYGF

SEQ ID NO: 1116 - protein, 5 aa, Unknown
  Description of Unknown cycloviolacin Y2 sequence
GNWGL

SEQ ID NO: 1117 - protein, 5 aa, Unknown
  Description of Unknown cycloviolacin Y3 sequence
GNWGL

SEQ ID NO: 1118 - protein, 4 aa, Unknown
  Description of Unknown cycloviolacin Y4 sequence
SSNV

SEQ ID NO: 1119 - protein, 4 aa, Unknown
  Description of Unknown cycloviolacin Y5 sequence
SDKV

SEQ ID NO: 1120 - protein, 4 aa, Unknown
  Description of Unknown cycloviolin A sequence
KNKV

SEQ ID NO: 1121 - protein, 4 aa, Unknown
  Description of Unknown cycloviolin B sequence
TSSQ

SEQ ID NO: 1122 - protein, 4 aa, Unknown
  Description of Unknown cycloviolin C sequence
KNKV

SEQ ID NO: 1123 - protein, 4 aa, Unknown
  Description of Unknown cycloviolin D sequence
KNKV

SEQ ID NO: 1124 - protein, 4 aa, Unknown
  Description of Unknown Globa A sequence
KTKV

SEQ ID NO: 1125 - protein, 4 aa, Unknown
  Description of Unknown Globa B sequence
KSKV

SEQ ID NO: 1126 - protein, 4 aa, Unknown
  Description of Unknown Globa C sequence
SNIV

SEQ ID NO: 1127 - protein, 4 aa, Unknown
  Description of Unknown Globa D sequence
KHMV

SEQ ID NO: 1128 - protein, 4 aa, Unknown
  Description of Unknown Globa E sequence
SWPV

SEQ ID NO: 1129 - protein, 4 aa, Unknown
  Description of Unknown Globa F sequence
KNKV

SEQ ID NO: 1130 - protein, 5 aa, Unknown
  Description of Unknown Glopa A sequence
KSDKK

SEQ ID NO: 1131 - protein, 5 aa, Unknown
  Description of Unknown Glopa B sequence
KSDKK

SEQ ID NO: 1132 - protein, 4 aa, Unknown
  Description of Unknown Glopa C sequence
VWPF

SEQ ID NO: 1133 - protein, 4 aa, Unknown
  Description of Unknown Glopa D sequence
VREV

SEQ ID NO: 1134 - protein, 4 aa, Unknown
  Description of Unknown Glopa E sequence
KDKV

SEQ ID NO: 1135 - protein, 4 aa, Unknown
  Description of Unknown hcf-1 sequence
RNRS

SEQ ID NO: 1136 - protein, 4 aa, Unknown
  Description of Unknown hcf-1 variant sequence
RNRS

SEQ ID NO: 1137 - protein, 4 aa, Unknown
  Description of Unknown hedyotide B1 sequence
QKGF

SEQ ID NO: 1138 - protein, 4 aa, Unknown
  Description of Unknown hedyotide B2 sequence
KNKI

SEQ ID NO: 1139 - protein, 4 aa, Unknown
  Description of Unknown htf-1 sequence
TNGS

SEQ ID NO: 1140 - protein, 4 aa, Unknown
  Description of Unknown Hyfl A sequence
KDKV

SEQ ID NO: 1141 - protein, 4 aa, Unknown
  Description of Unknown Hyfl B sequence
TAFL

SEQ ID NO: 1142 - protein, 4 aa, Unknown
  Description of Unknown Hyfl C sequence
TAFL

SEQ ID NO: 1143 - protein, 4 aa, Unknown
  Description of Unknown Hyfl D sequence
KSKV

SEQ ID NO: 1144 - protein, 4 aa, Unknown
  Description of Unknown Hyfl E sequence
RNHV

SEQ ID NO: 1145 - protein, 6 aa, Unknown
  Description of Unknown Hyfl F sequence
NHHDKV

SEQ ID NO: 1146 - protein, 4 aa, Unknown
  Description of Unknown Hyfl I sequence
KSKV

SEQ ID NO: 1147 - protein, 4 aa, Unknown
  Description of Unknown Hyfl J sequence
RNKV

SEQ ID NO: 1148 - protein, 4 aa, Unknown
  Description of Unknown Hyfl K sequence
KDKV

SEQ ID NO: 1149 - protein, 4 aa, Unknown
  Description of Unknown Hyfl L sequence
KDKV

SEQ ID NO: 1150 - protein, 4 aa, Unknown
  Description of Unknown Hyfl M sequence
KDNL

SEQ ID NO: 1151 - protein, 4 aa, Unknown
  Description of Unknown Hypa A sequence
KNKV

SEQ ID NO: 1152 - protein, 4 aa, Unknown
  Description of Unknown kalata B1 sequence
SWPV

SEQ ID NO: 1153 - protein, 5 aa, Unknown
  Description of Unknown kalata B10 sequence
SSWPI

SEQ ID NO: 1154 - protein, 5 aa, Unknown
  Description of Unknown kalata B10 linear sequence
SSWPI

SEQ ID NO: 1155 - protein, 4 aa, Unknown
  Description of Unknown kalata B11 sequence
TDPI

SEQ ID NO: 1156 - protein, 4 aa, Unknown
  Description of Unknown kalata B12 sequence
NYPI

SEQ ID NO: 1157 - protein, 5 aa, Unknown
  Description of Unknown kalata B13 sequence
DPWPV

SEQ ID NO: 1158 - protein, 5 aa, Unknown
  Description of Unknown kalata B14 sequence
DPWPV

SEQ ID NO: 1159 - protein, 4 aa, Unknown
  Description of Unknown kalata B15 sequence
TWPI

SEQ ID NO: 1160 - protein, 4 aa, Unknown
  Description of Unknown kalata B16 sequence
QDKV

SEQ ID NO: 1161 - protein, 4 aa, Unknown
  Description of Unknown kalata B17 sequence
KDQV

SEQ ID NO: 1162 - protein, 4 aa, Unknown
  Description of Unknown kalata B18 sequence
SNQV

SEQ ID NO: 1163 - protein, 4 aa, Unknown
  Description of Unknown kalata B19 sequence
SNKV

SEQ ID NO: 1164 - protein, 4 aa, Unknown
  Description of Unknown kalata B2 sequence
TWPI

SEQ ID NO: 1165 - protein, 5 aa, Unknown
  Description of Unknown kalata B3 sequence
DPWPI

SEQ ID NO: 1166 - protein, 4 aa, Unknown
  Description of Unknown kalata B4 sequence
SWPV

SEQ ID NO: 1167 - protein, 4 aa, Unknown
  Description of Unknown kalata B5 sequence
TDKV

SEQ ID NO: 1168 - protein, 5 aa, Unknown
  Description of Unknown kalata B6 sequence
SSWPI

SEQ ID NO: 1169 - protein, 4 aa, Unknown
  Description of Unknown kalata B7 sequence
SWPI

SEQ ID NO: 1170 - protein, 5 aa, Unknown
  Description of Unknown kalata B8 sequence
NKYRV

SEQ ID NO: 1171 - protein, 5 aa, Unknown
  Description of Unknown kalata B9 sequence
NTYRV

SEQ ID NO: 1172 - protein, 5 aa, Unknown
  Description of Unknown kalata B9 linear sequence
NTYRV

SEQ ID NO: 1173 - protein, 4 aa, Unknown
  Description of Unknown kalata S sequence
SWPV

SEQ ID NO: 1174 - protein, 5 aa, Unknown
  Description of Unknown MCoTI-I sequence
RGNGY

SEQ ID NO: 1175 - protein, 5 aa, Unknown
  Description of Unknown MCoTI-II sequence
RGNGY

SEQ ID NO: 1176 - protein, 5 aa, Unknown
  Description of Unknown MCoTI-III sequence
KENGY

SEQ ID NO: 1177 - protein, 5 aa, Unknown
  Description of Unknown MCoTI-IV sequence
QGNGY

SEQ ID NO: 1178 - protein, 5 aa, Unknown
  Description of Unknown MCoTI-V sequence
KGNGY

SEQ ID NO: 1179 - protein, 5 aa, Unknown
  Description of Unknown MCoTI-VI sequence
QGNGY

SEQ ID NO: 1180 - protein, 5 aa, Unknown
  Description of Unknown MCoTI-VII sequence
KGNGY

SEQ ID NO: 1181 - protein, 5 aa, Unknown
  Description of Unknown MCoTI-VIII sequence
LGNGY

SEQ ID NO: 1182 - protein, 4 aa, Unknown
  Description of Unknown mech 2 sequence
NWPI

SEQ ID NO: 1183 - protein, 4 aa, Unknown
  Description of Unknown mech 3 sequence
NWPI

SEQ ID NO: 1184 - protein, 4 aa, Unknown
  Description of Unknown mech 4 sequence
KSKV

SEQ ID NO: 1185 - protein, 4 aa, Unknown
  Description of Unknown mela 7 sequence
SYPI

SEQ ID NO: 1186 - protein, 4 aa, Unknown
  Description of Unknown Mra13 sequence
QGKV

SEQ ID NO: 1187 - protein, 4 aa, Unknown
  Description of Unknown Mra14a sequence
KNKV

SEQ ID NO: 1188 - protein, 4 aa, Unknown
  Description of Unknown Mra14b sequence
KSKV

SEQ ID NO: 1189 - protein, 4 aa, Unknown
  Description of Unknown Mra17 sequence
ESKV

SEQ ID NO: 1190 - protein, 4 aa, Unknown
  Description of Unknown Mra22 sequence
KNNV

SEQ ID NO: 1191 - protein, 4 aa, Unknown
  Description of Unknown Mra23 sequence
KNKV

SEQ ID NO: 1192 - protein, 4 aa, Unknown
  Description of Unknown Mra24 sequence
KRPV

SEQ ID NO: 1193 - protein, 4 aa, Unknown
  Description of Unknown Mra25 sequence
SWPI

SEQ ID NO: 1194 - protein, 4 aa, Unknown
  Description of Unknown Mra26 sequence
TWPV

SEQ ID NO: 1195 - protein, 4 aa, Unknown
  Description of Unknown Mra29 sequence
KSKV

SEQ ID NO: 1196 - protein, 4 aa, Unknown
  Description of Unknown Mra30 sequence
KSKV

SEQ ID NO: 1197 - protein, 4 aa, Unknown
  Description of Unknown Mra30 sequence
KSKV

SEQ ID NO: 1198 - protein, 4 aa, Unknown
  Description of Unknown Mra4 sequence
KSKV

SEQ ID NO: 1199 - protein, 4 aa, Unknown
  Description of Unknown Mra5 sequence
KSKV

SEQ ID NO: 1200 - protein, 5 aa, Unknown
  Description of Unknown Oak6 cyclotide 1 sequence
DPWPV

SEQ ID NO: 1201 - protein, 5 aa, Unknown
  Description of Unknown Oak6 cyclotide 2 sequence
DPWPV

SEQ ID NO: 1202 - protein, 5 aa, Unknown
  Description of Unknown Oak7 cyclotide sequence
DELRQ

SEQ ID NO: 1203 - protein, 4 aa, Unknown
  Description of Unknown Oak8 cyclotide sequence
SNKV

SEQ ID NO: 1204 - protein, 7 aa, Unknown
  Description of Unknown palicourein sequence
DDRSDGL

SEQ ID NO: 1205 - protein, 4 aa, Unknown
  Description of Unknown paltet 1 sequence
SYPV

SEQ ID NO: 1206 - protein, 4 aa, Unknown
  Description of Unknown Panitide L1 sequence
QYPI

SEQ ID NO: 1207 - protein, 4 aa, Unknown
  Description of Unknown Panitide L2 sequence
QYPI

SEQ ID NO: 1208 - protein, 4 aa, Unknown
  Description of Unknown Panitide L3 sequence
HTGI

SEQ ID NO: 1209 - protein, 4 aa, Unknown
  Description of Unknown Panitide L4 sequence
TAGI

SEQ ID NO: 1210 - protein, 4 aa, Unknown
  Description of Unknown Panitide L5 sequence
AYPI

SEQ ID NO: 1211 - protein, 4 aa, Unknown
  Description of Unknown Panitide L6 sequence
AYPI

SEQ ID NO: 1212 - protein, 4 aa, Unknown
  Description of Unknown Panitide L7 sequence
NFGI

SEQ ID NO: 1213 - protein, 5 aa, Unknown
  Description of Unknown Panitide L8 sequence
SAYPL

SEQ ID NO: 1214 - protein, 4 aa, Unknown
  Description of Unknown Parigidin-br1 sequence
KNKV

SEQ ID NO: 1215 - protein, 4 aa, Unknown
  Description of Unknown Phyb A sequence
SNKI

SEQ ID NO: 1216 - protein, 4 aa, Unknown
  Description of Unknown Phyb B sequence
RNKV

SEQ ID NO: 1217 - protein, 4 aa, Unknown
  Description of Unknown Phyb C sequence
RNKV

SEQ ID NO: 1218 - protein, 4 aa, Unknown
  Description of Unknown Phyb D sequence
TNHV

SEQ ID NO: 1219 - protein, 4 aa, Unknown
  Description of Unknown Phyb E sequence
SNKI

SEQ ID NO: 1220 - protein, 4 aa, Unknown
  Description of Unknown Phyb F sequence
SNKI

SEQ ID NO: 1221 - protein, 4 aa, Unknown
  Description of Unknown Phyb G sequence
RNKI

SEQ ID NO: 1222 - protein, 4 aa, Unknown
  Description of Unknown Phyb H sequence
RNKV

SEQ ID NO: 1223 - protein, 4 aa, Unknown
  Description of Unknown Phyb I sequence
SNKV

SEQ ID NO: 1224 - protein, 4 aa, Unknown
  Description of Unknown Phyb J sequence
SNKV

SEQ ID NO: 1225 - protein, 4 aa, Unknown
  Description of Unknown Phyb K sequence
LNKV

SEQ ID NO: 1226 - protein, 4 aa, Unknown
  Description of Unknown Phyb L sequence
VNSI

SEQ ID NO: 1227 - protein, 5 aa, Unknown
  Description of Unknown PS-1 sequence
SVANI

SEQ ID NO: 1228 - protein, 4 aa, Unknown
  Description of Unknown psybra 1 sequence
SWPI

SEQ ID NO: 1229 - protein, 4 aa, Unknown
  Description of Unknown psyle A sequence
KSKV

SEQ ID NO: 1230 - protein, 4 aa, Unknown
  Description of Unknown psyle B sequence
KDKL

SEQ ID NO: 1231 - protein, 4 aa, Unknown
  Description of Unknown psyle C sequence
SYPF

SEQ ID NO: 1232 - protein, 4 aa, Unknown
  Description of Unknown psyle D sequence
QNKV

SEQ ID NO: 1233 - protein, 4 aa, Unknown
  Description of Unknown psyle E sequence
KNKV

SEQ ID NO: 1234 - protein, 4 aa, Unknown
  Description of Unknown psyle F sequence
KNKV

SEQ ID NO: 1235 - protein, 4 aa, Unknown
  Description of Unknown psypoe 1 sequence
  MOD_RES (4)..(4): Any amino acid
GAYX

SEQ ID NO: 1236 - protein, 5 aa, Unknown
  Description of Unknown tricyclon A sequence
GEWKL

SEQ ID NO: 1237 - protein, 5 aa, Unknown
  Description of Unknown tricyclon B sequence
GEWKL

SEQ ID NO: 1238 - protein, 4 aa, Unknown
  Description of Unknown vaby A sequence
SWPI

SEQ ID NO: 1239 - protein, 4 aa, Unknown
  Description of Unknown vaby B sequence
TWPI

SEQ ID NO: 1240 - protein, 4 aa, Unknown
  Description of Unknown vaby C sequence
SWPV

SEQ ID NO: 1241 - protein, 5 aa, Unknown
  Description of Unknown vaby D sequence
DPWPV

SEQ ID NO: 1242 - protein, 5 aa, Unknown
  Description of Unknown vaby E sequence
DPWPV

SEQ ID NO: 1243 - protein, 5 aa, Unknown
  Description of Unknown varv peptide B sequence
DPWPM

SEQ ID NO: 1244 - protein, 4 aa, Unknown
  Description of Unknown varv peptide C sequence
SWPV

SEQ ID NO: 1245 - protein, 4 aa, Unknown
  Description of Unknown varv peptide D sequence
SWPV

SEQ ID NO: 1246 - protein, 4 aa, Unknown
  Description of Unknown varv peptide F sequence
SWPV

SEQ ID NO: 1247 - protein, 5 aa, Unknown
  Description of Unknown varv peptide G sequence
DPWPV

SEQ ID NO: 1248 - protein, 5 aa, Unknown
  Description of Unknown varv peptide H sequence
ETWPV

SEQ ID NO: 1249 - protein, 4 aa, Unknown
  Description of Unknown vhl-1 sequence
KNKV

SEQ ID NO: 1250 - protein, 5 aa, Unknown
  Description of Unknown vhl-2 sequence
DPWPV

SEQ ID NO: 1251 - protein, 4 aa, Unknown
  Description of Unknown vhr1 sequence
SNKV

SEQ ID NO: 1252 - protein, 4 aa, Unknown
  Description of Unknown Viba 1 sequence
SSKV

SEQ ID NO: 1253 - protein, 4 aa, Unknown
  Description of Unknown Viba 10 sequence
KDKV

SEQ ID NO: 1254 - protein, 4 aa, Unknown
  Description of Unknown Viba 11 sequence
KSKV

SEQ ID NO: 1255 - protein, 4 aa, Unknown
  Description of Unknown Viba 12 sequence
KDKV

SEQ ID NO: 1256 - protein, 4 aa, Unknown
  Description of Unknown Viba 13 sequence
KDKV

SEQ ID NO: 1257 - protein, 6 aa, Unknown
  Description of Unknown Viba 14 sequence
SLHTLE

SEQ ID NO: 1258 - protein, 4 aa, Unknown
  Description of Unknown Viba 15 sequence
SWPV

SEQ ID NO: 1259 - protein, 4 aa, Unknown
  Description of Unknown Viba 16 sequence
SWPI

SEQ ID NO: 1260 - protein, 4 aa, Unknown
  Description of Unknown Viba 17 sequence
SWPV

SEQ ID NO: 1261 - protein, 4 aa, Unknown
  Description of Unknown Viba 2 sequence
SSKV

SEQ ID NO: 1262 - protein, 4 aa, Unknown
  Description of Unknown Viba 3 sequence
SSKV

SEQ ID NO: 1263 - protein, 4 aa, Unknown
  Description of Unknown Viba 4 sequence
KSSV

SEQ ID NO: 1264 - protein, 4 aa, Unknown
  Description of Unknown Viba 5 sequence
KSKV

SEQ ID NO: 1265 - protein, 4 aa, Unknown
  Description of Unknown Viba 6 sequence
KSKV

SEQ ID NO: 1266 - protein, 4 aa, Unknown
  Description of Unknown Viba 7 sequence
KSKV

SEQ ID NO: 1267 - protein, 4 aa, Unknown
  Description of Unknown Viba 8 sequence
KNSR

SEQ ID NO: 1268 - protein, 4 aa, Unknown
  Description of Unknown Viba 9 sequence
KNKV

SEQ ID NO: 1269 - protein, 4 aa, Unknown
  Description of Unknown vibi A sequence
SYPI

SEQ ID NO: 1270 - protein, 4 aa, Unknown
  Description of Unknown vibi B sequence
SYPI

SEQ ID NO: 1271 - protein, 4 aa, Unknown
  Description of Unknown vibi C sequence
SWPV

SEQ ID NO: 1272 - protein, 4 aa, Unknown
  Description of Unknown vibi D sequence
SYPI

SEQ ID NO: 1273 - protein, 4 aa, Unknown
  Description of Unknown vibi E sequence
SNKV

SEQ ID NO: 1274 - protein, 4 aa, Unknown
  Description of Unknown vibi F sequence
KSKV

SEQ ID NO: 1275 - protein, 4 aa, Unknown
  Description of Unknown vibi G sequence
KSKV

SEQ ID NO: 1276 - protein, 4 aa, Unknown
  Description of Unknown vibi H sequence
KSKV

SEQ ID NO: 1277 - protein, 4 aa, Unknown
  Description of Unknown vibi I sequence
KSKV

SEQ ID NO: 1278 - protein, 4 aa, Unknown
  Description of Unknown vibi J sequence
KSKV

SEQ ID NO: 1279 - protein, 4 aa, Unknown
  Description of Unknown vibi K sequence
KSKV

SEQ ID NO: 1280 - protein, 4 aa, Unknown
  Description of Unknown vico A sequence
KNKV

SEQ ID NO: 1281 - protein, 4 aa, Unknown
  Description of Unknown vico B sequence
KNKV

SEQ ID NO: 1282 - protein, 4 aa, Unknown
  Description of Unknown vigno 1 sequence
SWPV

SEQ ID NO: 1283 - protein, 4 aa, Unknown
  Description of Unknown vigno 10 sequence
KSKV

SEQ ID NO: 1284 - protein, 4 aa, Unknown
  Description of Unknown vigno 2 sequence
SWPV

SEQ ID NO: 1285 - protein, 4 aa, Unknown
  Description of Unknown vigno 3 sequence
SWPV

SEQ ID NO: 1286 - protein, 4 aa, Unknown
  Description of Unknown vigno 4 sequence
SWPV

SEQ ID NO: 1287 - protein, 4 aa, Unknown
  Description of Unknown vigno 5 sequence
GWPV

SEQ ID NO: 1288 - protein, 5 aa, Unknown
  Description of Unknown vigno 6 sequence
KGSKV

SEQ ID NO: 1289 - protein, 4 aa, Unknown
  Description of Unknown vigno 7 sequence
KNKV

SEQ ID NO: 1290 - protein, 4 aa, Unknown
  Description of Unknown vigno 8 sequence
KSKV

SEQ ID NO: 1291 - protein, 4 aa, Unknown
  Description of Unknown vigno 9 sequence
KSKV

SEQ ID NO: 1292 - protein, 4 aa, Unknown
  Description of Unknown violacin A sequence
SYPV

SEQ ID NO: 1293 - protein, 4 aa, Unknown
  Description of Unknown violapeptide 1 sequence
SRPV

SEQ ID NO: 1294 - protein, 4 aa, Unknown
  Description of Unknown viphi A sequence
KSKV

SEQ ID NO: 1295 - protein, 4 aa, Unknown
  Description of Unknown viphi B sequence
SWPI

SEQ ID NO: 1296 - protein, 4 aa, Unknown
  Description of Unknown viphi C sequence
SSKV

SEQ ID NO: 1297 - protein, 4 aa, Unknown
  Description of Unknown viphi D sequence
SSKV

SEQ ID NO: 1298 - protein, 4 aa, Unknown
  Description of Unknown viphi E sequence
SNKV

SEQ ID NO: 1299 - protein, 4 aa, Unknown
  Description of Unknown viphi F sequence
SSKV

SEQ ID NO: 1300 - protein, 4 aa, Unknown
  Description of Unknown viphi G sequence
SNKV

SEQ ID NO: 1301 - protein, 4 aa, Unknown
  Description of Unknown viphi H sequence
SWGV

SEQ ID NO: 1302 - protein, 4 aa, Unknown
  Description of Unknown vitri A sequence
KSKV

SEQ ID NO: 1303 - protein, 4 aa, Unknown
  Description of Unknown vitri B sequence
SWPV

SEQ ID NO: 1304 - protein, 4 aa, Unknown
  Description of Unknown vitri C sequence
TWPV

SEQ ID NO: 1305 - protein, 4 aa, Unknown
  Description of Unknown vitri D sequence
NWPV

SEQ ID NO: 1306 - protein, 4 aa, Unknown
  Description of Unknown vitri E sequence
SWPV

SEQ ID NO: 1307 - protein, 4 aa, Unknown
  Description of Unknown vitri F sequence
KSKV

SEQ ID NO: 1308 - protein, 4 aa, Unknown
  Description of Unknown vitri peptide 1 sequence
KSKV

SEQ ID NO: 1309 - protein, 4 aa, Unknown
  Description of Unknown vitri peptide 18a sequence
KWPI

SEQ ID NO: 1310 - protein, 4 aa, Unknown
  Description of Unknown vitri peptide 18b sequence
VYRV

SEQ ID NO: 1311 - protein, 4 aa, Unknown
  Description of Unknown vitri peptide 2 sequence
SNKV

SEQ ID NO: 1312 - protein, 4 aa, Unknown
  Description of Unknown vitri peptide 20 sequence
SENV

SEQ ID NO: 1313 - protein, 4 aa, Unknown
  Description of Unknown vitri peptide 22a sequence
IYPV

SEQ ID NO: 1314 - protein, 4 aa, Unknown
  Description of Unknown vitri peptide 23 sequence
SWPL

SEQ ID NO: 1315 - protein, 5 aa, Unknown
  Description of Unknown vitri peptide 24a sequence
GDWKL

SEQ ID NO: 1316 - protein, 4 aa, Unknown
  Description of Unknown vitri peptide 3 sequence
SKNV

SEQ ID NO: 1317 - protein, 4 aa, Unknown
  Description of Unknown vitri peptide 30 sequence
NSSL

SEQ ID NO: 1318 - protein, 5 aa, Unknown
  Description of Unknown vitri peptide 36/37 sequence
GDWKL

SEQ ID NO: 1319 - protein, 4 aa, Unknown
  Description of Unknown vitri peptide 39 sequence
SWPV

SEQ ID NO: 1320 - protein, 4 aa, Unknown
  Description of Unknown vitri peptide 39 linear sequence
SWPV

SEQ ID NO: 1321 - protein, 4 aa, Unknown
  Description of Unknown vitri peptide 4 sequence
QSKV

SEQ ID NO: 1322 - protein, 4 aa, Unknown
  Description of Unknown vitri peptide 50 sequence
SHNV

SEQ ID NO: 1323 - protein, 4 aa, Unknown
  Description of Unknown vitri peptide 8 sequence
HESI

SEQ ID NO: 1324 - protein, 4 aa, Unknown
  Description of Unknown vitri peptide 94b sequence
DWPI

SEQ ID NO: 1325 - protein, 5 aa, Unknown
  Description of Unknown vitri peptide 9a/53 sequence
GSWAL

SEQ ID NO: 1326 - protein, 4 aa, Unknown
  Description of Unknown vocC sequence
SWPV

SEQ ID NO: 1327 - protein, 4 aa, Unknown
  Description of Unknown vodo M sequence
SWPV

SEQ ID NO: 1328 - protein, 4 aa, Unknown
  Description of Unknown vodo N sequence
SWPV

SEQ ID NO: 1329 - protein, 4 aa, Unknown
  Description of Unknown Vpf-1 sequence
RSKV

SEQ ID NO: 1330 - protein, 4 aa, Unknown
  Description of Unknown Vpl-1 sequence
SSMI

SEQ ID NO: 1331 - protein, 7 aa, Unknown
  Description of Unknown caripe 1 sequence
YRNGVIP

SEQ ID NO: 1332 - protein, 7 aa, Unknown
  Description of Unknown caripe 10 sequence
YRNGVIP

SEQ ID NO: 1333 - protein, 7 aa, Unknown
  Description of Unknown caripe 11 sequence
YRNGVIP

SEQ ID NO: 1334 - protein, 7 aa, Unknown
  Description of Unknown caripe 12 sequence
YRNGVIP

SEQ ID NO: 1335 - protein, 6 aa, Unknown
  Description of Unknown caripe 13 sequence
YRNGIP

SEQ ID NO: 1336 - protein, 6 aa, Unknown
  Description of Unknown caripe 2 sequence
YKNGIP

SEQ ID NO: 1337 - protein, 5 aa, Unknown
  Description of Unknown caripe 4 sequence
YLNLI

SEQ ID NO: 1338 - protein, 6 aa, Unknown
  Description of Unknown caripe 6 sequence
YLNGAI

SEQ ID NO: 1339 - protein, 6 aa, Unknown
  Description of Unknown caripe 7 sequence
YRNGIP

SEQ ID NO: 1340 - protein, 7 aa, Unknown
  Description of Unknown caripe 8 sequence
YRNGVIP

SEQ ID NO: 1341 - protein, 8 aa, Unknown
  Description of Unknown CD-1 sequence
KRNGADGF

SEQ ID NO: 1342 - protein, 7 aa, Unknown
  Description of Unknown chacur 1 sequence
TRNGLPV

SEQ ID NO: 1343 - protein, 6 aa, Unknown
  Description of Unknown chassatide C1 sequence
TRNGDA

SEQ ID NO: 1344 - protein, 7 aa, Unknown
  Description of Unknown chassatide C10 sequence
VNKNGEY

SEQ ID NO: 1345 - protein, 4 aa, Unknown
  Description of Unknown chassatide C11 sequence
YSIP

SEQ ID NO: 1346 - protein, 6 aa, Unknown
  Description of Unknown chassatide C12 sequence
VNKNEY

SEQ ID NO: 1347 - protein, 6 aa, Unknown
  Description of Unknown chassatide C13 sequence
YRNGFP

SEQ ID NO: 1348 - protein, 6 aa, Unknown
  Description of Unknown chassatide C14 sequence
YNNGIP

SEQ ID NO: 1349 - protein, 6 aa, Unknown
  Description of Unknown chassatide C15 sequence
YKNGIP

SEQ ID NO: 1350 - protein, 6 aa, Unknown
  Description of Unknown chassatide C16 sequence
YNNGVP

SEQ ID NO: 1351 - protein, 4 aa, Unknown
  Description of Unknown chassatide C17 sequence
YRIP

SEQ ID NO: 1352 - protein, 6 aa, Unknown
  Description of Unknown chassatide C18 sequence
YNNGIP

SEQ ID NO: 1353 - protein, 6 aa, Unknown
  Description of Unknown chassatide C2 sequence
YNNGIP

SEQ ID NO: 1354 - protein, 6 aa, Unknown
  Description of Unknown chassatide C3 sequence
YRNGIP

SEQ ID NO: 1355 - protein, 6 aa, Unknown
  Description of Unknown chassatide C4 sequence
TRNGAS

SEQ ID NO: 1356 - protein, 7 aa, Unknown
  Description of Unknown chassatide C5 sequence
YRNGVIP

SEQ ID NO: 1357 - protein, 7 aa, Unknown
  Description of Unknown chassatide C6 sequence
YRNGVIP

SEQ ID NO: 1358 - protein, 4 aa, Unknown
  Description of Unknown chassatide C7 sequence
YTIP

SEQ ID NO: 1359 - protein, 5 aa, Unknown
  Description of Unknown chassatide C8 sequence
YRAIP

SEQ ID NO: 1360 - protein, 6 aa, Unknown
  Description of Unknown chassatide C9 sequence
YNNGIP

SEQ ID NO: 1361 - protein, 6 aa, Unknown
  Description of Unknown circulin A sequence
YRNGIP

SEQ ID NO: 1362 - protein, 7 aa, Unknown
  Description of Unknown circulin B sequence
YRNGVIP

SEQ ID NO: 1363 - protein, 6 aa, Unknown
  Description of Unknown circulin C sequence
YRNGIP

SEQ ID NO: 1364 - protein, 6 aa, Unknown
  Description of Unknown circulin D sequence
YHDKIP

SEQ ID NO: 1365 - protein, 6 aa, Unknown
  Description of Unknown circulin E sequence
YHDKIP

SEQ ID NO: 1366 - protein, 5 aa, Unknown
  Description of Unknown circulin F sequence
YRAIP

SEQ ID NO: 1367 - protein, 6 aa, Unknown
  Description of Unknown cliotide T12 sequence
YRDGIP

SEQ ID NO: 1368 - protein, 8 aa, Unknown
  Description of Unknown cliotide T2 sequence
KKNGEFLK

SEQ ID NO: 1369 - protein, 6 aa, Unknown
  Description of Unknown cliotide T8 sequence
YNNGIP

SEQ ID NO: 1370 - protein, 6 aa, Unknown
  Description of Unknown cliotide T9 sequence
YNNGIP

SEQ ID NO: 1371 - protein, 8 aa, Unknown
  Description of Unknown cO26 sequence
AKDGSIPA

SEQ ID NO: 1372 - protein, 8 aa, Unknown
  Description of Unknown cO27 sequence
AKDGSIPA

SEQ ID NO: 1373 - protein, 7 aa, Unknown
  Description of Unknown cO28 sequence
FRDGLPV

SEQ ID NO: 1374 - protein, 6 aa, Unknown
  Description of Unknown cO29 sequence
YKNGIP

SEQ ID NO: 1375 - protein, 6 aa, Unknown
  Description of Unknown cO30 sequence
FKNGIP

SEQ ID NO: 1376 - protein, 7 aa, Unknown
  Description of Unknown cO31 sequence
TRNGLPV

SEQ ID NO: 1377 - protein, 7 aa, Unknown
  Description of Unknown cO32 sequence
TNDGAPV

SEQ ID NO: 1378 - protein, 7 aa, Unknown
  Description of Unknown cO33 sequence
TRDGLPV

SEQ ID NO: 1379 - protein, 7 aa, Unknown
  Description of Unknown cO34 sequence
TRDGLPV

SEQ ID NO: 1380 - protein, 7 aa, Unknown
  Description of Unknown cO35 sequence
TRDGLPV

SEQ ID NO: 1381 - protein, 7 aa, Unknown
  Description of Unknown cO36 sequence
THDGLPT

SEQ ID NO: 1382 - protein, 6 aa, Unknown
  Description of Unknown Cter 35 sequence
LNNGAF

SEQ ID NO: 1383 - protein, 7 aa, Unknown
  Description of Unknown Cter A sequence
YRNGVIP

SEQ ID NO: 1384 - protein, 6 aa, Unknown
  Description of Unknown Cter B sequence
YLNGVP

SEQ ID NO: 1385 - protein, 6 aa, Unknown
  Description of Unknown Cter C sequence
YLDGVP

SEQ ID NO: 1386 - protein, 6 aa, Unknown
  Description of Unknown Cter D sequence
YLNGIP

SEQ ID NO: 1387 - protein, 6 aa, Unknown
  Description of Unknown Cter E sequence
YLDGIP

SEQ ID NO: 1388 - protein, 6 aa, Unknown
  Description of Unknown Cter F sequence
YLDGIP

SEQ ID NO: 1389 - protein, 6 aa, Unknown
  Description of Unknown Cter G sequence
YNNGLP

SEQ ID NO: 1390 - protein, 6 aa, Unknown
  Description of Unknown Cter H sequence
YNDGLP

SEQ ID NO: 1391 - protein, 7 aa, Unknown
  Description of Unknown Cter I sequence
YINGTVP

SEQ ID NO: 1392 - protein, 7 aa, Unknown
  Description of Unknown Cter J sequence
YIDGTVP

SEQ ID NO: 1393 - protein, 5 aa, Unknown
  Description of Unknown Cter K sequence
YNHEP

SEQ ID NO: 1394 - protein, 5 aa, Unknown
  Description of Unknown Cter L sequence
YDHEP

SEQ ID NO: 1395 - protein, 7 aa, Unknown
  Description of Unknown Cter M sequence
MKNGLPT

SEQ ID NO: 1396 - protein, 7 aa, Unknown
  Description of Unknown Cter N sequence
LKNGSAF

SEQ ID NO: 1397 - protein, 6 aa, Unknown
  Description of Unknown Cter O sequence
YRNGIP

SEQ ID NO: 1398 - protein, 6 aa, Unknown
  Description of Unknown Cter P sequence
YRNGIP

SEQ ID NO: 1399 - protein, 6 aa, Unknown
  Description of Unknown Cter Q sequence
YRNGIP

SEQ ID NO: 1400 - protein, 6 aa, Unknown
  Description of Unknown Cter R sequence
YKNGIP

SEQ ID NO: 1401 - protein, 6 aa, Unknown
  Description of Unknown cyclopsychotride A sequence
YKNSIP

SEQ ID NO: 1402 - protein, 6 aa, Unknown
  Description of Unknown cycloviolacin H1 sequence
YRNGIP

SEQ ID NO: 1403 - protein, 7 aa, Unknown
  Description of Unknown cycloviolacin H2 sequence
YLNSAIA

SEQ ID NO: 1404 - protein, 7 aa, Unknown
  Description of Unknown cycloviolacin H3 sequence
TRNGLPV

SEQ ID NO: 1405 - protein, 5 aa, Unknown
  Description of Unknown cycloviolacin H4 sequence
YNGIP

SEQ ID NO: 1406 - protein, 5 aa, Unknown
  Description of Unknown cycloviolacin O1 sequence
YNGIP

SEQ ID NO: 1407 - protein, 6 aa, Unknown
  Description of Unknown cycloviolacin O10 sequence
YRNGIP

SEQ ID NO: 1408 - protein, 7 aa, Unknown
  Description of Unknown cycloviolacin O11 sequence
YKNGTLP

SEQ ID NO: 1409 - protein, 7 aa, Unknown
  Description of Unknown cycloviolacin O12 sequence
TRNGLPI

SEQ ID NO: 1410 - protein, 6 aa, Unknown
  Description of Unknown cycloviolacin O13 sequence
YRNGIP

SEQ ID NO: 1411 - protein, 8 aa, Unknown
  Description of Unknown cycloviolacin O14 sequence
AKNGSIPA

SEQ ID NO: 1412 - protein, 7 aa, Unknown
  Description of Unknown cycloviolacin O15 sequence
KKNGLVP

SEQ ID NO: 1413 - protein, 7 aa, Unknown
  Description of Unknown cycloviolacin O16 sequence
KKINGLP

SEQ ID NO: 1414 - protein, 6 aa, Unknown
  Description of Unknown cycloviolacin O17 sequence
YRNGIP

SEQ ID NO: 1415 - protein, 5 aa, Unknown
  Description of Unknown cycloviolacin O18 sequence
YNGIP

SEQ ID NO: 1416 - protein, 7 aa, Unknown
  Description of Unknown cycloviolacin O19 sequence
YKDGTLP

SEQ ID NO: 1417 - protein, 6 aa, Unknown
  Description of Unknown cycloviolacin O2 sequence
YRNGIP

SEQ ID NO: 1418 - protein, 6 aa, Unknown
  Description of Unknown cycloviolacin O20 sequence
YRDGIP

SEQ ID NO: 1419 - protein, 7 aa, Unknown
  Description of Unknown cycloviolacin O21 sequence
TRNGLPV

SEQ ID NO: 1420 - protein, 7 aa, Unknown
  Description of Unknown cycloviolacin O22 sequence
TRNGLPI

SEQ ID NO: 1421 - protein, 7 aa, Unknown
  Description of Unknown cycloviolacin O23 sequence
THNGLPT

SEQ ID NO: 1422 - protein, 7 aa, Unknown
  Description of Unknown cycloviolacin O24 sequence
THNGLPT

SEQ ID NO: 1423 - protein, 6 aa, Unknown
  Description of Unknown cycloviolacin O25 sequence
YFNDIF

SEQ ID NO: 1424 - protein, 6 aa, Unknown
  Description of Unknown cycloviolacin O3 sequence
YRNGIP

SEQ ID NO: 1425 - protein, 6 aa, Unknown
  Description of Unknown cycloviolacin O4 sequence
YRNGIP

SEQ ID NO: 1426 - protein, 6 aa, Unknown
  Description of Unknown cycloviolacin O5 sequence
YKNGTP

SEQ ID NO: 1427 - protein, 7 aa, Unknown
  Description of Unknown cycloviolacin O6 sequence
YKNGTLP

SEQ ID NO: 1428 - protein, 5 aa, Unknown
  Description of Unknown cycloviolacin O7 sequence
YNSIP

SEQ ID NO: 1429 - protein, 7 aa, Unknown
  Description of Unknown cycloviolacin O8 sequence
YKNGTLP

SEQ ID NO: 1430 - protein, 6 aa, Unknown
  Description of Unknown cycloviolacin O9 sequence
YRNGIP

SEQ ID NO: 1431 - protein, 7 aa, Unknown
  Description of Unknown cycloviolacin T1 sequence
TRNGIPV

SEQ ID NO: 1432 - protein, 10 aa, Unknown
  Description of Unknown cycloviolacin Y1 sequence
YGTNGGTIFD

SEQ ID NO: 1433 - protein, 10 aa, Unknown
  Description of Unknown cycloviolacin Y2 sequence
YGTNGGTIFD

SEQ ID NO: 1434 - protein, 10 aa, Unknown
  Description of Unknown cycloviolacin Y3 sequence
YGTNGGTIFD

SEQ ID NO: 1435 - protein, 6 aa, Unknown
  Description of Unknown cycloviolacin Y4 sequence
YLNGVP

SEQ ID NO: 1436 - protein, 5 aa, Unknown
  Description of Unknown cycloviolacin Y5 sequence
YNGIP

SEQ ID NO: 1437 - protein, 7 aa, Unknown
  Description of Unknown cycloviolin A sequence
YRNGVIP

SEQ ID NO: 1438 - protein, 6 aa, Unknown
  Description of Unknown cycloviolin B sequence
FKNGTA

SEQ ID NO: 1439 - protein, 6 aa, Unknown
  Description of Unknown cycloviolin C sequence
YRNGIP

SEQ ID NO: 1440 - protein, 6 aa, Unknown
  Description of Unknown cycloviolin D sequence
YRNGFP

SEQ ID NO: 1441 - protein, 6 aa, Unknown
  Description of Unknown Globa A sequence
YRNGIP

SEQ ID NO: 1442 - protein, 7 aa, Unknown
  Description of Unknown Globa B sequence
YRNGVIP

SEQ ID NO: 1443 - protein, 5 aa, Unknown
  Description of Unknown Globa C sequence
YRNAP

SEQ ID NO: 1444 - protein, 6 aa, Unknown
  Description of Unknown Globa D sequence
YRNGIP

SEQ ID NO: 1445 - protein, 8 aa, Unknown
  Description of Unknown Globa E sequence
KKNGSAFG

SEQ ID NO: 1446 - protein, 7 aa, Unknown
  Description of Unknown Globa F sequence
YKNGSFP

SEQ ID NO: 1447 - protein, 8 aa, Unknown
  Description of Unknown Glopa A sequence
YLNGGSIP

SEQ ID NO: 1448 - protein, 8 aa, Unknown
  Description of Unknown Glopa B sequence
YLNGGSVP

SEQ ID NO: 1449 - protein, 8 aa, Unknown
  Description of Unknown Glopa C sequence
SKNGDIPL

SEQ ID NO: 1450 - protein, 6 aa, Unknown
  Description of Unknown Glopa D sequence
RKDGVP

SEQ ID NO: 1451 - protein, 5 aa, Unknown
  Description of Unknown Glopa E sequence
YNGIP

SEQ ID NO: 1452 - protein, 6 aa, Unknown
  Description of Unknown hcf-1 sequence
MRNGIP

SEQ ID NO: 1453 - protein, 6 aa, Unknown
  Description of Unknown hcf-1 variant sequence
MRNGIP

SEQ ID NO: 1454 - protein, 6 aa, Unknown
  Description of Unknown hedyotide B1 sequence
YRNGTR

SEQ ID NO: 1455 - protein, 5 aa, Unknown
  Description of Unknown hedyotide B2 sequence
SSGIQ

SEQ ID NO: 1456 - protein, 6 aa, Unknown
  Description of Unknown htf-1 sequence
MRNGIP

SEQ ID NO: 1457 - protein, 6 aa, Unknown
  Description of Unknown Hyfl A sequence
YLNSIS

SEQ ID NO: 1458 - protein, 8 aa, Unknown
  Description of Unknown Hyfl B sequence
MKNGSPIQ

SEQ ID NO: 1459 - protein, 8 aa, Unknown
  Description of Unknown Hyfl C sequence
MKNGSPRQ

SEQ ID NO: 1460 - protein, 7 aa, Unknown
  Description of Unknown Hyfl D sequence
YYNGSVP

SEQ ID NO: 1461 - protein, 7 aa, Unknown
  Description of Unknown Hyfl E sequence
YLNGEIP

SEQ ID NO: 1462 - protein, 6 aa, Unknown
  Description of Unknown Hyfl F sequence
YWNSIS

SEQ ID NO: 1463 - protein, 6 aa, Unknown
  Description of Unknown Hyfl I sequence
YRNGIP

SEQ ID NO: 1464 - protein, 6 aa, Unknown
  Description of Unknown Hyfl J sequence
YFNGIA

SEQ ID NO: 1465 - protein, 6 aa, Unknown
  Description of Unknown Hyfl K sequence
YLNGTP

SEQ ID NO: 1466 - protein, 6 aa, Unknown
  Description of Unknown Hyfl L sequence
YLNGTP

SEQ ID NO: 1467 - protein, 7 aa, Unknown
  Description of Unknown Hyfl M sequence
YYNGNIP

SEQ ID NO: 1468 - protein, 5 aa, Unknown
  Description of Unknown Hypa A sequence
YNGIP

SEQ ID NO: 1469 - protein, 7 aa, Unknown
  Description of Unknown kalata B1 sequence
TRNGLPV

SEQ ID NO: 1470 - protein, 7 aa, Unknown
  Description of Unknown kalata B10 sequence
TRDGLPT

SEQ ID NO: 1471 - protein, 7 aa, Unknown
  Description of Unknown kalata B10 linear sequence
TRDGLPT

SEQ ID NO: 1472 - protein, 7 aa, Unknown
  Description of Unknown kalata B11 sequence
TRDGLPV

SEQ ID NO: 1473 - protein, 6 aa, Unknown
  Description of Unknown kalata B12 sequence
VKDGSL

SEQ ID NO: 1474 - protein, 7 aa, Unknown
  Description of Unknown kalata B13 sequence
TRDGLPV

SEQ ID NO: 1475 - protein, 7 aa, Unknown
  Description of Unknown kalata B14 sequence
TRDGLPV

SEQ ID NO: 1476 - protein, 7 aa, Unknown
  Description of Unknown kalata B15 sequence
TRDGLPV

SEQ ID NO: 1477 - protein, 5 aa, Unknown
  Description of Unknown kalata B16 sequence
YDGIP

SEQ ID NO: 1478 - protein, 5 aa, Unknown
  Description of Unknown kalata B17 sequence
YNGIP

SEQ ID NO: 1479 - protein, 6 aa, Unknown
  Description of Unknown kalata B18 sequence
YRNGVP

SEQ ID NO: 1480 - protein, 6 aa, Unknown
  Description of Unknown kalata B19 sequence
YKNGFP

SEQ ID NO: 1481 - protein, 7 aa, Unknown
  Description of Unknown kalata B2 sequence
TRDGLPV

SEQ ID NO: 1482 - protein, 7 aa, Unknown
  Description of Unknown kalata B3 sequence
TRDGLPT

SEQ ID NO: 1483 - protein, 7 aa, Unknown
  Description of Unknown kalata B4 sequence
TRDGLPV

SEQ ID NO: 1484 - protein, 6 aa, Unknown
  Description of Unknown kalata B5 sequence
YLNGTP

SEQ ID NO: 1485 - protein, 7 aa, Unknown
  Description of Unknown kalata B6 sequence
TRNGLPT

SEQ ID NO: 1486 - protein, 7 aa, Unknown
  Description of Unknown kalata B7 sequence
KRNGLPV

SEQ ID NO: 1487 - protein, 8 aa, Unknown
  Description of Unknown kalata B8 sequence
TKDGSVLN

SEQ ID NO: 1488 - protein, 8 aa, Unknown
  Description of Unknown kalata B9 sequence
TKDGSVFN

SEQ ID NO: 1489 - protein, 8 aa, Unknown
  Description of Unknown kalata B9 linear sequence
TKDGSVFN

SEQ ID NO: 1490 - protein, 7 aa, Unknown
  Description of Unknown kalata S sequence
TRNGLPV

SEQ ID NO: 1491 - protein, 8 aa, Unknown
  Description of Unknown MCoTI-I sequence
GSGSDGGV

SEQ ID NO: 1492 - protein, 8 aa, Unknown
  Description of Unknown MCoTI-II sequence
GSGSDGGV

SEQ ID NO: 1493 - protein, 4 aa, Unknown
  Description of Unknown MCoTI-III sequence
GQRA

SEQ ID NO: 1494 - protein, 8 aa, Unknown
  Description of Unknown MCoTI-IV sequence
GSGSDGGA

SEQ ID NO: 1495 - protein, 4 aa, Unknown
  Description of Unknown MCoTI-V sequence
GQRA

SEQ ID NO: 1496 - protein, 4 aa, Unknown
  Description of Unknown MCoTI-VI sequence
GQRA

SEQ ID NO: 1497 - protein, 8 aa, Unknown
  Description of Unknown MCoTI-VII sequence
GSGSDGGA

SEQ ID NO: 1498 - protein, 8 aa, Unknown
  Description of Unknown MCoTI-VIII sequence
GSGSDGGV

SEQ ID NO: 1499 - protein, 7 aa, Unknown
  Description of Unknown mech 2 sequence
YKDGIPT

SEQ ID NO: 1500 - protein, 7 aa, Unknown
  Description of Unknown mech 3 sequence
YKNGIPT

SEQ ID NO: 1501 - protein, 7 aa, Unknown
  Description of Unknown mech 4 sequence
YKDGSIP

SEQ ID NO: 1502 - protein, 7 aa, Unknown
  Description of Unknown mela 7 sequence
KKNGIPT

SEQ ID NO: 1503 - protein, 5 aa, Unknown
  Description of Unknown Mra13 sequence
YHGIP

SEQ ID NO: 1504 - protein, 7 aa, Unknown
  Description of Unknown Mra14a sequence
YKNGSIP

SEQ ID NO: 1505 - protein, 7 aa, Unknown
  Description of Unknown Mra14b sequence
YKNGTIP

SEQ ID NO: 1506 - protein, 7 aa, Unknown
  Description of Unknown Mra17 sequence
YKNGSIP

SEQ ID NO: 1507 - protein, 6 aa, Unknown
  Description of Unknown Mra22 sequence
TLNGVP

SEQ ID NO: 1508 - protein, 7 aa, Unknown
  Description of Unknown Mra23 sequence
YRNGVIP

SEQ ID NO: 1509 - protein, 7 aa, Unknown
  Description of Unknown Mra24 sequence
YKNGHPT

SEQ ID NO: 1510 - protein, 8 aa, Unknown
  Description of Unknown Mra25 sequence
TKNGSAIL

SEQ ID NO: 1511 - protein, 7 aa, Unknown
  Description of Unknown Mra26 sequence
YRNGHPI

SEQ ID NO: 1512 - protein, 7 aa, Unknown
  Description of Unknown Mra29 sequence
YKNGSIP

SEQ ID NO: 1513 - protein, 7 aa, Unknown
  Description of Unknown Mra30 sequence
YKNGSIP

SEQ ID NO: 1514 - protein, 6 aa, Unknown
  Description of Unknown Mra30 sequence
YRNGIP

SEQ ID NO: 1515 - protein, 7 aa, Unknown
  Description of Unknown Mra4 sequence
YKNGSIP

SEQ ID NO: 1516 - protein, 6 aa, Unknown
  Description of Unknown Mra5 sequence
YRNGIP

SEQ ID NO: 1517 - protein, 7 aa, Unknown
  Description of Unknown Oak6 cyclotide 1 sequence
TRNGLPV

SEQ ID NO: 1518 - protein, 7 aa, Unknown
  Description of Unknown Oak6 cyclotide 2 sequence
TRDGLPI

SEQ ID NO: 1519 - protein, 6 aa, Unknown
  Description of Unknown Oak7 cyclotide sequence
YKNGSH

SEQ ID NO: 1520 - protein, 6 aa, Unknown
  Description of Unknown Oak8 cyclotide sequence
YLNGVP

SEQ ID NO: 1521 - protein, 8 aa, Unknown
  Description of Unknown palicourein sequence
KRNGDPTF

SEQ ID NO: 1522 - protein, 7 aa, Unknown
  Description of Unknown paltet 1 sequence
TRNGLPI

SEQ ID NO: 1523 - protein, 6 aa, Unknown
  Description of Unknown Panitide L1 sequence
VRQLPI

SEQ ID NO: 1524 - protein, 6 aa, Unknown
  Description of Unknown Panitide L2 sequence
VRQLPI

SEQ ID NO: 1525 - protein, 5 aa, Unknown
  Description of Unknown Panitide L3 sequence
LKQAF

SEQ ID NO: 1526 - protein, 5 aa, Unknown
  Description of Unknown Panitide L4 sequence
LKQAF

SEQ ID NO: 1527 - protein, 6 aa, Unknown
  Description of Unknown Panitide L5 sequence
ARQLPI

SEQ ID NO: 1528 - protein, 6 aa, Unknown
  Description of Unknown Panitide L6 sequence
VRQLPI

SEQ ID NO: 1529 - protein, 5 aa, Unknown
  Description of Unknown Panitide L7 sequence
LKQAF

SEQ ID NO: 1530 - protein, 3 aa, Unknown
  Description of Unknown Panitide L8 sequence
VQD

SEQ ID NO: 1531 - protein, 8 aa, Unknown
  Description of Unknown Parigidin-br1 sequence
YYDGGSVP

SEQ ID NO: 1532 - protein, 6 aa, Unknown
  Description of Unknown Phyb A sequence
YRNGIG

SEQ ID NO: 1533 - protein, 6 aa, Unknown
  Description of Unknown Phyb B sequence
YRNGVP

SEQ ID NO: 1534 - protein, 6 aa, Unknown
  Description of Unknown Phyb C sequence
YKNGIP

SEQ ID NO: 1535 - protein, 6 aa, Unknown
  Description of Unknown Phyb D sequence
YKNGIP

SEQ ID NO: 1536 - protein, 6 aa, Unknown
  Description of Unknown Phyb E sequence
YRNGIP

SEQ ID NO: 1537 - protein, 6 aa, Unknown
  Description of Unknown Phyb F sequence
YRNGIP

SEQ ID NO: 1538 - protein, 6 aa, Unknown
  Description of Unknown Phyb G sequence
YRNGIP

SEQ ID NO: 1539 - protein, 6 aa, Unknown
  Description of Unknown Phyb H sequence
YRNGLP

SEQ ID NO: 1540 - protein, 6 aa, Unknown
  Description of Unknown Phyb I sequence
YKNGIP

SEQ ID NO: 1541 - protein, 6 aa, Unknown
  Description of Unknown Phyb J sequence
VKDSYT

SEQ ID NO: 1542 - protein, 6 aa, Unknown
  Description of Unknown Phyb K sequence
VRPSTD

SEQ ID NO: 1543 - protein, 8 aa, Unknown
  Description of Unknown Phyb L sequence
TYTNQSIS

SEQ ID NO: 1544 - protein, 7 aa, Unknown
  Description of Unknown PS-1 sequence
VRNGFIP

SEQ ID NO: 1545 - protein, 7 aa, Unknown
  Description of Unknown psybra 1 sequence
TKNGLPI

SEQ ID NO: 1546 - protein, 6 aa, Unknown
  Description of Unknown psyle A sequence
YFNGIA

SEQ ID NO: 1547 - protein, 6 aa, Unknown
  Description of Unknown psyle B sequence
YYDGIP

SEQ ID NO: 1548 - protein, 3 aa, Unknown
  Description of Unknown psyle C sequence
KKL

SEQ ID NO: 1549 - protein, 6 aa, Unknown
  Description of Unknown psyle D sequence
YRDGIP

SEQ ID NO: 1550 - protein, 7 aa, Unknown
  Description of Unknown psyle E sequence
YRDGVIP

SEQ ID NO: 1551 - protein, 7 aa, Unknown
  Description of Unknown psyle F sequence
YRDGVIP

SEQ ID NO: 1552 - protein, 7 aa, Unknown
  Description of Unknown psypoe 1 sequence
TRNGSVI

SEQ ID NO: 1553 - protein, 10 aa, Unknown
  Description of Unknown tricyclon A sequence
YGTNGGTIFD

SEQ ID NO: 1554 - protein, 10 aa, Unknown
  Description of Unknown tricyclon B sequence
YGENGGTIFD

SEQ ID NO: 1555 - protein, 7 aa, Unknown
  Description of Unknown vaby A sequence
TRNGLPV

SEQ ID NO: 1556 - protein, 7 aa, Unknown
  Description of Unknown vaby B sequence
TRNGLPV

SEQ ID NO: 1557 - protein, 7 aa, Unknown
  Description of Unknown vaby C sequence
TRNGLPV

SEQ ID NO: 1558 - protein, 7 aa, Unknown
  Description of Unknown vaby D sequence
TRNGLPV

SEQ ID NO: 1559 - protein, 7 aa, Unknown
  Description of Unknown vaby E sequence
TRNGLPV

SEQ ID NO: 1560 - protein, 7 aa, Unknown
  Description of Unknown varv peptide B sequence
SRNGLPV

SEQ ID NO: 1561 - protein, 7 aa, Unknown
  Description of Unknown varv peptide C sequence
TRNGVPI

SEQ ID NO: 1562 - protein, 7 aa, Unknown
  Description of Unknown varv peptide D sequence
TRNGLPI

SEQ ID NO: 1563 - protein, 7 aa, Unknown
  Description of Unknown varv peptide F sequence
TRNGVPI

SEQ ID NO: 1564 - protein, 7 aa, Unknown
  Description of Unknown varv peptide G sequence
SRNGVPV

SEQ ID NO: 1565 - protein, 7 aa, Unknown
  Description of Unknown varv peptide H sequence
SRNGLPV

SEQ ID NO: 1566 - protein, 6 aa, Unknown
  Description of Unknown vhl-1 sequence
YLNSIS

SEQ ID NO: 1567 - protein, 7 aa, Unknown
  Description of Unknown vhl-2 sequence
TRNGLPV

SEQ ID NO: 1568 - protein, 5 aa, Unknown
  Description of Unknown vhr1 sequence
YNGIP

SEQ ID NO: 1569 - protein, 6 aa, Unknown
  Description of Unknown Viba 1 sequence
YRNGIP

SEQ ID NO: 1570 - protein, 5 aa, Unknown
  Description of Unknown Viba 10 sequence
YNGIP

SEQ ID NO: 1571 - protein, 6 aa, Unknown
  Description of Unknown Viba 11 sequence
YRNGIP

SEQ ID NO: 1572 - protein, 5 aa, Unknown
  Description of Unknown Viba 12 sequence
YNGIP

SEQ ID NO: 1573 - protein, 5 aa, Unknown
  Description of Unknown Viba 13 sequence
YNTIP

SEQ ID NO: 1574 - protein, 6 aa, Unknown
  Description of Unknown Viba 14 sequence
VRNGRL

SEQ ID NO: 1575 - protein, 7 aa, Unknown
  Description of Unknown Viba 15 sequence
TRNGLPV

SEQ ID NO: 1576 - protein, 7 aa, Unknown
  Description of Unknown Viba 16 sequence
TRNGLPI

SEQ ID NO: 1577 - protein, 7 aa, Unknown
  Description of Unknown Viba 17 sequence
TRNGLPV

SEQ ID NO: 1578 - protein, 6 aa, Unknown
  Description of Unknown Viba 2 sequence
YRNGIP

SEQ ID NO: 1579 - protein, 6 aa, Unknown
  Description of Unknown Viba 3 sequence
YRNGIP

SEQ ID NO: 1580 - protein, 6 aa, Unknown
  Description of Unknown Viba 4 sequence
YRNGVP

SEQ ID NO: 1581 - protein, 6 aa, Unknown
  Description of Unknown Viba 5 sequence
YRNGIP

SEQ ID NO: 1582 - protein, 6 aa, Unknown
  Description of Unknown Viba 6 sequence
YRNGIP

SEQ ID NO: 1583 - protein, 7 aa, Unknown
  Description of Unknown Viba 7 sequence
YRNGVIP

SEQ ID NO: 1584 - protein, 6 aa, Unknown
  Description of Unknown Viba 8 sequence
QKNGAG

SEQ ID NO: 1585 - protein, 6 aa, Unknown
  Description of Unknown Viba 9 sequence
YRKGIP

SEQ ID NO: 1586 - protein, 7 aa, Unknown
  Description of Unknown vibi A sequence
TRNGLPV

SEQ ID NO: 1587 - protein, 7 aa, Unknown
  Description of Unknown vibi B sequence
TRNGLPV

SEQ ID NO: 1588 - protein, 7 aa, Unknown
  Description of Unknown vibi C sequence
TRNGLPV

SEQ ID NO: 1589 - protein, 7 aa, Unknown
  Description of Unknown vibi D sequence
TRNGLPV

SEQ ID NO: 1590 - protein, 5 aa, Unknown
  Description of Unknown vibi E sequence
YNGIP

SEQ ID NO: 1591 - protein, 7 aa, Unknown
  Description of Unknown vibi F sequence
YKNGTIP

SEQ ID NO: 1592 - protein, 7 aa, Unknown
  Description of Unknown vibi G sequence
YKNGTFP

SEQ ID NO: 1593 - protein, 7 aa, Unknown
  Description of Unknown vibi H sequence
YKNGLLP

SEQ ID NO: 1594 - protein, 6 aa, Unknown
  Description of Unknown vibi I sequence
YRNGIP

SEQ ID NO: 1595 - protein, 7 aa, Unknown
  Description of Unknown vibi J sequence
YKNGTFP

SEQ ID NO: 1596 - protein, 6 aa, Unknown
  Description of Unknown vibi K sequence
YRNGIP

SEQ ID NO: 1597 - protein, 7 aa, Unknown
  Description of Unknown vico A sequence
YYNGSIP

SEQ ID NO: 1598 - protein, 7 aa, Unknown
  Description of Unknown vico B sequence
YYNGSIP

SEQ ID NO: 1599 - protein, 7 aa, Unknown
  Description of Unknown vigno 1 sequence
VRNGLPL

SEQ ID NO: 1600 - protein, 7 aa, Unknown
  Description of Unknown vigno 10 sequence
YKDGTIP

SEQ ID NO: 1601 - protein, 8 aa, Unknown
  Description of Unknown vigno 2 sequence
VRDGSSPL

SEQ ID NO: 1602 - protein, 7 aa, Unknown
  Description of Unknown vigno 3 sequence
TRNGLPL

SEQ ID NO: 1603 - protein, 7 aa, Unknown
  Description of Unknown vigno 4 sequence
TRNGLPL

SEQ ID NO: 1604 - protein, 7 aa, Unknown
  Description of Unknown vigno 5 sequence
VRNGLPL

SEQ ID NO: 1605 - protein, 6 aa, Unknown
  Description of Unknown vigno 6 sequence
YRNGIP

SEQ ID NO: 1606 - protein, 7 aa, Unknown
  Description of Unknown vigno 7 sequence
YKNGTLP

SEQ ID NO: 1607 - protein, 6 aa, Unknown
  Description of Unknown vigno 8 sequence
YRNGIP

SEQ ID NO: 1608 - protein, 6 aa, Unknown
  Description of Unknown vigno 9 sequence
YRNGIP

SEQ ID NO: 1609 - protein, 5 aa, Unknown
  Description of Unknown violacin A sequence
KSAIS

SEQ ID NO: 1610 - protein, 7 aa, Unknown
  Description of Unknown violapeptide 1 sequence
  MOD_RES (2)..(2): Any amino acid
TXNGLPV

SEQ ID NO: 1611 - protein, 7 aa, Unknown
  Description of Unknown viphi A sequence
YKNGSIP

SEQ ID NO: 1612 - protein, 7 aa, Unknown
  Description of Unknown viphi B sequence
TRNGLPV

SEQ ID NO: 1613 - protein, 6 aa, Unknown
  Description of Unknown viphi C sequence
YINGVP

SEQ ID NO: 1614 - protein, 6 aa, Unknown
  Description of Unknown viphi D sequence
YRNGIP

SEQ ID NO: 1615 - protein, 7 aa, Unknown
  Description of Unknown viphi E sequence
YKNGSIP

SEQ ID NO: 1616 - protein, 7 aa, Unknown
  Description of Unknown viphi F sequence
YKNGSIP

SEQ ID NO: 1617 - protein, 7 aa, Unknown
  Description of Unknown viphi G sequence
YKNGSIP

SEQ ID NO: 1618 - protein, 5 aa, Unknown
  Description of Unknown viphi H sequence
YNGIP

SEQ ID NO: 1619 - protein, 6 aa, Unknown
  Description of Unknown vitri A sequence
YRNGIP

SEQ ID NO: 1620 - protein, 7 aa, Unknown
  Description of Unknown vitri B sequence
TTNGVPI

SEQ ID NO: 1621 - protein, 7 aa, Unknown
  Description of Unknown vitri C sequence
TRNGLPI

SEQ ID NO: 1622 - protein, 7 aa, Unknown
  Description of Unknown vitri D sequence
NRNGLPV

SEQ ID NO: 1623 - protein, 7 aa, Unknown
  Description of Unknown vitri E sequence
FRNGLPV

SEQ ID NO: 1624 - protein, 7 aa, Unknown
  Description of Unknown vitri F sequence
YKDGLTP

SEQ ID NO: 1625 - protein, 7 aa, Unknown
  Description of Unknown vitri peptide 1 sequence
YKNGLIP

SEQ ID NO: 1626 - protein, 7 aa, Unknown
  Description of Unknown vitri peptide 18a sequence
ERNGVPI

SEQ ID NO: 1627 - protein, 8 aa, Unknown
  Description of Unknown vitri peptide 18b sequence
SKDGSVFN

SEQ ID NO: 1628 - protein, 7 aa, Unknown
  Description of Unknown vitri peptide 2 sequence
YLNGSIP

SEQ ID NO: 1629 - protein, 8 aa, Unknown
  Description of Unknown vitri peptide 20 sequence
YRNGDLVP

SEQ ID NO: 1630 - protein, 7 aa, Unknown
  Description of Unknown vitri peptide 22a sequence
NRNGAPV

SEQ ID NO: 1631 - protein, 7 aa, Unknown
  Description of Unknown vitri peptide 23 sequence
TKNGLPT

SEQ ID NO: 1632 - protein, 10 aa, Unknown
  Description of Unknown vitri peptide 24a sequence
YGENGGTIFN

SEQ ID NO: 1633 - protein, 7 aa, Unknown
  Description of Unknown vitri peptide 3 sequence
YKNGSWP

SEQ ID NO: 1634 - protein, 6 aa, Unknown
  Description of Unknown vitri peptide 30 sequence
FRNGFA

SEQ ID NO: 1635 - protein, 10 aa, Unknown
  Description of Unknown vitri peptide 36/37 sequence
YGENGGTIFS

SEQ ID NO: 1636 - protein, 7 aa, Unknown
  Description of Unknown vitri peptide 39 sequence
TRNGAPI

SEQ ID NO: 1637 - protein, 7 aa, Unknown
  Description of Unknown vitri peptide 39 linear sequence
TRNGAPI

SEQ ID NO: 1638 - protein, 6 aa, Unknown
  Description of Unknown vitri peptide 4 sequence
YKDGTP

SEQ ID NO: 1639 - protein, 7 aa, Unknown
  Description of Unknown vitri peptide 50 sequence
YYNGDIP

SEQ ID NO: 1640 - protein, 5 aa, Unknown
  Description of Unknown vitri peptide 8 sequence
YRPTP

SEQ ID NO: 1641 - protein, 7 aa, Unknown
  Description of Unknown vitri peptide 94b sequence
KRNGVAV

SEQ ID NO: 1642 - protein, 9 aa, Unknown
  Description of Unknown vitri peptide 9a/53 sequence
YGQNGTIFD

SEQ ID NO: 1643 - protein, 7 aa, Unknown
  Description of Unknown vocC sequence
IRNGLPV

SEQ ID NO: 1644 - protein, 7 aa, Unknown
  Description of Unknown vodo M sequence
TRNGAPI

SEQ ID NO: 1645 - protein, 7 aa, Unknown
  Description of Unknown vodo N sequence
YRNGLPV

SEQ ID NO: 1646 - protein, 6 aa, Unknown
  Description of Unknown Vpf-1 sequence
YRNGIP

SEQ ID NO: 1647 - protein, 7 aa, Unknown
  Description of Unknown Vpl-1 sequence
YFNGSQS

SEQ ID NO: 1648 - protein, 52 aa, Homo sapiens
YRQSMNNFQGLRSFGCRFGTCTVQKLAHQIYQFTDKDKDNVAPRSKISPQGY

SEQ ID NO: 1649 - protein, 244 aa, Unknown
  Description of Unknown Adiponectin sequence
MLLLGAVLLLLALPGHDQETTTQGPGVLLPLPKGACTGWMAGIPGHPGHNGAPGRDGRDGTPGEKGEKGDPGLIGPKGDIGETGVPGAEGPRGFPGIQGRKGEPGEGAYVYRSAFSVGLETYVTIPNMPIRFTKIFYNQQNHYDGSTGKFHCNIPGLYYFAYHITVYMKDVKVSLFKKDKAMLFTYDQYQENNVDQASGSVLLHLEVGDQVWLQVYGEGERNGLYADNDNDSTFTGFLLYHDTN

SEQ ID NO: 1650 - protein, 39 aa, Unknown
  Description of Unknown Adrenocorticotropic hormone sequence
SYSMEHFRWGKPVGKKRRPVKVYPNGAEDESAEAFPLEF

SEQ ID NO: 1651 - protein, 110 aa, Unknown
  Description of Unknown Agouti signalling peptide sequence
HLPPEEKLRDDRSLRSNSSVNLLDVPSVSIVALNKKSKQIGRKAAEKKRSSKKEASMKKVVRPRTPLSAPCVATRNSCKPPAPACCDPCASCQCRFFRSACSCRVLSLNC

SEQ ID NO: 1652 - protein, 8 aa, Unknown
  Description of Unknown Allatostatin sequence
DRLYSFGL

SEQ ID NO: 1653 - protein, 37 aa, Unknown [sequence omitted]

SEQ ID NO: 1654 - protein, 8 aa, Unknown
  Description of Unknown Angiotensin sequence
DRVYIHPF

SEQ ID NO: 1655 - protein, 198 aa, Unknown
  Description of Unknown ANGPTL8 sequence
MPVPALCLLWALAMVTRPASAAPMGGPELAQHEELTLLFHGTLQLGQALNGVYRTTEGRLTKARNSLGLYGRTIELLGQEVSRGRDAAQELRASLLETQMEEDILQLQAEATAEVLGEVAQAQKVLRDSVQRLEVQLRSAWLGPAYREFEVLKAHADKQSHILWALTGHVQRQRREMVAQQHRLRQIQERLHTAALPA

SEQ ID NO: 1656 - protein, 140 aa, Unknown
  Description of Unknown Asprosin sequence
STNETDASNIEDQSETEANVSLASWDVEKTAIFAFNISHVSNKVRILELLPALTTLTNHNRYLIESGNEDGFFKINQKEGISYLHFTKKKPVAGTYSLQISSTPLYKKKELNQLEDKYDKDYLSGELGDNLKMKIQVLLH

SEQ ID NO: 1657 - protein, 28 aa, Unknown
  Description of Unknown Atrial natriuretic peptide sequence
SLRRSSCFGGRMDRIGAQSGLGCNSFRY

SEQ ID NO: 1658 - protein, 33 aa, Unknown
  Description of Unknown Big gastrin sequence
LGPQGPPHLVADPSKKQGPWLEEEEEAYGWMDF

SEQ ID NO: 1659 - protein, 191 aa, Bos sp.
AFPAMSLSGLFANAVLRAQHLHQLAADTFKEFERTYIPEGQRYSIQNTQVAFCFSETMPAPTGKNEAQQKSDLELLRISLLLIQSWLGPLQFLSRVFTNSLVFGTSDRVYEKLKDLEEGILALMRELEDGTPRRGQILKQTYDKFDTNMRSDDALLKNYGLLSCFRKDLHKTETYLRVMKCRRFGEASCAF

SEQ ID NO: 1660 - protein, 9 aa, Unknown
  Description of Unknown Bradykinin sequence
RPPGFSPFR

SEQ ID NO: 1661 - protein, 119 aa, Unknown
  Description of Unknown Brain-derived neurotrophic factor sequence
HSDPARRGELSVCDSISEWVTAADKKTAVDMSGGTVTVLEKVPVSKGQLKQYFYETKCNPMGYTKEGCRGIDKRHWNSQCRTTQSYVRALTMDSKKRIGWRFIRIDTSCVCTLTIKRGR

SEQ ID NO: 1662 - protein, 119 aa, Unknown [sequence omitted]

SEQ ID NO: 1663 - protein, 32 aa, Unknown
  Description of Unknown Calcitonin sequence
CGNLSTCMLGTYTQDFNKFHTFPQTAIGVGAP

SEQ ID NO: 1664 - protein, 200 aa, Unknown
  Description of Unknown Ciliary neurotrophic factor sequence
MAFTEHSPLTPHRRDLCSRSIWLARKIRSDLTALTESYVKHQGLNKNINLDSADGMPVASTDQWSELTEAERLQENLQAYRTFHVLLARLLEDQQVHFTPTEGDFHQAIHTLLLQVAAFAYQIEELMILLEYKIPRNEADGMPINVGDGGLFEKKLWGLKVLQELSQWTVRSIHDLRFISSHQTGIPARGSHYIANNKKM

SEQ ID NO: 1665 - protein, 41 aa, Unknown
  Description of Unknown Corticotropin-releasing hormone sequence
SQEPPISLDLTFHLLREVLEMTKADQLAQQAHSNRKLLDIA

SEQ ID NO: 1666 - protein, 21 aa, Unknown
  Description of Unknown Endothelin sequence
CSCSSLMDKECVYFCHLDIIW

SEQ ID NO: 1667 - protein, 326 aa, Unknown
  Description of Unknown Erythroferrone sequence
SPEPGAPSRSRARREPPPGNELPRGPGESRAGPAARPPEPTAERAHSVDPRDAWMLFVRQSDKGVNGKKRSRGKAKKLKFGLPGPPGPPGPQGPPGPIIPPEALLKEFQLLLKGAVRQRERAEPEPCTCGPAGPVAASLAPVSATAGEDDDDVVGDVLALLAAPLAPGPRAPRVEAAFLCRLRRDALVERRALHELGVYYLPDAEGAFRRGPGLNLTSGQYRAPVAGFYALAATLHVALGEPPRRGPPRPRDHLRLLICIQSRCQRNASLEAIMGLESSSELFTISVNGVLYLQMGQWTSVFLDNASGCSLTVRSGSHFSAVLLGV

SEQ ID NO: 1668 - protein, 192 aa, Unknown
  Description of Unknown FGF19 sequence
LAFSDAGPHVHYGWGDPIRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 1669 - protein, 16 aa, Unknown
  Description of Unknown Gastrin sequence
GPWLEEEEEAYGWMDF

SEQ ID NO: 1670 - protein, 25 aa, Unknown
  Description of Unknown Ghrelin sequence
FLSPEHQRVQQRKESKKPPAKLQPR

SEQ ID NO: 1671 - protein, 35 aa, Unknown; identical to SEQ ID NO: 361
  Description of Unknown Glucagon-like peptide-1 sequence

SEQ ID NO: 1672 - protein, 30 aa, Unknown
  Description of Unknown Glucagon-like peptide-1 sequence
HAEGTFTSDVSSYLEGQAAKEFIAWLVKGR

SEQ ID NO: 1673 - protein, 9 aa, Unknown
  Description of Unknown Gonadotropin sequence
HWSYGLRPG

SEQ ID NO: 1674 - protein, 92 aa, Unknown
  Description of Unknown Gonadotropin-releasing hormone sequence
MKPIQKLLAGLILLTWCVEGCSSQHWSYGLRPGGKRDAENLIDSFQEIVKEVGQLAETQRFECTTHQPRSPLRDLKGALESLIEEETGQKKI

SEQ ID NO: 1675 - protein, 174 aa, Unknown
  Description of Unknown Granulocyte colony-stimulating factor sequence
TPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 1676 - protein, 192 aa, Unknown
  Description of Unknown Growth hormone sequence
MFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGF

SEQ ID NO: 1677 - protein, 108 aa, Unknown
  Description of Unknown Growth hormone-releasing hormone sequence
MPLWVFFFVILTLSNSSHCSPPPPLTLRMRRYADAIFTNSYRKVLGQLSARKLLQDIMSRQQGESNQERGARARLGRQVDSMWAEQKQMELESILVALLQKHSRNSQG

SEQ ID NO: 1678 - protein, 52 aa, Unknown
  Description of Unknown Hepcidin sequence
QLAELQPQDRAGARASWMPMFQRRRRRDTHFPICIFCCGCCHRSKCGMCCKT

SEQ ID NO: 1679 - protein, 191 aa, Unknown
  Description of Unknown Homo sapiens
VQTVPLSRLFDHAMLQAHRAHQLAIDTYQEFEETYIPKDQKYSFLHDSQTSFCFSDSIPTPSNMEETQQKSNLELLRISLLLIESWLEPVRFLRSMFANNLVYDTSDSDDYHLLKDLEEGIQTLMGRLEDGSRRTGQILKQTYSKFDTNSHNHDALLKNYGLLYCFRKDMDKVETFLRMVQCRSVEGSCGF

SEQ ID NO: 1680 - protein, 42 aa, Unknown
  Description of Unknown Incretin sequence
YAEGTFISDYSIAMDKIHQQDFVNWLLAQKGKKNDWKHNITQ

SEQ ID NO: 1681 - protein, 86 aa, Unknown
  Description of Unknown Insulin sequence
FVNQHLCGSHLVEALYLVCGERGFFYTPKTRREAEDLQVGQVELGGGPGAGSLQPLALEGSLQKRGIVEQCCTSICSLYQLENYCN

SEQ ID NO: 1682 - protein, 21 aa, Unknown
  Description of Unknown Insulin aspart sequence
GIVEQCCTSICSLYQLENYCN

SEQ ID NO: 1683 - protein, 30 aa, Unknown
  Description of Unknown Insulin aspart sequence
FVNQHLCGSHLVEALYLVCGERGFFYTDKT

SEQ ID NO: 1684 - protein, 29 aa, Unknown
  Description of Unknown Insulin degludec sequence
FVNQHLCGSHLVEALYLVCGERGFFYTPK

SEQ ID NO: 1685 - protein, 21 aa, Unknown
  Description of Unknown Insulin degludec sequence
GIVEQCCTSICSLYQLENYCN

SEQ ID NO: 1686 - protein, 21 aa, Unknown
  Description of Unknown Insulin glargine sequence
GIVEQCCTSICSLYQLENYCG

SEQ ID NO: 1687 - protein, 32 aa, Unknown
  Description of Unknown Insulin glargine sequence
FVNQHLCGSHLVEALYLVCGERGFFYTPKTRR

SEQ ID NO: 1688 - protein, 21 aa, Unknown
  Description of Unknown Insulin lispro sequence
GIVEQCCTSICSLYQLENYCN

SEQ ID NO: 1689 - protein, 30 aa, Unknown
  Description of Unknown Insulin lispro sequence
FVNQHLCGSHLVEALYLVCGERGFFYTKPT

SEQ ID NO: 1690 - protein, 26 aa, Unknown
  Description of Unknown Insulin-like growth factor sequence
GPETLCGAELVDALQFVCGDRGFYFN

SEQ ID NO: 1691 - protein, 137 aa, Unknown
  Description of Unknown Insulin-like growth factor 1 sequence
MITPTVKMHTMSSSHLFYLALCLLTFTSSATAGPETLCGAELVDALQFVCGDRGFYFNKPTGYGSSSRRAPQTGIVDECCFRSCDLRRLEMYCAPLKPAKSARSVRAQRHTDMPKTQKEVHLKNASRGSAGNKNYRM

SEQ ID NO: 1692 - protein, 69 aa, Unknown [sequence omitted]

SEQ ID NO: 1693 - protein, 13 aa, Unknown
  Description of Unknown Leptin sequence
NVIQISNDLENLR

SEQ ID NO: 1694 - protein, 120 aa, Unknown
  Description of Unknown Limostatin sequence
MTTTMAAPQQQEVPHALLDIETPNQFNYSPSPLAQPDSLRSKPYFDFLSTLYAHDTAKSNLFRPYSVRQRRDADVQKLSRPRRAIVFRPLFVYKQQEIRKQEIRDRNAQRRHDLNRLQRV

SEQ ID NO: 1695 - protein, 31 aa, Unknown
  Description of Unknown Liraglutide sequence
HAEGTFTSDVSSYLEGQAAKEFIAWLVRGRG

SEQ ID NO: 1696 - protein, 17 aa, Unknown
  Description of Unknown Little gastrin I sequence
QGPWLEEEEEAYGWMDF

SEQ ID NO: 1697 - protein, 9 aa, Unknown
  Description of Unknown Luteinizing hormone sequence
HWSYGLRPG

SEQ ID NO: 1698 - protein, 13 aa, Unknown
  Description of Unknown Melanocyte-stimulating hormone sequence
  MOD_RES (4)..(4): Nle
SYSXEHFRWGKPV

SEQ ID NO: 1699 - protein, 13 aa, Unknown
  Description of Unknown Alpha-Melanocyte-stimulating hormone sequence
SYSMEHFRWGKPV

SEQ ID NO: 1700 - protein, 22 aa, Unknown
  Description of Unknown Beta-Melanocyte-stimulating hormone sequence
AEKKDEGPYRMEHFRWGSPPKD

SEQ ID NO: 1701 - protein, 11 aa, Unknown
  Description of Unknown Gamma-Melanocyte-stimulating hormone sequence
YVMGHFRWDRF

SEQ ID NO: 1702 - protein, 14 aa, Unknown
  Description of Unknown Minigastrin sequence
WLEEEEEAYGWMDF

SEQ ID NO: 1703 - protein, 32 aa, Unknown
  Description of Unknown N-terminal prohormone of brain natriuretic sequence
SPKMVQGSGCFGRKMDRISSSSGLGCKVLRRH

SEQ ID NO: 1704 - protein, 223 aa, Unknown
  Description of Unknown Nerve growth factor sequence
EPHSESNVPAGHTIPQAHWTKLQHSLDTALRRARSAPAAAIAARVAGQTRNITVDPRLFKKRRLRSPRVLFSTQPPREAADTQDLDFEVGGAAPFNRTHRSKRSSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVRRA

SEQ ID NO: 1705 - protein, 196 aa, Unknown
  Description of Unknown Neuropeptide VF precursor sequence
MEIISSKLFILLTLATSSLLTSNIFCADELVMSNLHSKENYDKYSEPRGYPKGERSLNFEELKDWGPKNVIKMSTPAVNKMPHSFANLPLRFGRNVQEERSAGATANLPLRSGRNMEVSLVRRVPNLPQRFGRTTTAKSVCRMLSDLCQGSMHSPCANDLFYSMTCQHQEIQNPDQKQSRRLLFKKIDDAELKQEK

SEQ ID NO: 1706 - protein, 257 aa, Unknown
  Description of Unknown Neurotrophin-3 sequence
MSILFYVIFLAYLRGIQGNNMDQRSLPEDSLNSLIIKLIQADILKNKLSKQMVDVKENYQSTLPKAEAPREPERGGPAKSAFQPVIAMDTELLRQQRRYNSPRVLLSDSTPLEPPPLYLMEDYVGSPVVANRTSRRKRYAEHKSHRGEYSVCDSESLWVTDKSSAIDIRGHQVTVLGEIKTGNSPVKQYFYETRCKEARPVKNGCRGIDDKHWNSQCKTSQTYVRALTSENNKLVGWRWIRIDTSCVCALSRKIGRT

SEQ ID NO: 1707 - protein, 131 aa, Unknown
  Description of Unknown Neurotrophin-4 sequence
MGVSETAPASRRGELAVCDAVSGWVTDRRTAVDLRGREVEVLGEVPAAGGSPLRQYFFETRCKADNAEEGGPGAGGGGCRGVDRRHWVSECKAKQSYVRALTADAQGRVGWRWIRIDTACVCTLLSRTGRA

SEQ ID NO: 1708 - protein, 21 aa, Unknown
  Description of Unknown NPH insulin sequence
GIVEQCCTSICSLYQLENYCN

SEQ ID NO: 1709 - protein, 30 aa, Unknown
  Description of Unknown NPH insulin sequence
FVNQHLCGSHLVEALYLVCGERGFFYTPKT

SEQ ID NO: 1710 - protein, 23 aa, Unknown
  Description of Unknown Obestatin sequence
FNAPFDVGIKLSGVQYQQHSQAL

SEQ ID NO: 1711 - protein, 49 aa, Unknown
  Description of Unknown Osteocalcin sequence
YLYQWLGAPVPYPDPLEPRREVCELNPDCDELADHIGFQEAYRRFYGPV

SEQ ID NO: 1712 - protein, 36 aa, Unknown
  Description of Unknown Parathyroid hormone sequence
KRSVSEIQLMHNLGKHLNSMERVEWLRKKLQDVHNF

SEQ ID NO: 1713 - protein, 36 aa, Unknown
  Description of Unknown Peptide YY sequence
YPIKPEAPGEDASPEELNRYYASLRHYLNLVTRQRY

SEQ ID NO: 1714 - protein, 37 aa, Unknown
  Description of Unknown Pramlintide sequence
KCNTATCATQRLANFLVHSSNNFGPILPPTNVGSNTY

SEQ ID NO: 1715 - protein, 117 aa, Unknown
  Description of Unknown Preprohormone sequence
MPSPGTVCSLLLLGMLWLDLAMAGSSFLSPEHQRVQQRKESKKPPAKLQPRALAGWLRPEDGGQAEGAEDELEVRFNAPFDVGIKLSGVQYQQHSQALGKFLQDILWEEAKEAPADK

SEQ ID NO: 1716 - protein, 199 aa, Unknown
  Description of Unknown Prolactin sequence
LPICPGGAARCQVTLRDLFDRAVVLSHYIHNLSSEMFSEFDKRYTHGRGFITKAINSCHTSSLATPEDKEQAQQMNQKDFLSLIVSILRSWNEPLYHLVTEVRGMQEAPEAILSKAVEIEEQTKRLLEGMELIVSQVHPETKENEIYPVWSGLPSLQMADEESRLSAYYNLLHCLRRDSHKIDNYLKLLKCRIIHNNNC

SEQ ID NO: 1717 - protein, 185 aa, Unknown
  Description of Unknown Relaxin sequence
MPRLFLFHLLEFCLLLNQFSRAVAAKWKDDVIKLCGRELVRAQIAICGMSTWSKRSLSQEDAPQTPRPVAEIVPSFINKDTETIIIMLEFIANLPPELKAALSERQPSLPELQQYVPALKDSNLSFEEFKKLIRNRQSEAADSNPSELKYLGLDTHSQKKRRPYVALFEKCCLIGCTKRSLAKYC

SEQ ID NO: 1718 - protein, 340 aa, Unknown
  Description of Unknown Renin sequence
LTLGNTTSSVILTNYMDTQYYGEIGIGTPPQTFKVVFDTGSSNVWVPSSKCSRLYTACVYHKLFDASDSSSYKHNGTELTLRYSTGTVSGFLSQDIITVGGITVTQMFGEVTEMPALPFMLAEFDGVVGMGFIEQAIGRVTPIFDNIISQGVLKEDVFSFYYNRDSENSQSLGGQIVLGGSDPQHYEGNFHYINLIKTGVWQIQMKGVSVGSSTLLCEDGCLALVDTGASYISGSTSSIEKLMEALGAKKRLFDYVVKCNEGPTLPDISFHLGGKEYTLTSADYVFQESYSSKKLCTLAIHAMDIPPPTGPTWALGATFIRKFYTEFDRRNNRIGFALAR

SEQ ID NO: 1719 - protein, 32 aa, Unknown
  Description of Unknown Salcatonin sequence
CSNLSTCVLGKLSQELHKLQTYPRTNTGSGTP

SEQ ID NO: 1720 - protein, 39 aa, Unknown
  Description of Unknown Sauvagine sequence
GPPISIDLSLELLRKMIEIEKQEKEKQQAANNRLLLDTI

SEQ ID NO: 1721 - protein, 27 aa, Unknown
  Description of Unknown Secretin sequence
HSDGTFTSELSRLREGARLQRLLQGLV

SEQ ID NO: 1722 - protein, 8 aa, Unknown
  Description of Unknown Sincalide sequence
AEEYEYPS

SEQ ID NO: 1723 - protein, 214 aa, Unknown
  Description of Unknown Stanniocalcin sequence
VAAQNSAEVVRCLNSALQVGCGAFACLENSTCDTDGMYDICKSFLYSAAKFDTQGKAFVKESLKCIANGVTSKVFLAIRRCSTFQRMIAEVQEECYSKLNVCSIAKRNPEAITEVVQLPNHFSNRYYNRLVRSLLECDEDTVSTIRDSLMEKIGPNMASLFHILQTDHCAQTHPRADFNRRRTNEPQKLKVLLRNLRGEEDSPSHIKRTSHESA

SEQ ID NO: 1724 - protein, 13 aa, Unknown
  Description of Unknown Temporin sequence
FVQWFSKFLGRIL

SEQ ID NO: 1725 - protein, 13 aa, Unknown
  Description of Unknown Temporin sequence
FLPLIGRVLSGIL

SEQ ID NO: 1726 - protein, 116 aa, Unknown
  Description of Unknown Thyroid-stimulating hormone sequence
MDYYRKYAAIFLVTLSVFLHVLHSAPDVQDCPECTLQENPFFSQPGAPILQCMGCCFSRAYPTPLRSKKTMLVQKNVTSESTCCVAKSYNRVTVMGGFKVENHTACHCSTCYYHKS

SEQ ID NO: 1727 - protein, 40 aa, Unknown
  Description of Unknown Urocortin sequence
DNPSLSIDLTFHLLRTLLELARTQSQRERAEQNRIIFDSV

SEQ ID NO: 1728 - protein, 38 aa, Unknown
  Description of Unknown Urocortin II sequence
IVLSLDVPIGLLQILLEQARARAAREQATTNARILARV

SEQ ID NO: 1729 - protein, 38 aa, Unknown
  Description of Unknown Urocortin III sequence
FTLSLDVPTNIMNLLFNIAKAKNLRAQAAANAHLMAQI

SEQ ID NO: 1730 - protein, 28 aa, Unknown
  Description of Unknown Vasoactive intestinal sequence
HSDAVFTDNYTRLRKQMAVKKYLNSILN

SEQ ID NO: 1731 - protein, 8 aa, Unknown
  Description of Unknown Pleurostatin sequence
VRPYLVAF

SEQ ID NO: 1732 - protein, 7 aa, Unknown
  Description of Unknown Ascalin sequence
YQCGQGG

SEQ ID NO: 1733 - protein, 7 aa, Unknown
  Description of Unknown Microcin C7 sequence
MRTGNAN

SEQ ID NO: 1734 - protein, 7 aa, Unknown
  Description of Unknown Colutellin-A sequence
VISIIPV

SEQ ID NO: 1735 - protein, 7 aa, Unknown
  Description of Unknown E07LKK sequence
LKLLKKL

SEQ ID NO: 1736 - protein, 8 aa, Unknown
  Description of Unknown Bacteriocin sequence
ASILTNAS

SEQ ID NO: 1737 - protein, 7 aa, Unknown
  Description of Unknown Antimicrobial peptide 3 sequence
CSYTMEA

SEQ ID NO: 1738 - protein, 5 aa, Unknown
  Description of Unknown Antimicrobial protein 2 sequence
SPGGA

SEQ ID NO: 1739 - protein, 6 aa, Unknown
  Description of Unknown PAF26 sequence
RKKWFW

SEQ ID NO: 1740 - protein, 6 aa, Unknown
  Description of Unknown Anionic peptide SAAP sequence
DDDDDD

SEQ ID NO: 1741 - protein, 6 aa, Unknown
  Description of Unknown Combi-2 sequence
FRWWHR

SEQ ID NO: 1742 - protein, 6 aa, Unknown
  Description of Unknown Combi-1 sequence
RRWWRF

SEQ ID NO: 1743 - protein, 8 aa, Unknown
  Description of Unknown Jelleine-I sequence
PFKLSLHL

SEQ ID NO: 1744 - protein, 7 aa, Unknown
  Description of Unknown Microcin 7 sequence
MRTGNAD

SEQ ID NO: 1745 - protein, 5 aa, Unknown
  Description of Unknown EP5-1 sequence
ACSAG

SEQ ID NO: 1746 - protein, 5 aa, Unknown
  Description of Unknown EP2 sequence
AMVSS

SEQ ID NO: 1747 - protein, 5 aa, Unknown
  Description of Unknown EP3 sequence
AMVGT

SEQ ID NO: 1748 - protein, 7 aa, Unknown
  Description of Unknown JCpep7 sequence
KVFLGLK

SEQ ID NO: 1749 - protein, 7 aa, Unknown
  Description of Unknown Lantibiotic carnocin-UI49 sequence
GSEIQPR

SEQ ID NO: 1750 - protein, 8 aa, Unknown
  Description of Unknown Oligoventin sequence
QPFSLERW

SEQ ID NO: 1751 - protein, 5 aa, Artificial Sequence
  Description of Artificial Sequence Synthetic peptide
CPFVC

SEQ ID NO: 1752 - protein, 6 aa, Unknown
  Description of Unknown KCM11 sequence
TWWRWW

SEQ ID NO: 1753 - protein, 6 aa, Unknown
  Description of Unknown KCM12 sequence
KWRWIW

SEQ ID NO: 1754 - protein, 6 aa, Unknown
  Description of Unknown KCM21 sequence
KWWWRW

SEQ ID NO: 1755 - protein, 6 aa, Unknown
  Description of Unknown KRS22 sequence
WRWFIH

SEQ ID NO: 1756 - protein, 10 aa, Artificial Sequence
  Description of Artificial Sequence Synthetic peptide
RRRRDDSSDD

SEQ ID NO: 1757 - protein, 8 aa, Unknown
  Description of Unknown Neuropeptide VF precursor sequence
VPNLPQRF